(19) United States
(12) Patent Application Publication
(10) Pub. No.: US 2018/0135134 A1
(43) Pub. Date: May 17, 2018

(54) TARGETED SELECTION OF PATIENTS FOR TREATMENT WITH CORTISTATIN DERIVATIVES

(71) Applicant: President and Fellows of Harvard College, Cambridge, MA (US)

(72) Inventors: Matthew D. Shair, Lexington, MA (US); Henry Efrem Pelish, Newton, MA (US); Ioana IIinca Nitulescu, Cambridge, MA (US)

(73) Assignee: President and Fellows of Harvard College, Cambridge, MA (US)

(21) Appl. No.: 15/807,277

(22) Filed: Nov. 8, 2017

Related U.S. Application Data
(63) Continuation of application No. PCT/US2016/031188, filed on May 6, 2016.
(60) Provisional application No. 62/298,352, filed on Feb. 22, 2016.
(60) Provisional application No. 62/187,656, filed on Jul. 1, 2015.
(60) Provisional application No. 62/158,936, filed on May 8, 2015.

(51) Int. Cl. C12Q 1/6886; A61K 31/4725; A61P 35/00

(52) U.S. Cl. CPC C12Q 1/6886; A61K 31/4725; C12Q 2600/106; C12Q 2600/158; A61P 35/00

(57) ABSTRACT

A method for the targeted selection and treatment of patients with a tumor or cancer, comprising (i) determining whether the patient has a RUNX1 pathway impairment; and if so (ii) administering an effective amount of a cortistatin or its pharmaceutically acceptable salt or oxide, optionally in a pharmaceutically acceptable composition.

### RELATED APPLICATIONS

This application is Continuation of International Application No. PCT/US2016/031188, filed in the United States Patent and Trademark Office, Receiving Office on May 6, 2016, which claims the benefit of provisional U.S. Application No. 62/158,936, filed May 8, 2015, provisional U.S. Application No. 62/187,656, filed Jul. 1, 2015, and provisional U.S. Application No. 62/298,352, filed Feb. 22, 2016. The entirety of these applications are hereby incorporated by reference for all purposes.

### INCORPORATION BY REFERENCE

The contents of the text file named “15020-010US1_sequence_listing_ST25.txt” which was created on Jan. 15, 2018, and is 2.19 KB in size, are hereby incorporated by reference in their entirety.

### BACKGROUND

U.S. Pat. No. 9,127,019 titled “Cortistatin Analogs and Synthesis Thereof” filed by Flyer, et. al., and assigned to the President and Fellows of Harvard College describes analogs of Cortistatins A, J, K, and L having the general Formula I and salts thereof, and the synthesis thereof, wherein R₁, R₂, R₃, R₄, n, and m are as described therein.

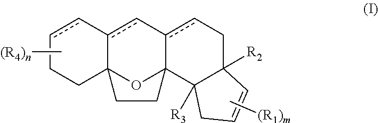

The '019 patent discloses that such compounds are anti-angiogenic and can be used to treat proliferative diseases.

WO 2015/100420 titled “Cortistatin Analogs and Syntheses and Uses Thereof” filed by Shair, et al., and also assigned to the President and Fellows of Harvard College describes further analogs of Cortistatin and methods and compositions that include the described cortistatin analogs to treat proliferative disorders such as cancer, and in particular, a hematopoietic cancer such as leukemia, multiple myeloma (MM), acute myelocytic leukemia (AML), a myeloproliferative neoplasm, acute lymphoblastic leukemia (ALL), chronic myeolcytic leukemia (CML) and primary myelofibrosis (PMF). More generally, the '420 application describes a method to treat a condition associated with CDK8 and/or CDK19 kinase activity, that includes administering an effective amount of a disclosed compound or its pharmaceutically acceptable salt, quaternary amine, or N-oxide. CDK8 and its regulatory subunit cyclin C are components of the RNA polymerase II haloenyme complex, which phosphorylates the carboxy-terminal of the largest subunit of RNA polymerase II. CDK8 regulates transcription by targeting the CDK7/cyclin H subunits of the general transcription factor TFIIH.

Other synthetic and biological descriptions of Cortistatin A and analogs of Cortistatin A have been described in: Chiu et al., Chemistry (2015), 21: 14287-14291, titled “Formal Total Synthesis of (+)-Cortistatins A and J”; Valente et al., Current HIV Research (2015), 13: 64-79, titled “Didehydro-Cortistatin A Inhibits HIV-1 Tat Mediated Neuroinflammation and Prevents Potentiation of Cocaine Reward in Tat Transgenic Mice”; Motomasa et al., Chemical & Pharma. Bulletin (2013), 61: 1024-1029 titled “Synthetic Studies of Cortistatin A Analog from the CD-ring Fragment of Vitamin D2”; Valente et al., Cell Host & Microbe (2012), 12: 97-108 titled “An Analog of the Natural Steroidal Alkaloid Cortistatin A Potently Suppress Tat-dependent HIV Transcription”; Motomasa et al., ACS Med. Chem. Lett. (2012), 3: 673-677 titled “Creation of Readily Accessible and Orally Active Analog of Cortistatin A”; Danishefsky et al., Tetrahedron (2011) 67: 10249-10260 titled “Synthetic Studies Toward (+)-Cortistatin A”; Motomasa et al., Heterocycles (2011), 83: 1535-1552, titled “Synthetic Study of Carbocyclic Core of Cortistatin A, an Anti-angiogenic Steroidal Alkaloid from Marine Sponge”; Motomasa et al., Org. Lett. (2011), 13: 3514-3517, titled “Stereoselective Synthesis of Core Structure of Cortistatin A”; Baran et al., JACS (2011), 133: 8014-8027, titled “Scalable Synthesis of Cortistatin A and Related Structures”; Hirama et al., JOC (2011), 76: 2408-2425, titled “Total Synthesis of Cortistatins A and J”; Zhai et al., Org. Lett. (2010), 22: 5135-5137, titled “Concise Synthesis of the Oxapentacyclic Core of Cortistatin A”; Stoltz et al., Org. Biomol. Chem. (2010), 13: 2915-2917, titled “Efforts Toward Rapid Construction of the Cortistatin A Carbocyclic Core via Enyne-ene Metathesis”; Sarpong et al., Tetrahedron (2010), 66: 4696-4700, titled “Formal Total Synthesis of (±)-Cortistatin A”; Nicolaou et al., Angewandte Chemie (2009), 48: 8952-8957, titled “Cortistatin A is a High-Affinity Ligand of Protein Kinases ROCK, CDK8, and CDK11”.

U.S. Patent Application Publication US2013/0217014 and PCT Application WO2013/122609 titled “Methods of Using CDK8 Antagonists” filed by Firestein, et al., and assigned to Genentech, describes the use of CDK8 antagonists against various cancers. As described therein, as part of the mediator complex CDK8 has a conserved function in transcription as described by Taatjes, D. J., Trends Biochem Sci 35, 315-322 (2010); and Conaway, R. C. and Conaway, J. W., Curr Opin Genet Dev 21, 225-230 (2011). CDK8 has also been reported as an oncogene in both colon cancer (Firestein R. et al., Nature 455:547-51 (2008); Morris E. J. et al., Nature 455:552-6 (2008); Starr T. K. et al., Science 323:1747-50 (2009)) and melanoma (Kapoor A. et al., Nature 468:1105-9 (2010)). CDK8 is upregulated and amplified in a subset of human colon tumors and is known to transform immortalized cells and is required for colon cancer proliferation in vitro. Similarly, CDK8 has also been found to be overexpressed and essential for proliferation in melanoma. Kapoor, A. et al., Nature 468, 1105-1109 (2010). CDK8 has been shown to regulate several signaling pathways that are key regulators of both ES pluripotency and cancer. CDK8 activates the Wnt pathway by promoting expression of 13-Catenin target genes (Firestein, R. et al., Nature 455, 547-551 (2008)) or by inhibiting E2F1, a potent inhibitor of P3-Catenin transcriptional activity. Morris, E. J. et al., Nature 455, 552-556 (2008). CDK8 promotes Notch target gene expression by phosphorylating the Notch intracellular domain, activating Notch enhancer complexes at target genes. Fryer C. J. et al., Mol Cell 16:509-20 (2004).

It is known that tumors and cancer even within a narrow category can be heterogenous. See for example, Meacham, et al., Tumor heterogeneity and cancer cell plasticity, Nature Vol. 501, 328-337 (19 Sep. 2013). Due to the fact that specific tumor types can be caused by a range of genetic abnormalities and as a result can express or suppress key proteins, resulting in a range of phenotypes, not all tumors or cancers within the narrow class will respond to the same drug therapy. Even for the most active oncology drugs, it is expected that there will be responders and non-responders.

Therefore, it would be advantageous to provide a method to determine which tumor and cancer cells will respond best to cortistatin therapy.

It would also be advantageous to be able to achieve the targeted selection of patients who have tumors or cancer that will respond best to cortistatin treatment.

It would further be advantageous to be able to achieve the targeted selection of patients who have tumors or cancer that will respond best to cortistatin treatment, wherein the tumor or cancer has a hematopoietic lineage.

### SUMMARY OF THE INVENTION

In the first embodiment of the invention, it has been discovered that cortistatins are particularly useful to treat tumors and cancers that have an impairment of the Runt-related transcription factor 1 (RUNX1) transcriptional program. Based on this discovery, methods are presented for the targeted selection and treatment of patients more likely to respond to cortistatin therapy, that includes (i) determining whether the patient has a RUNX1 pathway impairment; and if so (ii) administering an effective amount of a cortistatin derivative, including for example, one described herein, or its pharmaceutically acceptable salt and/or composition. The RUNX1 impairment, for example, may be the result of a RUNX1 point mutation, a chromosomal translocation involving the RUNX1 gene, or a mutation resulting in destabilization or increased degradation of the RUNX1 protein.

In one aspect, a method is provided for the treatment of a RUNX1-impaired tumor or cancer by administration of an effective amount of a cortistatin in a manner and dosage that produces a sufficient upregulation of proteins normally transcribed by RUNX1 to cause differentiation or maturation of the tumor or cancer in a manner that renders the cells more normal, less virulent, or in a state of arrested growth or apoptotic.

For example, a method for predicting the response of a patient with a tumor or cancer to treatment with a cortistatin, that includes the steps of obtaining a sample of the tumor or cancer from the patient and determining the expression level or amount of one or more biomarkers in the biological sample from a patient wherein the biomarker(s) is selected from the group consisting of ACSL1, ADORA2B, ADRB1, AMPD3, ARRDC4, BCL2, BCL2A1, CBFβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5 and then determining whether the expression level or amount assessed in is outside of the range of corresponding normal cells, for example, above or below that found in corresponding normal cells or is above or below a certain quantity that is associated with an increased or decreased clinical benefit to a patient; and then optionally treating the patient with an effective amount of the cortistatin, or its pharmaceutically acceptable salt or oxide, optionally in a pharmaceutically acceptable composition thereof. In an alternative embodiment, the method includes comparing the expression of selected genes to the expression of the same genes in a control set of samples comprising a representative number of patients or a predictive animal model that exhibit response to a cortistatin and a representative number of patients that exhibit no or a poor response to a cortistatin to determine if the patient is likely to respond to cortistatin therapy.

A kit for the determination of whether a patient will respond successfully to cortistatin therapy is also provided that can include a probe that anneals with the polynucleotide of a biomarker or combination of biomarkers under stringent conditions or an antibody that binds to a biomarker protein. The kit can include primers for amplifying DNA complementary to RNA encoded specifically by the gene, and optionally a thermostable DNA polymerase. In one embodiment, the primers hybridize under standard stringent conditions to RNA encoded by the selected gene(s) or to the complement thereof.

The selected biomarkers, in one aspect may be one or a combination of GATA1, GATA2, C/EBPα, FLI1, FOG1, ETS1, PU.1, RUNX1 and CBFα. Alternatively, the selected biomarker is one or a combination of BCL2, CCNA1, CD44, C/EBPα, CBFβ, CSF1, CXCL10, CXCR4, ETS1, ETS2, FLI1, FOG1, FCER1A, GATA1, GATA2, GFI1B, HEB, IRF1, IRF8, JAG1, LMO2, LTB, NFE2, NOTCH2, PU.1, SLA, SOCS1, TAL1, and TNF. In a different embodiment, the selected biomarker is one or a combination of constitutive STAT1-pS727, a WT1 mutation, TET2 mutation, IDH1 mutation, IDH2 mutation, MLL-rearrangement, C/EBPα mutation, CBFP3 rearrangement, PU.1 mutation, GATA 1 or 2 mutation, ERG translocation, TLX1 overexpression and TLX3 activation.

A method is also provided for the targeted selection and treatment of patients likely to respond to cortistatin therapy, that includes (i) determining whether the patient has one or a combination of biomarkers selected from ER-positive, loss of function of VHL mutation (VHL-negative), HER2 overexpression, EGFR mutation, MET mutation, a biomarker for neuroblastoma; EWS-FLI1, STAT1-pS727, STAT1, or an inactivating mutation in ETV1, FLI1 SMC3, SMC1A, RAD21, or STAG2 and if so (ii) administering an effect amount of a cortistatin derivative, including for example, one described herein, or its pharmaceutically acceptable salt, oxide and/or composition.

In another aspect, at least two, three, four, five or more of any of the biomarkers described herein are used in the method of targeted selection for the treatment of a tumor or cancer with an effective amount of a cortistatin, or its salt, n-oxide and/or a pharmaceutically acceptable composition thereof.

Nonlimiting hematopoietic lineage tumors or cancers that can be treated, for example, may be selected from Acute lymphoblastic leukemia (ALL), Acute myeloid leukemia (AML), Chronic lymphoblastic leukemia (CLL), B-cell acute lymphoblastic leukemia (B-ALL), childhood B-ALL, Chronic myeloid leukemia, Acute monocytic leukemia, Acute megakaryoblastic leukemia, Hodgkin's lymphoma, Non-Hodgkin's lymphoma, Burkitt's lymphoma, AID S-related lymphoma, Chronic myeloproliferative disorder, Primary central nervous system lymphoma, T-cell lymphoma, Hairy cell leukemia and Multiple myeloma (MM).

The invention includes treating cells that are precursor cells to a hematopoietic tumor or cancer, such as found in myelodysplastic syndrome (MDS).

The tumor or cancer may also be of a non-hematopoeitic lineage, such as breast cancer, ovarian cancer, endometrioid carcinoma, squamous cell cancer, angiosarcoma, colon cancer, gastrointestinal tumors, metastatis-prone solid tumors, clear cell carcinoma, renal cell carcinoma, or esophageal cancer.

Thus, this disclosure provides a method for overcoming inactivating RUNX1 mutations based on the surprising discovery that inhibition of CDK8 and CDK19 with a cortistatin including but not limited to those cortistatins disclosed herein, reverses the effect of the inactivating RUNX1 mutation by causing an upregulation of RUNX1 target genes. Because of this surprising effect, cortistatins can be used to treat malignancies associated with inactivating RUNX1 mutations, for example, by administering the CDK8/19 inhibitor and/or a cortistatin or cortistatin analog thereof to a subject having a cancer associated with an inactivating RUNX1 mutation.

By illustration, it has been discovered that cortistatins potently inhibit proliferation of a number of AML cell lines with 50% maximal growth inhibitory concentrations (GI₅₀s) of less than 10 nM. Cell line sensitivity was consistent with RUNX1 transcriptional program dependence. Sensitive cell lines include those containing fusions that directly inhibit RUNX1 or transcription of its target genes (SKNO-1, ME-1, MOLM-14, MV4;11) as well as megakaryoblastic leukemia cell lines with truncated GATA-1 protein GATA-1s (CMK-86 and MEG-01). Unlike in megakaryopoieis, RUNX1 expression rapidly declines during terminal differentiation of erythrocytes consistent with an insensitivity of erythroleukemia lines to cortistatins.

Cortistatins upregulate RUNX1 target genes including CEBPA, IRF8 and NFE2. By gene set enrichment analysis (GSEA), it was determined that (i) cortistatins upregulate genes in SET-2, MOLM-14 and MV4;11 cell lines that are repressed by expression of RUNX1-RUNX1T1 in hematopoietic stem cells; (ii) cortistatins upregulate genes in MOLM-14 and MV4;11 cells that are reduced in expression in the Kasumi-1 AML cell line upon siRNA-mediated knockdown of RUNX1; and (iii) cortistatins upregulate genes in MOLM-14 cells that increase in expression upon siRNA-mediated knockdown of RUNX1-RUNX1T1 in Kasumi-1 cells. RUNX1 was recruited to loci upregulated by cortistatin treatment.

Some aspects of this disclosure provide methods for diagnosing a cancer sensitive to treatment in a subject with a CDK8/19 inhibitor and/or a cortistatin or cortistatin analog thereof, the method comprising (a) determining whether the subject has a cancer that exhibits impaired RUNX1 activity; and (b) identifying the subject as a subject having a cancer sensitive to treatment with the compound if the subject is determined to harbor a cancer exhibiting impaired RUNX1 activity. In some embodiments, the method further comprises administering a CDK8/19 inhibitor and/or a cortistatin or cortistatin analog thereof to the subject in an amount effective to treat the cancer.

In some embodiments of the diagnostic and therapeutic methods provided herein, the cancer is a hematologic cancer associated with an inactivating RUNX1 mutation. In some embodiments, the cancer is a leukemia, for example, acute myeloid leukemia (AML), myelodysplastic syndrome (MDS), acute lymphoblastic leukemia (ALL), chronic myelogenous leukemia (CML) and chronic myelomonocytic leukemia (CMML). In some embodiments, the acute lymphoblastic leukemia is T-cell acute lymphoblastic leukemia, childhood precursor B-ALL, or B-cell acute lymphoblastic leukemia. In some embodiments, the cancer is breast cancer, ovarian cancer, endometrioid carcinoma, or squamous cell cancer.

Some aspects of this disclosure provide pharmaceutical compositions and kits comprising a cortistatin or a pharmaceutically acceptable salt, quaternary amine salt, or N-oxide thereof, e.g., for use as a medicament in the treatment of a cancer exhibiting impaired RUNX1 activity, wherein the cortistatin is of Formula (A-1), (A-1′), (A-1″), (A-2′), (A-2″), (A-3′), (A-3″), (D1′), (D1″), (D2′), (D2″), (E1′), (E1″), (E2′), (E2″), (G1′), or (G1″), or a pharmaceutically acceptable salt thereof.

This summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 displays the relationship between the mediator complex and various transcriptional regulators. CDK8 and CDK19 associate with Mediator and regulate transcription. RUNX1 binds to enhancer elements, including Super-Enhancers, and acts in concert with transcription factors that include but are not limited to TAL1, C/EBPalpha, CBFbeta, FLI1, ETS1, FOG1, GATA1 and PU.1. Many of these transcription factors have been found to be mutated in certain patients with AML, including RUNX1, C/EBPalpha and GATA1. Treatment with CDK8/19 inhibitor cortistatin A increases expression of RUNX1 target genes and Super-Enhancer-associated genes. Many RUNX1 target genes that increase in expression upon cortistatin A treatment are also Super-Enhancer-associated genes.

FIG. 2 is a gene enrichment analysis of RUNX1 target genes in AML plotted against their interaction with cortistatin A. Cortistatin A upregulates RUNX1 target genes in AML, gene Set Enrichment Analysis (GSEA) mountain plot showing that 3 h 25 nM cortistatin A treatment upregulates genes in MOLM-14 cells that are upregulated in Kasumi-1 cells upon knockdown of RUNX1-RUNX1T1 (also known as AML1-ETO).

FIG. 3 is a bar graph of the percent of cells with megakaryocytic marker CD41 and CD61 in the presence of vehicle, 50 nM cortistatin A or 50 ng/mL PMA. Treatment with CDK8/19 inhibitor cortistatin A induces differentiation of SET-2 cells as measured by an increases in megakaryocytic markers CD41 and CD61. CD41 and CD61 (vehicle vs. CA, p=0.04 and 0.005, respectively, two-tailed t-test) on SET-2 cells after 3 days of indicated treatment (mean±s.e.m., n=3).

FIG. 4 is a graph of the theoretical cell number versus days of cortistatin A treatment. Treatment with CDK8/19 inhibitor cortistatin A inhibits the proliferation of SET-2 cells. The plot of cell number over time for CA (mean±s.e.m., n=3) shows a dose-dependent effect.

FIG. 5 is a synergy plot for the inhibition of proliferation of MPN/AML cell lines SET-2 and UKE-1 where the combination index is plotted against the ratio of the combination of CDK8/19 inhibitor cortistatin A (CA) to JAK1/2 inhibitor ruxolitinib. The plot shows that CDK8/19 inhibition synergizes with JAK1/2 inhibition. Synergy was determined using the method of Chou-Talalay (CalcuSyn).

FIG. 6 is a graph of spleen weight in mice with AML at various doses of cortistatin A. Cortistatin A treatment prevents spleen weight increase in female NOD-SCID-IL2Rcγ^{null} (NSG) mice bearing a disseminated MV4;11-mCLP leukemia that have been treated with cortistatin A once daily by IP administration for 15 days. Dots represent values for individual mice an additional 15 days after stopping cortistatin A treatment and 37 days after tail vein injection of 2 million MV4;11-mCLP cells. Dotted lines mark the range within 1 standard deviation of mean for the related healthy 8-week-old female NOD-SCID mice and were obtained from the Mouse Phenome Database 22903 (The Jackson Laboratory).

FIG. 7A is a plot of kinase activity in terms of percent remaining versus 294 recombinant kinases at a 600 nM cortistatin A. Cortistatin A selectively inhibits CDK8/19 as measured by kinase assay profiling (wildtype-profiler, ProQinase). These kinome-wide profiling studies show that CDK8/19 inhibitor cortistatin A is highly selective for CDK8/19.

FIG. 7B is a plot of native kinase activity in % inhibition at 1,000 nM cortistatin A. Cortistatin A selectively inhibits CDK8/19 as measured by a Native Kinase Profiling assay (KiNativ, ActivX Biosciences). These kinome-wide profiling studies show that CDK8/19 inhibitor cortistatin A is highly selective for CDK8/19.

FIG. 8 is a graph of kinase activity in percent versus concentration of cortistatin A on a logarithmic scale. The graph shows that cortistatin A potently inhibits CDK8/Cyclin C in vitro.

FIG. 9 is a graph of % growth versus cortistatin A concentration (nM, logarithmic scale) for WT and mutated CDK8 and CDK19. The drug resistant alleles confirm AML cell growth requires CDK8/19 kinase activity. This shows that CDK8/19 inhibitor cortistatin A inhibits the proliferation of MOLM-14 cells by inhibiting CDK8/19. Mutation of tryptophan 105 (W105) in CDK8 and CDK19 confers cortistatin A resistance to CDK8 and CDK19. Therefore, MOLM-14 cells are able to proliferate in the presence of cortistatin A upon expression of CDK8 W105M or CDK19 W105M.

FIG. 10 analysis of MV4;11 AML mice on Day 30 shows that treatment with CDK8/19 inhibitor cortistatin A has fewer leukemia cells in the lungs, as measured by haematoxylin and eosin staining.

FIG. 11 is a gene enrichment analysis of genes with increased RUNX1 density plotted against their interaction with cortistatin A. Cortistatin A upregulates genes in SET-2, MOLM-14 and MV4;11 cell lines that are repressed by expression of RUNX1-RUNX1T1 in hematopoietic stem cells (HSCs).

FIG. 12 is a western blot showing that Cas9 can also be used to knock out an endogenous gene BCL2L11. Compared to non-targeting controls, sgRNAs #1 and #5, which targeted only the EL and L isoforms, strongly reduced the gene product Bim. sgRNA #4 targeted all three isoforms, albeit with a lower efficiency. sgRNAs #2 and #3 targeted an intron and did not reduce Bim.

FIG. 13 shows that in cells expressing Cas9 and sgRNA #1 or #3 against ZsGREEN, the green fluorescence was reduced to a level similar to that of the control non-fluorescent cells. Sequencing of the ZsGREEN locus (SEQ. ID NO. 1-9) in cells expressing sgRNA #1 revealed indels at the expected cleavage site.

FIG. 14 is a screening workflow where (A) Cas9 is stably expressed in cell lines of interest using blasticidin selection and then (B) a library is introduced of lentiviral plasmids encoding sgRNAs against approximately 18,000 human genes and on puromycin for 7 days, after which (C) day 0 of the screen commences and cells are treated with vehicle or CA for 14 days. (D) The distribution of each sgRNA in the day 0 reference, day 14 vehicle-treated and day 14 CA-treated populations is determined. sgRNAs that are significantly enriched or depleted in the CA-treated arm are representative biomarkers for CDK8/19 inhibition.

FIG. 15A, FIG. 15B, and FIG. 15C are graphs of growth level measured in % for various cell lines in the presence of 100 nM cortistatin A.

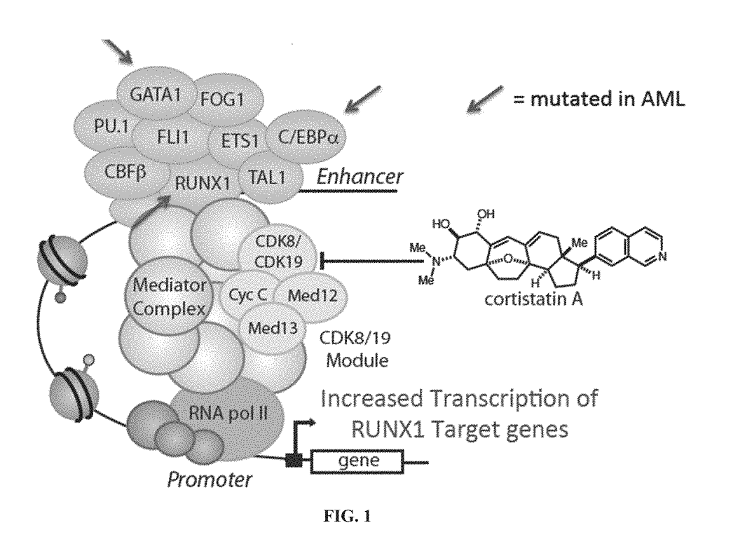

### DETAILED DESCRIPTION OF THE INVENTION

The present invention includes at least the following features:
A) A method for the targeted selection and treatment of patients with a tumor or cancer likely to respond to cortistatin therapy, that includes (i) determining whether the patient has a RUNX1 pathway impairment; and if so (ii) administering an effect amount of a cortistatin derivative, including for example, one described herein, or its pharmaceutically acceptable salt, oxide and/or composition.
B) A method for the treatment of a RUNX1-impaired tumor or cancer by administration of an effective amount of a cortistatin in a manner and dosage that produces a sufficient upregulation of proteins normally transcribed by RUNX1 to cause differentiation of the tumor or cancer in a manner that renders the cells more normal, less virulent, more mature, with arrested growth or apoptosis.
C) The method of A) or B) that includes the use of a kit for the determination of whether a patient will respond successfully to cortistatin therapy that includes a probe that anneals with the polynucleotide of a biomarker or combination of biomarkers under stringent conditions or an antibody that binds to a biomarker protein.
D) A method for predicting the response of a patient with a tumor or cancer to treatment with a cortistatin, that includes the steps of:
   a. Obtaining a sample of the tumor or cancer from the patient;
   b. Determining the expression level or amount of one or more biomarkers in the biological sample from a patient wherein the biomarker(s) is selected from the group consisting of ACSL1, ADORA2B, ADRB1, AMPD3, ARRDC4, BCL2, BCL2A1, CBFβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5;
   c. Determining whether the expression level or amount assessed in b. is outside of the range of corresponding normal cells, for example, above or below that found in corresponding normal cells or is above or below a certain quantity that is associated with an increased or decreased clinical benefit to a patient; and
   d. Optionally treating the patient with an effective amount of the cortistatin, or its pharmaceutically acceptable salt or oxide, optionally in a pharmaceutically acceptable composition thereof.
E) A method for selecting a patient with a tumor or cancer for treatment with a cortistatin that includes:
   a. Obtaining a sample of the patient's tumor or cancer;
   b. Detecting the expression level or amount of one or more biomarkers in the biological sample from the patient wherein the biomarker(s) is selected from the group consisting of ACSL1, ADORA2B, ADRB1, AMPD3, ARRDC4, BCL2, BCL2A1, CBFβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5;
   c. Comparing the expression determined in step b. to the expression of the same genes in a control set of samples comprising a representative number of patients or a predictive animal model that exhibit response to a cortistatin and a representative number of patients that exhibit no or a poor response to a cortistatin to determine if the patient is likely to respond to cortistatin therapy; and
   d. Administering an effective amount of the cortistatin, or its pharmaceutically acceptable salt or oxide, optionally in a pharmaceutically acceptable composition thereof if the patient is determined to be likely to respond to the therapy.
F) The method of A) through E), that includes a kit for assessing the level of expression of the selected gene(s) diagnostic for RUNX1 pathway impairment, primers for amplifying DNA complementary to RNA encoded specifically by the gene, and optionally a thermostable DNA polymerase.
G) The method of F) wherein each of the primers hybridizes under standard stringent conditions to RNA encoded by the selected gene(s) or to the complement thereof.
H) The methods of A) through G), wherein the selected biomarker is one or a combination of GATA1, GATA2, C/EBPα, FLI1, FOG1, ETS1, PU.1, RUNX1, and CBFα.
I) The methods of A) through G), wherein the selected biomarker is one or a combination of BCL2, CCNA1, CD44, C/EBPα, CBFβ, CSF1, CXCL10, CXCR4, ETS1, ETS2, FLI1, FOG1, FCER1A, GATA1, GATA2, GFI1B, HEB, IRF1, IRF8, JAG1, LMO2, LTB, NFE2, NOTCH2, PU.1, SLA, SOCS1, TAL1, and TNF.
J) The methods of A) through G), wherein the selected biomarker is one or a combination of constitutive STAT1-pS727, a WT1 mutation, TET2 mutation, IDH1 mutation, IDH2 mutation, MLL-rearrangement, C/EBPα mutation, CBFP3 rearrangement, PU.1 mutation, GATA 1 or 2 mutation, ERG translocation, TLX1 overexpression and TLX3 activation.
K) The methods of A) through J), comprising using at least two biomarkers independently selected from the list in D), H), I) and J).
L) The methods of A) through J), comprising using at least three biomarkers independently selected from the list in D), H), I) and J).
M) The methods of A) through J), comprising using at least four biomarkers independently selected from the list in D), H), I) and J).
N) The methods of A) through M), wherein the tumor or cancer is of hematopoietic lineage.
O) The methods of N), wherein the hematopoietic lineage tumor or cancer is selected from acute lymphoblastic leukemia (ALL), Acute myeloid leukemia (AML), Chronic lymphoblastic leukemia (CLL), B-cell acute lymphoblastic leukemia (B-ALL), childhood B-ALL, Chronic myeloid leukemia, Acute monocytic leukemia, Acute megakaryoblastic leukemia, Hodgkin's lymphoma, Non-Hodgkin's lymphoma, Burkitt's lymphoma, AIDS-related lymphoma, Chronic myeloproliferative disorder, Primary central nervous system lymphoma, T-cell lymphoma, Hairy cell leukemia and Multiple myeloma (MM), or wherein the cells are precursor cells to a hematopoietic tumor or cancer, such as in myelodysplastic syndrome (MDS).
P) The methods of A) through M), wherein the tumor or cancer is of a non-hematopoeitic lineage.
Q) The method of P), wherein the tumor or cancer is breast cancer, ovarian cancer, endometrioid carcinoma, squamous cell cancer, angiosarcoma, colon cancer, gastrointestinal tumors, metastatis-prone solid tumors, clear cell carcinoma, renal cell carcinoma, or esophageal cancer.
R) The methods of A) through Q), wherein the cortistatin administered to the patient is selected from a compound of Formula (A-1), (A-1′), (A-1″), (A-2′), (A-2″), (A-3′), (A-3″), (D1′), (D1″), (D2′), (D2″), (E1′), (E1″), (E2′), (E2″), (G1′), or (G1″).
S) The methods of A) through Q), wherein the cortistatin administered to the patient is:

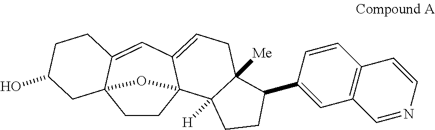

T) The methods of A) through Q), wherein the cortistatin administered to the patient is a natural cortistatin.
U) The methods of A) through Q), wherein the cortistatin administered to the patient is selected from known cortistatin derivatives.
V) The methods of A) through V), wherein the RUNX1 impairment is a result of a RUNX1 point mutation, a chromosomal translocation involving the RUNX1 gene, or a mutation resulting in destabilization or increased degradation of the RUNX1 protein.
W) The methods of A) through V), wherein the RUNX1 transcription factor impairment results in decreased expression of genes under the control of the RUNX1.
X) A method for the targeted selection and treatment of patients likely to respond to cortistatin therapy, that includes (i) determining whether the patient has one or a combination of biomarkers selected from ER-positive, loss of function of VHL mutation (VHL-negative), HER2 overexpression, EGFR mutation, MET mutation, a biomarker for neuroblastoma; EWS-FLI1, STAT1-pS727, STAT1, or an inactivating mutation in ETV1, FLI1, SMC3, SMC1A, RAD21, or STAG2 and if so (ii) administering an effect amount of a cortistatin derivative, including for example, one described herein, or its pharmaceutically acceptable salt, oxide and/or composition.
Y) The method of X), Z) or AA) that includes the use of a kit for the determination of whether a patient will respond successfully to cortistatin therapy that includes a probe that anneals with the polynucleotide of a biomarker or combination of biomarkers under stringent conditions or an antibody that binds to a biomarker protein.
Z) A method for predicting the response of a patient with a tumor or cancer to treatment with a cortistatin, that includes the steps of:
   a. Obtaining a sample of the tumor or cancer from the patient;
   b. Determining the expression level or amount of one or more biomarkers in the biological sample from a patient wherein the biomarker(s) is selected from the group consisting of ER-positive, loss of function of VHL mutation (VHL-negative), HER2 overexpression, EGFR mutation, MET mutation, a biomarker for neuroblastoma; EWS-FLI1, STAT1-pS727, STAT1, or an inactivating mutation in ETV1, FLI1, SMC3, SMC1A, RAD21, or STAG2;
   c. Determining whether the expression level or amount assessed in b. is outside the range in corresponding normal cells, for example, above or below that found in corresponding normal cells or is above or below a certain quantity that is associated with an increased or decreased clinical benefit to a patient; and
   d. Optionally treating the patient with an effective amount of the cortistatin, or its pharmaceutically acceptable salt or oxide, optionally in a pharmaceutically acceptable composition thereof.
AA) A method for selecting a patient who will respond to treatment with a cortistatin that includes:
   a. Obtaining a sample of the patient's tumor or cancer;
   b. Detecting the expression level or amount of one or more biomarkers in the biological sample from the patient wherein the biomarker(s) is selected from the group consisting of ER-positive, loss of function of VHL mutation (VHL-negative), HER2 overexpression, EGFR mutation, MET mutation, a biomarker for neuroblastoma; STAT1-pS727, STAT1, EWS-FLI1, or an inactivating mutation in ETV1, FLI1, SMC3, SMC1A, RAD21, or STAG2;
   c. Comparing the expression determined in step b. to the expression of the same genes in a control set of samples comprising a representative number of patients or a predictive animal model that exhibit response to a cortistatin and a representative number of patients that exhibit no or a poor response to a cortistatin to determine if the patient is likely to respond to cortistatin therapy; and
   d. Administering an effective amount of the cortistatin, or its pharmaceutically acceptable salt or oxide, optionally in a pharmaceutically acceptable composition thereof if the patient is determined to be likely to respond to the therapy.
BB) The methods of X) through AA), that includes a kit diagnostic for the selected genes comprising primers for amplifying DNA complementary to RNA encoded specifically by the gene, and optionally a thermostable DNA polymerase.
CC) The methods of X) through AA), that includes a kit wherein each of the primers hybridizes under standard stringent conditions to RNA encoded by the gene or to the complement thereof.
DD) The methods of X) through AA), wherein the tumor or cancer is of hematopoietic lineage.
EE) The methods of DD), wherein the hematopoietic lineage tumor or cancer is selected from acute lymphoblastic leukemia (ALL), B-cell acute lymphoblastic leukemia (B-ALL), childhood B-ALL, Acute myeloid leukemia (AML), Chronic lymphoblastic leukemia (CLL), B-cell acute lymphoblastic leukemia (B-ALL), childhood B-ALL, Chronic myeloid leukemia, Acute monocytic leukemia, Acute megakaryoblastic leukemia, Hodgkin's lymphoma, Non-Hodgkin's lymphoma, Burkitt's lymphoma, AIDS-related lymphoma, Chronic myeloproliferative disorder, Primary central nervous system lymphoma, T-cell lymphoma, Hairy cell leukemia and Multiple myeloma (MM), or wherein the cells are precursor cells to a hematopoietic tumor or cancer, such as in myelodysplastic syndrome (MDS).
FF) The methods of V) through AA), wherein the tumor or cancer is of a non-hematopoeitic lineage.
GG) The method of FF), wherein the tumor or cancer is breast cancer, ovarian cancer, endometrioid carcinoma, squamous cell cancer angiosarcoma, colon cancer, gastrointestinal tumors, metastatis-prone solid tumors, clear cell carcinoma, renal cell carcinoma, or esophageal cancer.
HH) A method for the targeted selection and treatment of patients with a tumor or cancer likely to respond to anti-CDK8/19 therapy, that includes (i) determining whether the patient has a RUNX1 pathway impairment; and if so (ii) administering an effect amount of a CDK8/19 inhibitor, including for example, one described herein, or its pharmaceutically acceptable salt, oxide and/or composition.
II) A method for the treatment of a RUNX1-impaired tumor or cancer by administration of an effective amount of a CDK8/19 inhibitor in a manner and dosage that produces a sufficient upregulation of proteins transcribed by RUNX1 to cause differentiation of the tumor or cancer in a manner that renders the cells more normal, less virulent, with induced maturation, with arrested cell growth or apoptotic.
JJ) The method of HH), II), KK) or LL) that includes the use of a kit for the determination of whether a patient will respond successfully to anti-CDK8/19 therapy that includes a probe that anneals with the polynucleotide of a biomarker or combination of biomarkers under stringent conditions or an antibody that binds to a biomarker protein.
KK) A method for predicting the response of a patient with a tumor or cancer to treatment with a CDK8/19 inhibitor, that includes the steps of:
   a. Obtaining a sample of the tumor or cancer from the patient;
   b. Determining the expression level or amount of one or more biomarkers in the biological sample from a patient wherein the biomarker(s) is selected from the group consisting of ACSL1, ADORA2B, ADRB1, AMPD3, ARRDC4, BCL2, BCL2A1, CBFβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5;
   c. Determining whether the expression level or amount assessed in b. is outside the range in corresponding normal cells, for example, above or below that found in corresponding normal cells or is above or below a certain quantity that is associated with an increased or decreased clinical benefit to a patient; and
   d. Optionally treating the patient with an effective amount of the CDK8/19 inhibitor, or its pharmaceutically acceptable salt or oxide, optionally in a pharmaceutically acceptable composition thereof.
LL) A method for selecting a patient with a tumor or cancer for treatment with a CDK8/19 inhibitor that includes:
   a. Obtaining a sample of the patient's tumor or cancer;
   b. Detecting the expression level or amount of one or more biomarkers in the biological sample from the patient wherein the biomarker(s) is selected from the group consisting of ACSL1, ADORA2B, ADRB1, AMPD3, ARRDC4, BCL2, BCL2A1, CBFPβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5;
   c. Comparing the expression determined in step b. to the expression of the same genes in a control set of samples comprising a representative number of patients or a predictive animal model that exhibit response to a CDK8/19 inhibitor and a representative number of patients that exhibit no or a poor response to a CDK8/19 inhibitor to determine if the patient is likely to respond to anti-CDK8/19 therapy; and
   d. Administering an effective amount of the CDK8/19 inhibitor, or its pharmaceutically acceptable salt or oxide, optionally in a pharmaceutically acceptable composition thereof if the patient is determined to be likely to respond to the therapy.
MM) The method of HH) through LL), that includes a kit comprising a set of selected genes diagnostic for RUNX1 pathway impairment, primers for amplifying DNA complementary to RNA encoded specifically by the gene, and optionally a thermostable DNA polymerase.
NN) The method of HH) through LL), that includes a kit comprising a set of primers consisting of, for each gene of a selected set of genes diagnostic for RUNX1 pathway impairment, primers for amplifying DNA complementary to RNA encoded specifically by the gene, wherein each of the primers hybridizes under standard stringent conditions to RNA encoded by the gene or to the complement thereof.
OO) The methods of HH) through NN), wherein the selected biomarker is one or a combination of GATA1, GATA2, C/EBPα, FLI1, FOG1, ETS1, PU.1, and CBFα.
PP) The methods of HH) through NN), wherein the selected biomarker is one or a combination of BCL2, CCNA1, CD44, C/EBPα, CBFβ, CSF1, CXCL10, CXCR4, ETS1, ETS2, FLI1, FOG1, FCER1A, GATA1, GATA2, GFI1B, HEB, IRF1, IRF8, JAG1, LMO2, LTB, NFE2, NOTCH2, PU.1, SLA, SOCS1, TAL1, and TNF.
QQ) The methods of HH) through NN), wherein the selected biomarker is one or more of constitutive STAT1-pS727, a WT1 mutation, TET2 mutation, IDH1 mutation, IDH2 mutation, MLL-rearrangement, C/EBPα mutation, CBFP3 rearrangement, PU.1 mutation, GATA 1 or 2 mutation, ERG translocation, TLX1 overexpression and TLX3 activation.
RR) The methods of HH) through NN), comprising using at least two biomarkers independently selected from the list in KK), OO) and PP).
SS) The methods of HH) through QQ), comprising using at least three biomarkers independently selected from the list in KK), OO) and PP).
TT) The methods of HH) through QQ), comprising using at least four biomarkers independently selected from the list in KK), OO) and PP).
UU) The method of HH) through TT) that includes the use of a kit for the determination of whether a patient will respond successfully to CDK8/19 therapy that includes a probe that anneals with the polynucleotide of a biomarker or combination of biomarkers under stringent conditions or an antibody that binds to a biomarker protein.
VV) A method for predicting the response of a patient with a tumor or cancer to treatment with a CDK8/19 inhibitor, that includes the steps of:
   a. Obtaining a sample of the tumor or cancer from the patient;
   b. Determining the expression level or amount of one or more biomarkers in the biological sample from a patient wherein the biomarker(s) is selected from the group consisting of ER-positive, loss of function of VHL mutation (VHL-negative), HER2 overexpression, EGFR mutation, MET mutation, a biomarker for neuroblastoma; EWS-FLI1, STAT1-pS727, STAT1, or an inactivating mutation in ETV1, FLI1, SMC3, SMC1A, RAD21, or STAG2;
   c. Determining whether the expression level or amount assessed in b. is above or below that found in corresponding normal cells, for example, is above or below a certain quantity that is associated with an increased or decreased clinical benefit to a patient; and
   d. Optionally treating the patient with an effective amount of the CDK8/19 inhibitor, or its pharmaceutically acceptable salt or oxide, optionally in a pharmaceutically acceptable composition thereof.
WW) A method for selecting a patient with a tumor or cancer who will respond to treatment with a CDK8/19 inhibitor that includes:
   a. Obtaining a sample of the patient's tumor or cancer;
   b. Detecting the expression level or amount of one or more biomarkers in the biological sample from the patient wherein the biomarker(s) is selected from the group consisting of ER-positive, loss of function of VHL mutation (VHL-negative), HER2 overexpression, EGFR mutation, MET mutation, a biomarker for neuroblastoma; EWS-FLI1, STAT1-pS727, STAT1, or an inactivating mutation in ETV1, FLI1, SMC3, SMC1A, RAD21, or STAG2;
   c. Comparing the expression determined in step b. to the expression of the same genes in a control set of samples comprising a representative number of patients or a predictive animal model that exhibit response to a CDK8/19 inhibitor and a representative number of patients that exhibit no or a poor response to a CDK8/19 inhibitor to determine if the patient is likely to respond to cortistatin therapy; and
   d. Administering an effective amount of the CDK8/19 inhibitor, or its pharmaceutically acceptable salt or oxide, optionally in a pharmaceutically acceptable composition thereof if the patient is determined to be likely to respond to the therapy.
XX) The method of VV) through WW), that includes a kit diagnostic for the selected genes comprising primers for amplifying DNA complementary to RNA encoded specifically by the gene, and optionally a thermostable DNA polymerase.
YY) The method of VV) through WW), that includes a kit comprising a set of primers consisting of, for each gene of the selected set of genes, primers for amplifying DNA complementary to RNA encoded specifically by the gene, wherein each of the primers hybridizes under standard stringent conditions to RNA encoded by the gene or to the complement thereof.
ZZ) The methods of VV) through YY), wherein the tumor or cancer is of hematopoietic lineage.
AAA) The method of ZZ) wherein the hematopoietic lineage tumor or cancer is selected from acute lymphoblastic leukemia (ALL), Acute myeloid leukemia (AML), Chronic lymphoblastic leukemia (CLL), B-cell acute lymphoblastic leukemia (B-ALL), childhood B-ALL, Chronic myeloid leukemia, Acute monocytic leukemia, Acute megakaryoblastic leukemia, Hodgkin's lymphoma, Non-Hodgkin's lymphoma, Burkitt's lymphoma, AIDS-related lymphoma, Chronic myeloproliferative disorder, Primary central nervous system lymphoma, T-cell lymphoma, Hairy cell leukemia and Multiple Myeloma (MM), or wherein the cells are precursor cells to a hematopoietic tumor or cancer, such as in myelodysplastic syndrome (MDS).
BBB) The method of VV) through YY), wherein the tumor or cancer is of non-hematopoeitic lineage.
CCC) The method of BBB), wherein the tumor or cancer is breast cancer, ovarian cancer, endometrioid carcinoma, squamous cell cancer angiosarcoma, colon cancer, gastrointestinal tumors, metastatis-prone solid tumors, clear cell carcinoma, renal cell carcinoma, or esophageal cancer. The methods of A) through CCC), further comprising treating the patient with a second active agent.
DDD) The methods of A) through CCC), further comprising treating the patient with a second active agent, wherein the second active agent is selected from a BET inhibitor, PI3K inhibitor, Raf inhibitor, BTK inhibitor, Bcl-2 inhibitor, CDK7 inhibitor, MEK inhibitor or Syk inhibitor.
EEE) The methods of A) through CCC), further comprising treating the patient with a second active agent, wherein the second active agent is a PD-1 inhibitor selected from nivolumab (BMS), pembrolizumab (Merck), pidilizumab (CureTech/Teva), AMP-244 (Amplimmune/GSK), BMS-936559 (BMS), and MEDI4736 (Roche/Genentech).
FFF) The methods of A) through CCC), further comprising treating the patient with at least one additional active agent, wherein the second active agent is a BET inhibitor selected from JQ1, I-BET 151 (a.k.a. GSK1210151A), I-BET 762 (a.k.a. GSK525762), OTX-015 (a.k.a. MK-8268, IUPAC 6H-Thieno[3,2-f][1,2,4]triazolo[4,3-a][1,4]diazepine-6-acetamide, 4-(4-chlorophenyl)-N-(4-hydroxyphenyl)-2,3,9-trimethyl-), TEN-010, CPI-203, CPI-0610, RVX-208, and LY294002.
GGG) The methods of A) through CCC), further comprising treating the patient with a second active agent, wherein the additional active agent is an immunomodulatory agent.
HHH) The methods of A) through CCC), wherein the additional active agent is an anti-PD1 antibody.
III) The methods of A) through CCC), wherein the additional active agent is an anti-CTLA-4 compound such as ipilimumab (Yervoy) or tremelimumab.
JJJ) A kit as described in any of the embodiments above.
KKK) A combination dosage form of a cortistatin and a least one other active agent, which is used in combination with a diagnostic for patient selection.

The invention is further described in the sections below: Cortistatins (Section I), CDK8/18 Inhibitors (Section II), Selection of Patients Based on sample biomarker analysis (Section III), Diagnostics and Kits (Section IV), Methods and Pharmaceutical Compositions (Section V), Combinations (Section VI), and Examples (Section VI).

### I. CORTISTATINS

The term “cortistatin” or “cortistatin derivative” or “cortistatin analog” as used herein refers to a compound that is an inhibitor of CDK8/19 and has the core general ring structure of one of the known naturally occurring cortistatins (Cortistatins A, B, C, D, E, F, G, H, I, J, K or L) or is described in one of the Formulas below, or is otherwise known in the art as a cortistatin derivative, including in any of the references described in the Background. The cortistatin can be used if desired in the form of a pharmaceutically acceptable salt, including a quarternary ammonium salt, an N-oxide and/or in a pharmaceutically acceptable composition.

A. Cortistatin Analogs

In certain embodiments, the cortistatin or analog thereof is a compound of Formula (A-1) (A-1′), (A-1″), (A-2′), (A-2″), (A-3′), (A-3″), (D1′), (D1″), (D2′), (D2″), (E1′), (E1″), (E2′), (E2″), (G1′), or (G1″):

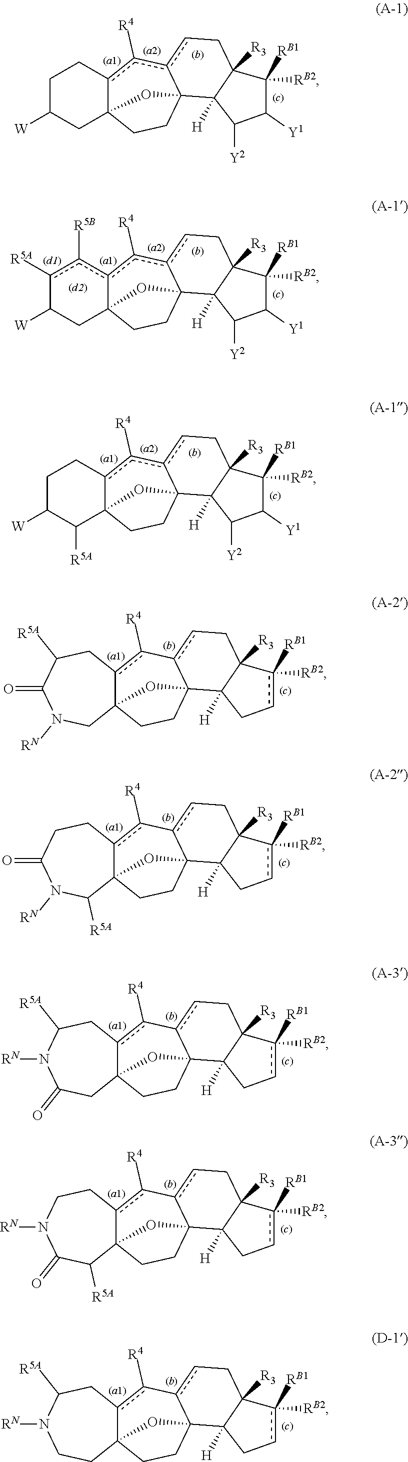

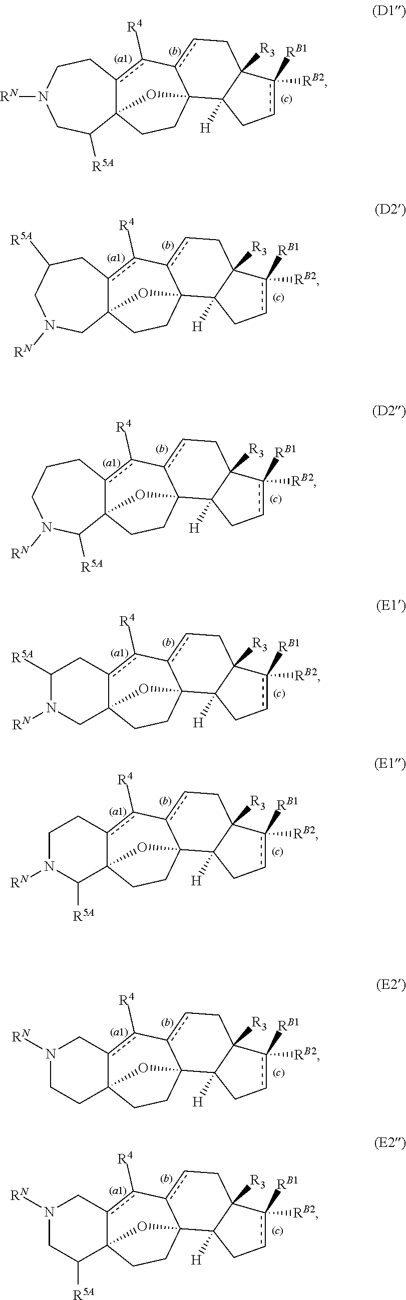

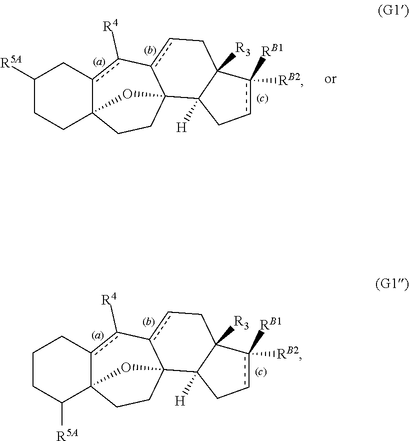

or a pharmaceutically acceptable salt, quaternary amine salt, or N-oxide thereof;
wherein:
W is —N(R¹)(R²), —OR^{O}, ═O, or ═N(R¹);
R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, —SR^{A}, —N(R^{A})₂, —C(═O)R^{A}, —C(═O)OR^{A}, —C(═O)N(R^{A})₂, —S(═O)₂R^{A}, or a nitrogen protecting group;
R² is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —C(═O)R^{A}, —C(═O)OR^{A}, —C(═O)N(R^{A})₂, —S(═O)₂R^{A}, or a nitrogen protecting group;
or R¹ and R² are joined to form an optionally substituted heterocyclyl or optionally substituted heteroaryl;
R^{O} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —C(═O)R^{A}, —C(═O)OR^{A}, —C(═O)N(R^{A})₂, or an oxygen protecting group;
R^{N} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, —C(═O)R^{A}, —C(═O)OR^{A}, —C(═O)N(R^{A})₂, —S(═O)₂R^{A}, or a nitrogen protecting group;
R³ is hydrogen or optionally substituted alkyl;
R⁴ is hydrogen, halogen, optionally substituted alkyl, or —Si(R^{A})₃;
R^{5A} is hydrogen, halogen, optionally substituted alkyl, —OR^{A}, —OC(═O)R^{A}, —OC(═O)OR^{A}, —OC(═O)N(R^{A})₂, —OS(═O)₂R^{A}, —N₃, —N(R^{A})₂, —NR^{A}C(═O)R^{A}, —NR^{A}C(═O)OR^{A}, —NR^{A}C(═O)N(R^{A})₂, —NR^{A}S(═O)₂R^{A}, or —C(R^{A})₃;
R^{5B} is hydrogen, halogen optionally substituted alkyl, or —OR^{A};
each instance of [symbol] , represents a single or double bond, as valency permits, providing:
   a. when [symbol] designated as (b) represents a double bond, then [symbol] designated as (a2) represents a single bond,
   b. when [symbol] designated as (c) represents a double bond, then one of R^{B1} and R^{B2} is absent and one of Y¹ and Y² is absent,
   c. when [symbol] designated as (c) represents a single bond, then both R^{B1} and R^{B2} are present and both of Y¹ and Y² are present,
   d. when [symbol] designated as (a1) represents a double bond, then [symbol] designated as (d2) and (a2) each represent single bonds,
   e. when [symbol] designated as (a2) represents a double bond, then [symbol] designated as (a1) and (b) each represent single bonds,
   f. when [symbol] designated as (d1) represents a double bond, then [symbol] designated as (d2) represents a single bond, and
   g. when [symbol] designated as (d2) represents a double bond, then [symbol] designated as (a1) and (d1) each represent single bonds,
each instance of R^{B1} and R^{B2} is, independently, hydrogen, -L₁-R^{B3}, or —X^{A}RA wherein X^{A} is —O—, —S—, or —N(R^{A})—; or R^{B1} and R^{B2} are joined to form an oxo group, provided that at least one of R^{B1} and R^{B2} is not hydrogen;
L₁ is a bond, —CH(CH₃)(CH₂)₂—, —CH(CH₃)—CH═CH—, —C(═O)—, —C(═O)O—, —C(═O)S—, —C(═O)N(R^{L})—, or —N(R^{L})—(C(R^{LL})₂)ₚ—, wherein R^{L} is hydrogen, optionally substituted alkyl, or a nitrogen protecting group, each instance of R^{LL} is independently hydrogen, halogen, or optionally substituted alkyl, and p is 0, 1, or 2;
R^{B3} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, provided that when L₁ is a bond, then R^{B3} is not hydrogen;
each instance of R^{A} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, carbonyl, silyl, an oxygen protecting group when attached to oxygen, a sulfur protecting group when attached to sulfur, or a nitrogen protecting group when attached to nitrogen; optionally when attached to N the two R^{A} groups may be joined to form an optionally substituted heterocyclyl or optionally substituted heteroaryl ring; and optionally when R^{B1} and R^{B2} are each —X^{A}RA then two R^{A} groups may be joined to form an optionally substituted heterocyclyl ring;
each instance of Y¹ and Y² is hydrogen, or Y¹ is hydrogen and Y² is —OH, or Y¹ and Y² are joined to form an oxo (═O) group;

In one embodiment, the present invention includes compounds of Formulas (A-1), (A-1′), (A-1″), (A-2′), (A-2″), (A-3′), (A-3″), (D1′), (D1″), (D2′), (D2″), (E1′), (E1″), (E2′), (E2″), (G1′), or (G1″), and additional active compounds described herein, and the use of these compounds with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures. Alternatively or in addition, isotopes of carbon, e.g., ¹³C and ¹⁴C, may be used. A typical isotopic substitution is deuterium for hydrogen at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one embodiments deuterium is 90, 95 or 99% enriched at a desired location. Unless otherwise stated, the enrichment at any point is above natural abundance and enough to alter a detectable property of the drug in a human.

In one embodiment, the substitution of a hydrogen atom for a deuterium atom occurs within an R group when at least one of the variables within the R group is hydrogen (e.g., ²H or D) or alkyl (e.g., CHD, CD₂, CD₃). For example, when any of R groups are, or contain for example through substitution, methyl, ethyl, or another alkyl group, the alkyl residue can be deuterated, e.g., CD₃, CH₂CD₃ or CD₂CD₃. In certain other embodiments, when any of the above mentioned R groups are hydrogen, the hydrogen may be isotopically enriched as deuterium (i.e., ²H).

In some embodiments, R^{B1} is deuterium. In some embodiments, R^{B1} comprises an isotopically enriched atom (e.g., ²H, ³H, ¹³C, ¹⁴C, ¹⁸F). In some embodiments, R^{B2} is deuterium. In some embodiments, R^{B2} comprises an isotopically enriched atom (e.g., ²H, ³H, ¹³C, ¹⁴C, ¹⁸F). In some embodiments, Y¹ is deuterium. In some embodiments, Y¹ comprises an isotopically enriched atom (e.g., ²H, ³H, ¹³C, ¹⁴C, ¹⁸F). In some embodiments, Y² is deuterium. In some embodiments, Y² comprises an isotopically enriched atom (e.g., ²H, ³H, ¹³C, ¹⁴C, ¹⁸F). In some embodiments, R³ is deuterium. In some embodiments, R³ comprises an isotopically enriched atom (e.g., ²H, ³H, ¹³C, ¹⁴C, ¹⁸F). In some embodiments, R⁴ is deuterium. In some embodiments, R⁴ comprises an isotopically enriched atom (e.g., ²H, ³H, ¹³C, ¹⁴C, ¹⁸F). In some embodiments, R^{5A} is deuterium. In some embodiments, R^{5A} comprises an isotopically enriched atom (e.g., ²H, ³H, ¹³C, ¹⁴C, ¹⁸F). In some embodiments, R^{5B} is deuterium. In some embodiments, R^{5B} comprises an isotopically enriched atom (e.g., ²H, ³H, ¹³C, ¹⁴C, ¹⁸F). In some embodiments, R^{N} is deuterium. In some embodiments, R^{N} comprises an isotopically enriched atom (e.g., ²H, ³H, ¹³C, ¹⁴C, ¹⁸F). In some embodiments, W comprises an isotopically enriched atom (e.g., ²H, ³H, ¹³C, ¹⁴C, ¹⁸F). In some embodiments, R^{O} is deuterium. In some embodiments, R^{O} comprises an isotopically enriched atom (e.g., ²H, ³H, ¹³C, ¹⁴C, ¹⁸F). In some embodiments, R¹ or R² is deuterium. In some embodiments, R¹ or R² comprises an isotopically enriched atom (e.g., ²H, ³H, ¹³C, ¹⁴C, ¹⁸F). In some embodiments, a hydrogen on ring A (see below) is substituted with deuterium. In some embodiments, a hydrogen on ring B is substituted with deuterium. In some embodiments, a hydrogen on ring C is substituted with deuterium. In some embodiments, a hydrogen on ring D is substituted with deuterium.

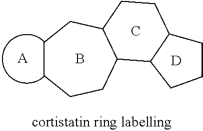

In some embodiments, R⁵ or another position of ring A is deuterated by trapping of an enolate with a deuterium source, such as D₂O or a deuterated acid. In some embodiments, a position of ring B, C, or D is deuterated by reduction of double bond (a), (b), or (c) respectively with a deuterium source (e.g., D₂, HD, a deuterated borohydride). In some embodiments, a position of ring D is deuterated by trapping of an enolate (e.g., for a compound of Formula (XXI)) with a deuterium source, such as D₂O or a deuterated acid.

Quaternary Amine Salts and N-Oxides A “quaternary amine salt” as used herein refers to an amino group wherein the nitrogen atom comprises four valence bonds (e.g., is substituted with four groups which may be hydrogen and/or non-hydrogen groups) such that the nitrogen atom is positively charged and the charge is balanced (neutralized) with a counteranion (e.g., X^{C} as defined herein).

An “N-oxide” as used herein refers to an amino group wherein the nitrogen atom comprises four valence bonds (e.g., is substituted with four groups which may be hydrogen and/or non-hydrogen groups, wherein one group directly attached to the nitrogen atom is an oxidyl group (—O^{⊖})) such that the nitrogen atom is positively charged, and wherein the oxidyl group balances (neutralizes) the positive charge of the nitrogen atom.

It should be understood that any one of Formula (A-1), (A-1′), (A-1″), (A-2′), (A-2″), (A-3′), or (A-3″) may comprise quaternary amine salt and/or N-oxide groups at any position where an amino group may be located.

In particular, compounds of Formula (A-1′) or (A-2″), wherein W is —N(R¹)(R²), may comprise a quaternary amine salt or N-oxide group at the C₃ position (also referred to as a “quaternary C3-amine salt” and “C3-N-oxide”), which comprises the amino group —NR₁R₂ attached to Ring A.

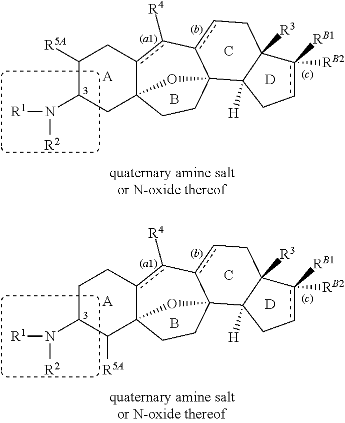

In certain embodiments, the amino group

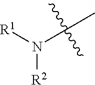

at the C₃ position may be a quaternary amine salt formula

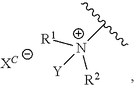

e.g., to provide a compound of Formula (A-QA′) or (A-QA″):

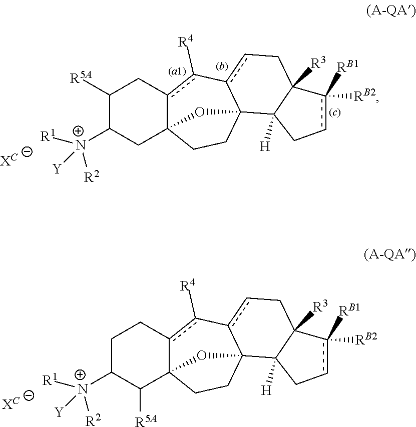

wherein [symbol] , R¹, R², R³, R⁴, R^{5A}, R^{B1}, and R^{B2} are as defined herein; and
wherein:
Y is optionally substituted alkyl, optionally substituted alkenyl, optionally substituted alkynyl, optionally substituted carbocyclyl, optionally substituted heterocyclyl, optionally substituted aryl, or optionally substituted heteroaryl; and
X^{C} is a counteranion.

A quaternary C3-amine salt may be formed by reaction of the free C3-amine with a group Y—X^{C}, wherein Y is defined above (e.g., optionally substituted alkyl, optionally substituted alkenyl, optionally substituted alkynyl, optionally substituted carbocyclyl, or optionally substituted heterocyclyl), and X^{C} is a leaving group as defined herein. The counterion X^{C} resulting therefrom may be exchanged with another counterion X^{C} by ion exchange methods, e.g., ion exchange chromatography. Exemplary X^{C} counterions include but are not limited to halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like). In certain embodiments, Y is optionally substituted alkyl (e.g., methyl). In certain embodiments, X^{C} is a halide ion.

In certain embodiments, the quaternary amine salt of Formula (A-QA′) or (A-QA″) is the beta (A-1-QA′) or (A-1-QA″) or alpha (A-2-QA′) or (A-2-QA″) isomer of the following Formula:

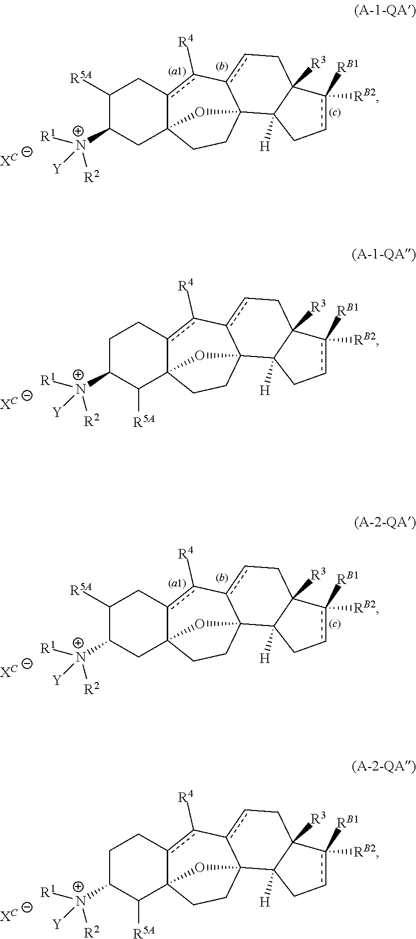

wherein [symbol] , R¹, R², R³, R⁴, R^{5A}, R^{B1}, and R^{B2} are as defined herein.

Alternatively, in certain embodiments, the amino group

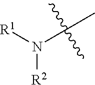

at the C₃ position may be an N-oxide of formula

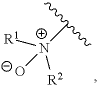

e.g., to provide a compound of Formula (A-NO′) or (A-NO″):

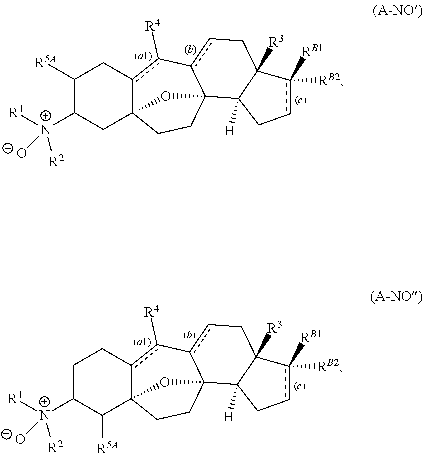

wherein [symbol] , R¹, R², R³, R⁴, R^{5A}, R^{B1}, and R^{B2} are as defined herein.

In certain embodiments, the N-oxide of Formula (A-NO′) or (A-NO″) is the beta (A-1-NO′) or (A-1-NO″) or alpha (A-2-NO′) or (A-2-NO″) isomer of the following Formula:

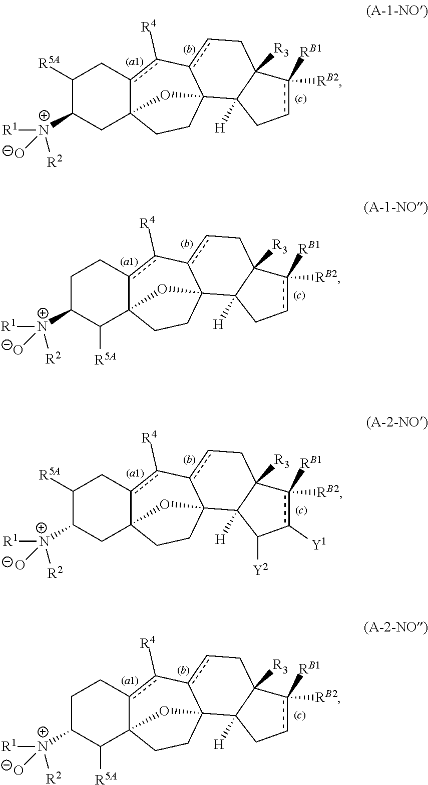

wherein [symbol] , R¹, R², R³, R⁴, R^{5A}, R^{B1}, and R^{B2} are as defined herein.

### Compounds of Formula (A-1) or (A-1″)

As generally defined herein, in certain embodiments, a cortistatin or cortistatin analog thereof is a compound of Formula (A-1′) or (A-1″):

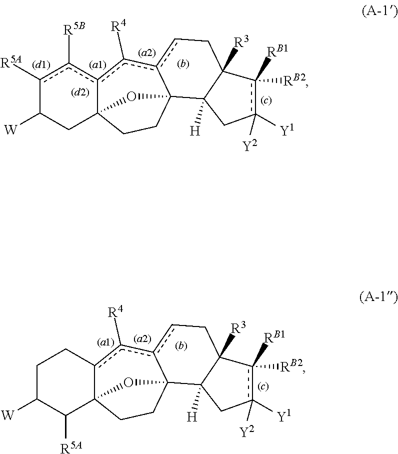

or a pharmaceutically acceptable salt, quaternary amine salt, or N-oxide thereof; wherein W is —N(R¹)(R²), —OR^{O}, ═O, or ═N(R¹).

In certain embodiments, W is —N(R¹)(R²) to provide a compound of Formula

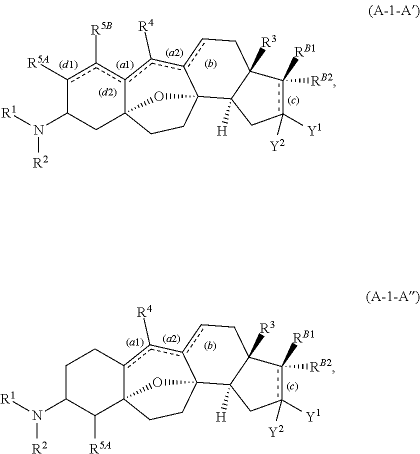

or a pharmaceutically acceptable salt, quaternary amine salt, or N-oxide thereof.

In certain embodiments, the compound of Formula (A-1-A′) or (A-1-A″) is of Formula:

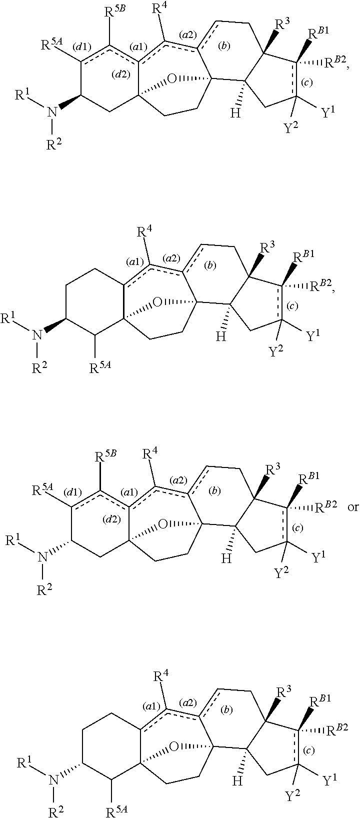

or a pharmaceutically acceptable salt, quaternary amine salt, or N-oxide thereof. Compounds of Formula (A-1-A′) or (A-1-A″) encompasses cortistatins (i.e., naturally occurring cortistatins) such as cortistatin A, B, C, D, E, F, G, H, J, K, and L wherein R^{5A} and R^{5B} are each independently —OR^{A} or wherein at least one instance of [symbol] designated as (d1) or (d2) represents a double bond.

For example, in certain embodiments, wherein R^{5A} and R^{5B} are each independently —OR^{A}, the cortistatin of Formula (A-1-A′) or (A-1-A″) is selected from the group consisting of:

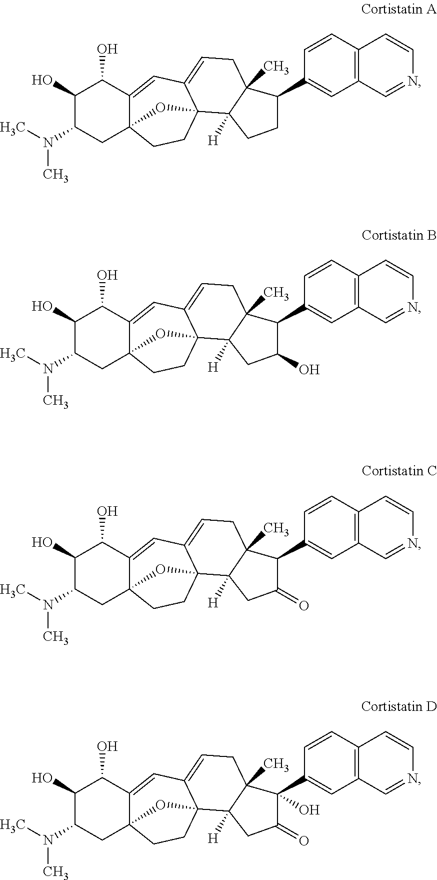 [SMILES: CN(C)[C@H]1C[C@@]23CC[C@@]4(O2)C(=CC[C@]2(C)[C@@H](c5ccc6ccncc6c5)CC[C@H]24)C=C3[C@@H](O)[C@@H]1O ; CN(C)[C@H]1C[C@@]23CC[C@@]4(O2)C(=CC[C@@]2(C)[C@H]4C[C@H](O)[C@@H]2c2ccc4ccncc4c2)C=C3[C@@H](O)[C@@H]1O ; CN(C)[C@H]1C[C@@]23CC[C@@]4(O2)C(=CC[C@]2(C)[C@@H](c5ccc6ccncc6c5)C(=O)C[C@H]24)C=C3[C@@H](O)[C@@H]1O ; CN(C)[C@H]1C[C@@]23CC[C@@]4(O2)C(=CC[C@@]2(C)[C@H]4CC(=O)[C@]2(O)c2ccc4ccncc4c2)C=C3[C@@H](O)[C@@H]1O]

and pharmaceutically acceptable salts, quaternary amine salts, and N-oxides thereof.

In certain embodiments, wherein at least one instance of [symbol] designated as (d1) or (d2) represents a double bond, the cortistatin of Formula (A-1-A′) or (A-1-A″) is selected from the group consisting of:

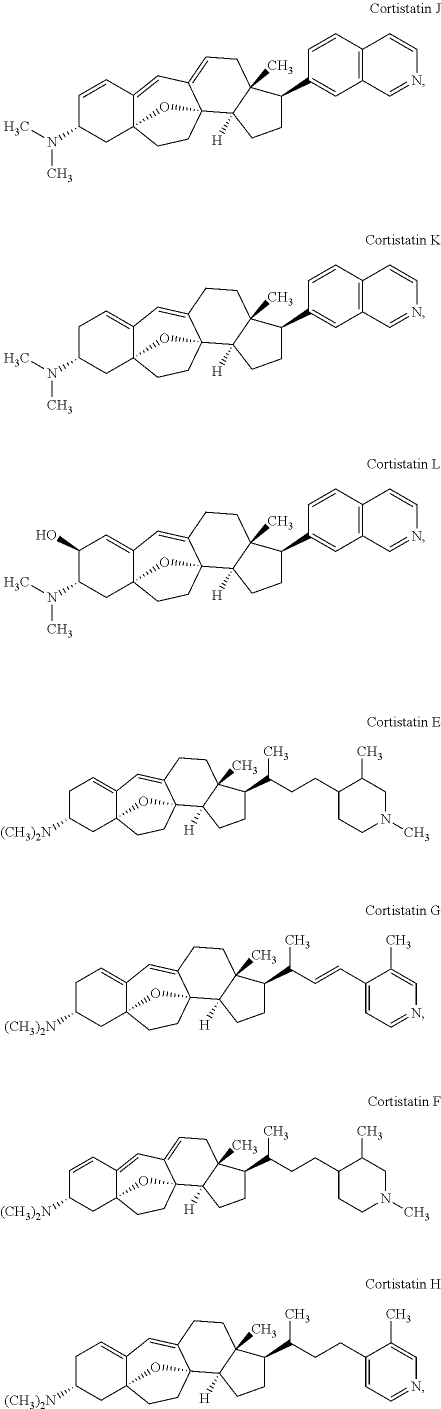

and pharmaceutically acceptable salts, quaternary amine salts, and N-oxides thereof.

In certain embodiments, wherein R^{5A} and R^{5B} are each independently —OR^{A}, or at least one instance of [symbol] designated as (d1) or (d2) represents a double bond, the cortistatin analog of Formula (A-1-A′) or (A-1-A″) is selected from the group consisting of:

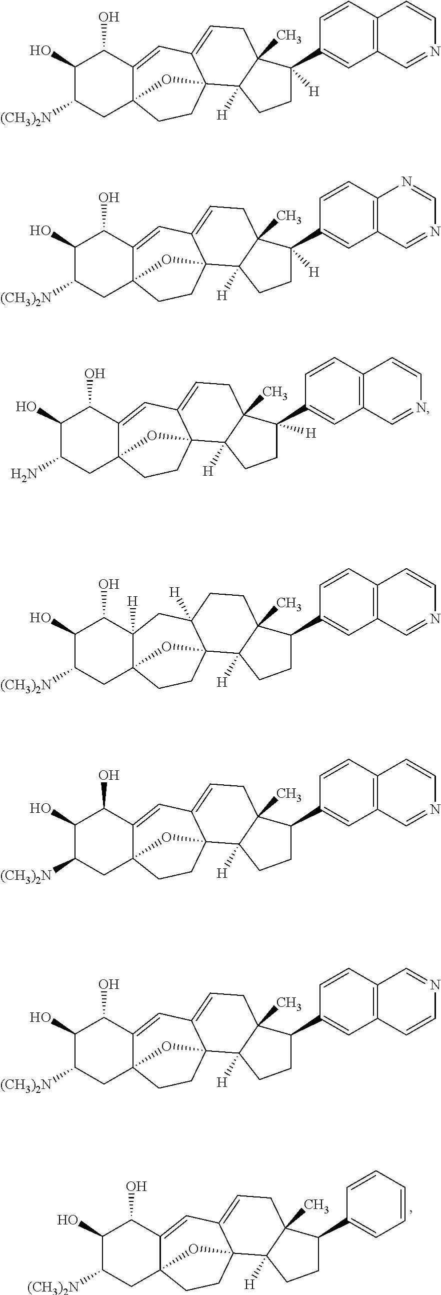

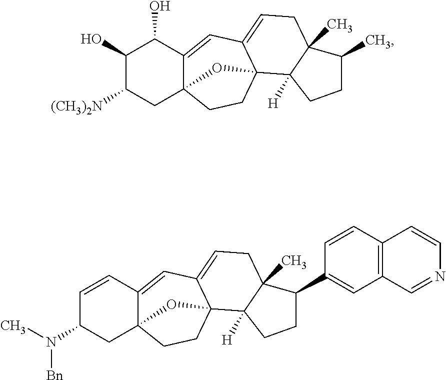

and pharmaceutically acceptable salts, quaternary amine salts, and N-oxides thereof.

The synthesis of natural cortistatins and various cortistatin analogs of Formula (A-1-A′) or (A-1-A″), as depicted above, wherein R^{5A} and R^{5B} are each independently —OR^{A}, or wherein at least one instance of [symbol] designated as (d1) or (d2) represents a double bond, is described in WO/2010/024930, incorporated herein by reference.

Installation of R^{5A} at either carbon alpha to the cyclic ketone may be accomplished during the synthesis of a natural cortistatin or cortistatin analog is installed via an enolate trapping reaction of the ketone. The ketone may be trapped as the enolate, followed by subsequent oxidation or amination of the double bond, or reaction of the double bond with an electrophilic carbon C(R^{A})₃-LG, wherein LG is a leaving group, to provide a substituted ketone product, wherein R⁵ is —OR^{A}, —OC(═O)R^{A}, —OC(═O)OR^{A}, —OC(═O)N(R^{A})₂, —OS(═O)₂R^{A}, —N₃, —N(R^{A})₂, —NR^{A}C(═O)R^{A}, —NR^{A}C(═O)OR^{A}, —NR^{A}C(═O)N(R^{A})₂, —NR^{A}S(═O)₂R^{A}, or —C(R^{A})₃. Exemplary conditions contemplated for enolate trapping include a combination of a base (e.g., lithium diisopropyl amide (LDA)) and a trapping reagent P₁-LG, wherein P₁ is silyl and LG is a leaving group (e.g., such as trimethylsilyl chloride).

Exemplary oxidative conditions, e.g., to install a —OR^{A}, —OC(═O)R^{A}, —OC(═O)OR^{A}, —OC(═O)N(R^{A})₂, or —OS(═O)₂RA group at the R⁵ position include treating the trapped enolate with an oxidant, such as meta-chloroperoxybenzoic acid (MCPBA), MoOOPh, or DMSO, to provide substituted ketone wherein R⁵ is —OH, followed by optional protection, e.g., via treatment of the compound wherein R⁵ is —OH with a compound of formula R^{A}-LG, LG-C(═O)R^{A}, LG-C(═O)OR^{A}, LG-C(═O)N(R^{A})₂, or LG-S(═O)₂R^{A}, wherein LG is a leaving group, to provide a compound wherein R⁵ is —OR^{A} (wherein R^{A} is a non-hydrogen group), —OC(═O)R^{A}, —OC(═O)OR^{A}, —OC(═O)N(R^{A})₂, or —OS(═O)₂R^{A}.

Exemplary aminating conditions, e.g., to install an —N₃, —N(R^{A})₂, —NR^{A}C(═O)R^{A}, —NR^{A}C(═O)OR^{A}, —NR^{A}C(═O)N(R^{A})₂, or —NR^{A}S(═O)₂R^{A} group at the R⁵ position include treating the trapped enolate with a compound N₃-LG wherein LG is a leaving group (e.g., such as trisylazide) to provide substituted ketone wherein R⁵ is —N₃. The substituted ketone wherein R⁵ is —N₃ may be treated with a reducing agent (e.g., such as PPh₃) to provide a compound wherein R⁵ is —NH₂, followed by optional protection, e.g., via treatment of the compound wherein R⁵ is —NH₂ with a compound of formula R^{A}-LG, LG-C(═O)R^{A}, LG-C(═O)OR^{A}, LG-C(═O)N(R^{A})₂, or LG-S(═O)₂R^{A}, wherein LG is a leaving group, to provide a compound wherein R⁵ is —N(R^{A})₂ (wherein at least one of R^{A} is a non-hydrogen group), —NR^{A}C(═O)R^{A}, —NR^{A}C(═O)OR^{A}, —NR^{A}C(═O)N(R^{A})₂, or —NR^{A}S(═O)₂R^{A}.

In certain embodiments, each instance of [symbol] (d1) and (d2) represents a single bond. In certain embodiments, R^{5B} is hydrogen and each instance of [symbol] (d1) and (d2) represents a single bond. The synthesis of cortistatin analogs, wherein R^{5B} is hydrogen and each instance of [symbol] (d1) and (d2) represents a single bond, is described in PCT/US2014/072365, incorporated herein by reference.

In certain embodiments, wherein W is —N(R¹)(R²), R^{5B} is hydrogen and each instance of [symbol] (d1) and (d2) represents a single bond, provided is a compound of Formula:

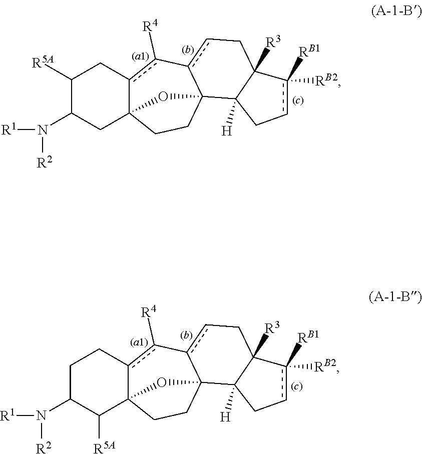

or a pharmaceutically acceptable salt, quaternary amine salt, or N-oxide thereof.

In certain embodiments, the compound of Formula (A-1-B′) or (A-1-B″) is of Formula:

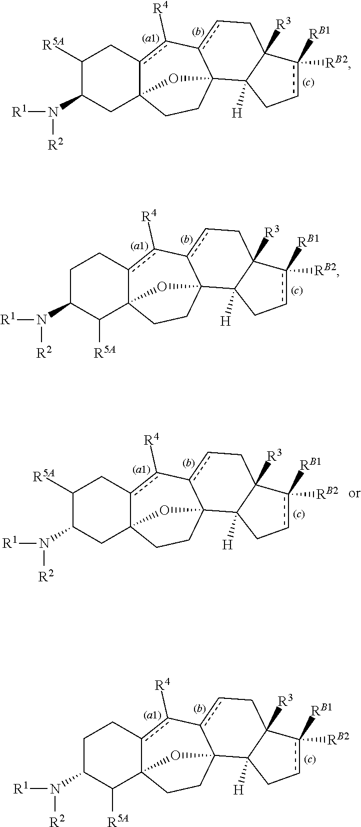

or a pharmaceutically acceptable salt, quaternary amine salt, or N-oxide thereof.

In certain embodiments, wherein W is ═O, R^{5B} is hydrogen and each instance of [symbol] (d1) and (d2) represents a single bond, provided is a compound of formula:

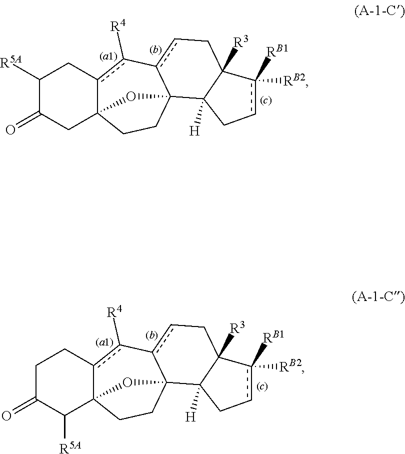

or a pharmaceutically acceptable salt, quaternary amine salt, or N-oxide thereof.

In certain embodiments, wherein W is —OR^{O}, R^{5B} is hydrogen and each instance of [symbol] (d1) and (d2) represents a single bond, provided is a compound of Formula:

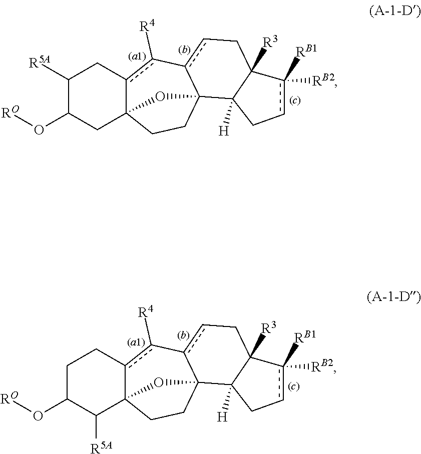

or a pharmaceutically acceptable salt, quaternary amine salt, or N-oxide thereof.

In certain embodiments, the compound of Formula (A-1-D′) or (A-1-D″) is of Formula:

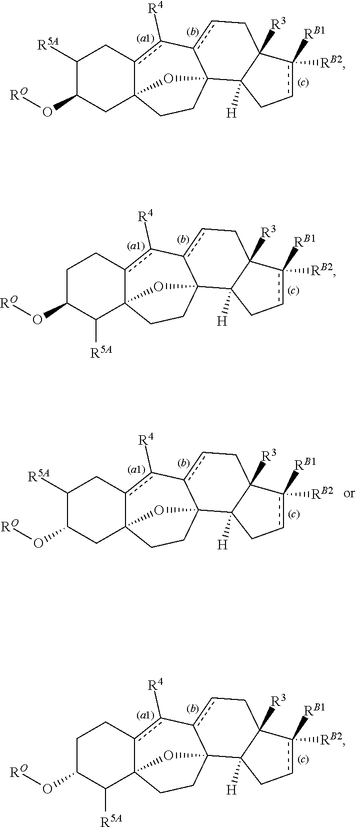

or a pharmaceutically acceptable salt, quaternary amine salt, or N-oxide thereof.

In certain embodiments, wherein W is ═N(R¹), R^{5B} is hydrogen and each instance of [symbol] (d1) and (d2) represents a single bond, provided is a compound of formula:

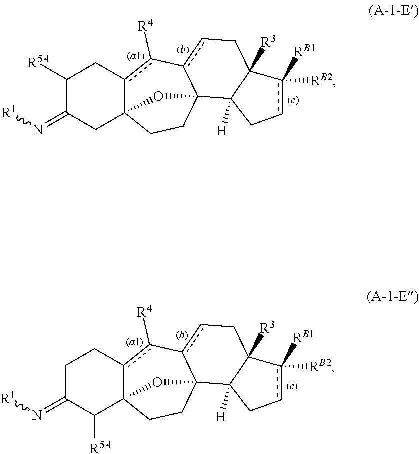

or a pharmaceutically acceptable salt, quaternary amine salt, or N-oxide thereof.

In certain embodiments, the compound of Formula (A-1-E′) or (A-1-E″) instance of formula:

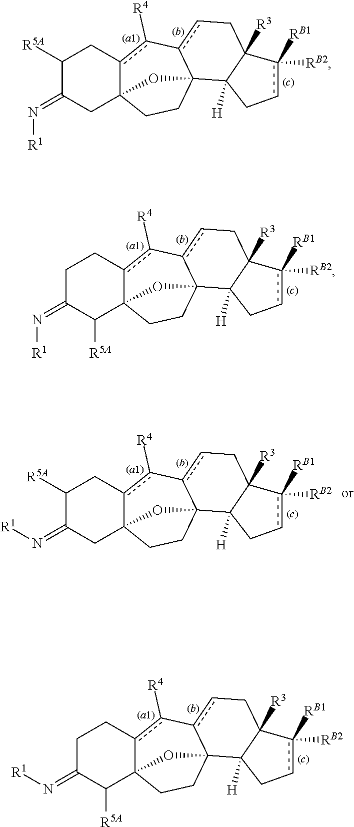

or a pharmaceutically acceptable salt, quaternary amine salt, or N-oxide thereof.
Groups R¹ and R²

As generally defined herein, in certain embodiments of Formula (A-1-A′), (A-1-B′), (A-1-E′), (A-1-A″), (A-1-B″), or (A-1-E″), R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, —SR^{A}, —N(R^{A})₂, —C(═O)R^{A}, —C(═O)OR^{A}, —C(═O)N(R^{A})₂, —S(═O)₂R^{A}, or a nitrogen protecting group.

Furthermore, in certain embodiments of Formula (A-1-A′), (A-1-A″), (A-1-B′), and (A-1-B″), R² is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —C(═O)R^{A}, —C(═O)OR^{A}, —C(═O)N(R^{A})₂, —S(═O)₂R^{A}, or a nitrogen protecting group.

In certain embodiments, at least one of R¹ and R² is hydrogen. In certain embodiments, both of R¹ and R² is hydrogen. In certain embodiments, one of R¹ and R² is hydrogen and the other is a non-hydrogen group, e.g., optionally substituted alkyl. In certain embodiments, R¹ is hydrogen.

In certain embodiments, at least one of R¹ and R² is optionally substituted alkyl, e.g., optionally substituted C₁₋₆alkyl. In certain embodiments, each instance of R¹ and R² is independently optionally substituted alkyl. In certain embodiments, R¹ is optionally substituted alkyl, e.g., optionally substituted C₁₋₆alkyl. In certain embodiments, R¹ and/or R² is optionally substituted C₁alkyl, optionally substituted C₂alkyl, optionally substituted C₃alkyl, optionally substituted C₄alkyl, optionally substituted C₅alkyl, or optionally substituted C₆alkyl. In certain embodiments, R¹ and/or R² is optionally substituted methyl (C₁), optionally substituted ethyl (C₂), optionally substituted n-propyl (C₃), optionally substituted isopropyl (C₃), optionally substituted n-butyl (C₄), or optionally substituted t-butyl (C₄). In certain embodiments, R¹ and/or R² is alkyl substituted with one or more halogen substituents (e.g., fluoro). In certain embodiments, R¹ and/or R² is —CH₃ or —CF₃. In certain embodiments, each instance of R¹ and R² is independently —CH₃ or —CF₃. In certain embodiments, R¹ and/or R² is alkyl substituted with one or more halogen (e.g., fluoro), amino (—NH₂), substituted amino, hydroxyl (—OH), substituted hydroxyl, thiol (—SH), substituted thiol, or sulfonyl substituents. In certain embodiments, R¹ and/or R² is alkyl substituted with an optionally substituted carbocyclyl (e.g., cyclopropyl) or optionally substituted heterocyclyl (e.g., oxetanyl) ring.

For example, in certain embodiments, at least one of R¹ and R² is a group of formula:

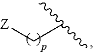

e.g., to provide a compound of formula:

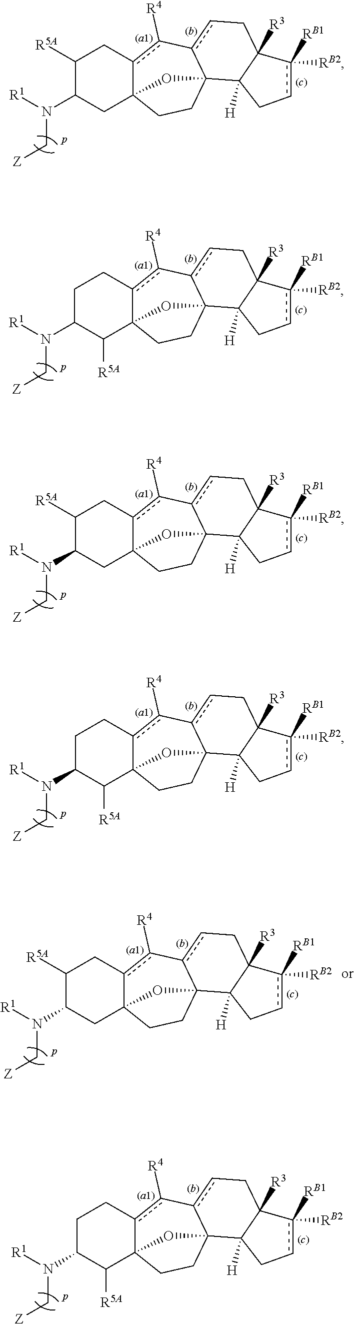

or a pharmaceutically acceptable salt, quaternary amine salt, or N-oxide thereof,
wherein [symbol] , R¹, R³, R⁴, R^{5A}, R^{B1}, and R^{B2} are as defined herein; and
wherein:
p is 1, 2, 3, 4, 5, or 6; and
Z is —CH₂X^{Z}, —CH(X^{Z})₂, —C(X^{Z})₃, —OR^{Z}, —SR^{Z}, —N(R^{Z})₂, —S(O)₂N(R^{Z})₂,

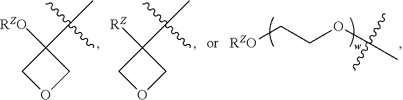

wherein each instance of R^{Z} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —C(═O)R^{Z}, —C(═O)OR^{Z}, —C(═O)N(R^{Z})₂, an oxygen protecting group when attached to oxygen, a sulfur protecting group when attached to sulfur, or a nitrogen protecting group when attached to nitrogen, optionally when attached to N the two R^{Z} groups may be joined to form an optionally substituted heterocyclyl or optionally substituted heteroaryl ring;
each instance of X^{Z} is independently fluoro, chloro, bromo, or iodo; and
w is an integer between 1 and 10, inclusive.

In certain embodiments, both instances of R¹ and R² are independently a group of formula

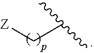

In certain embodiments, p is 1. In certain embodiments, p is 2. In certain embodiments, p is 3. In certain embodiments, w is 1, 2, 3, or 4. In certain embodiments, R^{Z} is hydrogen or optionally substituted alkyl (e.g., —CH₃). In certain embodiments, Z is —OR^{Z}, e.g., —OH or —OR^{Z} wherein R^{Z} is a non-hydrogen group, e.g., wherein R^{Z} is optionally substituted alkyl such as —CH₃. In certain embodiments, Z is —N(R^{Z})₂, e.g., —NH₂, —NHR^{Z}, or —N(R^{Z})₂ wherein R^{Z} is a non-hydrogen group, e.g., wherein R^{Z} is optionally substituted alkyl such as —CH₃. In certain embodiments, Z is —CH₂X^{Z}, —CH(X^{Z})₂, —C(X^{Z})₃, e.g., wherein X^{Z} is fluoro. In certain embodiments, Z is —S(O)₂N(R^{Z})₂, e.g., —S(O)₂NH₂ or —S(O)₂NHCH₃.

In certain embodiments, R¹ and R² are joined to form an optionally substituted heterocyclyl, e.g., an optionally substituted 3-6 membered heterocyclyl. In certain embodiments, R¹ and R² are joined to form an optionally substituted 3-membered heterocyclyl, an optionally substituted 4-membered heterocyclyl, optionally substituted 5-membered heterocyclyl, or an optionally substituted 6-membered heterocyclyl. In certain embodiments, R¹ and R² are joined to form an optionally substituted 3-membered heterocyclyl, i.e., an optionally substituted aziridinyl. In certain embodiments, R¹ and R² are joined to form an optionally substituted 4-membered heterocyclyl, e.g., an optionally substituted azetidinyl. In certain embodiments, R¹ and R² are joined to form an optionally substituted 5-membered heterocyclyl, e.g., an optionally substituted pyrrolidinyl or optionally substituted imidazolidine-2,4-dione. In certain embodiments, R¹ and R² are joined to form an optionally substituted 6-membered heterocyclyl, e.g., an optionally substituted piperidinyl, optionally substituted tetrahydropyranyl, optionally substituted dihydropyridinyl, optionally substituted thianyl, optionally substituted piperazinyl, optionally substituted morpholinyl, optionally substituted dithianyl, optionally substituted dioxanyl, or optionally substituted triazinanyl.

For example, in certain embodiments, R¹ and R² are joined to form a group of formula:

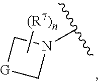

e.g., to provide a compound of formula:

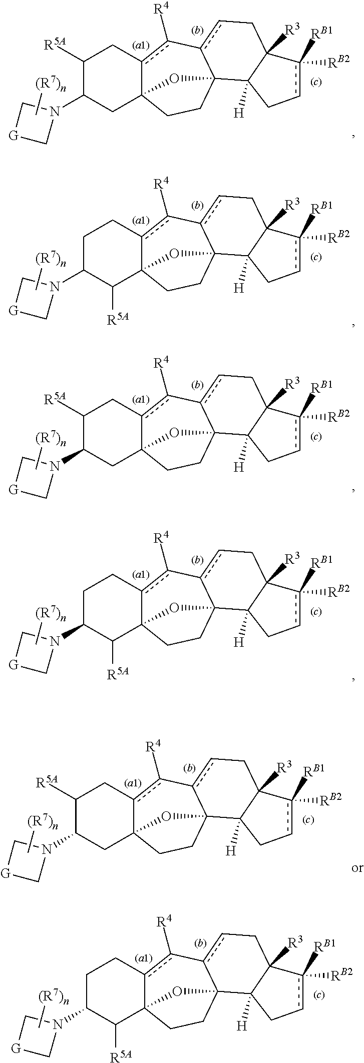

or a pharmaceutically acceptable salt, quaternary amine salt, or N-oxide thereof, wherein R³, R⁴, R^{5A}, R^{B1}, and R^{B2} are as defined herein; and [symbol] ,
wherein:
G is —O—, —S—, —NH—, —NR⁷—, —CH₂—, —CH(R⁷)—, or —C(R⁷)₂—;
each instance of R⁷ is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, amino, substituted amino, hydroxyl, substituted hydroxyl, thiol, substituted thiol, carbonyl, sulfonyl, sulfinyl, or a nitrogen protecting group when attached to a nitrogen atom;
optionally wherein two R⁷ groups are joined to form an optionally substituted carbocyclyl, optionally substituted heterocyclyl, optionally substituted aryl, an optionally substituted heteroaryl ring, or an oxo (═O) group; and
n is 0, 1, 2, 3, or 4.

In certain embodiments, R¹ and R² are joined to form a group of formula:

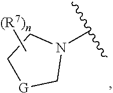

e.g., to provide a compound of formula:

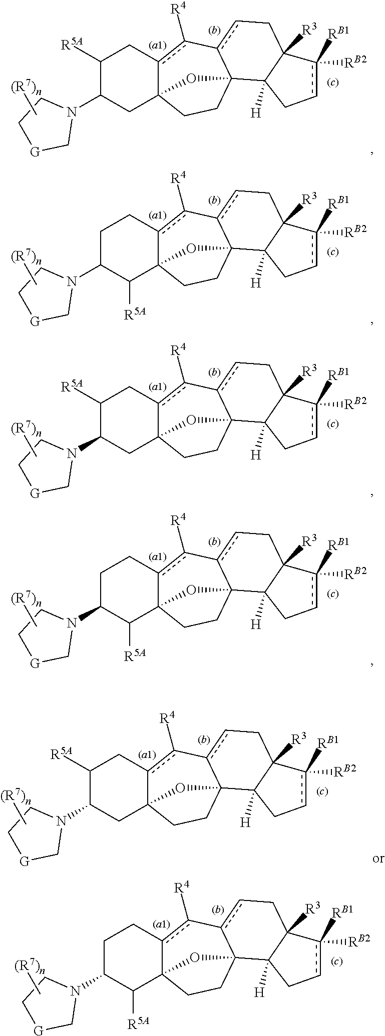

or a pharmaceutically acceptable salt, quaternary amine salt, or N-oxide thereof, wherein [symbol] ,
R³, R⁴, R^{5A}, R^{B1}, and R^{B2} are as defined herein; and [symbol]
wherein:
G is —O—, —S—, —NH—, —NR⁷—, —CH₂—, —CH(R⁷)—, or —C(R⁷)₂—;
each instance of R⁷ is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, amino, substituted amino, hydroxyl, substituted hydroxyl, thiol, substituted thiol, carbonyl, sulfonyl, sulfinyl, or a nitrogen protecting group when attached to a nitrogen atom;
optionally wherein two R⁷ groups are joined to form an optionally substituted carbocyclyl, optionally substituted heterocyclyl, optionally substituted aryl, an optionally substituted heteroaryl ring, or an oxo (═O) group; and
n is 0, 1, 2, 3, or 4.

In certain embodiments, R¹ and R² are joined to form a group of formula:

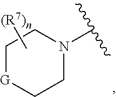

e.g., to provide a compound of formula:

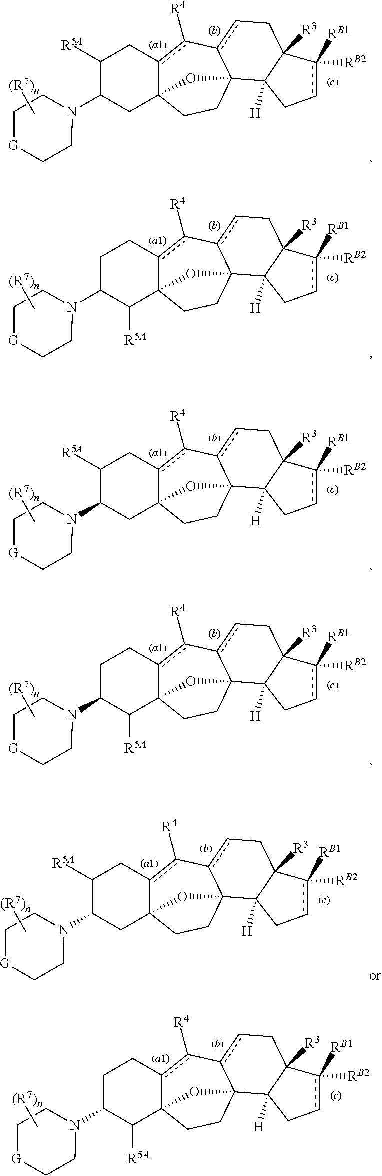

or a pharmaceutically acceptable salt, quaternary amine salt, or N-oxide thereof, wherein [symbol] ,
R³, R⁴, R^{5A}, R^{B1}, and R^{B2} are as defined herein; and [symbol]
wherein:
G is —O—, —S—, —NH—, —N—, —CH₂—, —CH(R⁷)—, or —C(R⁷)₂—;
each instance of R⁷ is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, amino, substituted amino, hydroxyl, substituted hydroxyl, thiol, substituted thiol, carbonyl, sulfonyl, sulfinyl, or a nitrogen protecting group when attached to a nitrogen atom;
optionally wherein two R⁷ groups are joined to form an optionally substituted carbocyclyl, optionally substituted heterocyclyl, optionally substituted aryl, an optionally substituted heteroaryl ring, or an oxo (═O) group; and
n is 0, 1, 2, 3, or 4.

In certain embodiments, n is 0, and the ring system formed by the joining of R¹ and R² is not substituted with an R⁷ group as defined herein. In certain embodiments, n is 1, 2, 3, or 4, and the ring system is substituted with 1, 2, 3, or 4, R⁷ groups as defined herein. In certain embodiments, n is 1. In certain embodiments, n is 2. In certain embodiments, n is 3. In certain embodiments, n is 4.

In certain embodiments, wherein n is not 0 (i.e., n is 1, 2, 3, or 4) and at least one R⁷ is attached to a carbon atom, the R⁷ is halogen (e.g., fluoro), hydroxyl, substituted hydroxyl, or carbonyl (e.g., —CO₂H). In certain embodiments, wherein n is not 0 (i.e., n is 1, 2, 3, or 4) and two R⁷ groups are attached to the same carbon atom, the two R⁷ groups are each halogen, e.g., fluoro. In certain embodiments, wherein n is not 0 (i.e., n is 1, 2, 3, or 4) and two R⁷ groups are attached to the same carbon atom, the two R⁷ groups are joined to form an optionally substituted carbocyclyl ring or optionally substituted heterocyclyl ring (e.g., optionally substituted oxetanyl ring). In certain embodiments, wherein n is not 0 (i.e., n is 1, 2, 3, or 4) and two R⁷ groups are attached to a different carbon atom, the two R⁷ groups are joined to form an optionally substituted carbocyclyl ring or optionally substituted heterocyclyl ring.

In certain embodiments, G is —O—. In certain embodiments, G is —NR⁷—, e.g., wherein R⁷ is optionally substituted alkyl (e.g., —CH₃). In certain embodiments, G is —CH(R⁷)— or —C(R⁷)₂— wherein at least one R⁷ is hydroxyl, substituted hydroxyl, or carbonyl (e.g., —CO₂H).

In certain embodiments, the group is

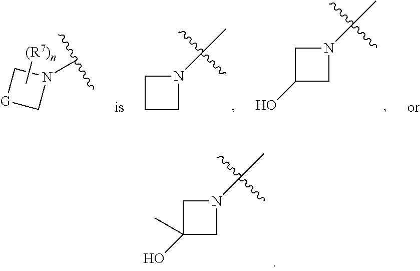

In certain embodiments, the group

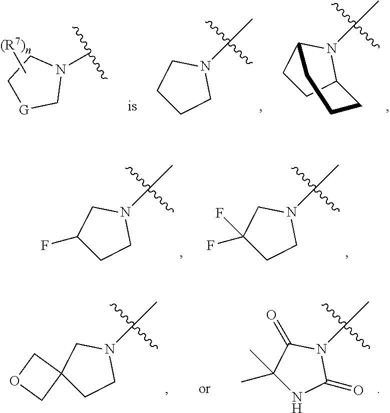

In certain embodiments, the group

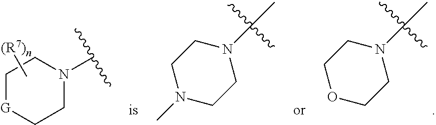

In certain embodiments, R¹ is —S(═O)₂R^{A} and R² is optionally substituted alkyl.

### Group R^{O}

As generally defined herein, R^{O} is hydrogen or optionally substituted alkyl, optionally substituted alkenyl, optionally substituted alkynyl, optionally substituted carbocyclyl, optionally substituted heterocyclyl, optionally substituted aryl, optionally substituted heteroaryl, —C(═O)R^{A}, —C(═O)OR^{A}, —C(═O)N(R^{A})₂, or an oxygen protecting group.

In certain embodiments, R^{O} is hydrogen.

In certain embodiments, R^{O} is optionally substituted alkyl, e.g., optionally substituted C₁₋₆alkyl, e.g., optionally substituted C₁alkyl, optionally substituted C₂alkyl, optionally substituted C₃alkyl, optionally substituted C₄alkyl, optionally substituted C₅alkyl, or optionally substituted C₆alkyl. In certain embodiments, R^{O} is optionally substituted methyl (C₁), optionally substituted ethyl (C₂), optionally substituted n-propyl (C₃), optionally substituted isopropyl (C₃), optionally substituted n-butyl (C₄), or optionally substituted t-butyl (C₄). In certain embodiments, R^{O} is alkyl substituted with one or more halogen substituents (e.g., fluoro). In certain embodiments, R^{O} is —CH₃ or —CF₃. In certain embodiments, R^{O} is alkyl substituted with one or more halogen (e.g., fluoro), amino (—NH₂), substituted amino, hydroxyl (—OH), substituted hydroxyl, thiol (—SH), substituted thiol, or sulfonyl substituents. In certain embodiments, R^{O} is alkyl substituted with an optionally substituted carbocyclyl (e.g., cyclopropyl) or optionally substituted heterocyclyl (e.g., oxetanyl) ring.

For example, in certain embodiments, R^{O} is a group of formula:

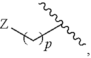

e.g., to provide a compound of formula:

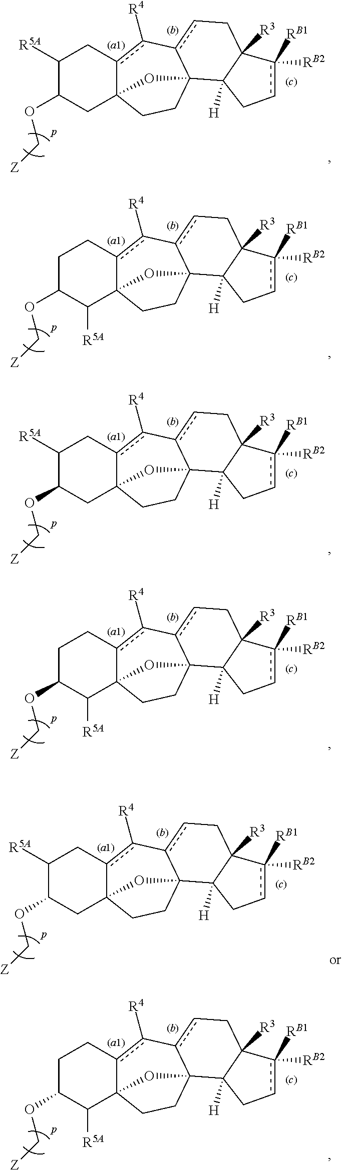

or a pharmaceutically acceptable salt, quaternary amine salt, or N-oxide thereof, where [symbol] R³, R⁴, R^{5A}, R^{B1}, and R^{B2} are as defined herein; and
wherein:
p is 1, 2, 3, 4, 5, or 6; and
Z is —CH₂X^{Z}, —CH(X^{Z})₂, —C(X^{Z})₃, —OR^{Z}, —SR^{Z}, —N(R^{Z})₂, —S(O)₂N(R^{Z})₂,

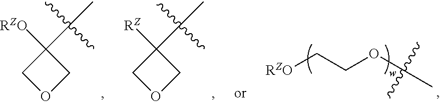

wherein each instance of R^{Z} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —C(═O)R^{Z}, —C(═O)OR^{Z}, —C(═O)N(R^{Z})₂, an oxygen protecting group when attached to oxygen, a sulfur protecting group when attached to sulfur, or a nitrogen protecting group when attached to nitrogen, optionally when attached to N the two R^{Z}groups may be joined to form an optionally substituted heterocyclyl or optionally substituted heteroaryl ring;
each instance of X^{Z} is independently fluoro, chloro, bromo, or iodo; and
w is an integer between 1 and 10, inclusive.

In certain embodiments, p is 1. In certain embodiments, p is 2. In certain embodiments, p is 3. In certain embodiments, w is 1, 2, 3, or 4. In certain embodiments, R^{Z} is hydrogen or optionally substituted alkyl (e.g., —CH₃). In certain embodiments, Z is —OR^{Z}, e.g., —OH or —OR^{Z} wherein R^{Z} is a non-hydrogen group, e.g., wherein R^{Z} is optionally substituted alkyl such as —CH₃. In certain embodiments, Z is —N(R^{Z})₂, e.g., —NH₂, —NHR^{Z}, or —N(R^{Z})₂ wherein R^{Z} is a non-hydrogen group, e.g., wherein R^{Z} is optionally substituted alkyl such as —CH₃. In certain embodiments, Z is —CH₂X^{Z}, —CH(X^{Z})₂, —C(X^{Z})₃, e.g., wherein X^{Z} is fluoro. In certain embodiments, Z is —S(O)₂N(R^{Z})₂, e.g., —S(O)₂NH₂ or —S(O)₂NHCH₃.

In certain embodiments of Formula (A-1-D′) or (A-1-D″), R^{O} is —C(═O)R^{A}, —C(═O)OR^{A}, or —C(═O)N(R^{A})₂. In certain embodiments, R^{A} is hydrogen or optionally substituted alkyl (e.g., —CH₃). For example, in certain embodiments, R^{O} is —C(═O)CH₃, —C(═O)OCH₃, —C(═O)N(CH₃)₂, or —C(═O)NHCH₃.

In certain embodiments, R^{O} is an oxygen protecting group.

Group R³, R⁴, R^{5A}, R^{5B} and bonds of formula

[symbol]

As generally defined herein, R³ is hydrogen or optionally substituted alkyl.

In certain embodiments, R³ is hydrogen. In certain embodiments, R³ is optionally substituted alkyl, e.g., methyl (—CH₃).

As generally defined herein, R⁴ is hydrogen, halogen, optionally substituted alkyl, or —Si(R^{A})₃. In certain embodiments, R⁴ is hydrogen. In certain embodiments, R⁴ is optionally substituted alkyl, e.g., methyl. In certain embodiments, R⁴ is —Si(R^{A})₃, e.g., wherein each instance of R^{A} is independently optionally substituted alkyl or optionally substituted phenyl.

As generally defined herein, R^{5A} is hydrogen, halogen, optionally substituted alkyl, —OR^{A}, —OC(═O)R^{A}, —OC(═O)OR^{A}, —OC(═O)N(R^{A})₂, —OS(═O)₂R^{A}, —N₃, —N(R^{A})₂, —NR^{A}C(═O)R^{A}, —NR^{A}C(═O)OR^{A}, —NR^{A}C(═O)N(R^{A})₂, —NR^{A}S(═O)₂R^{A}, or —C(R^{A})₃. In certain embodiments, R^{5A} is hydrogen. In certain embodiments, R^{5A} is a non-hydrogen group. In certain embodiments, R^{5A} is halogen (e.g., bromo, iodo, chloro). In certain embodiments, R^{5A} is optionally substituted alkyl (e.g., —CH₃). In certain embodiments, R^{5A} is —OR^{A} (e.g., —OH, —OCH₃).

In certain embodiments, R⁵Ais hydrogen, halogen, optionally substituted alkyl, or —OR^{A}.

In certain embodiments, R^{5A} is —OR^{A}, —OC(═O)R^{A}, —OC(═O)OR^{A}, —OC(═O)N(R^{A})₂, —OS(═O)₂R^{A}.

In certain embodiments, R^{5A} is —N₃, —N(R^{A})₂, —NR^{A}C(═O)R^{A}, —NR^{A}C(═O)OR^{A}, —NR^{A}C(═O)N(R^{A})₂, or —NR^{A}S(═O)₂R^{A}.

In certain embodiments, R^{5A} is —C(R^{A})₃.

In certain embodiments, the group R^{5A} is in the alpha (down) configuration. In certain embodiments, the group R^{5A} is in the beta (up) configuration.

As generally defined herein R^{5B} is hydrogen, halogen, optionally substituted alkyl, or —OR^{A}. In certain embodiments, R^{5B} is hydrogen. In certain embodiments, R^{5B} is a non-hydrogen group. In certain embodiments, R^{5B} is halogen (e.g., bromo, iodo, chloro). In certain embodiments, R^{5B} is optionally substituted alkyl, e.g., methyl. In certain embodiments, R^{5B} is —OR^{A}, e.g., —OH. In certain embodiments, R^{5B} is not —OR^{A}.

In certain embodiments, at least one instance of R^{5A} and R^{5B} is hydrogen. In certain embodiments, R^{5A} is hydrogen and R^{5B} is non-hydrogen. In certain embodiments, R^{5A} is non-hydrogen and R^{5B} is hydrogen. In certain embodiments, each instance of R^{5A} and R^{5B} is hydrogen.

In certain embodiments, at least one instance of R^{5A} and R^{5B} is halogen (e.g., bromo, iodo, chloro). In certain embodiments, at least one instance of R^{5A} and R^{5B} is optionally substituted alkyl, e.g., methyl.

In certain embodiments, at least one instance of R^{5A} and R^{5B} is —OR^{A}, e.g., —OH. In certain embodiments, R^{5A} is —OR^{A}, e.g., —OH and R^{5B} is hydrogen. In certain embodiments, R^{5A} is hydrogen and R^{5B} is —OR^{A}, e.g., —OH. In certain embodiments, each instance of R^{5A} and R^{5B} is —OR^{A}, e.g., —OH. In certain embodiments, neither instance of R^{5A} and R^{5B} is —OR^{A}.

As generally each instance of [symbol] , designated as (a1), (a2), (b), (c), (d1), and (d2) represents a single or double bond, as valency permits, providing:
when [symbol] designated as (c) represents a double bond, then one of R^{B1} and R^{B2} is absent and one of Y¹ and Y² is absent,
when [symbol] designated as (c) represents a single bond, then both R^{B1} and R^{B2} are present and both of Y¹ and Y² are present,
when [symbol] designated as (a1) represents a double bond, then [symbol] designated as (d2) and (a2) each represent single bonds,
when [symbol] designated as (a2) represents a double bond, then [symbol] designated as (a1) and (b) each represent single bonds, and
when [symbol] designated as (d2) represents a double bond, then [symbol] designated as (a1) and (d1) each represent single bonds.

In certain embodiments, the bond [symbol] designated as (a2) is a single bond. In certain embodiments, the bond [symbol] designated as (a1) is a double bond. In certain embodiments, the bond [symbol] designated as (b) is a double bond. In certain embodiments, each instance of [symbol] designated as (a1) and (b) is a double bond. In certain embodiments, the bond [symbol] designated as (c) is a single bond. In certain embodiments, the bond [symbol] designated as (d2) is a single bond. In certain embodiments, the bond [symbol] designated as (d1) is a single bond.

In certain embodiments R³ is methyl, R⁴ is hydrogen, R^{5A} is hydrogen, and the bond designated (c) is a single bond.

In other embodiments R³ is methyl, R⁴ is hydrogen, the bond designated (c) is a double bond, and R^{B2} is absent.

Groups R^{B1} and R^{B2}

As generally defined herein, each instance of R^{B1} and R^{B2} is, independently, hydrogen, -L₁-R^{B3}, or —X^{A}R^{A} wherein X^{A} is —O—, —S—, or —N(R^{A})—; or R^{B1} and R^{B2} are joined to form an oxo group, provided that at least one of R^{B1} and R^{B2} is not hydrogen;
L₁ is a bond, —CH(CH₃)(CH₂)₂—, —CH(CH₃)—CH═CH—, —C(═O)—, —C(═O)O—, —C(═O)S—, —C(═O)N(R^{L})—, or —N(R^{L})—(C(R^{LL})₂)ₚ—, wherein R^{L} is hydrogen, optionally substituted alkyl, or a nitrogen protecting group, each instance of R^{LL} is independently hydrogen, halogen, or optionally substituted alkyl, and p is 0, 1, or 2; and
R^{B3} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, provided that when L₁ is a bond, then R^{B3} is not hydrogen.

In certain embodiments, at least one instance of R^{B1} and R^{B2} is -L₁-R^{B3}. In certain embodiments, when [symbol] designated as (c) represents a single bond, then R^{B1} is -L₁-R^{B3} and R^{B2} is hydrogen or —X^{A}R^{A} (e.g., —OR^{A}).

In certain embodiments, L₁ is a bond, and R^{B3} is optionally substituted alkyl, optionally substituted alkenyl, optionally substituted alkynyl, optionally substituted carbocyclyl, optionally substituted heterocyclyl, optionally substituted aryl, or optionally substituted heteroaryl.

In certain embodiments, R^{B3} is a cyclic group, e.g., R^{B3} is optionally substituted carbocyclyl, optionally substituted heterocyclyl, optionally substituted aryl, or optionally substituted heteroaryl. In certain embodiments, R^{B3} is a nonaromatic cyclic group, e.g., in certain embodiments, R^{B3} is optionally substituted carbocyclyl or optionally substituted heterocyclyl. In certain embodiments, R^{B3} is an aromatic cyclic group, e.g., in certain embodiments, R^{B3} is optionally substituted aryl or optionally substituted heteroaryl.

In certain embodiments, R^{B3} is optionally substituted aryl, e.g., optionally substituted C₆-14aryl. In certain embodiments, R^{B3} is optionally substituted phenyl. In certain embodiments, R^{B3} is optionally substituted naphthyl. In certain embodiments, R^{B3} is optionally substituted phenyl fused to an optionally substituted heterocyclyl ring; such as an optionally substituted phenyl tetrahydroisoquinolinyl. It is understood in reference to optionally substituted aryl ring systems comprising a fused heterocyclyl ring that the point of attachment to the parent molecule is on the aryl (e.g., phenyl) ring.

In certain embodiments, R^{B3} is optionally substituted heteroaryl, e.g., optionally substituted 5-14 membered heteroaryl. In certain embodiments, R^{B3} is an optionally substituted 5-membered heteroaryl or an optionally substituted 6-membered heteroaryl. In certain embodiments, R^{B3} is an optionally substituted bicyclic heteroaryl, e.g., an optionally substituted 5,6-bicyclic heteroaryl, or optionally substituted 6,6-bicyclic heteroaryl. In certain embodiments, R^{B3} is an optionally substituted 5,6-bicyclic heteroaryl or optionally substituted 6,6-bicyclic heteroaryl ring system selected from the group consisting of optionally substituted naphthyridinyl, optionally substituted pteridinyl, optionally substituted quinolinyl, optionally substituted isoquinolinyl, optionally substituted cinnolinyl, optionally substituted quinoxalinyl, optionally substituted phthalazinyl, and optionally substituted quinazolinyl. In certain embodiments, the point of attachment of R^{B3} is via a nitrogen atom.

In certain embodiments, wherein R^{B3} is an optionally substituted aryl or optionally substituted heteroaryl, -L₁-R^{B3} is selected from the group consisting of:

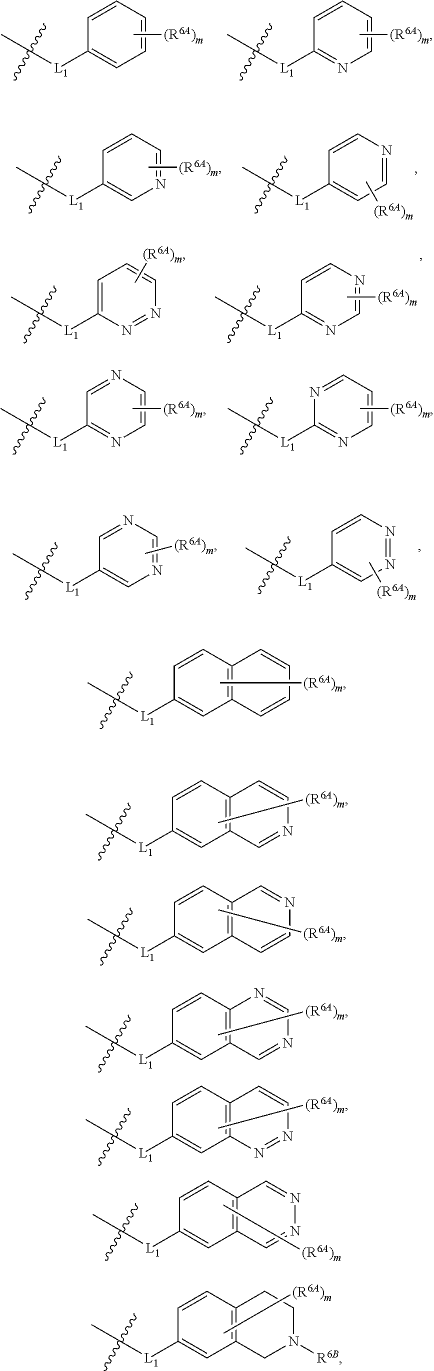

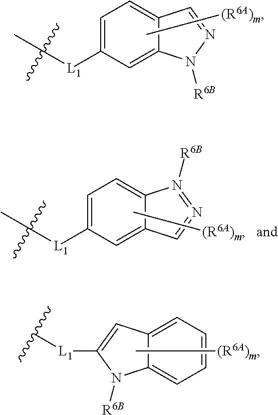

wherein:
each instance of R^{6A} is independently halogen, —NO₂N, —CN, —OR^{6C}, SR^{6C}, N(R^{6C})₂, —C(═O)R^{6C}, —C(═O)OR^{6C}, —C(═O)N(R^{6C})₂, optionally substituted alkyl, optionally substituted alkenyl, optionally substituted alkynyl, optionally substituted carbocyclyl, optionally substituted heterocyclyl, optionally substituted aryl, or optionally substituted heteroaryl;
each instance of R^{6B} is independently hydrogen, optionally substituted alkyl, or a nitrogen protecting group when attached to nitrogen;
wherein each instance of R^{6C} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to oxygen, a sulfur protecting group when attached to sulfur, or a nitrogen protecting group when attached to nitrogen, optionally when attached to N the two R^{6C} groups may be joined to form an optionally substituted heterocyclyl or optionally substituted heteroaryl ring; and
m is 0 or an integer between 1 and 4, inclusive.

In certain embodiments, m is 0. In certain embodiments, m is 1, 2, 3, or 4. In certain embodiments, wherein m is 1, 2, 3, or 4, at least one R^{6A} is halogen (e.g., fluoro), —OR^{6C}, —SR^{6C}, or —N(R^{6C})₂.

In certain embodiments, L₁ is a bond or —C(═O)N(R^{L})—, wherein R^{L} is hydrogen or an optionally substituted alkyl (e.g., methyl), and R^{B3} is optionally substituted aryl or optionally substituted heteroaryl, as described herein.

### Compounds of Formula (A-2′), (A-2″), (A-3′), (A-3″), (D1′), (D1″), (D2′), (D2″), (E1′), (E1″), (E2′), (E2″), (G1′), and (G1″)

As generally defined herein, in certain embodiments, a cortistatin or cortistatin analog thereof is a compound of Formula:

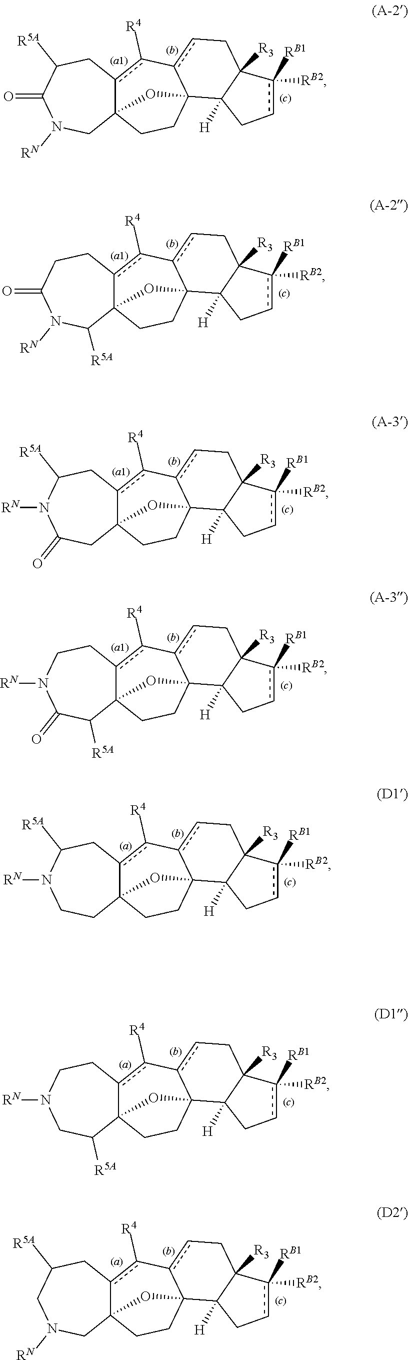

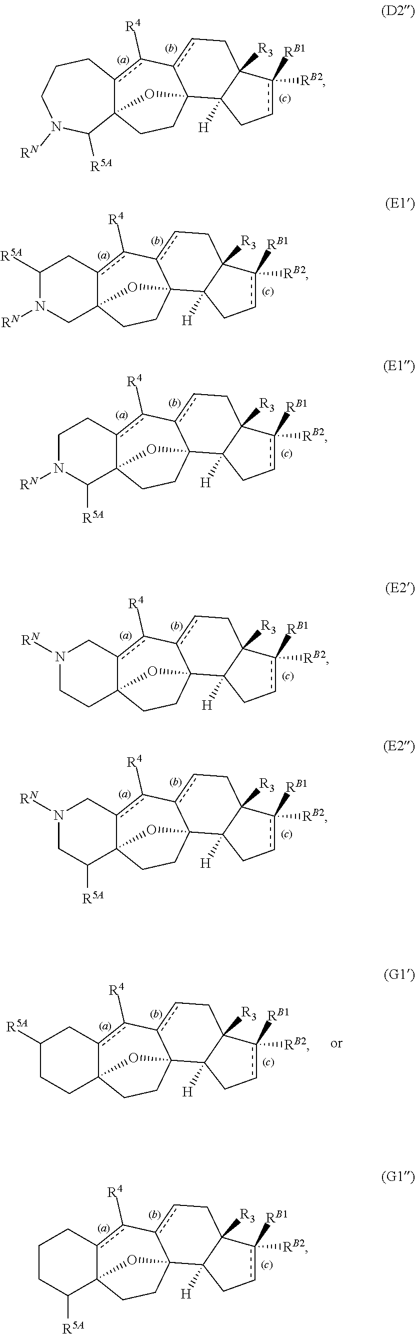

or a pharmaceutically acceptable salt, quaternary amine salt, or N-oxide thereof.

As generally defined herein, R^{N} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, C(═O)R^{A}, —C(═O)OR^{A}, —C(═O)N(R^{A})₂, —S(═O)₂R^{A}, or a nitrogen protecting group.

In certain embodiments R^{N} is optionally substituted alkyl, e.g., optionally substituted C₁₋₆alkyl, e.g., optionally substituted C₁alkyl, optionally substituted C₂alkyl, optionally substituted C₃alkyl, optionally substituted C₄alkyl, optionally substituted C₅alkyl, or optionally substituted C₆alkyl. In certain embodiments, R^{O} is optionally substituted methyl (C₁), optionally substituted ethyl (C₂), optionally substituted n-propyl (C₃), optionally substituted isopropyl (C₃), optionally substituted n-butyl (C₄), or optionally substituted t-butyl (C₄).

In certain embodiments R^{N} is —C(═O)R^{A}, —C(═O)OR^{A}, or —C(═O)N(R^{A})₂. In certain embodiments, R^{A} is hydrogen or optionally substituted alkyl (e.g., —CH₃). For example, in certain embodiments, R^{N} is —C(═O)CH₃, —C(═O)OCH₃, —C(═O)N(CH₃)₂, or —C(═O)NHCH₃.

In certain embodiments R^{N} is a nitrogen protecting group.

In certain embodiments R^{N} is hydrogen.

In certain embodiments, the compound of Formula (A-2′) or (A-2″) is of formula:

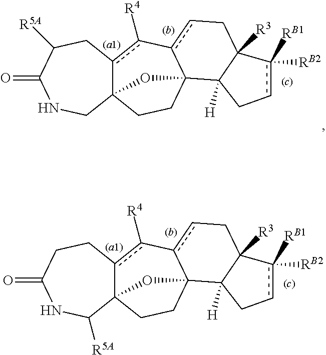

or a pharmaceutically acceptable salt, quaternary amine salt, or N-oxide thereof.

In certain embodiments, the compound of Formula (A-3′) or (A-3″) is of formula:

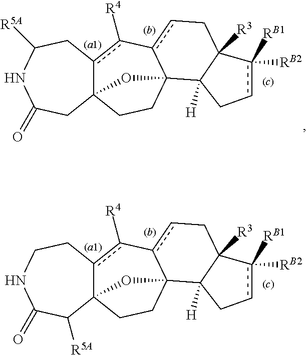

or a pharmaceutically acceptable salt, quaternary amine salt, or N-oxide thereof.

In certain embodiments, the compound is of Formula (G1′) or (G1″). Compounds of Formula (G1′) or (G1″) may be prepared reduction of the ketone of a Compound of Formula (A-1′) or (A-1″) as depicted in the below scheme.

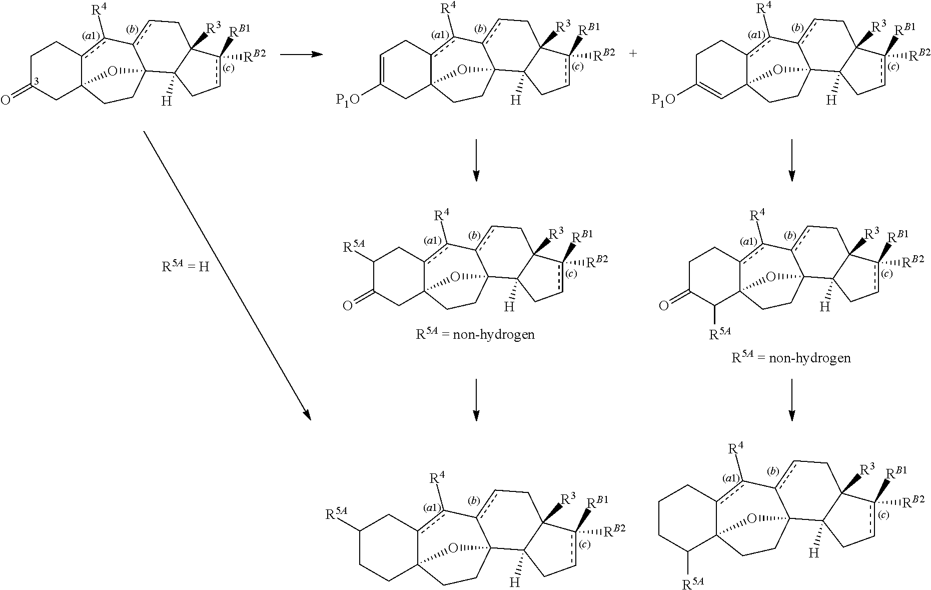

For example, the starting material ketone may be optionally trapped as the enolate (e.g., via treatment with base and a P₁-LG group, wherein P₁ is silyl and LG is a leaving group), followed by subsequent oxidation or amination of the double bond, or reaction of the double bond with an electrophilic carbon C(R^{A})₃-LG, wherein LG is a leaving group, to provide a substituted ketone product, wherein R⁵ is a non-hydrogen group, such as halogen, OR^{A}, OC(═O)R^{A}, —OC(═O)OR^{A}, —OC(═O)N(R^{A})₂, —OS(═)₂R^{A}, —N₃, —N(R^{A})₂, —NR^{A}C(═O)R^{A}, —NR^{A}C(═O)OR^{A}, —NR^{A}C(═O)N(R^{A})₂, —NR^{A}S(═O)₂R^{A}, or —C(R^{A})₃.

The ketone may be reduced under Wolff-Kishner reductive conditions to provide compounds of Formula (G1′) and (G1″). Exemplary Wolff-Kishner conditions are described in Furrow, M. E.; Myers, A. G. (2004). “Practical Procedures for the Preparation of N-tert-ButyIdimethylsilyihydrazones and Their Use in Modified Wolff-Kishner Reductions and in the Synthesis of Vinyl Halides andgem-Dihalides” Journal of the American Chemical Society 126 (17): 5436-5445, incorporated herein by reference.

### Exemplary Compounds

Various combinations of certain embodiments are further contemplated herein.

For example, in certain embodiments wherein the group -L₁R^{B3} is a group of formula:

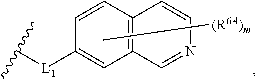

and wherein L₁ is a bond, provided are compounds of Formula:

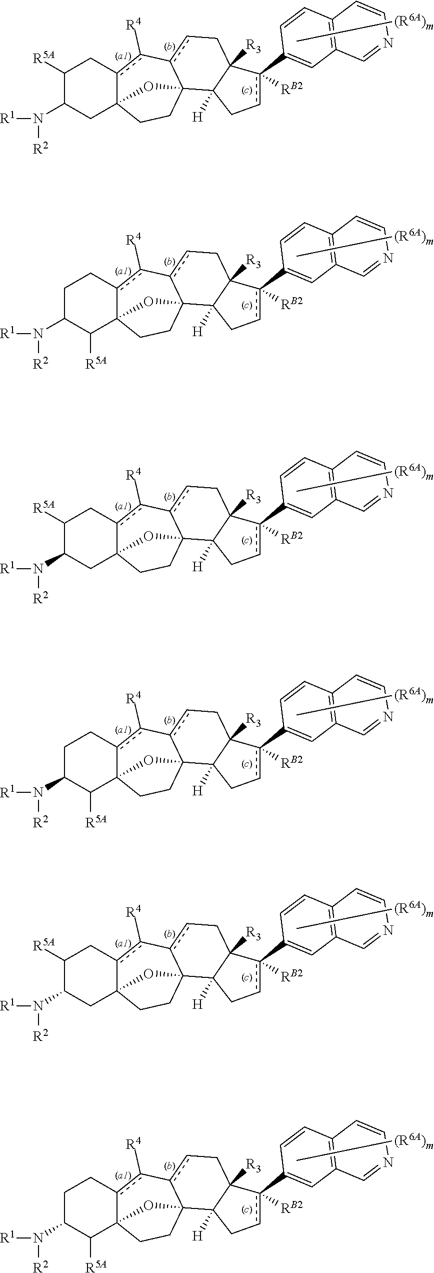

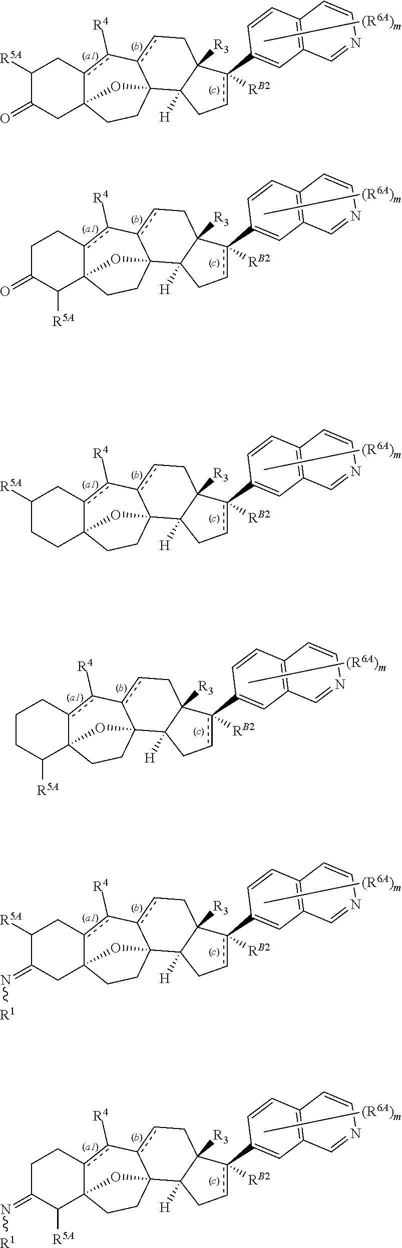

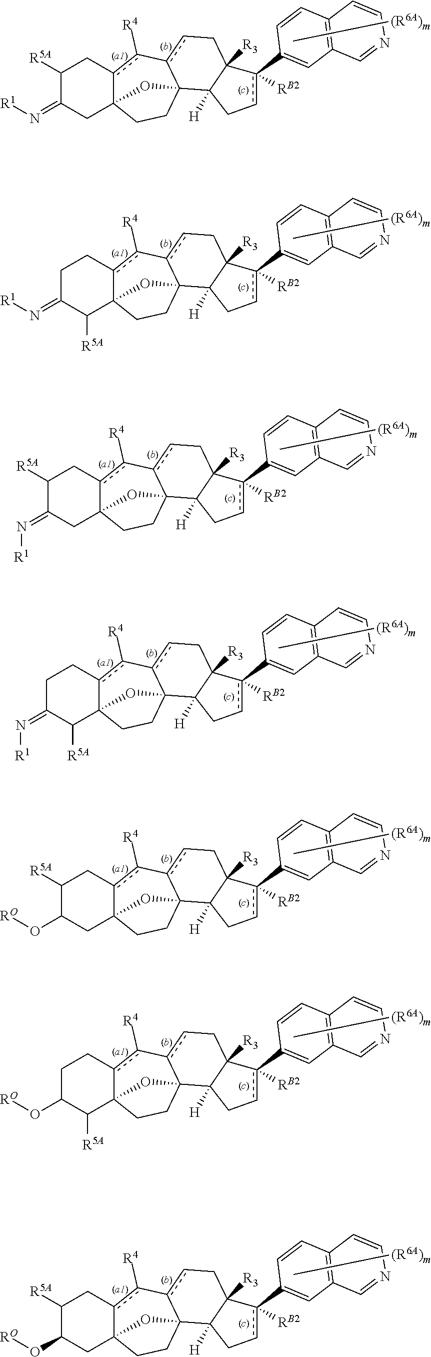

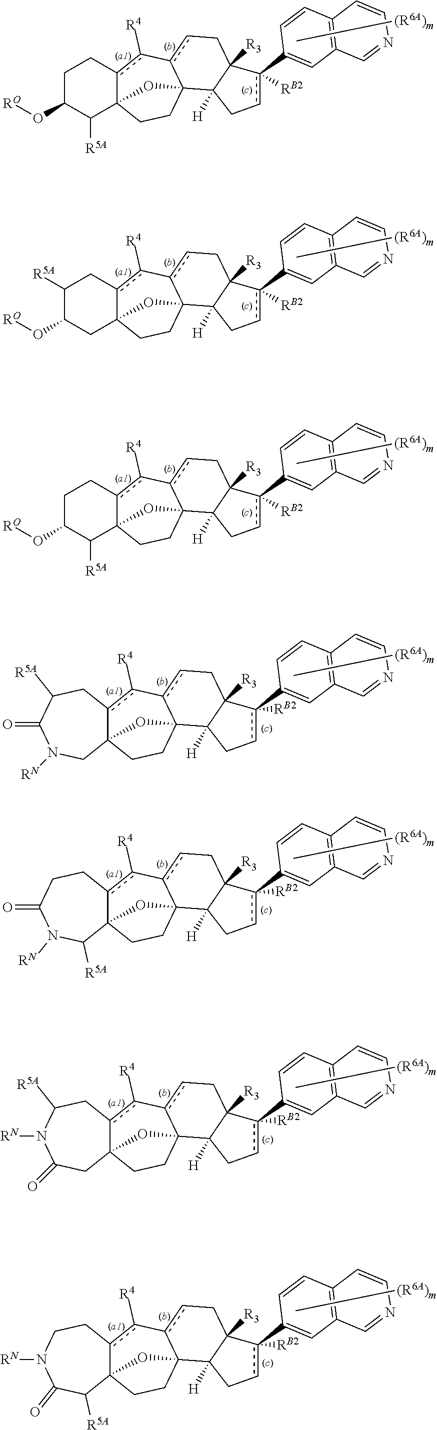

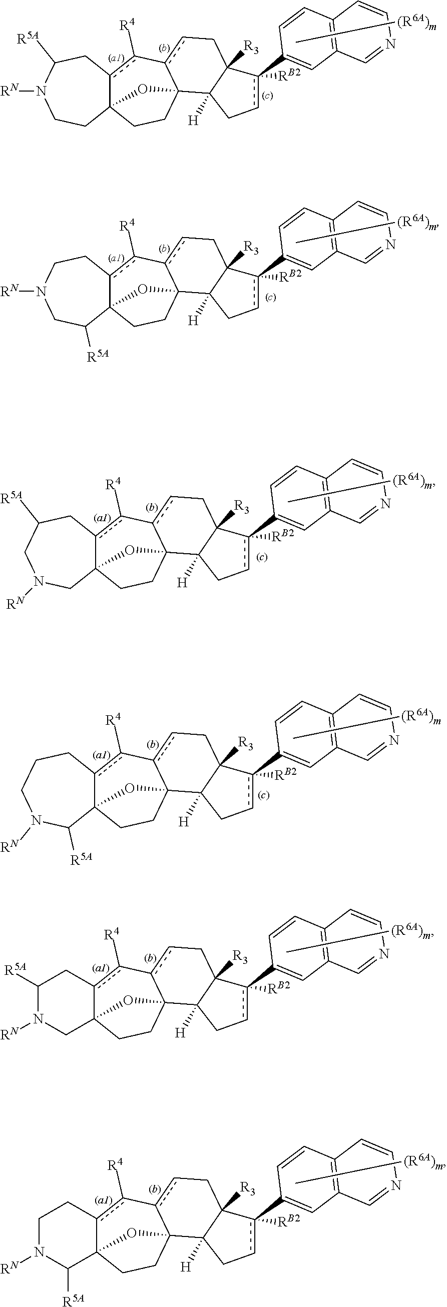

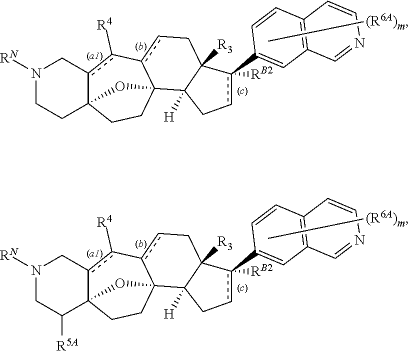

or a pharmaceutically acceptable salt, quaternary amine salt, or N-oxide thereof; wherein [symbol] , R^{N}, R¹, R², R³, R⁴, R^{5A}, R^{6A}, and m are as defined herein.

In certain embodiments, wherein R¹ and R² are joined to form an optionally substituted heterocyclyl, provided is a compound of Formula:

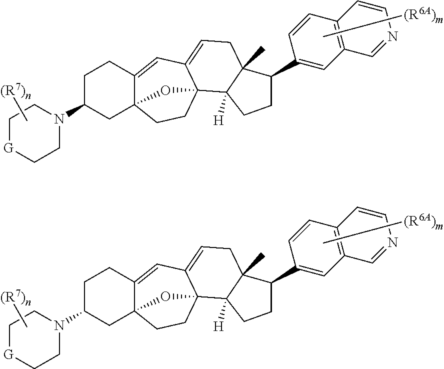

or a pharmaceutically acceptable salt, quaternary amine salt, or N-oxide thereof; wherein R⁷, R^{6A}, n and m are as defined herein. In certain embodiments, G is O. In certain embodiments, G is N—CH₃. In certain embodiments, m is 0. In certain embodiments, m is 1. In certain embodiments, n is 0. In certain embodiments, n is 1.

In certain embodiments, wherein R¹ and R² are joined to form an optionally substituted heterocyclyl, provided is a compound of Formula:

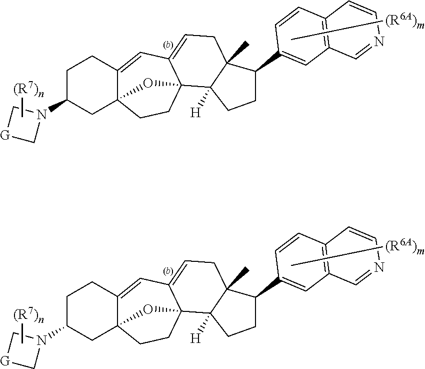

or a pharmaceutically acceptable salt, quaternary amine salt, or N-oxide thereof, wherein R⁷, R^{6A}, n and m are as defined herein. In certain embodiments, G is —CH₂—. In certain embodiments, m is 0. In certain embodiments, m is 1. In certain embodiments, n is 0. In certain embodiments, n is 1.

In certain embodiments, wherein each of R¹ and R² are —CH₃, provided is a compound of formula:

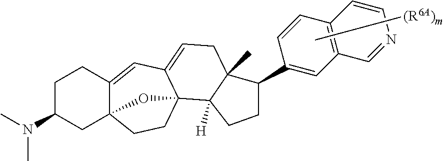

or a pharmaceutically acceptable salt, quaternary amine salt, or N-oxide thereof; wherein R^{6A}, and m are as defined herein. In certain embodiments, m is 0. In certain embodiments, m is 1.

In certain embodiments, wherein one of R¹ and R² is hydrogen, and the other of R¹ and R² is —CH₃, provided is a compound of formula:

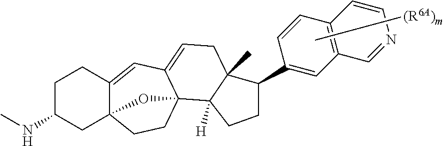

or a pharmaceutically acceptable salt, quaternary amine salt, or N-oxide thereof; wherein R^{6A}, and m are as defined herein. In certain embodiments, m is 0. In certain embodiments, m is 1.

Exemplary compounds of Formula (A-1-B′) or (A-1-B″) include, but are not limited to:

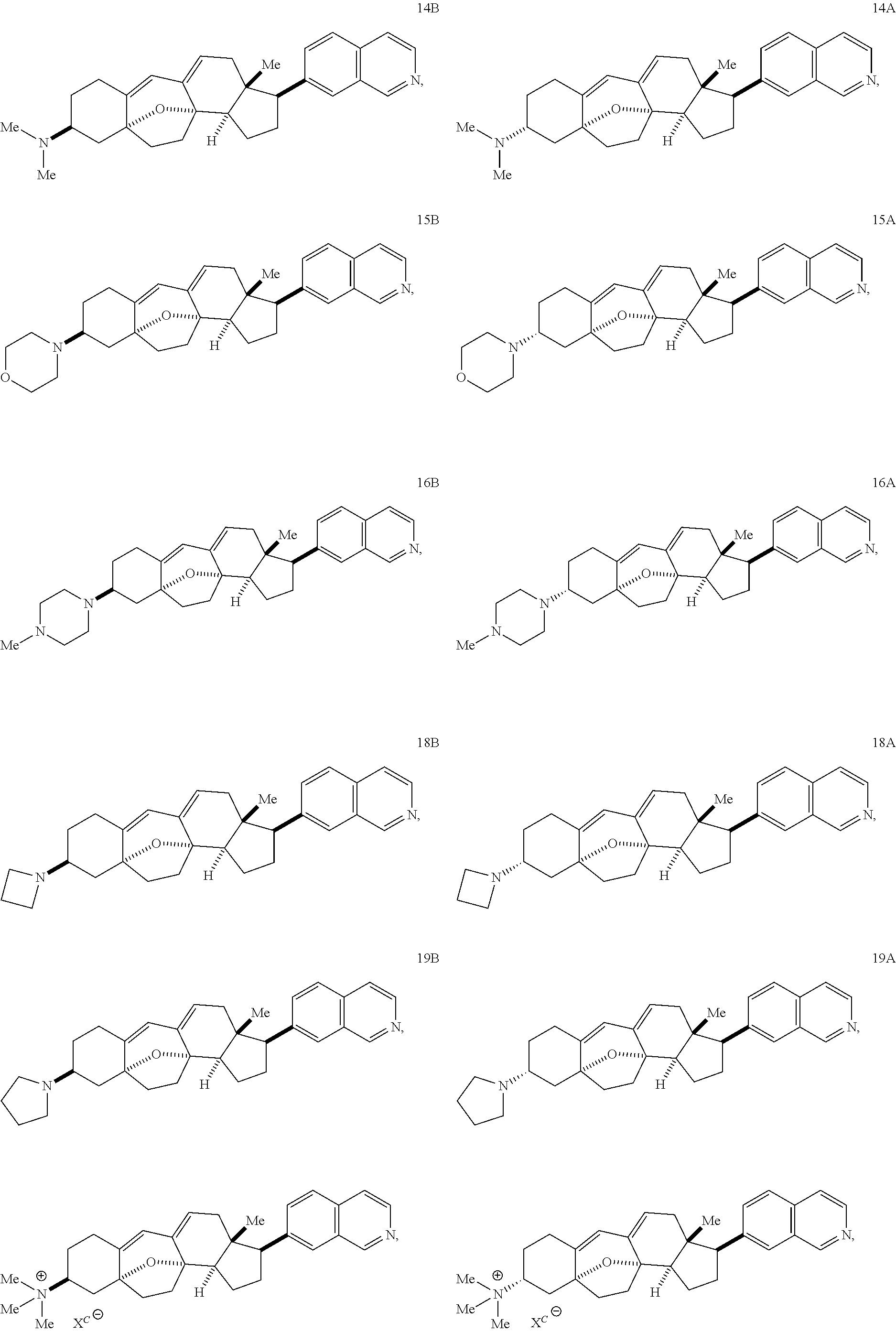

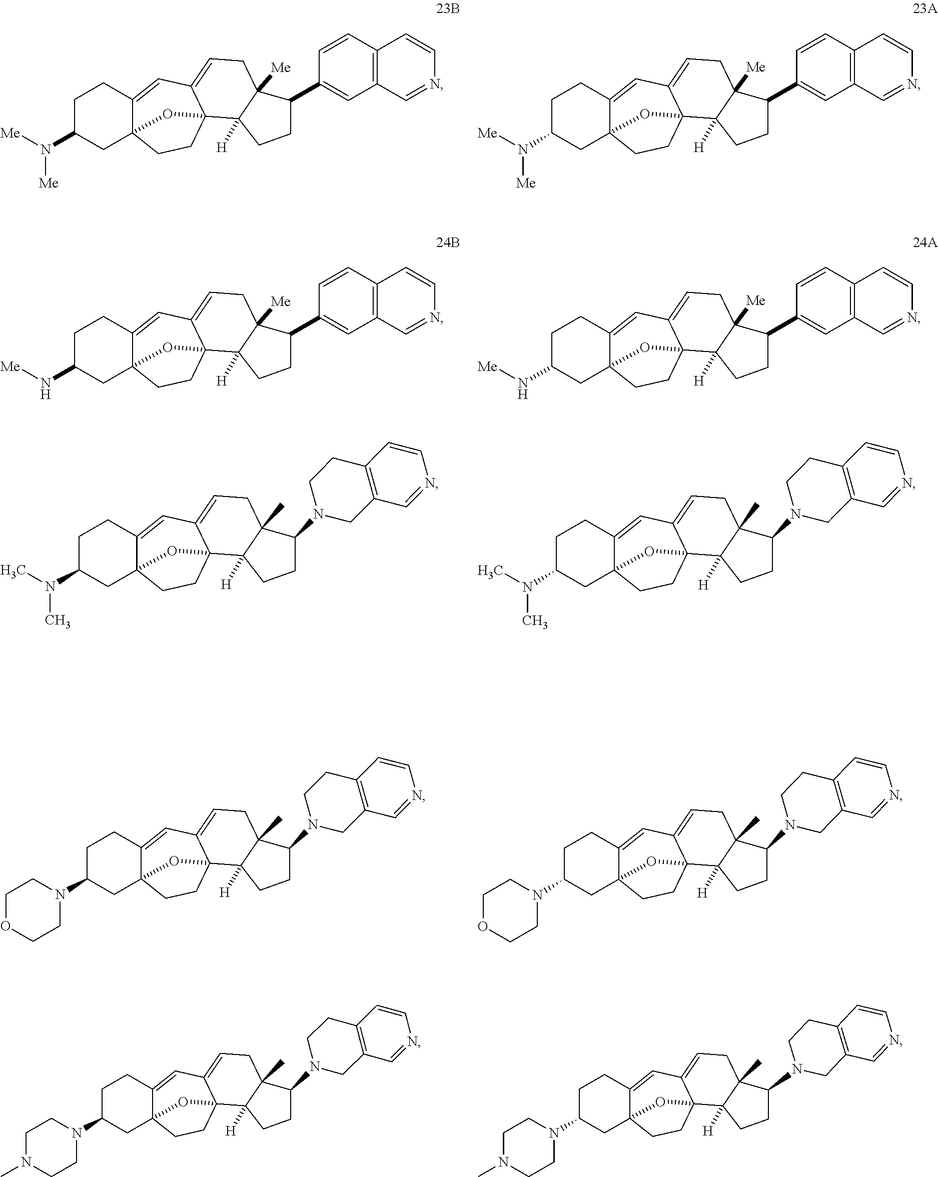

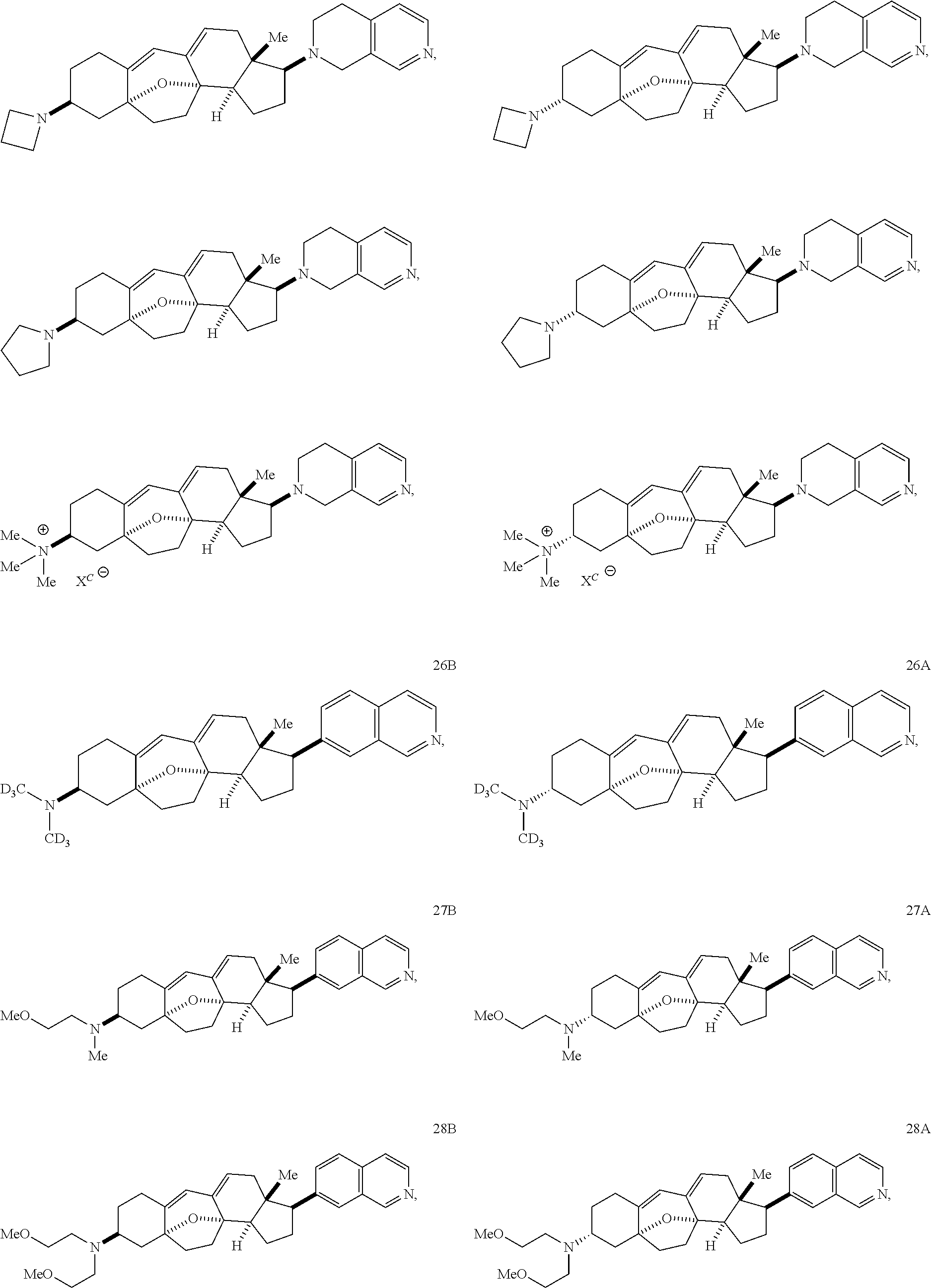

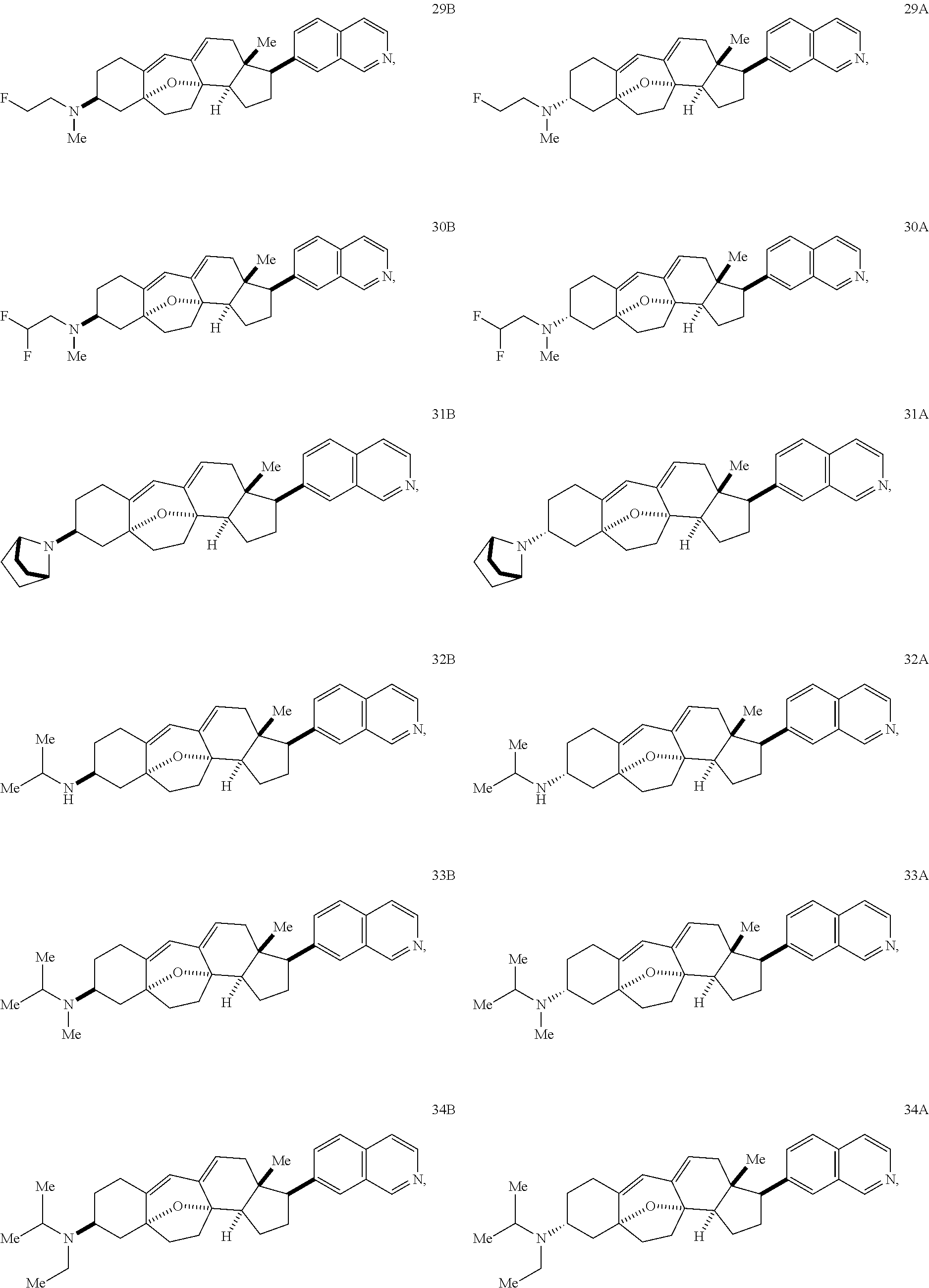

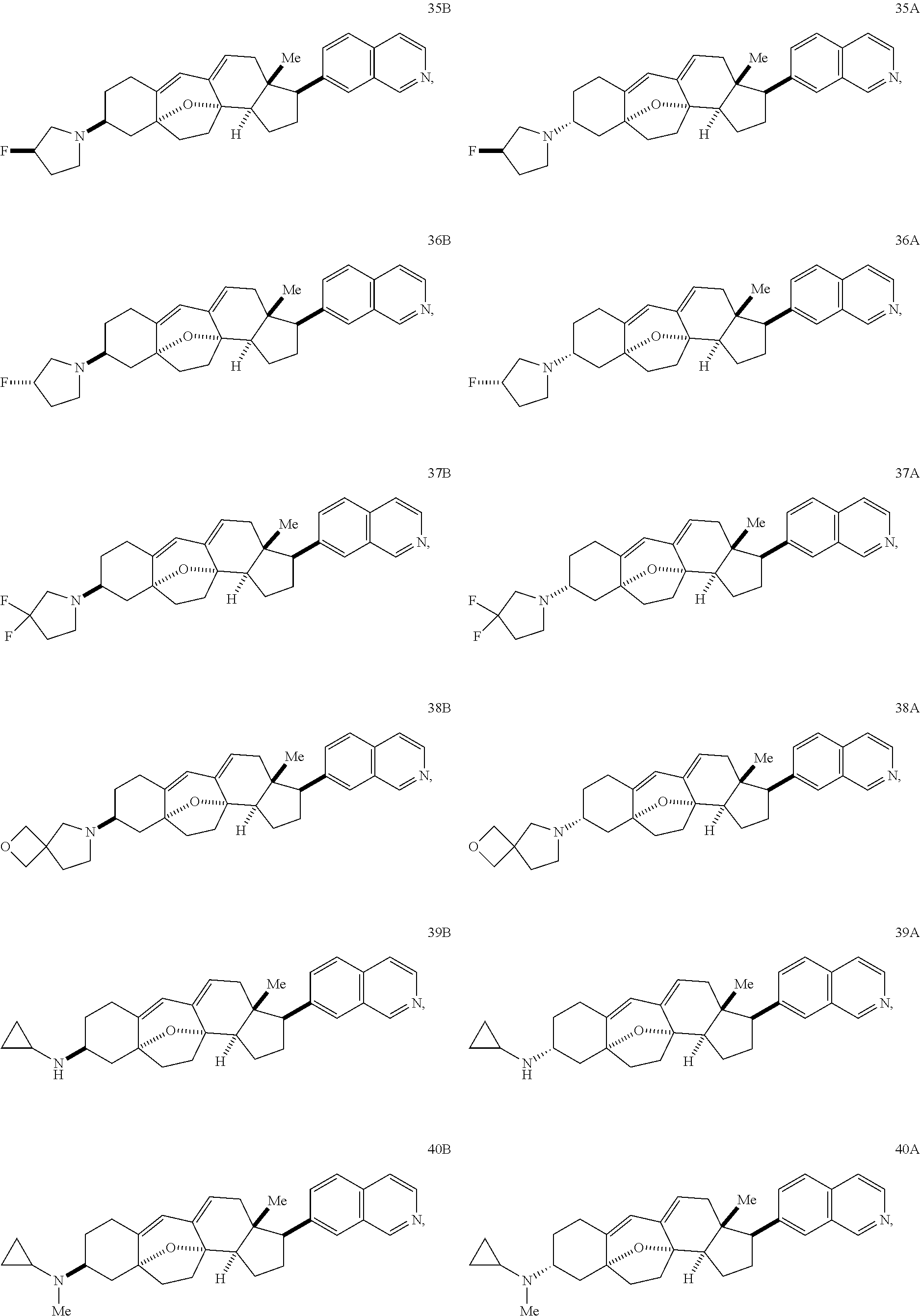

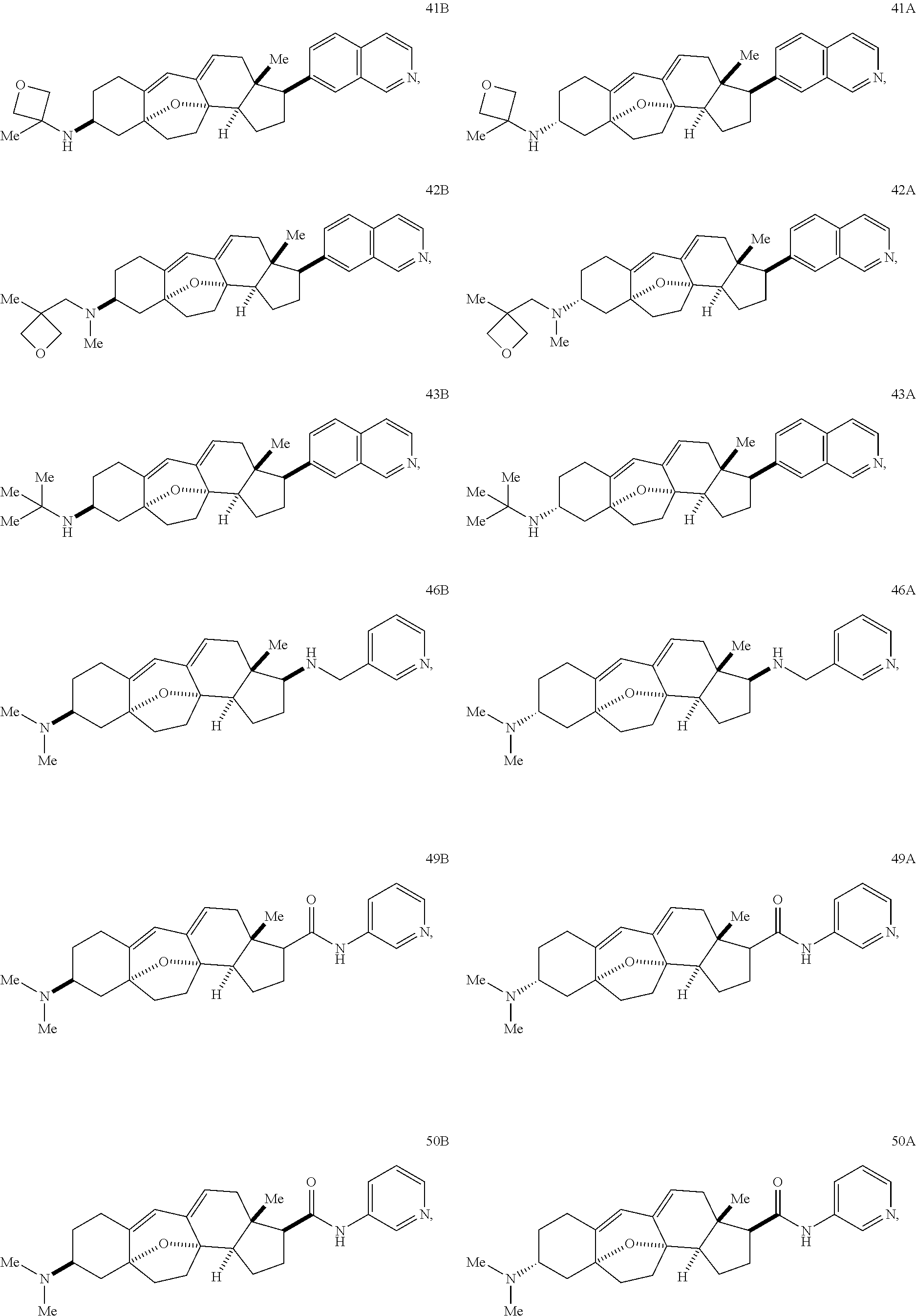

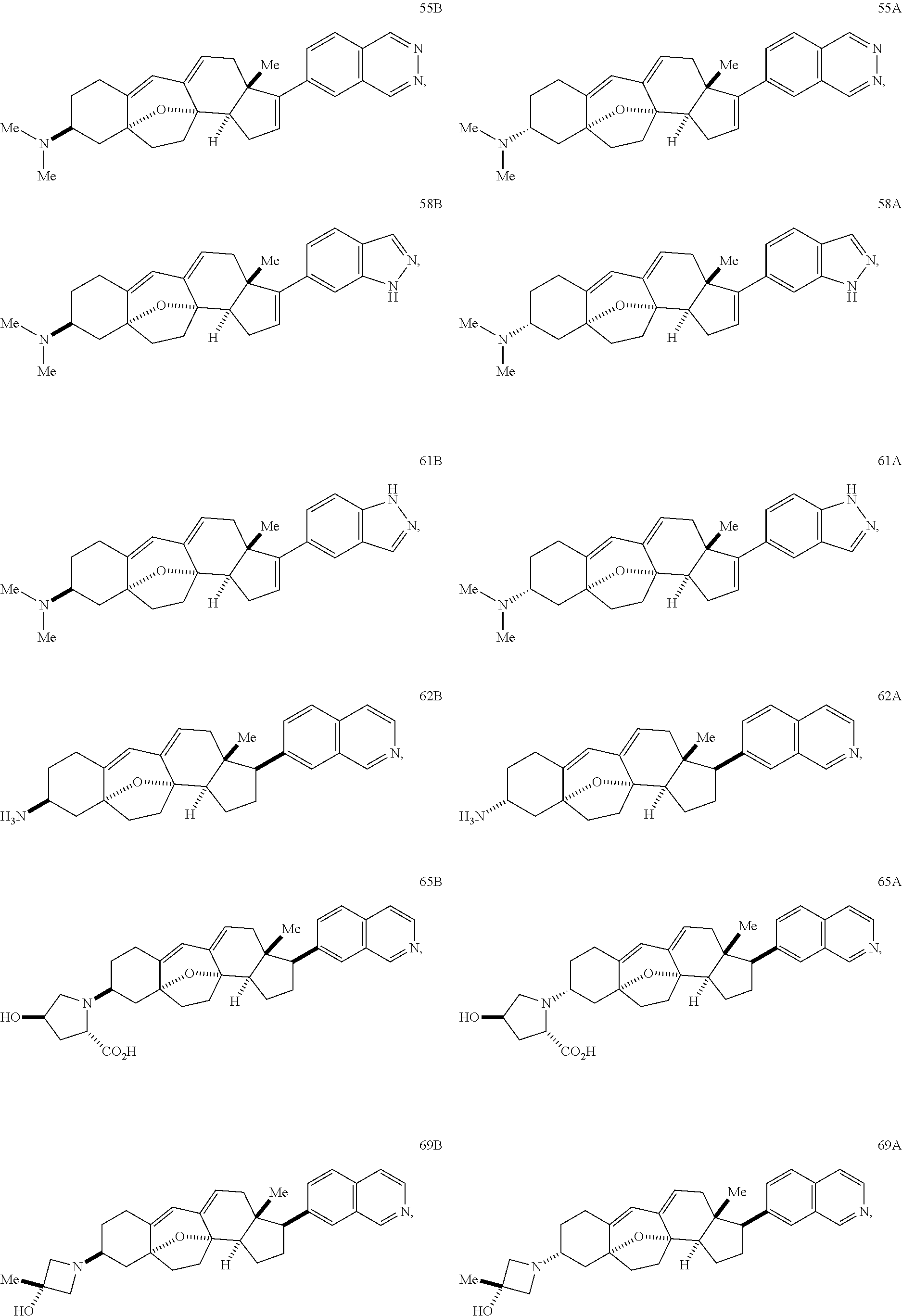

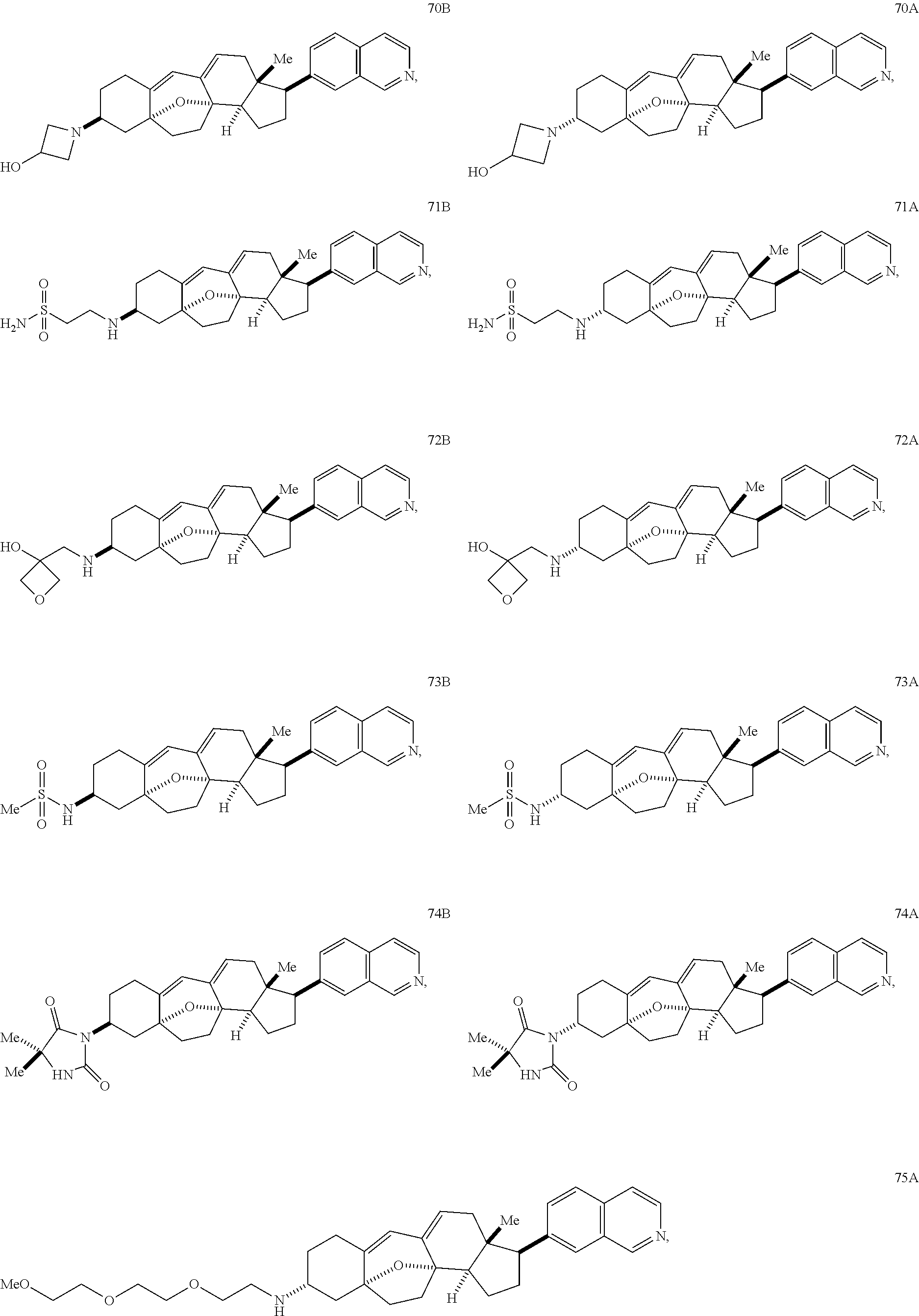

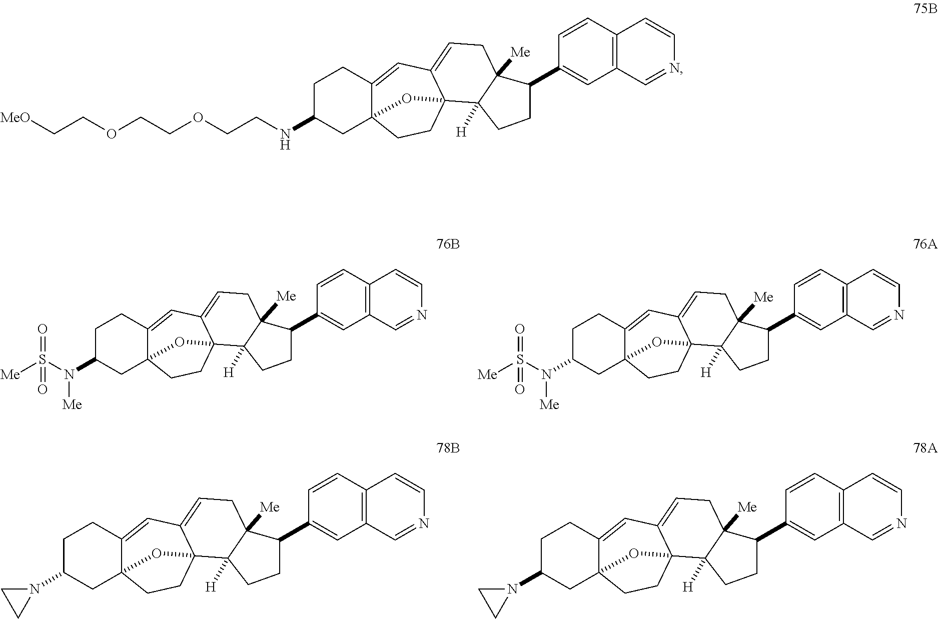

and pharmaceutically acceptable salts, quaternary amine salts thereof, and N-oxides thereof, e.g., N-oxides of formula:

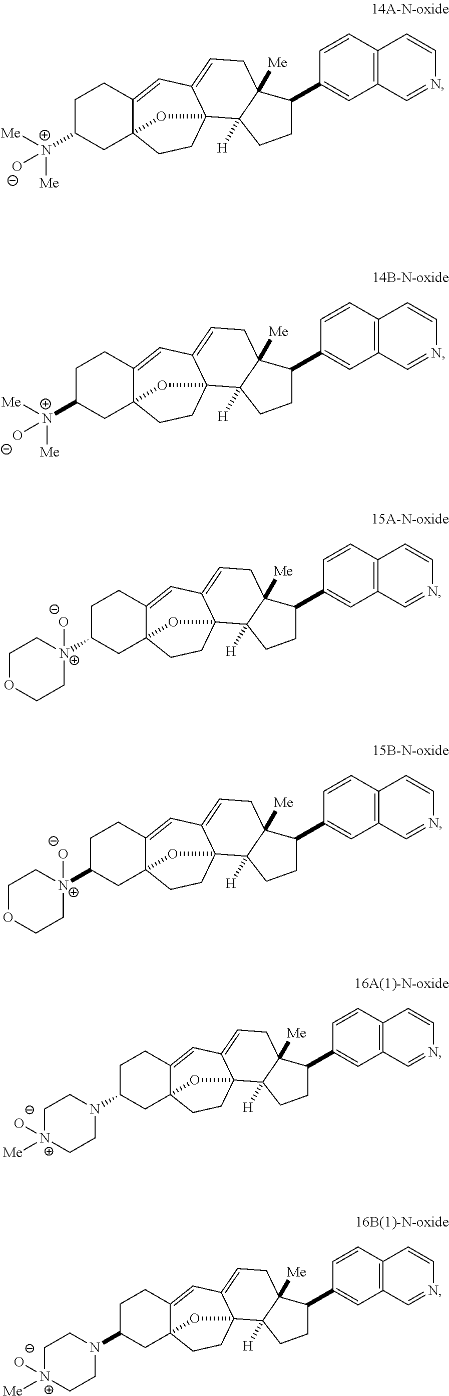

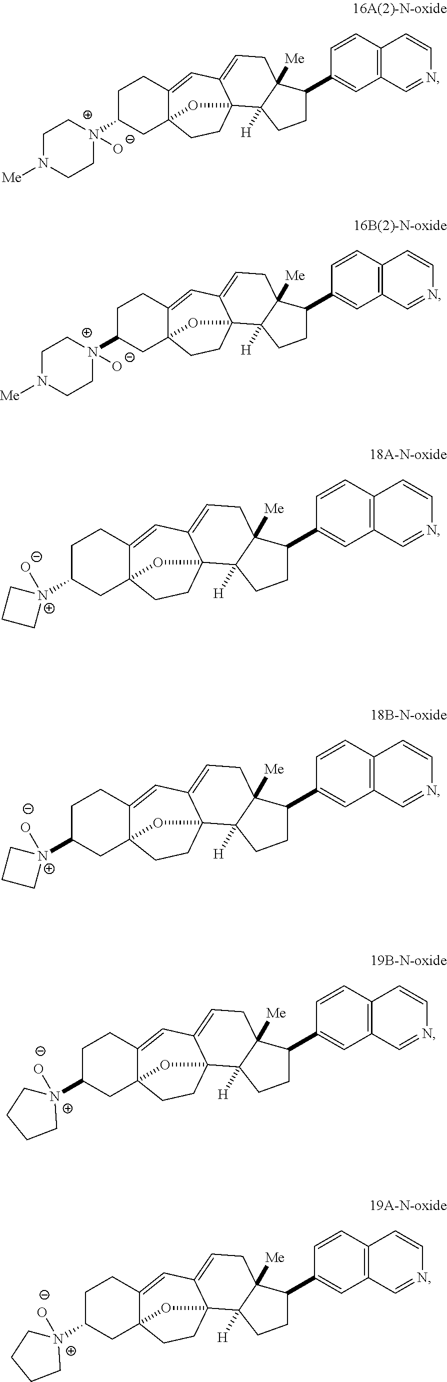

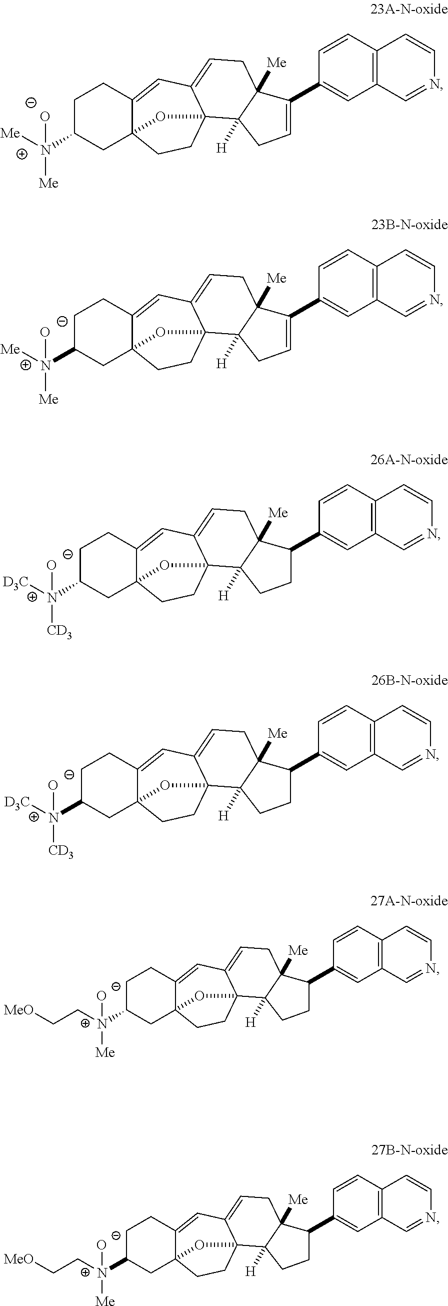

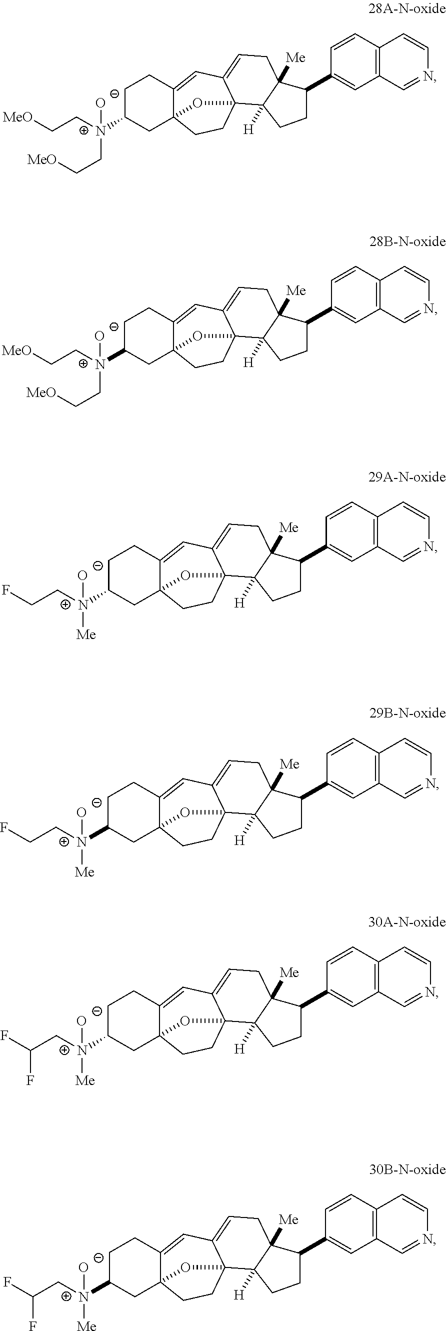

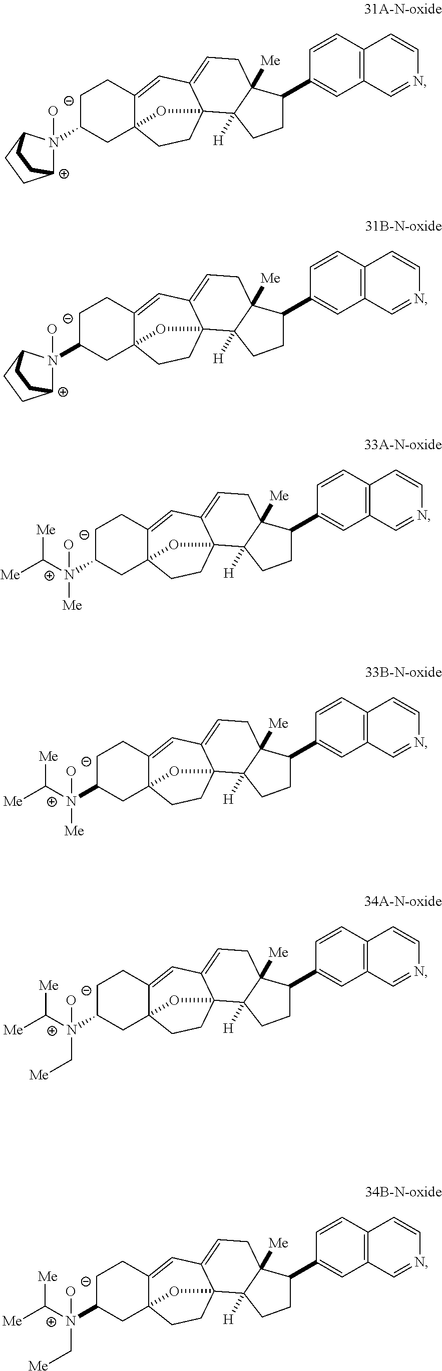

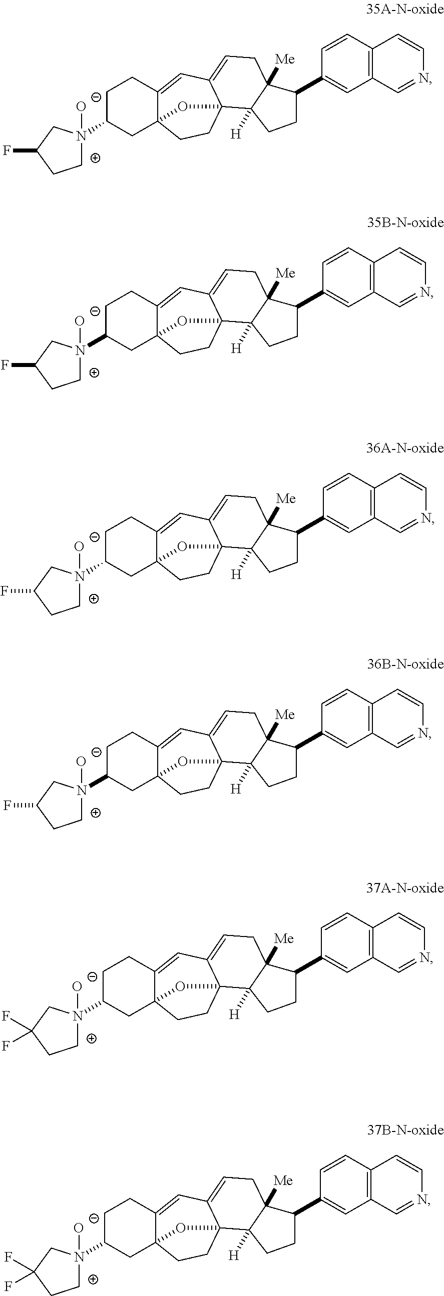

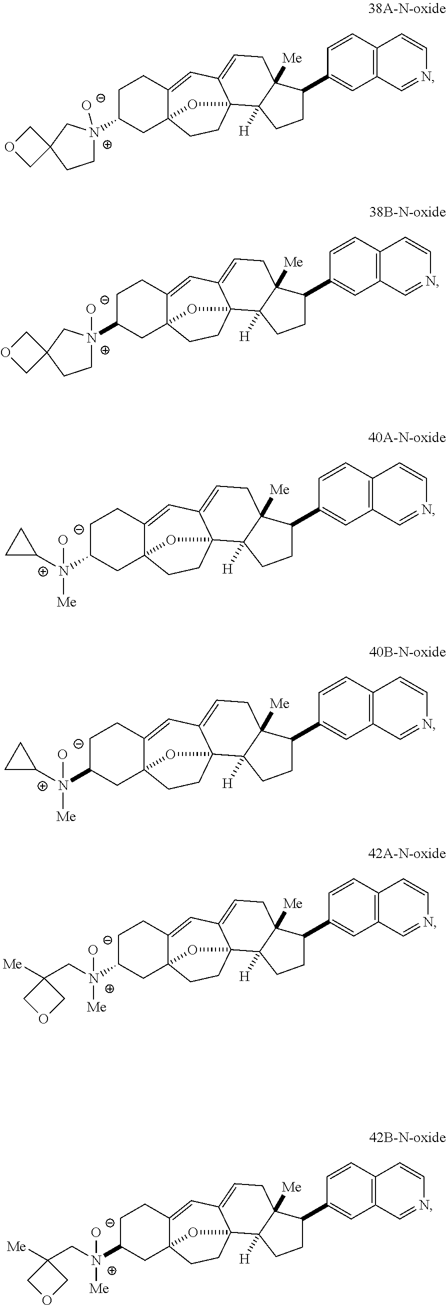

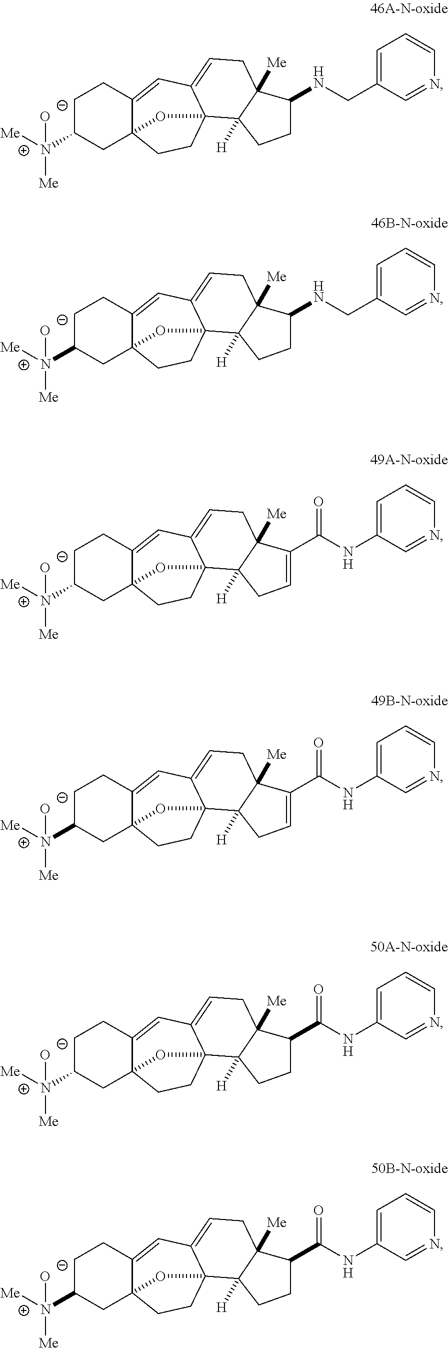

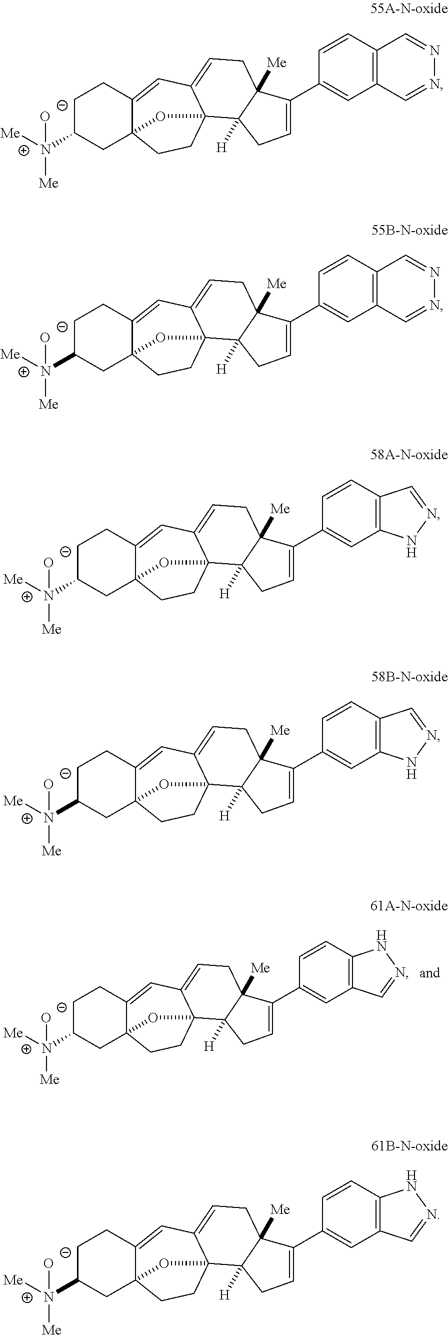

Exemplary compounds of Formula (A-1-C′) or (A-1-C″) include, but are not limited to:

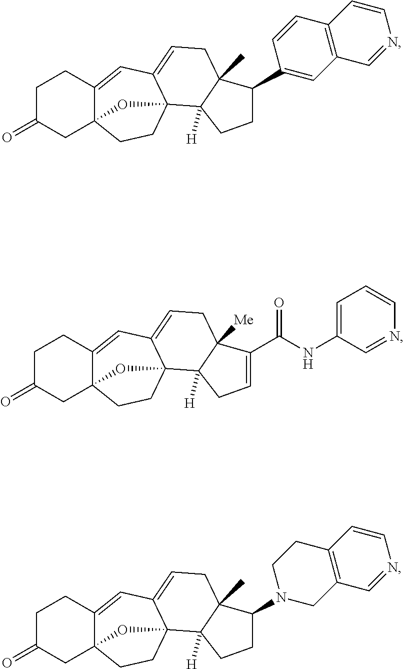 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CCC(=O)C[C@]45CC[C@]3(O5)[C@@H]1CC=C2C(=O)Nc1cccnc1 ; C[C@]12CC=C3C=C4CCC(=O)C[C@]45CC[C@]3(O5)[C@@H]1CC[C@@H]2N1CCc2ccncc2C1]

and pharmaceutically acceptable salts, quaternary amine salts, or N-oxides thereof.

Exemplary compounds of Formula (A-1-D′) or (A-1-D″) include, but are not limited to:

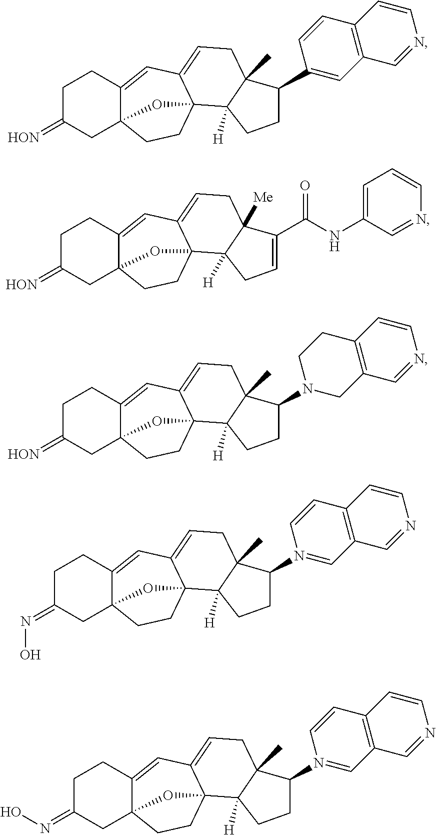

and pharmaceutically acceptable salts thereof.

Exemplary compounds of Formula (A-1-E′) or (A-1-E″) include, but are not limited to:

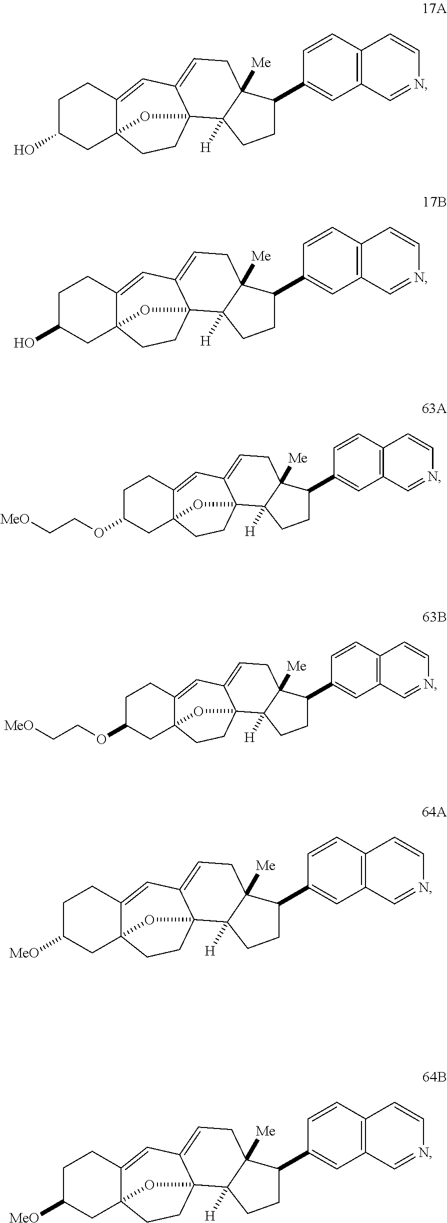

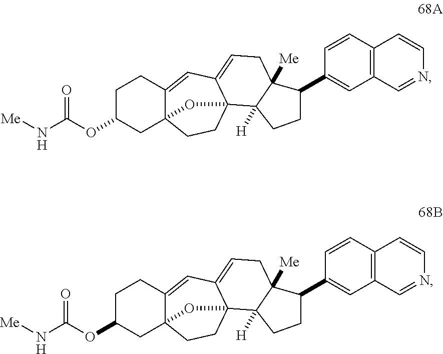

and pharmaceutically acceptable salts thereof.

Exemplary compounds of Formula (A-2′) or (A-2″) and (A-3′) or (A-3″) include, but are not limited to:

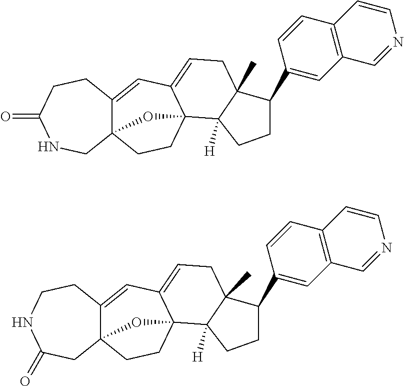 [SMILES: C[C@]12CC=C3C=C4CCC(=O)NC[C@]45CC[C@]3(O5)[C@@H]1CC[C@@H]2c1ccc2ccncc2c1 ; C[C@]12CC=C3C=C4CCNC(=O)C[C@]45CC[C@]3(O5)[C@@H]1CC[C@@H]2c1ccc2ccncc2c1]

and pharmaceutically acceptable salts thereof.

Exemplary compounds of Formula (D1′) or (D1″) include, but are not limited to:

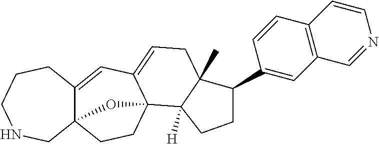 [SMILES: C[C@]12CC=C3C=C4CCCNC[C@]45CC[C@]3(O5)[C@@H]1CC[C@@H]2c1ccc2ccncc2c1]

and pharmaceutically acceptable salts thereof.

Exemplary compounds of Formula (D2′) or (D2″) include, but are not limited to:

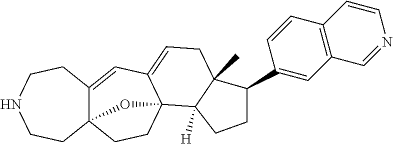 [SMILES: C[C@]12CC=C3C=C4CCNCC[C@]45CC[C@]3(O5)[C@@H]1CC[C@@H]2c1ccc2ccncc2c1]

and pharmaceutically acceptable salts thereof.

Exemplary compounds of Formula (E1′) or (E1″) include, but are not limited to:

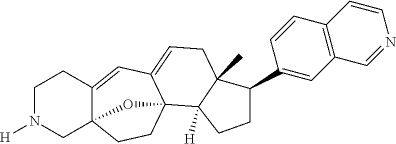 [SMILES: C[C@]12CC=C3C=C4CCNC[C@]45CC[C@]3(O5)[C@@H]1CC[C@@H]2c1ccc2ccncc2c1]

and pharmaceutically acceptable salts thereof.

Exemplary compounds of Formula (E2′) or (E2″) include, but are not limited to:

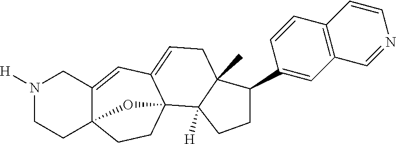 [SMILES: C[C@]12CC=C3C=C4CNCC[C@]45CC[C@]3(O5)[C@@H]1CC[C@@H]2c1ccc2ccncc2c1]

and pharmaceutically acceptable salts thereof.

B. Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. Also contemplated are stereoisomers featuring either a Z or E configuration, or mixture thereof, about a double bond. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures. The mixture may contain two enantiomers, two diastereomers, or a mixture of diastereomers and enantiomers.

If, for instance, a particular enantiomer of a compound described herein is desired, it may be prepared by asymmetric synthesis, or by derivation with a chiral auxiliary, where the resulting diastereomeric mixture is separated and the auxiliary group cleaved to provide the pure desired enantiomers. In some embodiments, a compound described herein is prepared by asymmetric synthesis with an enzyme. Enantiomers and diastereomers may be separated by means of fractional crystallization or chromatography (e.g., HPLC with a chiral column). Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

In some embodiments, the carbon to which R^{B1} or R^{B2} is attached is in the (S) configuration. In some embodiments, the carbon to which R^{B1} or R^{B2} is attached is in the (R) configuration. In some embodiments, the carbon to which R^{B1} or R^{B2} is attached is in the same configuration as a naturally occurring cortistatin (e.g., cortistatin A, cortistatin B). In some embodiments, the carbon to which R^{B1} or R^{B2} is attached is in the opposite configuration as a naturally occurring cortistatin (e.g., cortistatin A, cortistatin B). In some embodiments, the carbon to which Y¹ or Y² is attached is in the (S) configuration. In some embodiments, the carbon to which Y¹ or Y² is attached is in the (R) configuration. In some embodiments, the carbon to which Y¹ or Y² is attached is in the same configuration as a naturally occurring cortistatin (e.g., cortistatin A, cortistatin B). In some embodiments, the carbon to which Y¹ or Y² is attached is in the opposite configuration as a naturally occurring cortistatin (e.g., cortistatin A, cortistatin B). In some embodiments, the carbon to which R³ is attached is in the (S) configuration. In some embodiments, the carbon to which R³ is attached is in the (R) configuration. In some embodiments, the carbon to which R³ is attached is in the same configuration as a naturally occurring cortistatin (e.g., cortistatin A, cortistatin B). In some embodiments, the carbon to which R³ is attached is in the opposite configuration as a naturally occurring cortistatin (e.g., cortistatin A, cortistatin B). In some embodiments, the carbon to which R^{5B} is attached is in the (S) configuration. In some embodiments, the carbon to which R^{5B} is attached is in the (R) configuration. In some embodiments, the carbon to which R^{5B} is attached is in the same configuration as a naturally occurring cortistatin (e.g., cortistatin A, cortistatin B).

In some embodiments, the carbon to which R^{5B} is attached is in the opposite configuration as a naturally occurring cortistatin (e.g., cortistatin A, cortistatin B). In some embodiments, the carbon to which R^{5A} is attached is in the (S) configuration. In some embodiments, the carbon to which R^{5A} is attached is in the (R) configuration. In some embodiments, the carbon to which R^{5A} is attached is in the same configuration as a naturally occurring cortistatin (e.g., cortistatin A, cortistatin B). In some embodiments, the carbon to which R^{5A} is attached is in the opposite configuration as a naturally occurring cortistatin (e.g., cortistatin A, cortistatin B). In some embodiments, the carbon to which W is attached is in the (S) configuration. In some embodiments, the carbon to which W is attached is in the (R) configuration. In some embodiments, the carbon to which W is attached is in the same configuration as a naturally occurring cortistatin (e.g., cortistatin A, cortistatin B). In some embodiments, the carbon to which W is attached is in the opposite configuration as a naturally occurring cortistatin (e.g., cortistatin A, cortistatin B).

In some embodiments, the carbon to which R^{B1} is attached is in the (R) configuration. In some embodiments, R^{B1} comprises an isotopically enriched atom (e.g., ²H, ³H, ¹³C, ¹⁴C, ¹⁸F). In some embodiments, R^{B2} is deuterium. In some embodiments, R^{B2} comprises an isotopically enriched atom (e.g., ²H, ³H, ¹³C, ¹⁴C, ¹⁸F). In some embodiments, Y¹ is deuterium. In some embodiments, Y¹ comprises an isotopically enriched atom (e.g., ²H, ³H, ¹³C, ¹⁴C, ¹⁸F). In some embodiments, Y² is deuterium. In some embodiments, Y² comprises an isotopically enriched atom (e.g., ²H, ³H, ¹³C, ¹⁴C, ¹⁸F). In some embodiments, R³ is deuterium. In some embodiments, R³ comprises an isotopically enriched atom (e.g., ²H, ³H, ¹³C, ¹⁴C, ¹⁸F). In some embodiments, R⁴ is deuterium. In some embodiments, R⁴ comprises an isotopically enriched atom (e.g., ²H, ³H, ¹³C, ¹⁴C, ¹⁸F). In some embodiments, R^{5A} is deuterium. In some embodiments, R^{5A} comprises an isotopically enriched atom (e.g., ²H, ³H, ¹³C, ¹⁴C, ¹⁸F). In some embodiments, R^{5B} is deuterium. In some embodiments, R^{5B} comprises an isotopically enriched atom (e.g., ²H, ³H, ¹³C, ¹⁴C, ¹⁸F). In some embodiments, R^{N} is deuterium. In some embodiments, R^{N} comprises an isotopically enriched atom (e.g., ²H, ³H, ¹³C, ¹⁴C, ¹⁸F). In some embodiments, W comprises an isotopically enriched atom (e.g., ²H, ³H, ¹³C, ¹⁴C, ¹⁸F). In some embodiments, R^{O} is deuterium. In some embodiments, R^{O}comprises an isotopically enriched atom (e.g., ²H, ³H, ¹³C, ¹⁴C, ¹⁸F). In some embodiments, R¹ or R² is deuterium. In some embodiments, R¹ or R² comprises an isotopically enriched atom (e.g., ²H, ³H, ¹³C, ¹⁴C, ¹⁸F). In some embodiments, a hydrogen on ring A (see below) is substituted with deuterium. In some embodiments, a hydrogen on ring B is substituted with deuterium. In some embodiments, a hydrogen on ring C is substituted with deuterium. In some embodiments, a hydrogen on ring D is substituted with deuterium.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic,” as used herein, refers to alkyl, alkenyl, alkynyl, and carbocyclic groups. Likewise, the term “heteroaliphatic” as used herein, refers to heteroalkyl, heteroalkenyl, heteroalkynyl, and heterocyclic groups.

As used herein, “alkyl” refers to a radical of a straight-chain or branched saturated hydrocarbon group having from 1 to 10 carbon atoms (“C₁₋₁₀alkyl”). In some embodiments, an alkyl group has 1 to 9 carbon atoms (“C₁₋₉ alkyl”). In some embodiments, an alkyl group has 1 to 8 carbon atoms (“C₁₋₉ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀alkyl (e.g., —CH₃). In certain embodiments, the alkyl group is a substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

As used herein, “heteroalkyl” refers to an alkyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀alkyl. In certain embodiments, the heteroalkyl group is a substituted heteroC₁₋₁₀alkyl.

As used herein, “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₅), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “heteroalkenyl” refers to an alkenyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”).

In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

As used herein, “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “heteroalkynyl” refers to an alkynyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 9 ring carbon atoms (“C₃₋₉ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C7), bicyclo[2.2.2]octanyl (C₅), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C9), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-14 carbocyclyl. In certain embodiments, the carbocyclyl group is a substituted C₃-14 carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 9 ring carbon atoms (“C₃₋₉ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₅). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃-10 cycloalkyl. In certain embodiments, the cycloalkyl group is a substituted C₃-10 cycloalkyl.

As used herein, “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydro-benzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14□ electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆-14 aryl.

“Aralkyl” is a subset of “alkyl” and refers to an alkyl group, as defined herein, substituted by an aryl group, as defined herein, wherein the point of attachment is on the alkyl moiety.

As used herein, “heteroaryl” refers to a radical of a 5-14 membered monocyclic or polycyclic (e.g., bicyclic, tricyclic) 4n+2 aromatic ring system (e.g., having 6, 10, or 14 it electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as defined herein, substituted by a heteroaryl group, as defined herein, wherein the point of attachment is on the alkyl moiety.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂₀R^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —OP(═O)(R^{aa})₂, —OP(═O)(OR^{cc})₂, —NR^{bb}P(═O)(OR^{cc})₂, —P(R^{cc})₂, —OP(R^{cc})₂, —B(R^{aa})₂, —B(OR^{cc})₂, —BR^{aa}(OR^{cc}), C₁₋₁₀alkyl, C₁₋₁₀perhaloalkyl, C₂₋₁₀ alkenyl, C₂₋₁₀ alkynyl, heteroC₁₋₁₀alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂Ra, ═NR^{bb}, or ═NOR^{cc};
each instance of R^{aa} is, independently, selected from C₁₋₁₀alkyl, C₁₋₁₀perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}—P(═O)(R^{aa})₂, C₁₋₁₀ alkyl, C₁₋₁₀perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{dd} groups;
each instance of R^{cc} is, independently, selected from hydrogen, C₁₋₁₀alkyl, C₁₋₁₀perhaloalkyl, C₂₋₁₀ alkenyl, C₂₋₁₀ alkynyl, heteroC₁₋₁₀alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂-alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂-alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, an exemplary substituent is selected from the group consisting of halogen, —CN, —NO₂, —N₃, —SO₂H, —SO₃H, —OH, —OR^{aa}, —N(R^{bb})₂, —SH, —SR^{aa}, —SR^{cc}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, C₁₋₁₀alkyl, C₁₋₁₀perhaloalkyl, C₂₋₁₀ alkenyl, C₂₋₁₀ alkynyl, heteroC₁₋₁₀alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{dd} groups.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

As used herein, a “leaving group” is an art-understood term referring to a molecular fragment that departs with a pair of electrons in heterolytic bond cleavage, wherein the molecular fragment is an anion or neutral molecule. See, for example, Smith, March Advanced Organic Chemistry 6th ed. (501-502). Exemplary leaving groups include, but are not limited to, halo (e.g., chloro, bromo, iodo) and —OSO₂R^{aa}, wherein R^{aa} as defined herein. The group —OSO₂R^{aa} encompasses leaving groups such as tosyl, mesyl, and besyl, wherein R^{aa} is optionally substituted alkyl (e.g., —CH₃) or optionally substituted aryl (e.g., phenyl, tolyl).

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(═O)(R^{aa})₂, and —OP(═O)(OR^{cc})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

As used herein, the term, “amino” refers to the group —NH₂. The term “substituted amino,” by extension, refers to a monosubstituted amino or a disubstituted amino, as defined herein. In certain embodiments, the “substituted amino” is a monosubstituted amino or a disubstituted amino group.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, and —NHP(═O)(OR_{cc})₂, wherein R^{aa}, R^{bb} and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, and —NR^{bb}P(═O)(OR^{cc})₂, wherein R^{aa}, R^{bb}, and R^{cc} are as defined herein, with the proviso that the nitrogen atom directly attached to the parent molecule is not substituted with hydrogen.

As used herein, the term “sulfonyl” refers to a group selected from —SO₂N(R^{bb})₂, —SO₂R^{aa}, and —SO₂OR^{aa}, wherein R^{aa} and R^{bb} are as defined herein.

As used herein, the term “sulfinyl” refers to the group —S(═O)R^{aa}, wherein R^{aa} is as defined herein.

As used herein, the term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “silyl” refers to the group —Si(R^{aa})₃, wherein R^{aa} is as defined herein.

As used herein, the term “oxo” refers to the group ═O, and the term “thiooxo” refers to the group ═S.

Nitrogen atoms can be substituted or unsubstituted as valency permits, and include primary, secondary, tertiary, and quarternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(R^{aa})₂, C₁₋₁₀ alkyl, C₁₋₁₀perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5, R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀alkenyl, C₂₋₁₀ alkynyl, heteroC₁₋₁₀alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5, R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(0-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methyl sulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, β-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethyl silylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethyl silyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(═O)(R^{aa})₂, and —P(═O)(OR^{cc})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethyl silyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethyl silylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(═O)(R^{aa})₂, and —P(═O)(OR^{cc})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The invention is not intended to be limited in any manner by the above exemplary listing of substituents.

C. Exemplary Syntheses of Cortistatin Analogs

The synthesis initially is contemplated using a compound of Formula (I) as starting material. Oxidation (e.g., DDQ, MnO2) of estrone (wherein R³ is —CH₃) or norestrone (wherein R³ is H) (I) provides the compound of Formula (III). See, e.g., Stephan et al., Steroid, 1995, 60, 809-811. The compound of Formula (III) is protected as an acetal or ketal (e.g., via reaction with HX^{A}R^{A}, or HX^{A}R^{A}—R^{A}X^{A}H, wherein the two R^{A} groups are joined, wherein R^{B1} and R^{B2} are each independently —X^{A}R^{A}) to give a mixture (e.g., 1:1 mixture) of (IV)-A and (IV)-B. Exemplary conditions contemplated for protection include PTSA and ethylene glycol, PTSA and CH(OMe)₃, PTSA and CH(OEt)₃, and PTSA and 2,2-dimethyl-1,3-propandiol). The protected compounds are then alkylated (e.g., methylated) using an alkylating agent (e.g., Me₂SO₄ and K₂CO₃, EtN(i-Pr)₂ and TMS-diazomethane) to afford (V)-A and (V)-B, wherein E is optionally substituted alkyl. See Scheme 5.

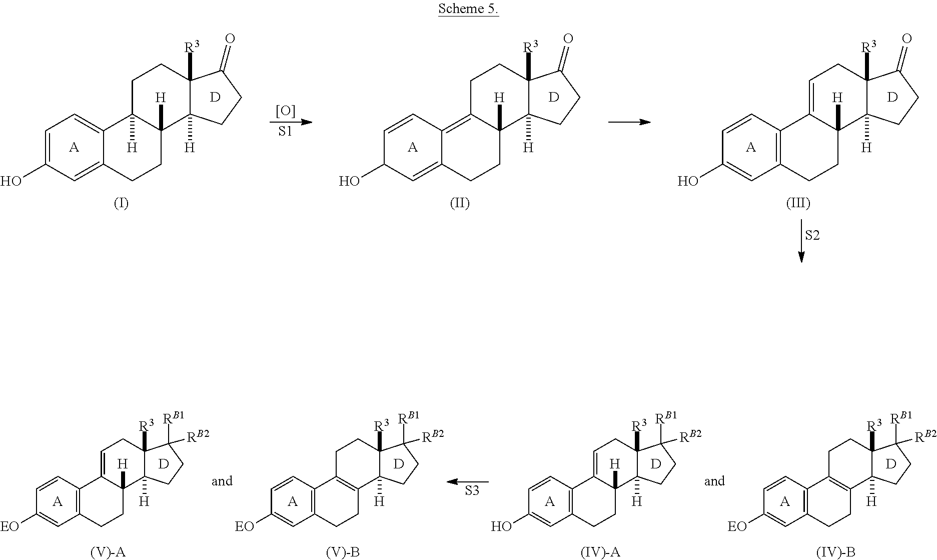

Scheme 6 provides other exemplary routes to provide a compound of Formula (IV-B), e.g., wherein R³ is —CH₃. For example, the compound of Formula (V)-B is achieved as racemic mixtures from 6-methoxy-1-tetralone in four steps as described in Scheme 6(A). For the Grignard reaction, see, e.g., Saraber et al., Tetrahedron, 2006, 62, 1726-1742. For hydrogenation, see, e.g., Sugahara et al., Tetrahedron Lett, 1996, 37, 7403-7406. Scheme 6(B) shows method to obtain enantiopure Torgov's intermediate by chiral resolution. See, e.g., Bucourt et al., J. Bull. Soc. Chim. Fr. (1967) 561-563. Scheme 6(C) provides another method of preparing enantiopure Torgov's intermediate aided by enzymatic reduction. See, e.g., Gibian et al., Tetrahedron Lett. (1966) 7:2321-2330.

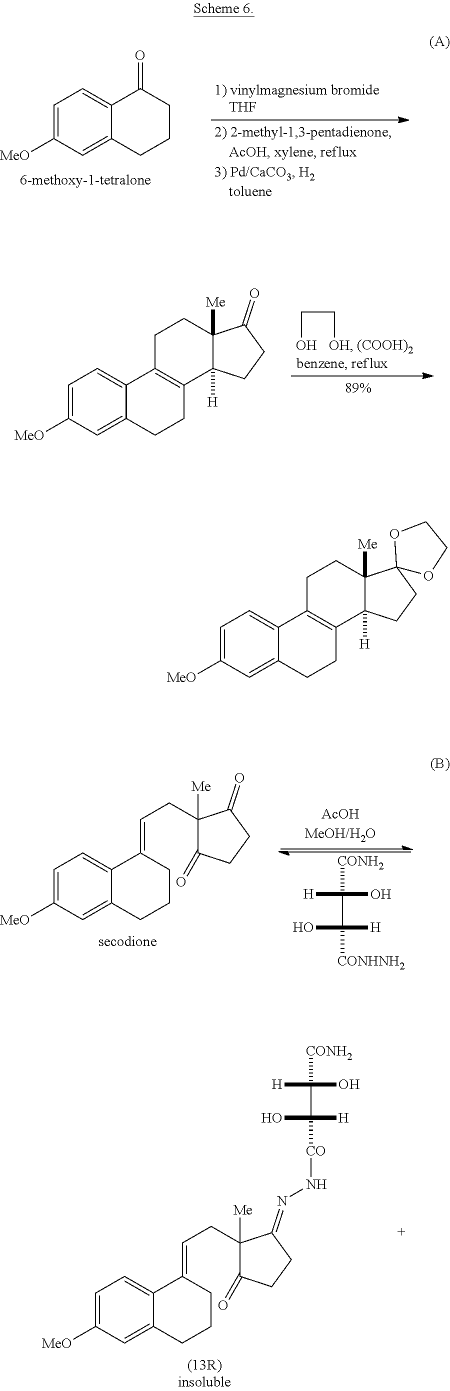

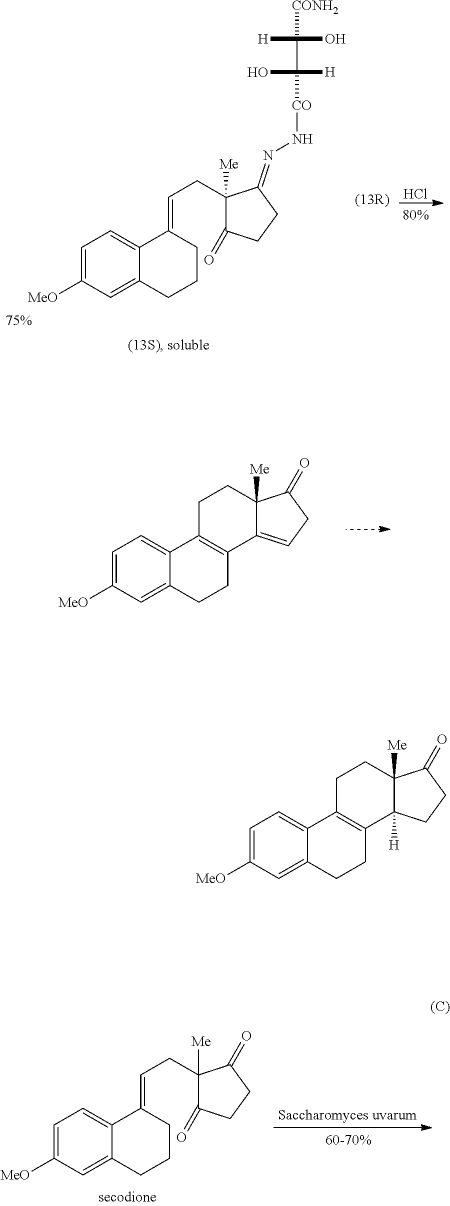 [SMILES: COc1ccc2c(c1)CCC/C2=C\C[C@]1(C)C(=O)CC/C1=N\NC(=O)[C@H](O)[C@@H](O)C(N)=O ; Cl ; COc1ccc2c(c1)CCC1=C2CC[C@]2(C)C(=O)CC=C12 ; COc1ccc2c(c1)CCC1=C2CC[C@]2(C)C(=O)CC[C@@H]12 ; COc1ccc2c(c1)CCC/C2=C\CC1(C)C(=O)CCC1=O]

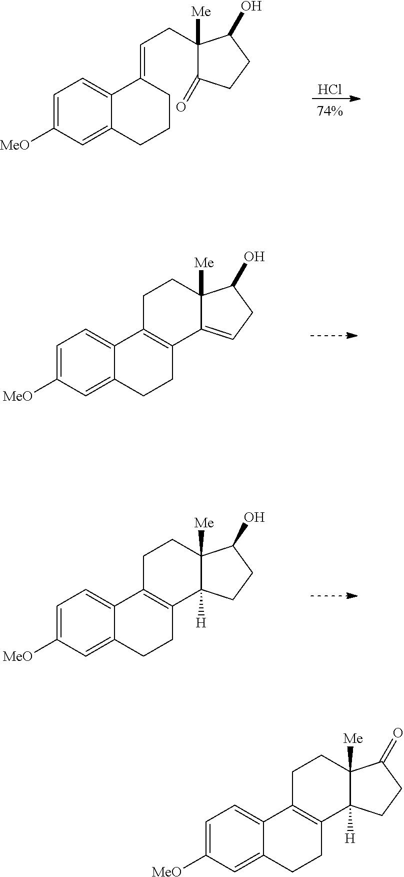 [SMILES: COc1ccc2c(c1)CCC/C2=C\C[C@@]1(C)C(=O)CC[C@@H]1O ; Cl ; COc1ccc2c(c1)CCC1=C2CC[C@@]2(C)C1=CC[C@@H]2O ; COc1ccc2c(c1)CCC1=C2CC[C@]2(C)[C@@H](O)CC[C@@H]12 ; COc1ccc2c(c1)CCC1=C2CC[C@]2(C)C(=O)CC[C@@H]12]

With compounds of Formula (IV-A) and (IV-B) in hand, epoxidation/epoxide opening/epoxidation reactions are conducted (e.g., MMPP, mCPBA) in one-pot to provide the compound of Formula (IX-A) and (IX-B), which are under equilibrium with (IX-A) as a major compound. See Schemes 7A and 7B.

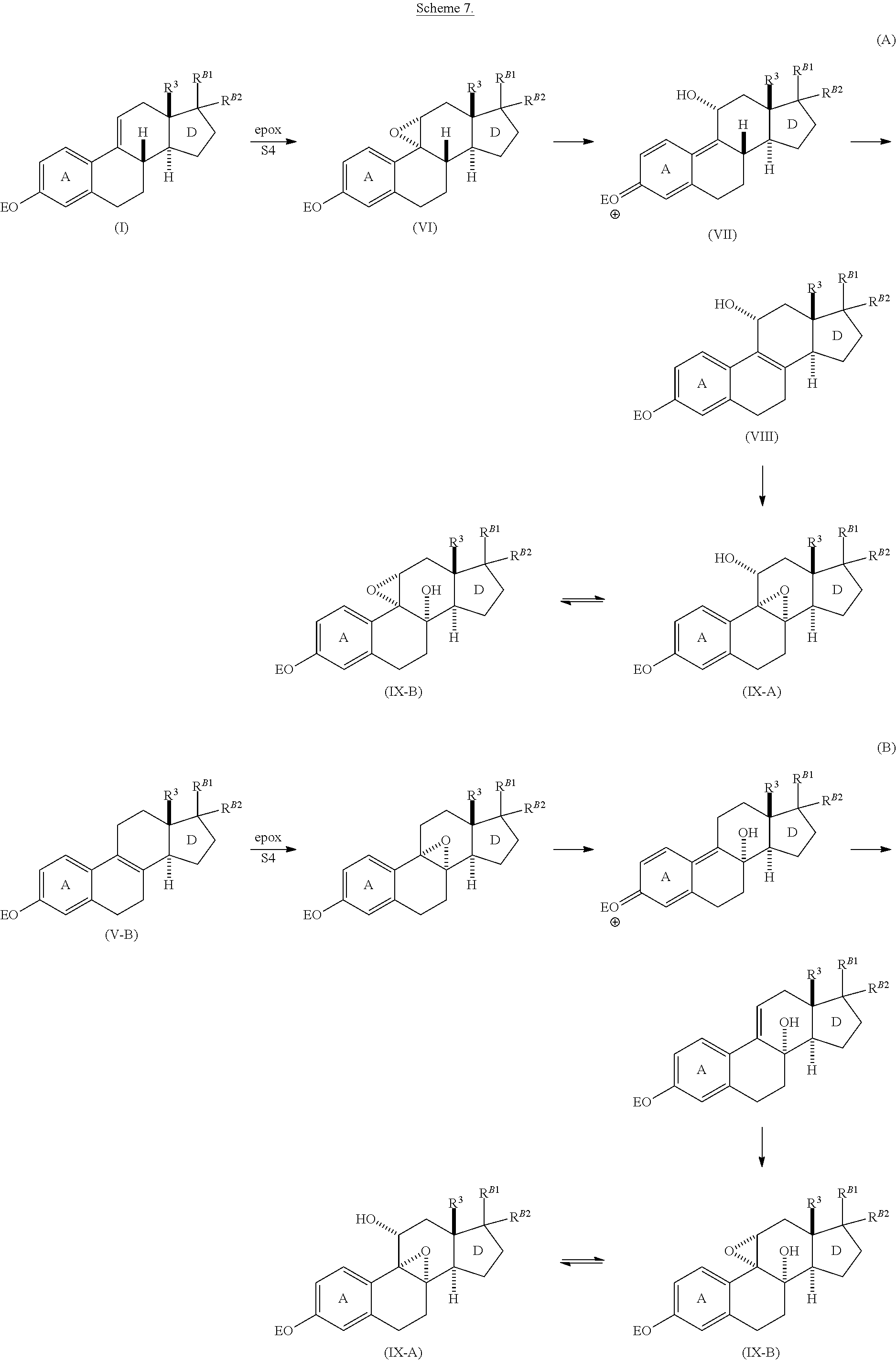

The compound of Formula (IX-A) and (IX-B) are exposed to Birch reduction condition (e.g., Li/NH₃ and t-BuOH, Na/NH₃ and t-BuOH) to give dearomatized compound (X). C3 of A-ring is then protected as an acetal or ketal (e.g., via reaction with HX^{A}R^{A}, or HX^{A}R^{A}—R^{A}X^{A}H, wherein the two R^{A} groups are joined, and wherein R^{B1} and R^{B2} are each independently —X^{A}R^{A}) to afford the compound (XI). Exemplary protection conditions include PTSA and ethylene glycol, PTSA and CH(OMe)₃, PTSA and CH(OEt)₃, and PTSA and 2,2-dimethyl-1,3-propandiol. See Scheme 8.

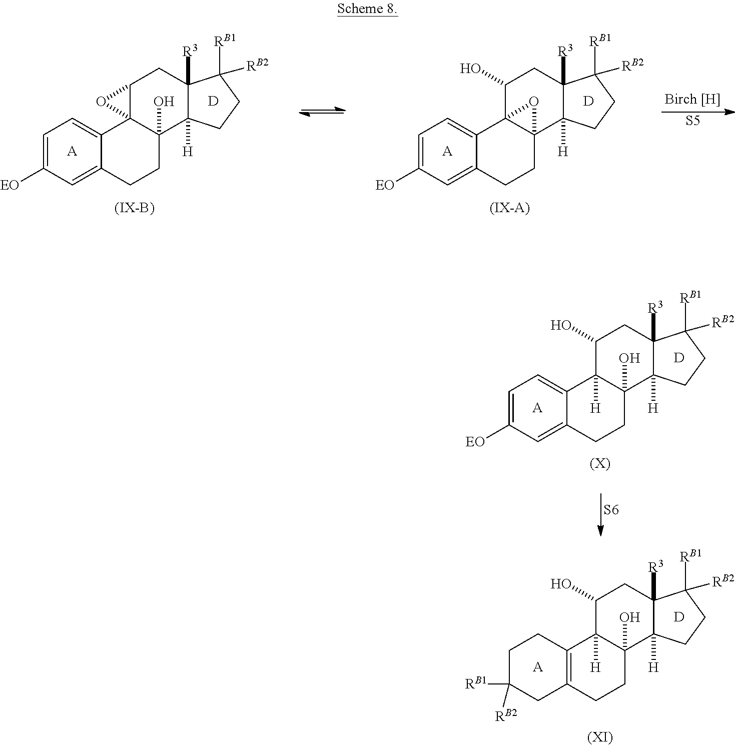

The compound (XI) is converted to a compound of Formula (XIII) through etherification (e.g., NBS, NIS, e.g., wherein X is Br or I). This compound is then oxidized (e.g., SO₃:Py/DMSO and triethylamine, IBX, (COCl)₂/DMSO and triethylamine) to provide the compound of Formula (XIV). This compound is then treated with base (e.g., DBU, triethylamine) to provide the compound of Formula (XV). This compound is then reduced (e.g., NaBH₄ and CeCl₃, L-selectride) to provide the compound of Formula (XVI). See Scheme 9.

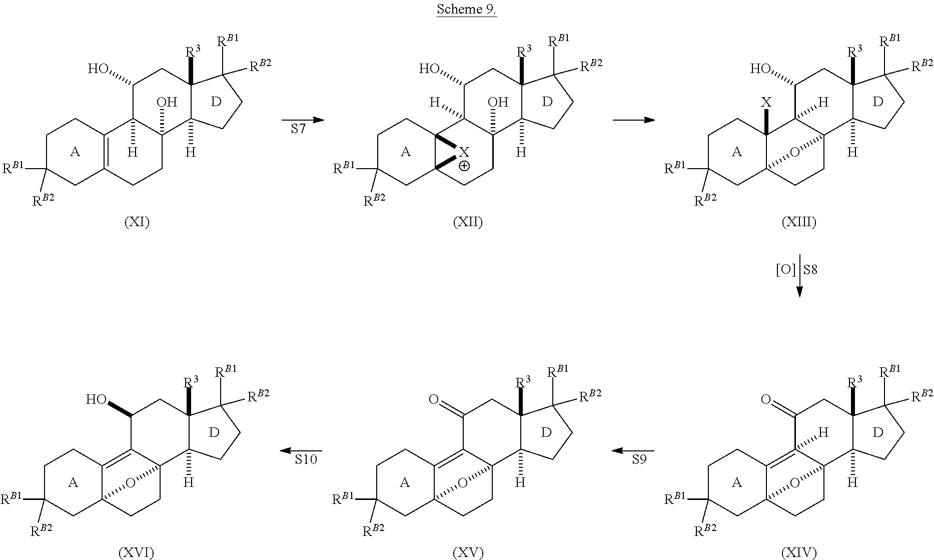

The compound of Formula (XVI) is then treated with cyclopropanation reagents (e.g., ZnEt₂ and ClCH₂I, ZnEt₂ and CH₂I₂, Zn—Cu and CH₂I₂) to provide a compound of Formula (XVII). The alcohol of the cyclopropanated product is activated, wherein LG¹ is a sulfonyl (e.g., the alcohol is treated with Tf₂O, MsCl, to provide an activated alcohol wherein LG¹ is Tf or Ms) and treated with base (e.g., 2,6-di-t-butyl-4-methylpyridine, 2,6-lutidine, triethylamine) to provide the compound of Formula (XX). See, e.g., Magnus et al., Org. Lett. 2009, 11, 3938-3941. See Scheme 10.

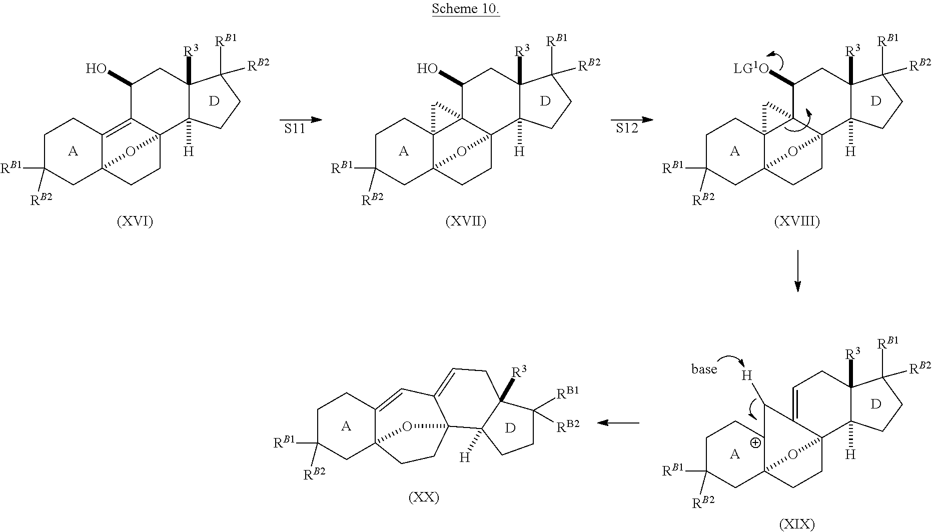

Protecting group on D-ring of the compound of Formula (XX) is then deprotected under acidic conditions (e.g., PTSA and acetone/water, TFA/water) to provide the ketone intermediate of Formula (XXI). This product is treated with a compound of Formula R^{B1}-M (e.g., R^{B1}—CeCl₂, R^{B1}—Mg) which is prepared from R^{B1}—X (e.g., R^{B1}—Br, R^{B1}—I) to provide a compound of Formula (XXII), whereinR^{B1} is a non-hydrogen group as defined herein. The compound of Formula (XXII) is activated (e.g., TFAA and pyridine, PhNCS and KH) to provide a compound of Formula (XXIII). Reduction of the compound of Formula (XXIII) (e.g., AIBN and Bu3SnH) provides the compound of Formula (XXIV). For steps S14, S15 and S16, see, e.g., Flyer et al., Nature. Chem. 2010, 2, 886-892, and Yamashita et al., J. Org. Chem. 2011, 76, 2408-2425. See Scheme 11A.

Compound (XXIV) may also be prepared from (XX) through conversion to an activated alcohol, wherein LG² is a sulfonyl (e.g., the alcohol is treated with Tf₂O, MsCl, to provide an activated alcohol wherein LG² is Tf or Ms; by triflation, e.g., KHMDS and PhNTf₂, LiHMDS and PhNTf₂, Tf₂O and 2,6-di-t-butyl-4-methylpyridine) followed by palladium-catalyzed cross coupling with R^{B1}-M, wherein M is a substituted boron (e.g., such as —B(R′)₂, wherein each R′ is —OR″ or alkyl wherein the alkyl and R″ is alkyl or may be joined to form a ring) to provide the compound of Formula (XXVI). Exemplary palladium-catalyzed cross coupling conditions include, but are not limited to, R^{B1}—B(pin), R^{B1}-(9-BBN—H), R^{B1}-OBBD, or R^{B1}—B(cat), and Pd(PPh₃)₄ and Na₂CO₃, or Pd(dppf)Cl₂ and K₃PO₄) (pin=pinacol; cat=catechol; OBBD=9-oxa-10-brabicyclo[3.3.2]decane; 9-BBN—H=9-broabicyclo[3.3.1]nonane). See, e.g., Nicolaou et al., J. Am. Chem. Soc. 2009, 131, 10587-10597. Hydrogenation of C16-C17 double bond (e.g., Pd/C and H₂, Raney Ni and H₂) gives the compound of Formula (XXIV). See Scheme 11B.

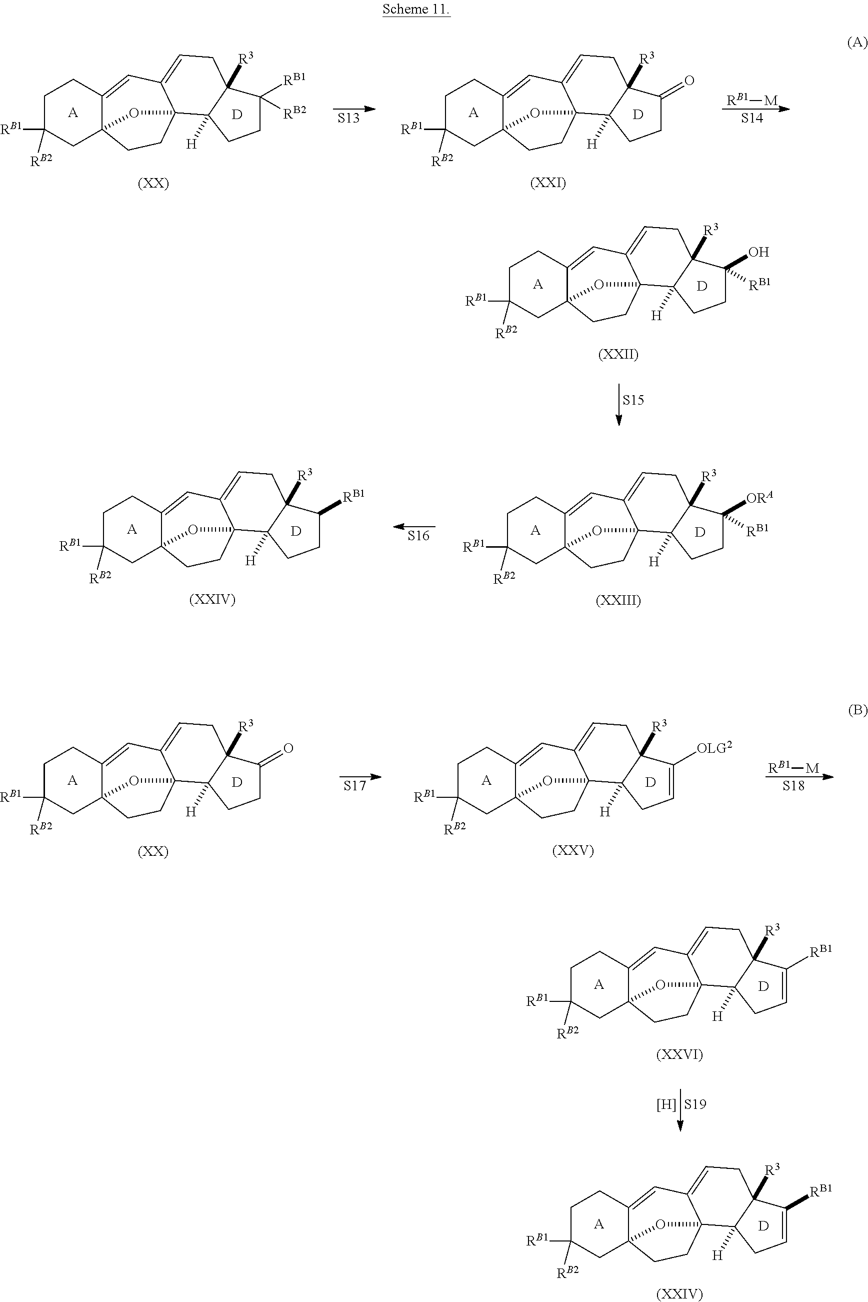

Any one of the compounds of Formula (XXVI) or (XXIV) may then be deprotected (e.g., PTSA and acetone/water, TFA/water, HCl) and the resulting ketone may be trapped as the enolate, followed by subsequent oxidation or amination of the double bond, or reaction of the double bond with an electrophilic carbon C(R^{A})₃-LG, wherein LG is a leaving group, to provide a substituted ketone product, wherein R⁵ is —OR^{A}, —OC(═O)R^{A}, —OC(═O)OR^{A}, —OC(═O)N(R^{A})₂, —OS(═O)₂R^{A}, —N₃, —N(R^{A})₂, —NR^{A}C(═O)R^{A}, —NR^{A}C(═O)OR^{A}, —NR^{A}C(═O)N(R^{A})₂, —NR^{A}S(═O)₂R^{A}, or —C (R^{A})₃. See Schemes 12A and 12B. Exemplary conditions contemplated for enolate trapping include a combination of a base (e.g., lithium diisopropyl amide (LDA)) and a trapping reagent P1-LG, wherein P₁ is silyl and LG is a leaving group (e.g., such as trimethylsilyl chloride).

Exemplary oxidative conditions, e.g., to install a —OR^{A}, —OC(═O)R^{A}, —OC(═O)OR^{A}, —OC(═O)N(R^{A})₂, or —OS(═O)₂R^{A} group at the R⁵ position include treating the trapped enolate with an oxidant, such as meta-chloroperoxybenzoic acid (MCPBA), MoOOPh, or DMSO, to provide a substituted ketone wherein R⁵ is —OH, followed by optional protection, e.g., via treatment of the compound wherein R⁵ is —OH with a compound of formula R^{A}-LG, LG-C(═O)R^{A}, LG-C(═O)OR^{A}, LG-C(═O)N(R^{A})₂, or LG-S(═O)₂R^{A}, wherein LG is a leaving group, to provide a compound wherein R⁵ is —OR^{A} (wherein R^{A} is a non-hydrogen group), —OC(═O)R^{A}, —OC(═O)OR^{A}, —OC(═O)N(R^{A})₂, or —OS(═O)₂R^{A}.

Exemplary aminating conditions, e.g., to install an —N₃, —N(R^{A})₂, —NR^{A}C(═O)R^{A}, —NR^{A}C(═O)OR^{A}, —NR^{A}C(═O)N(R^{A})₂, or —NR^{A}S(═O)₂R^{A} group at the R⁵ position include treating the trapped enolate with a compound N₃-LG wherein LG is a leaving group (e.g., such as trisylazide) to provide substituted ketone wherein R⁵ is —N₃. The substituted ketone wherein R⁵ is —N₃ may be treated with a reducing agent (e.g., such as PPh₃) to provide a compound wherein R⁵ is —NH₂, followed by optional protection, e.g., via treatment of the compound wherein R⁵ is —NH₂ with a compound of formula R^{A}-LG, LG-C(═O)R^{A}, LG-C(═O)OR^{A}, LG-C(═O)N(R^{A})₂, or LG-S(═O)₂R^{A}, wherein LG is a leaving group, to provide a compound wherein R⁵ is —N(R^{A})₂ (wherein at least one of R^{A} is a non-hydrogen group), —NR^{A}C(═O)R^{A}, —NR^{A}C(═O)OR^{A}, —NR^{A}C(═O)N(R^{A})₂, or —NR^{A}S(═O)₂R^{A}.

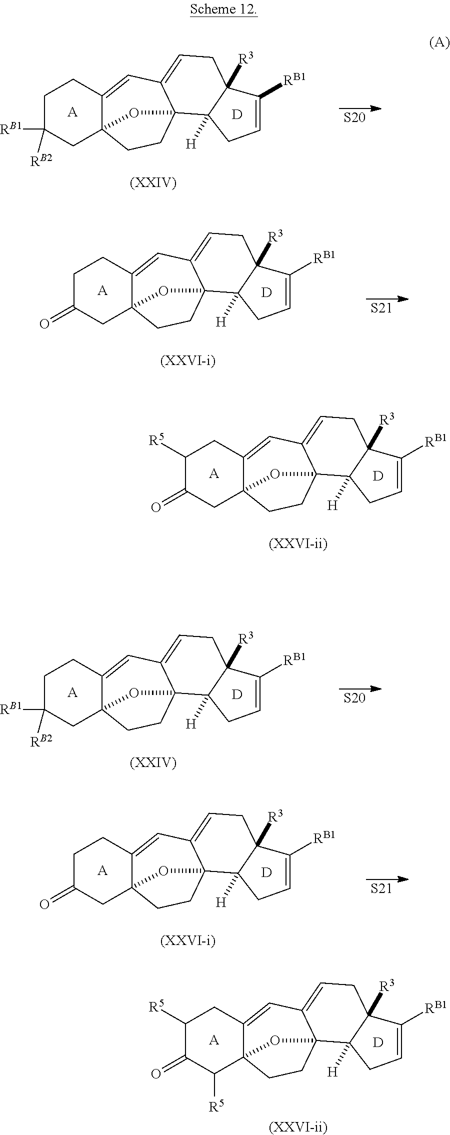

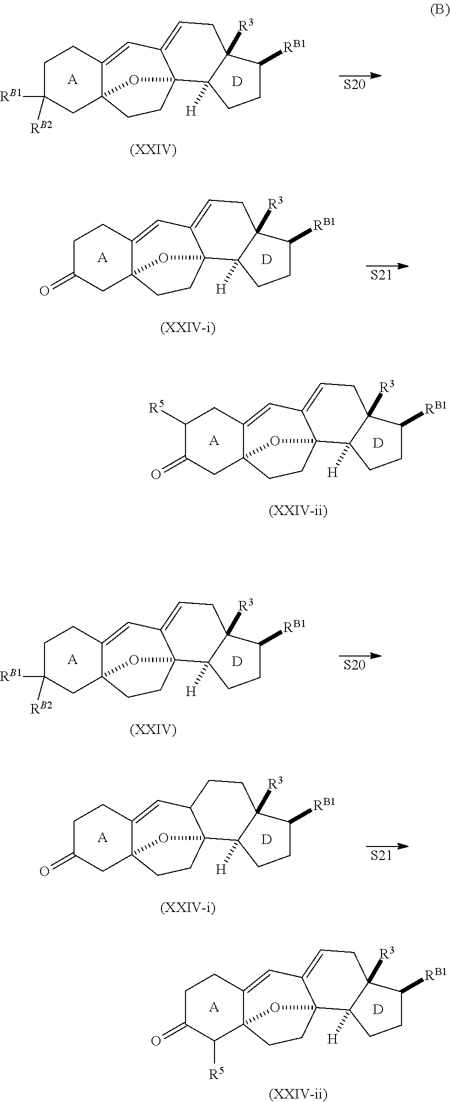

The ketone compounds as provided in Scheme 12(A) and 12(B) can then be treated with an amine of formula H₂NR¹ to form the condensation products, imines, as depicted in Step S22. The ketone compounds can also be treated with an amine of formula HNR¹R², or salt thereof, under reductive amination conditions to provide the aminated products, as depicted in Step S23. Exemplary reductive amination conditions include, but are not limited to, NaCNBH₃, NaCN(9BBN)H, or NaBH(OAc)₃ under acidic pH (e.g., pH of 3). The aminated products can further be oxidized to the corresponding N-oxide, as depicted in Step S25. Exemplary oxidizing conditions include, but are not limited to, H₂O₂, mCPBA, or DMDO. See Schemes 13A to 13D.

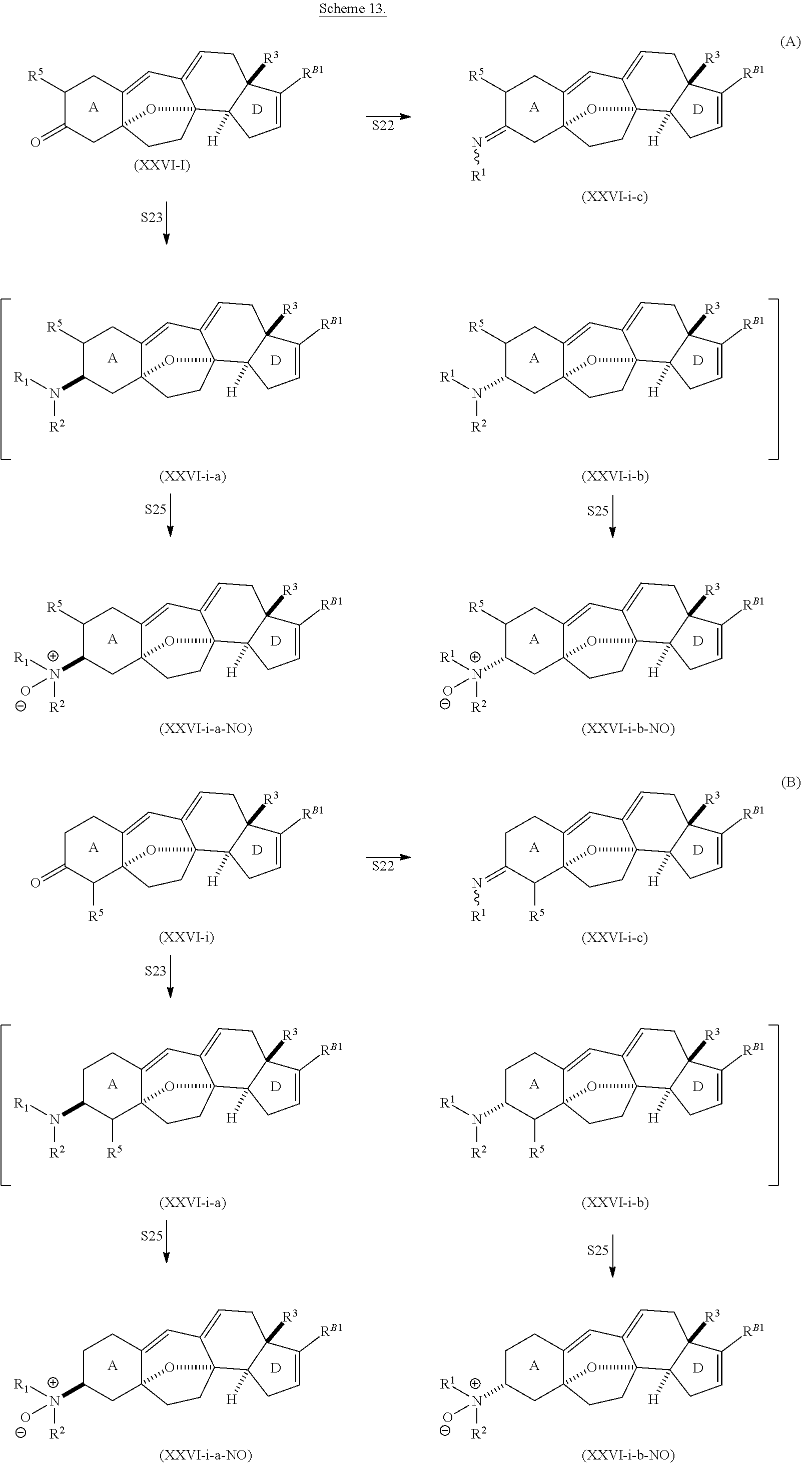

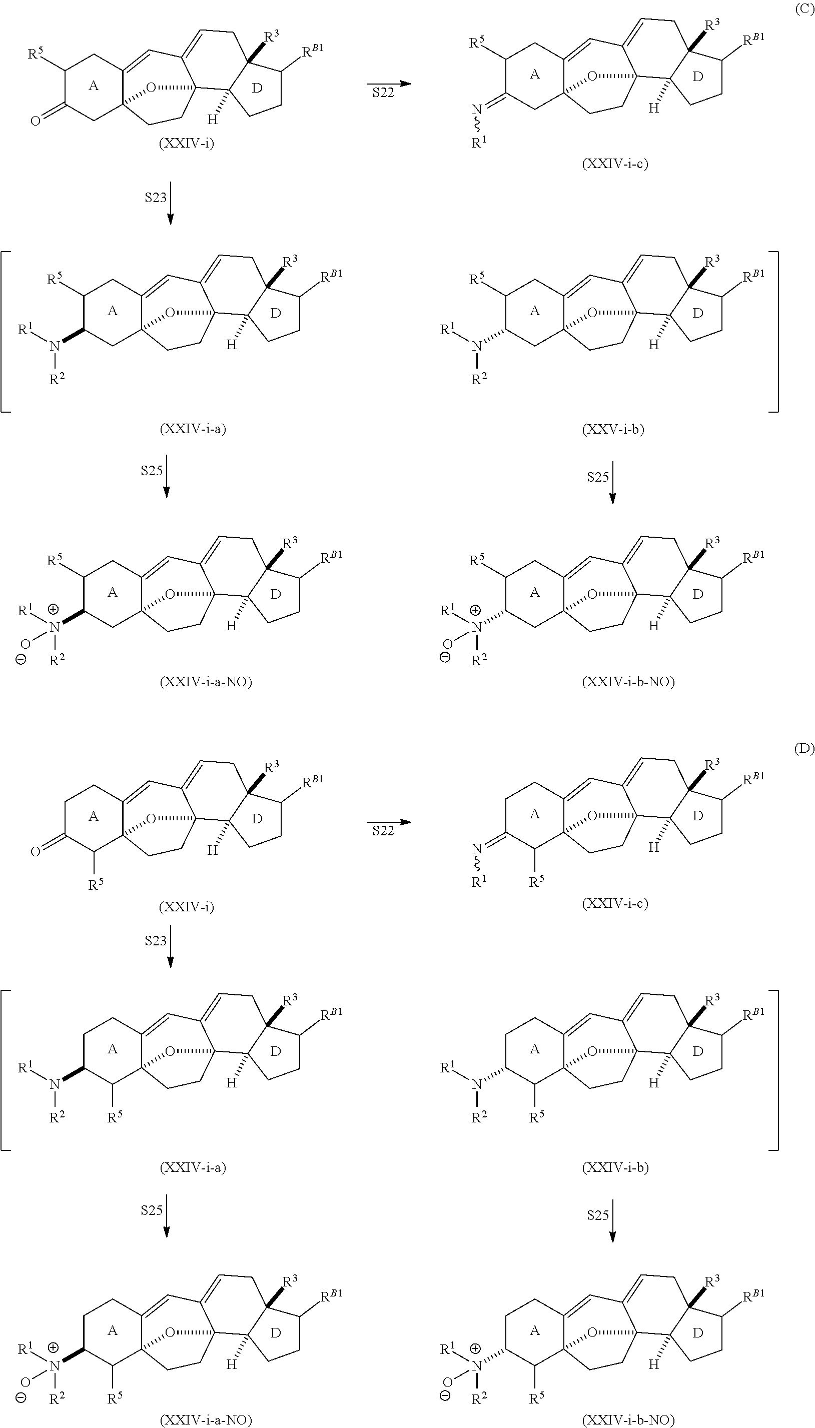

The keto compound can also be converted to the compound of Formula (XXV-i) through palladium-catalyzed carbonylative amination with CO and HN(R^{L})R^{B3} (e.g., Pd(PPh₃)₄ and triethylamine, Pd(dppf)Cl₂ and triethylamine). Conditions for the following steps to get to the compound of Formula (XXV-i), (XXV-iv), and (XXV-v) are the same as described previously. See Scheme 14.

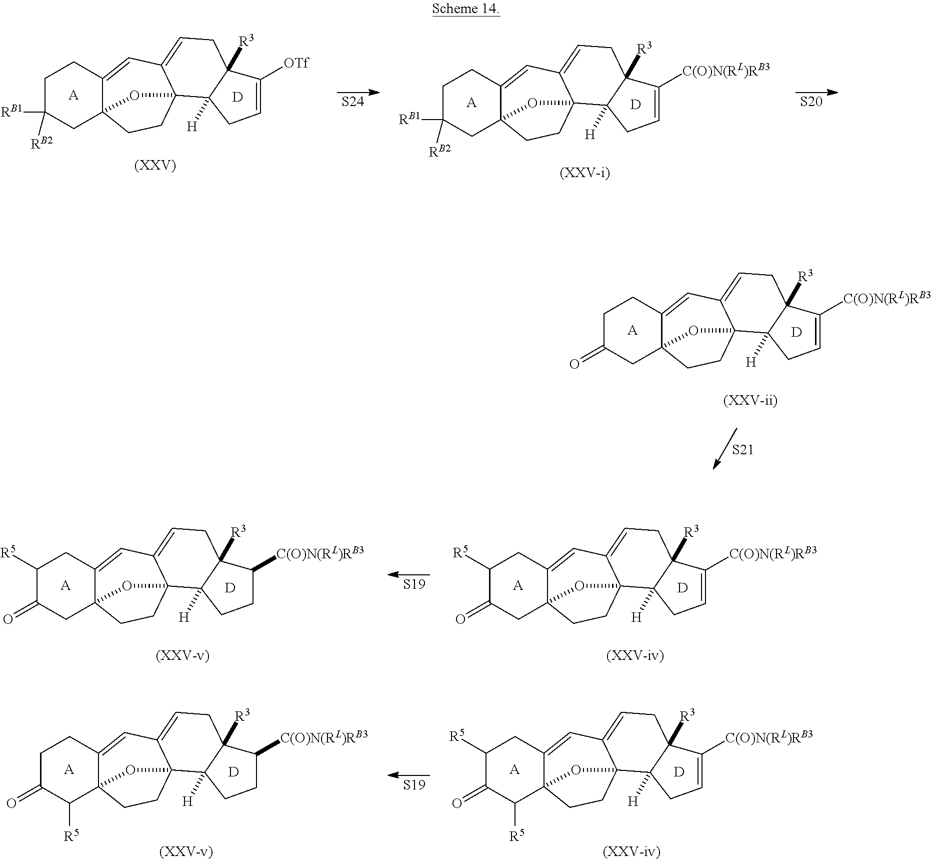

The ketone compounds as provided in Scheme 14 can then be converted to the corresponding imines, amines, and N-oxides, as described previously. See Scheme 15A and 15B.

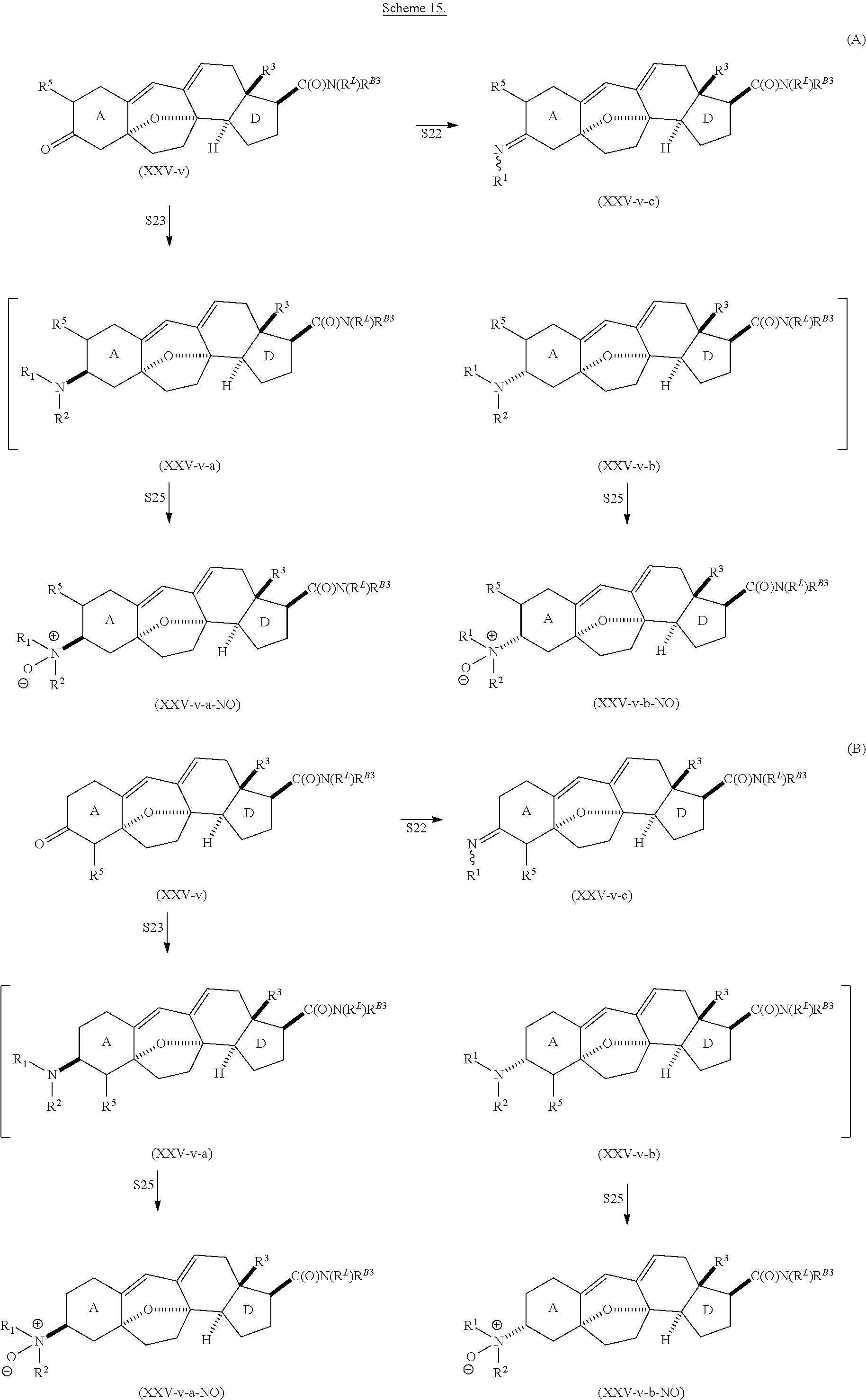

The monoketone compound (XXI) can be reductively aminated with HNR^{B4}R^{B5} (e.g., 1,2,3,4-tetrahydro-[2,7]naphthyridine) under conditions previously described to provide the compound of Formular (XXVII). Compound (XXVII) can be converted to the corresponding imines, amines, and N-oxides, as described previously. See Schemes 16(A) and 16(B).

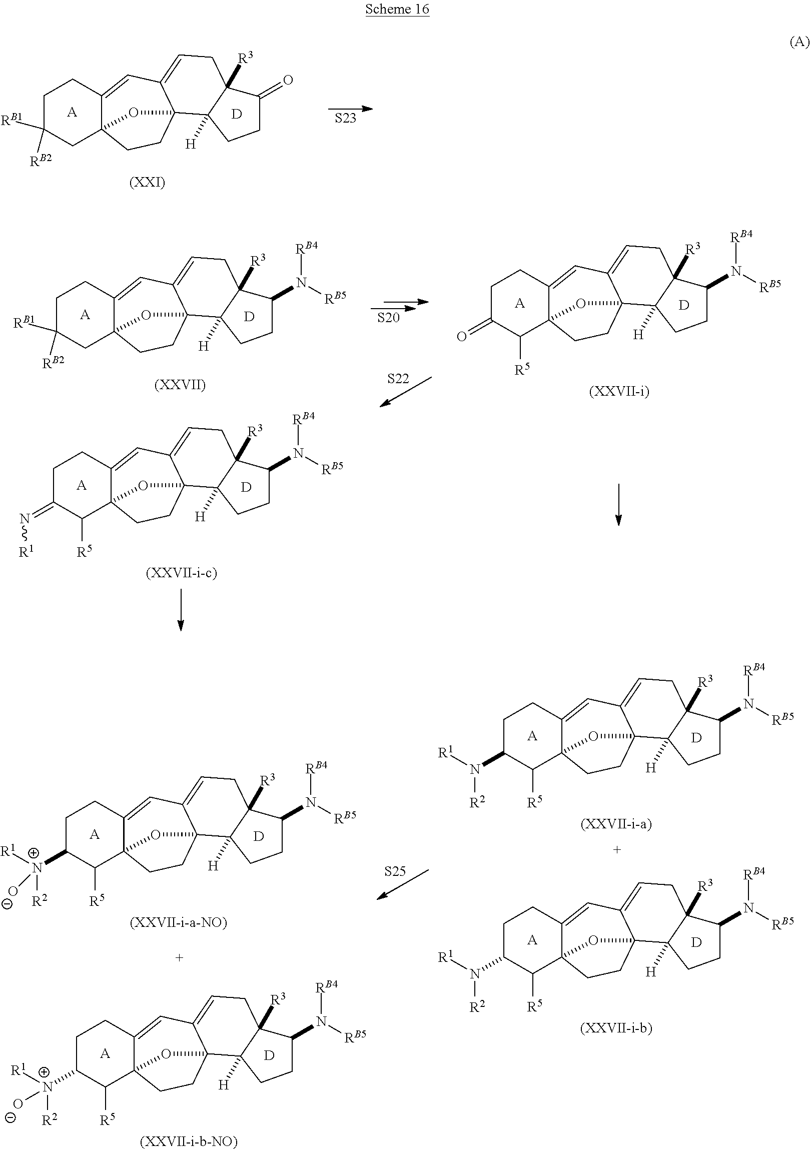

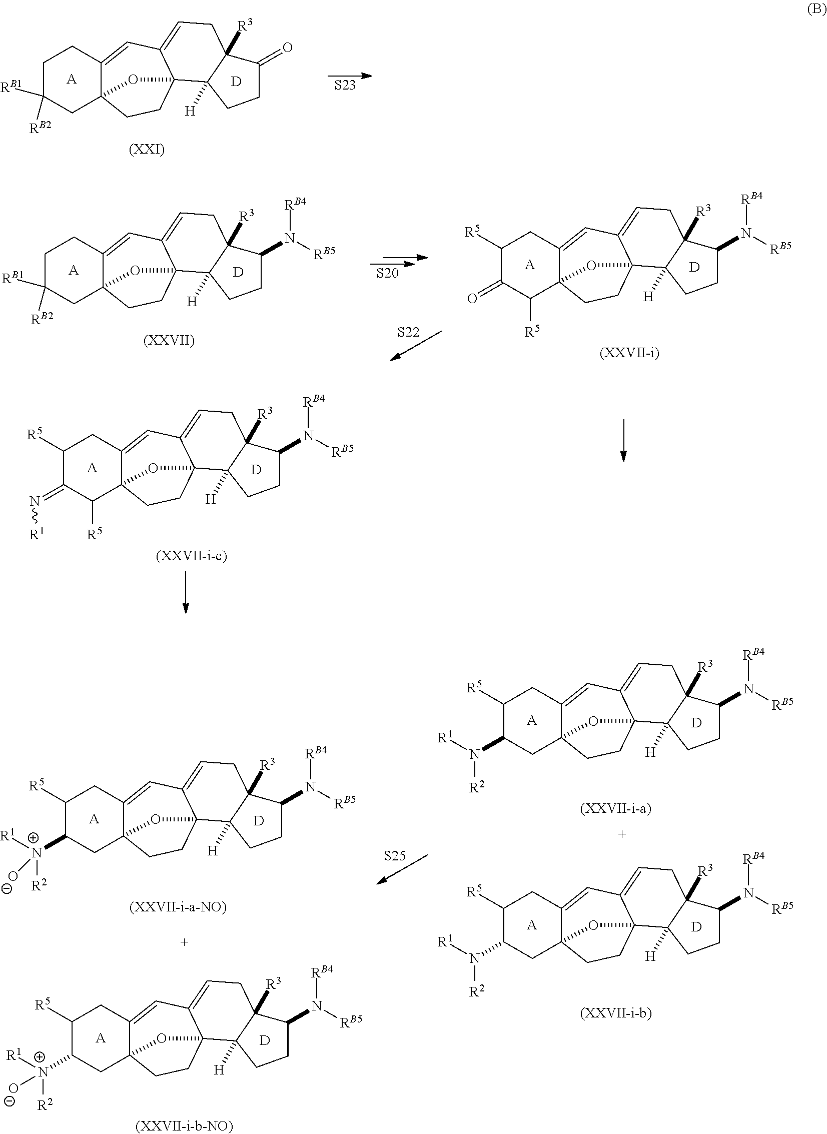

The ketone may be further synthetically manipulated to provide other compounds of interest. For example, the ketone may be reduced (as depicted in step S26) in the presence of a reducing agent to provide the C-3 hydroxylated compound. See Schemes 17 (A) and (B). Exemplary reducing agents include L-selectride, K-selectride, diisobutylaluminum hydride (DIBALH), and lithium aluminum hydride (LAH). Furthermore, various reducing agents will preferentially generate one C-3 hydroxylated compounds as the major isomer over the other, e.g., using L-selectride the beta isomer is preferably generated as the major isomer, while using lithium aluminum hydride (LAH) the alpha is preferably generated as the major isomer.

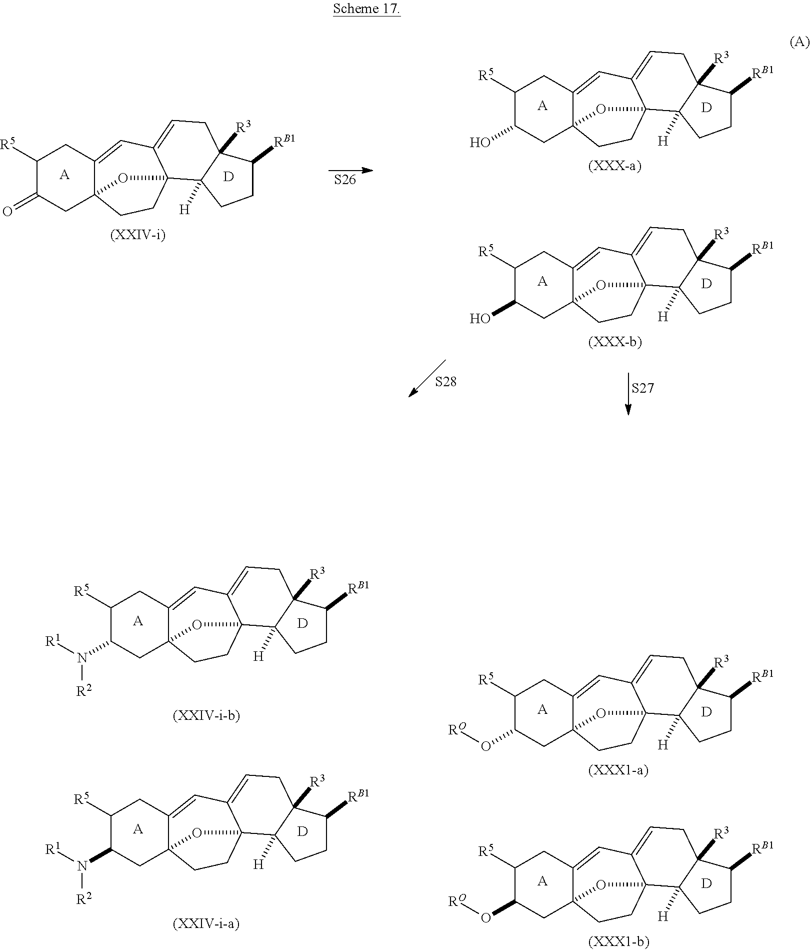

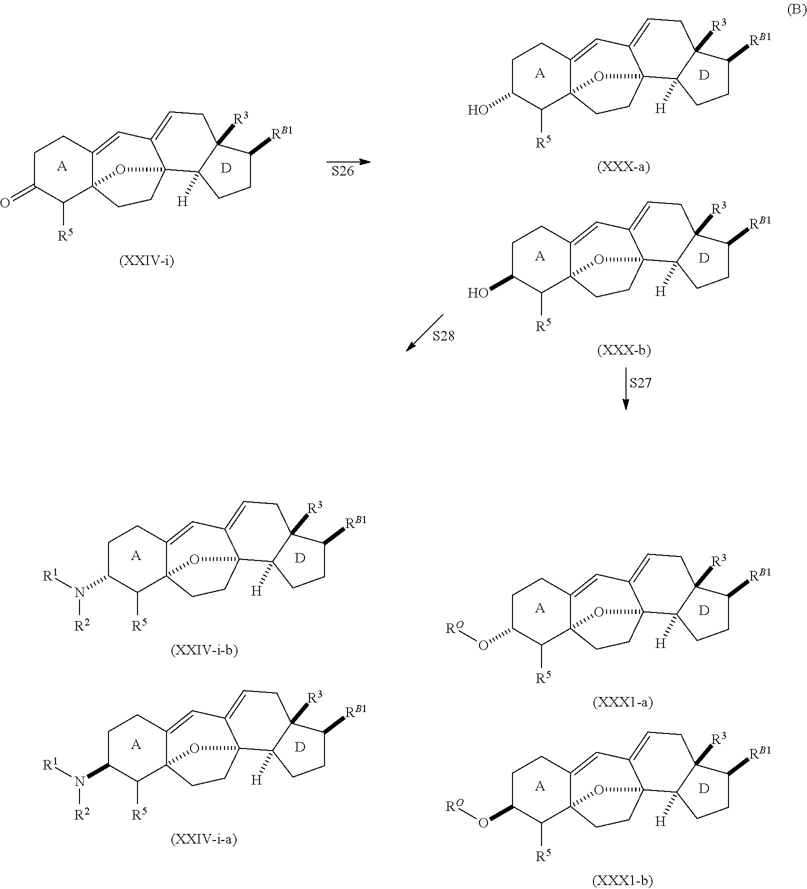

The C-3 hydroxylated compound may then be activated (e.g., by reaction with a group LG-C(═O)R^{A}, wherein LG is a leaving group, either prior to commencing the reaction or in situ (during the reaction) via substitution with a group of formula —C(═O)R^{A} under Mitsunobu reaction conditions (e.g., with HOC(═O)R^{A}, diethylazodicarboxylate (DEAD) or diisopropyl azodicarboxylate (DIAD), and PPh₃)) and then treated with an amine of formula NHR¹R² to provide a compound of Formula (XXIV) with inverted C3 stereochemistry as the major isomer (as depicted in step S28). See Schemes 17(A) and (B). Alternatively, the C-3 hydroxlated compound of Formula (XXX1) may be treated with base and a compound of formula R^{O}-LG, wherein LG is a leaving group, to provide a protected C3-hydroxyl compound with retention of C3-stereochemistry as the major isomer (as depicted in step S27).

The ketone of ring A may be further synthetically manipulated to provide compounds as described herein. Taking the ketone of formula (XXIV-i) as an example, the ketone may be converted to the free oxime (see, e.g., Scheme 18) or a substituted oxime wherein R^{O} is a non-hydrogen group (see, e.g., Scheme 19), and then converted via the Beckmann rearrangement to provide the desired lactam products. For example, the free oxime may be generated from the ketone upon treatment with hydroxylamine NH₂OH, and may, under suitable rearrangement conditions (e.g. acidic conditions, e.g., H₂SO₄, HCl, AcOH) directly provide the lactam products. see, e.g., Scheme 18.

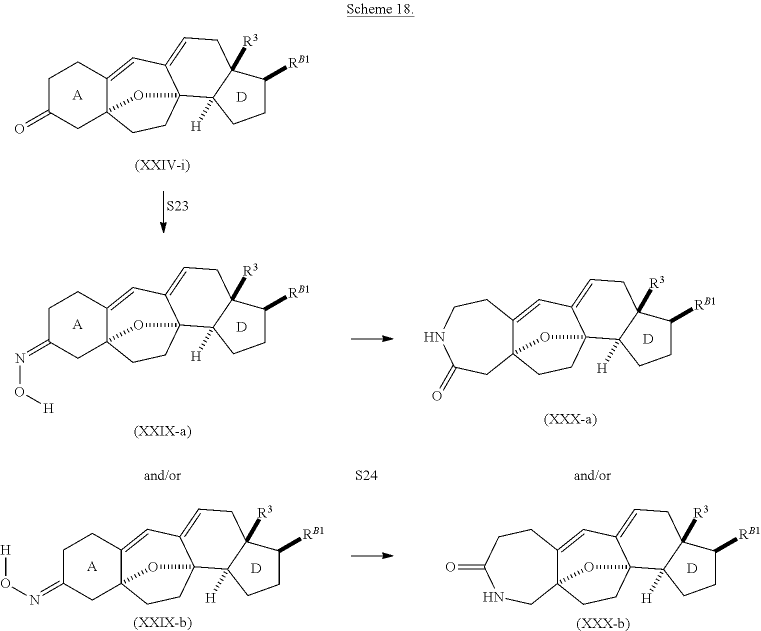

Alternatively, the substituted oxime, wherein R^{O} is a non-hydrogen group, may be generated from the ketone in a one-step process (S26), e.g., upon treatment with a substituted hydroxyl amine NH₂OR^{O}, wherein R^{O} is a non-hydrogen group, or may be generated via a two-step process (S23) and (S27), e.g., first by treatment with hydroxyl amine, NH₂OH, followed by treatment with a compound of formula R^{O}-LG, wherein R^{O} is a non-hydrogen group and LG is a leaving group. See, e.g., Scheme 18. Exemplary leaving groups (LG) include halo (e.g., chloro, bromo, iodo) and —OSO₂R^{aa}, wherein R^{aa} as defined herein. The group —OSO₂R^{aa} encompasses leaving groups such as tosyl, mesyl, and besyl, wherein R^{aa} is optionally substituted alkyl (e.g., —CH₃) or optionally substituted aryl (e.g., phenyl, tolyl). Exemplary compounds of formula R^{O}-LG include LG-C(═O)R^{A}, LG-C(═O)OR^{A}, LG-C(═O)N(R^{A})₂, LG-S(═O)₂R^{A}, LG-Si(R^{A})₃, LG-P(═O)(R^{A})₂, LG-P(═O)(OR^{A})₂, LG-P(═O)(NR^{A})₂, LG-P(═O)₂R^{A}, LG-P(═O)₂(OR^{A}), or LG-P(═O)₂N(R^{A})₂, wherein LG is as defined herein. Specifically contemplated compounds of formula LG-S(═O)₂R^{A} include Cl—S(═O)₂CH₃ (MsCl), Cl—S(═O)₂C₆H₄-(ₚCH₃) (TsCl), and Cl—S(═O)₂C₆H₅ (BsCl). The substituted oxime may, under suitable rearrangement conditions (e.g. acidic conditions, e.g., H₂SO₄, HCl, AcOH) directly provide the lactam products.

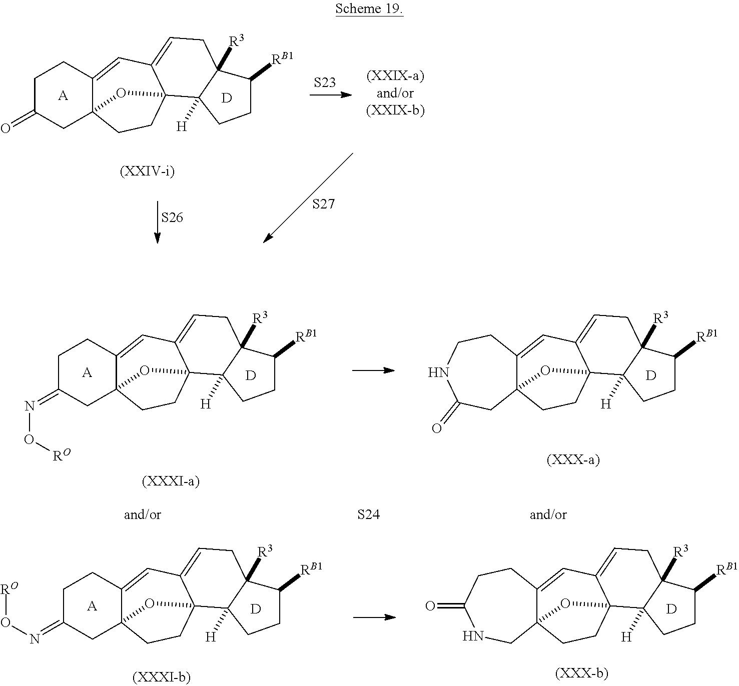

Alternatively, the ketone may be reduced (as depicted in step S30) under Wolff-Kishner reductive conditions to provide compounds of Formula (G1′) and (G1″). See Scheme 20. Exemplary Wolff-Kishner conditions are described in Furrow, M. E.; Myers, A. G. (2004) “Practical Procedures for the Preparation of N-tert-Butyldimethylsilylhydrazones and Their Use in Modified Wolff-Kishner Reductions and in the Synthesis of Vinyl Halides andgem-Dihalides”. Journal of the American Chemical Society 126 (17): 5436-5445, incorporated herein by reference.

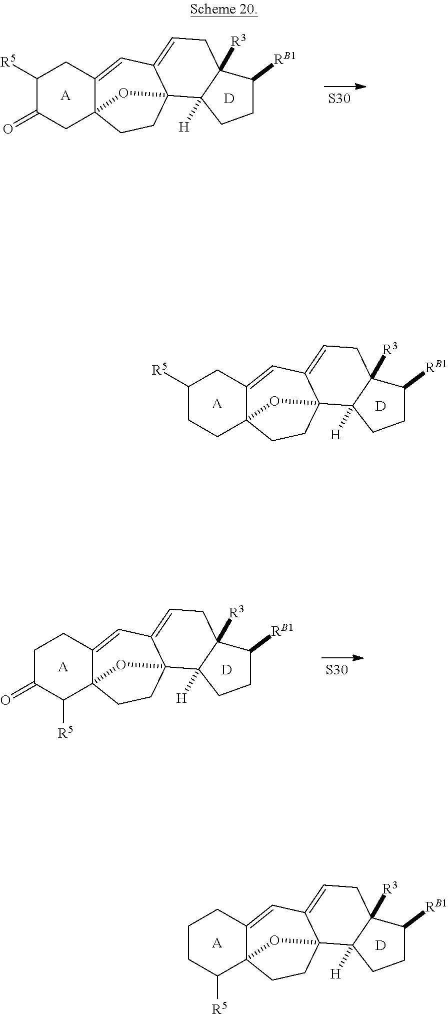

As understood herein, the oxime produced via the above described reactions may comprise a single oxime C3 isomeric product, or a mixture of both oxime C3 isomeric products. It is also generally understood that the Beckmann rearrangement proceeds by a trans [1,2]-shift; thus, in any given reaction, production of a mixture of lactam products, and wherein one lactam is the major product, is contemplated.

The lactam products may then be reduced to the azepine product using a variety of conditions, e.g., for example, use of hydrides (e.g., lithium aluminum hydride), the Clemmenson reduction (e.g., Zn(Hg)/HCl), and the Wolff-Kishner reduction (e.g., hydrazine and base (e.g., KOH), with heat). See, e.g., Scheme 21.

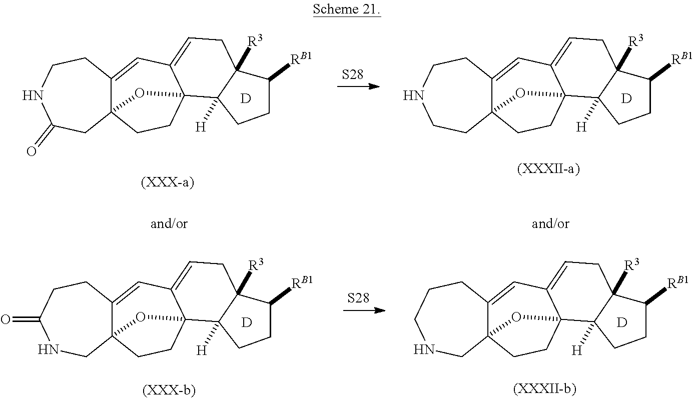

The compound of Formula (E1′) or (E1″) may be synthesized via hydrolysis of the lactam to the carboxylic acid, followed by decarboxylative halogenation, wherein X is chlorine, bromine, or iodine, and subsequent cyclization. See, e.g., Scheme 22A and 22B.

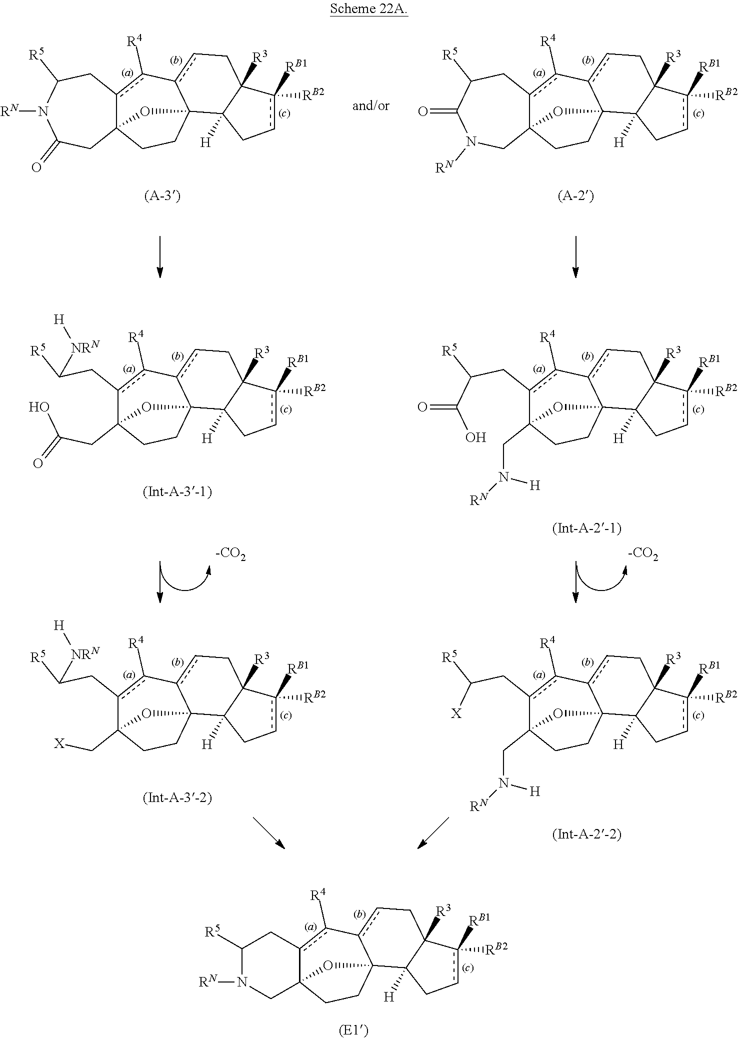

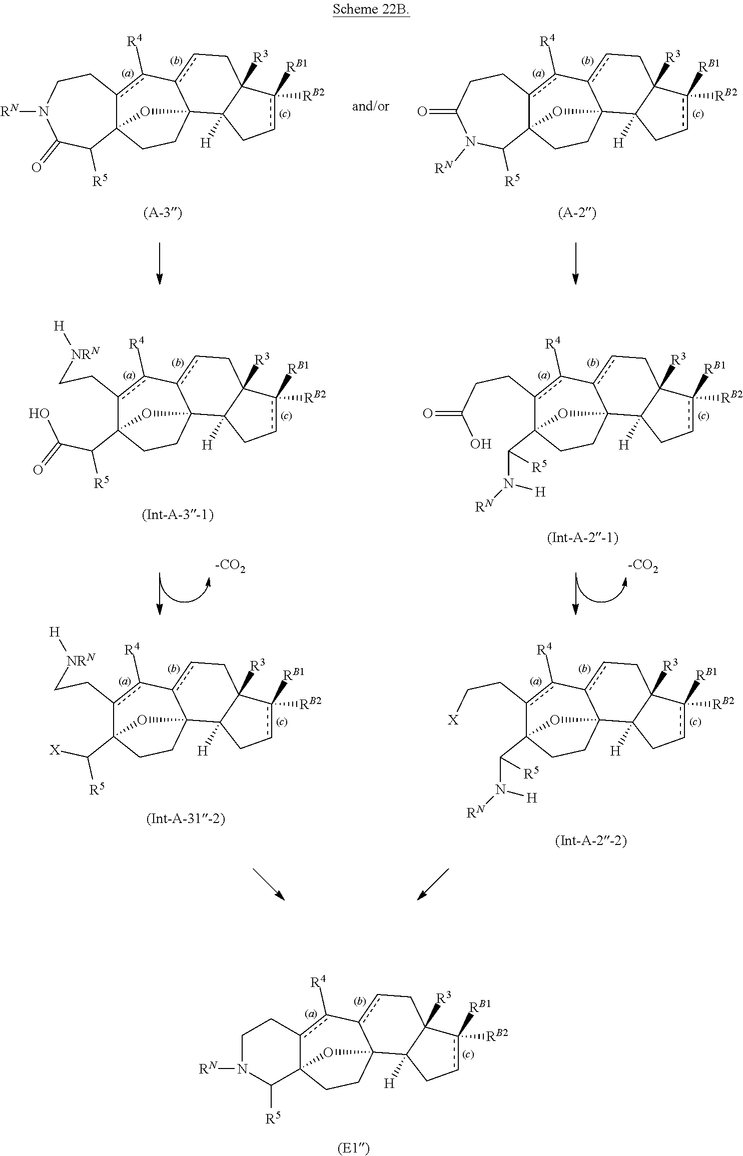

The compound of Formula (E2′) or (E2″) may be synthesized via enol trapping of the ketone of Formula (B*′) or (B*″), wherein R^{O} is a non-hydrogen group as defined herein, oxidative cleavage of the alkenyl moiety, formation of an acyl azide followed by the Curtius rearrangement to provide the amino moiety, which is subsequently cyclized to provide a lactam, reduced to the piperadinyl product wherein R^{N} is hydrogen, which may be optionally protected by a non-hydrogen group R^{N}. See, e.g., Scheme 23A or 23B.

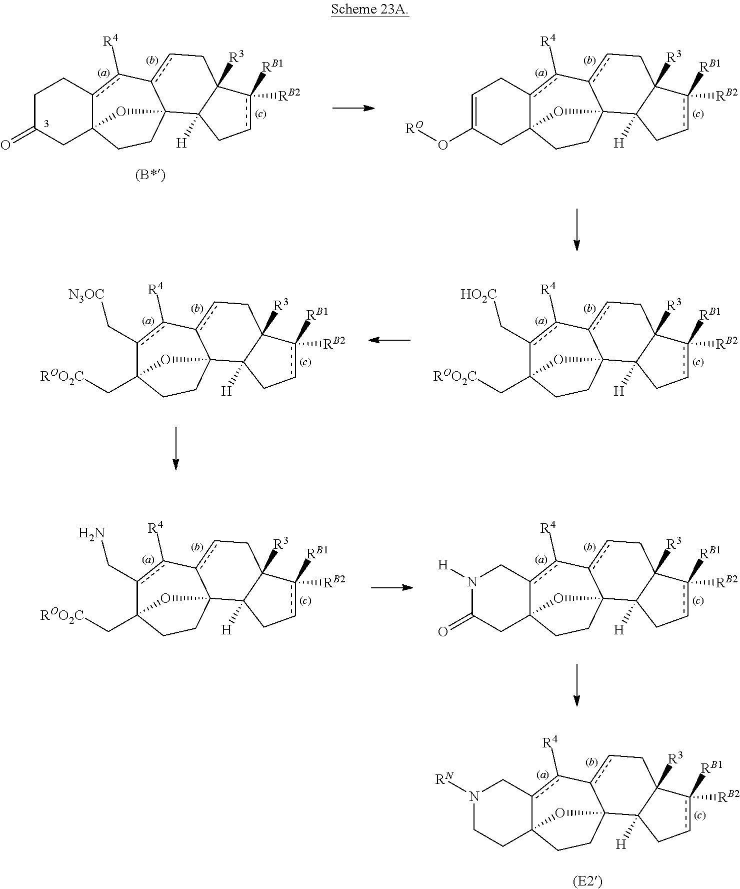

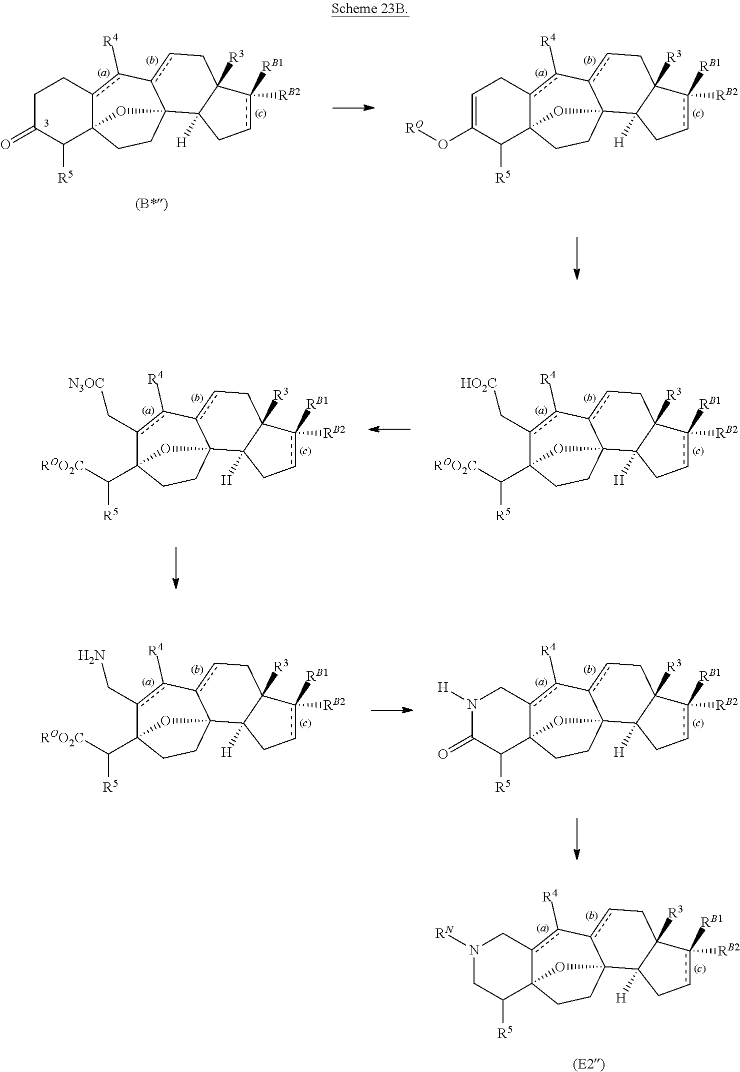

As used herein, a “major isomer” refers to the isomer that is produced in excess of the other isomer, i.e., greater than 50% of the sum of the two isomers produced from the reaction, e.g., greater than 60%, 70%, 80%, 90%, or 95% of the sum of the two isomers produced from the reaction.

D. Representative Syntheses of Cortistatin Analogs

### Materials and Instrumentation:

All reactions were performed in flame-dried glassware under a positive pressure of argon unless otherwise noted. Flash column chromatography was performed as described by Still et al., J. Org. Chem. 1978, 43, 2923-2925 employing silica gel 60 (40-63 μm, Whatman).

Commercial reagents and solvents were used as received with the following exceptions: tetrahydrofuran (THF), dichloromethane (CH₂C12) were degassed with argon and passed through a solvent purification system (designed by J. C. Meyer of Glass Contour) utilizing alumina columns as described by Pangborn et al., Organometallics 1996, 15, 1518-1520. Pyridine and triethylamine were distilled over calcium hydride before use. The Celite used was Celite® 545, purchased from J. T. Baker. The molarities of n-butyllithium solutions were determined by titration using 1,10-phenanthroline as an indicator (average of three determinations).

¹H NMR spectra were recorded with a Varian INOVA-600 or Varian INOVA-500 spectrometer. Proton chemical shifts are reported in parts per million (6 scale) and are calibrated using residual undeuterated solvent as an internal reference (CDCl₃: δ 7.26 (CHCl₃), C₆D₆: δ 7.15 (C₆D₅H)). Data for ¹H NMR spectra are reported as follows: chemical shift (δ ppm) (multiplicity, coupling constant (Hz), integration). Multiplicities are reported as follows: s=singlet, d=doublet, t=triplet, q=quartet, m=multiplet, br=broad, app=apparent, or combinations thereof. ¹³C NMR spectra were recorded with a Varian INOVA-500 spectrometer. High-resolution mass spectra (HRMS) were obtained from the Harvard University Mass Spectrometry Laboratory where electrospray ionization (ESI) mass spectroscopy (MS) experiments were performed on an Agilent 6210 TOF LC/MS instrument.

### Example S1. Synthesis of Keystone Starting Material

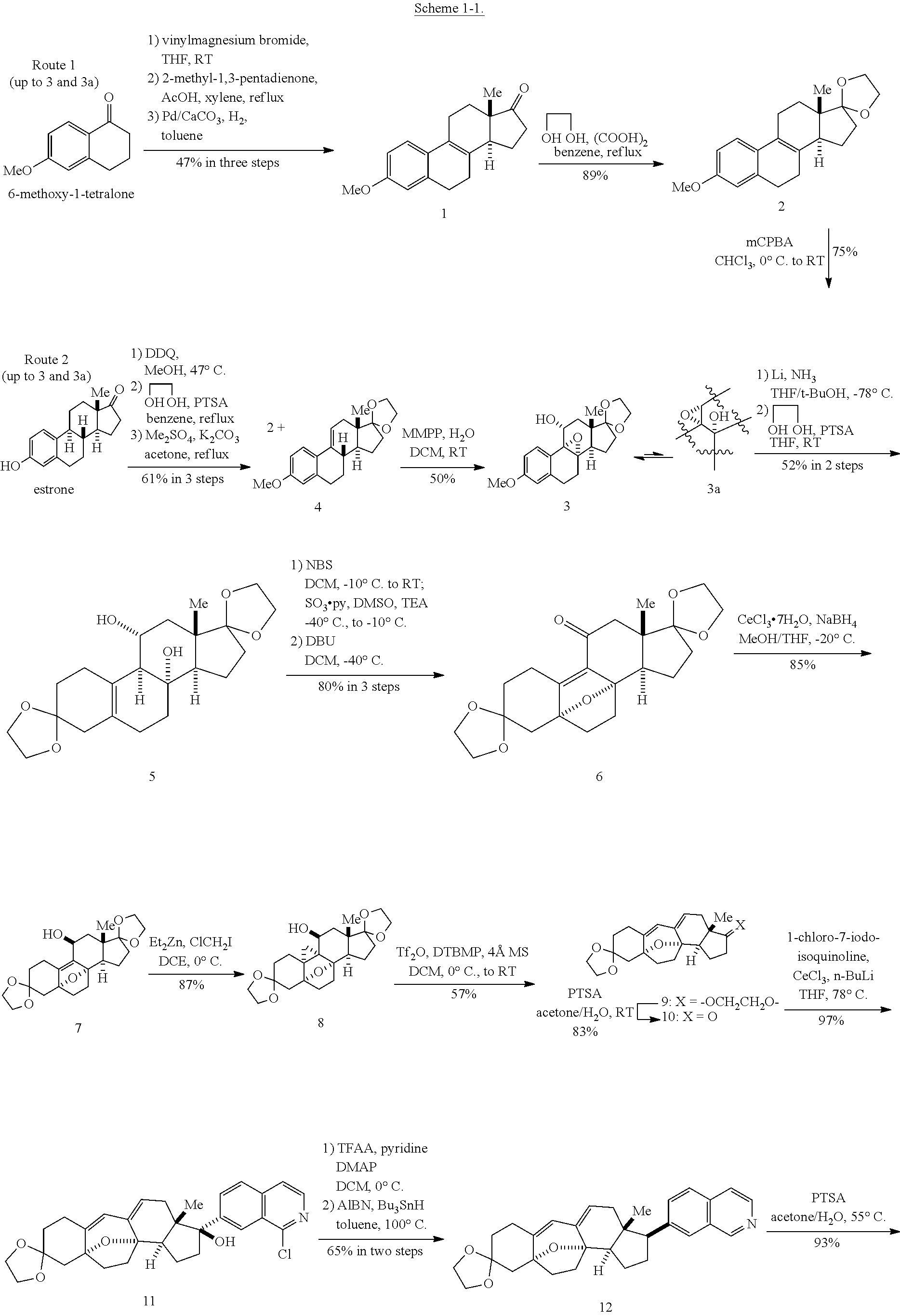

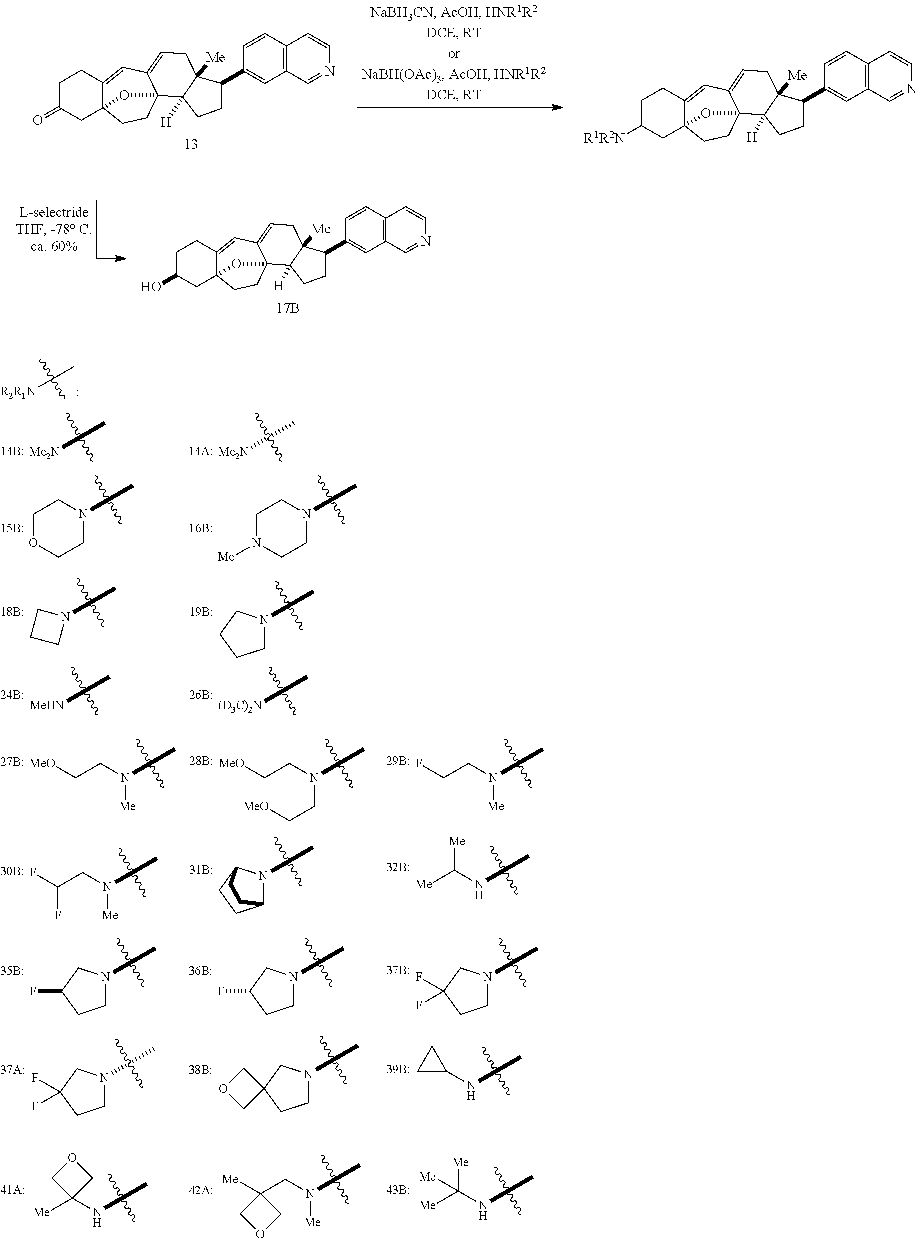

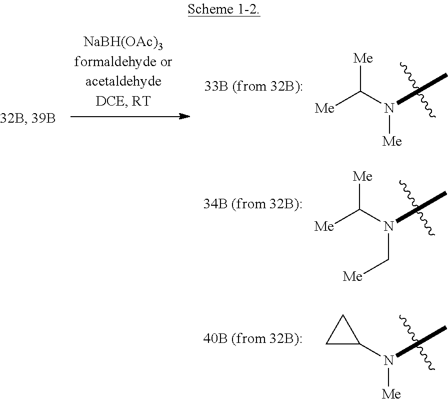

Route 1: Synthesis of 8,9-Unsaturated Methoxyethyleneketone from 6-Methoxy-1-Tetralone (Compound 1)

The Grignard reaction was done with 20.0 g (113 mmol, 1.00 equiv) of 6-methoxy-1-tetralone and the product was used without purification by flash chromatography. See, e.g., Saraber et al., Tetrahedron 2006, 62, 1726-1742. To a solution of Grignard reaction product and 2-methyl-1,3-pentadienone (12.8 g, 114 mmol, 1.01 equiv) in xylene (140 mL) was added AcOH (64.6 mL, 1.13 mol, 10.0 equiv) and the reaction mixture was warmed to reflux. After 2 h, the reaction was allowed to cool to room temperature and the concentrated under reduced pressure. The mixture of 1:1 of toluene and ethyl ether was added to dissolve the solid residue and the mixture was filtered. The filtrate was washed sequentially with saturated NaHCO₃ solution (200 mL) and brine, dried over MgSO₄, and concentrated under reduced pressure. The residue was purified by flash chromatography (silica gel, eluent: 20:1:1 Hexanes:EtOAc:DCM) to afford the Torgov's diene. Spectral data was consistent with those previously reported. See, e.g., Soorukram, D.; Knochel, P. Org. Lett. 2007, 9, 1021-1023. The Torgov's diene was converted to 8,9-unsaturated methoxyethyleneketone compound 1 (15.0 g, 47% over 3 steps) based on the literature known procedure. See, e.g., Sugahara et al., Tetrahedron Lett. 1996, 37, 7403-7406.

### Route 1: Synthesis of 8,9-Unsaturated Methoxyethyleneketal (Compound 2)

To a solution of compound 1 (15.0 g, 53.1 mmol, 1.0 equiv) in benzene (215 mL) and ethylene glycol (72 mL) was added oxalic acid (2.30 g, 12.1 mmol, 0.22 equiv). The reaction mixture was allowed to warm to reflux and water was trapped by Dean-Stark apparatus. After 16 h, the reaction was cool to room temperature and saturated NaHCO₃ solution (150 mL) was added. The organic and aqueous layers were separated and the aqueous phase was extracted with ethyl acetate (2×200 mL). The combined organic phases were washed with brine (150 mL) and dried over Na₂SO₄. The solvent was evaporated under reduced pressure and the residue was purified by flash chromatography (silica gel, eluent: 15:1 Hexanes:EtOAc) to provide 8,9-unsaturated methoxyethyleneketal compound 2 (15.5 g, 89%). ¹H NMR (500 MHz, CDCl₃) Shift=7.13 (d, J=8.3 Hz, 1H), 6.73-6.67 (m, 2H), 4.05-3.85 (m, 4H), 3.79 (s, 3H), 2.82-2.65 (m, 2H), 2.52-2.45 (m, 2H), 2.23-2.17 (m, 2H), 2.14 (ddd, J=2.2, 11.6, 14.0 Hz, 1H), 1.99-1.82 (m, 4H), 1.64 (td, J=4.2, 12.2 Hz, 1H), 1.49 (dq, J=6.8, 11.6 Hz, 1H), 0.86 (s, 3H). HRMS (ESI) (m/z) calculated for C₂₁H₂₇O₃ [M+H]⁺: 327.1955, found 327.1947.

### Route 1: Synthesis of Epoxy Alcohols 3 and 3a

A solution of 8,9-unsaturated ethyleneketal 2 (1.63 g, 5.00 mmol, 1.0 equiv) in CHC13 (50 mL) was cooled to 0° C. and mCPBA (77% max, 2.46 g, 11.0 mmol, 2.2 equiv) was added. The reaction mixture was stirred for 10 min at 0° C. and warmed to room temperature. After additional 50 min, 10% Na₂S₂O₃ solution (40 mL) and saturated NaHCO₃ solution (40 mL) were sequentially added. The organic and aqueous layers were separated and the aqueous phase was extracted with dichloromethane (3×50 mL). The combined organic phases were washed with brine (50 mL), dried over Na₂SO₄, and concentrated under reduced pressure. The residue was purified by flash chromatography (silica gel, eluent: 3:1→1:1 Hexanes:EtOAc) to afford epoxy alcohol 3 and 3a (1.40 g, 75%). 3 and 3a were under equilibration in any solvent, with a major of 3. H NMR was analyzed for epoxy alcohol 3. Where indicted, cortistatin analogs (12, 13, 14A, 14B, 15B, 16B, and 17B) were applied to the biological experiments as racemic mixtures constructed from 6-methoxy-1-tetralone.

¹H NMR (500 MHz, CDCl₃) Shift=7.77 (d, J=8.3 Hz, 1H), 6.76 (dd, J=2.0, 8.3 Hz, 1H), 6.63 (d, J=2.0 Hz, 1H), 4.78 (dd, J=7.8, 9.8 Hz, 1H), 3.95-3.87 (m, 4H), 3.78 (s, 3H), 2.84 (dt, J=5.9, 14.4 Hz, 1H), 2.49 (dd, J=4.4, 15.1 Hz, 1H), 2.36-2.29 (m, 1H), 2.26 (dd, J=5.9, 14.2 Hz, 2H), 2.06 (t, J=11.7 Hz, 1H), 1.97 (dd, J=7.3, 12.2 Hz, 1H), 1.94-1.88 (m, 2H), 1.75 (dt, J=5.4, 14.2 Hz, 1H), 1.63-1.53 (m, 1H), 1.46 (t, J=11.0 Hz, 1H), 0.75 (s, 3H). HRMS (ESI) (m/z) calculated for C₂₁H₂₇O₅ [M+H]⁺: 359.1853, found 359.1852.

### Route 2: Synthesis of 8,9 and 9,11-Unsaturated Methoxyethyleneketal Compounds 2 and 4

The DDQ oxidation was done with 22.0 g (81.4 mmol, 1.0 equiv) of estrone and the product was used without purification by flash chromatography. See, e.g., Stephan et al., Steroid. 1995, 60, 809-811. To a solution of 9,11-unsaturated estrone in benzene (375 mL) was added ethylene glycol (110 mL, 1.99 mol, 24.4 equiv) and PTSA (3.00 g, 16.3 mmol, 0.20 equiv). The reaction mixture was warmed to reflux and water was trapped by Dean-Stark apparatus. After 18 h, the reaction was allowed to cool to room temperature and saturated NaHCO₃ solution (300 mL) was applied. The aqueous phase was extracted with ethyl acetate (2×300 mL) and the combined organic phases were washed with brine (200 mL). The organic phase was dried (Na₂SO₄) and the solvent was evaporated under reduced pressure. The product was used in the next step without further purification.

The ethylene ketal (mixture of the 8,9 and 9,11-unsaturated regioisomers) was dissolved in acetone (420 mL) and K₂CO₃ (22.5 g, 163 mmol, 2.00 equiv) was added. This was followed by the addition of Me₂SO₄ (9.30 mL, 97.6 mmol, 1.20 equiv) and the reaction mixture was warmed to reflux. After 18 h, the reaction was allowed to cool to room temperature and the acetone was evaporated. 2M NaOH solution was added (300 mL) and the aqueous phase was extracted with ethyl acetate (2×300 mL). The combined organic phases were dried (Na₂SO₄) and the solvent was evaporated under reduced pressure. The residue was purified by flash chromatography (silica gel, eluent: 15:1 Hexanes:EtOAc) to afford mixture of 8,9 and 9,11-unsaturated methoxyethylene ketal compounds 2 and 4 (16.3 g, 61% in three steps, -4:5 mixture of 8,9-unsaturated:9,11-unsaturated regioisomers).

For 9,11-unsaturated isomer, only distinguishable peaks were assigned: ¹H NMR (500 MHz, CDCl₃) Shift=7.53 (d, J=8.8 Hz, 1H), 6.60 (d, J=2.0 Hz, 1H), 6.13 (td, J=2.6, 5.0 Hz, 1H), 3.79 (s, 3H), 2.59 (td, J=3.2, 17.6 Hz, 1H), 2.09-2.00 (m, 3H), 1.45-1.33 (m, 2H), 0.90 (s, 3H). HRMS (ESI) (m/z) calculated for C₂₁H₂₇O₃ [M+H]⁺: 327.1955, found 327.1951.

### Route 2: Epoxy Alcohol Compounds 3 and 3a

To a solution of mixture of 8,9 and 9,11-unsaturated ethylene ketal compounds 2 and 4 (15.7 g, 48.1 mmol, 1.00 equiv) in dichloromethane (700 mL) was added magnesium monoperoxyphthalate hexahydrate (68.4 g, 111 mmol, 2.30 equiv) and water (4.8 mL). The reaction mixture was stirred for 20 h at room temperature and then quenched with the mixture of 10% aqueous Na₂S₂O₃ (300 mL) and saturated NaHCO₃ solution (300 mL). The organic and aqueous layers were separated and the aqueous phase was extracted with dichloromethane (2×500 mL). The combined organic phases were washed with brine (300 mL) and dried (Na₂SO₄). The solvent was evaporated under reduced pressure and the residue was purified by flash chromatography (silica gel, eluent: 3:1-2:1 Hexanes:EtOAc) to provide epoxy alcohol 3 and 3a (8.60 g, 50%). Spectral data was consistent with epoxy alcohol 3 and 3a constructed from 8,9-unsaturated methoxyethylene ketal 2.

### Synthesis of Diol Compound 5

Ammonia gas was condensed (240 mL) and to the liquid ammonia was added L₁ (3.90 g, 565 mmol, 25.0 equiv) at −78° C. After stirring for 30 min, epoxy alcohol 3 and 3a (8.10 g, 22.6 mmol, 1.0 equiv) in THF (110 mL) was cannulated and stirred additional 1.5 h at that temperature. To the reaction mixture was added the mixture of t-BuOH (32 mL) and THF (16 mL) at −78° C. and stirred additional 20 min at that temperature. The mixture of t-BuOH (92 mL) and THF (38 mL) was added followed by benzene (50 mL) and water (50 mL) at −78° C., and the flask was opened to gently evaporate liquid ammonia by removing the cooling bath. Water (200 mL) was added and the aqueous phase was extracted with ethyl acetate (2×250 mL). The combined organic phases were washed with brine (150 mL), dried (Na₂SO₄), and concentrated under reduced pressure. The product was used in the next step without further purification.

To a solution of Birch reduction product in THF (300 mL) and ethylene glycol (75 mL) was added PTSA (430 mg, 2.26 mmol, 0.10 equiv). The reaction mixture was stirred for 30 min at room temperature and saturated NaHCO₃ solution (200 mL) was added. The organic and aqueous layers were separated and the aqueous phase was extracted with ethyl acetate (4×250 mL). The combined organic phases were washed with brine (200 mL) and dried (Na₂SO₄). The solvent was evaporated under reduced pressure and the residue was purified by flash chromatography (silica gel, eluent: 4:1 Hexanes:EtOAc -100% EtOAc -10:1 EtOAc:MeOH) to provide diol 5 (4.60 g, 52%).

¹H NMR (500 MHz, C₆D₆) Shift=3.67-3.42 (m, 9H), 3.25-3.14 (m, 1H), 2.40 (dd, J=5.9, 13.2 Hz, 1H), 2.31 (br. s, 2H), 2.23-2.09 (m, 2H), 2.03 (t, J=10.7 Hz, 1H), 1.97-1.90 (m, 2H), 1.89 (dd, J=8.3, 14.2 Hz, 1H), 1.85-1.75 (m, 4H), 1.66-1.50 (m, 4H), 1.00 (s, 3H). HRMS (ESI) (m/z) calculated for C₂₂H₃₂NaO₆ [M+Na]⁺: 415.2091, found 415.2076.

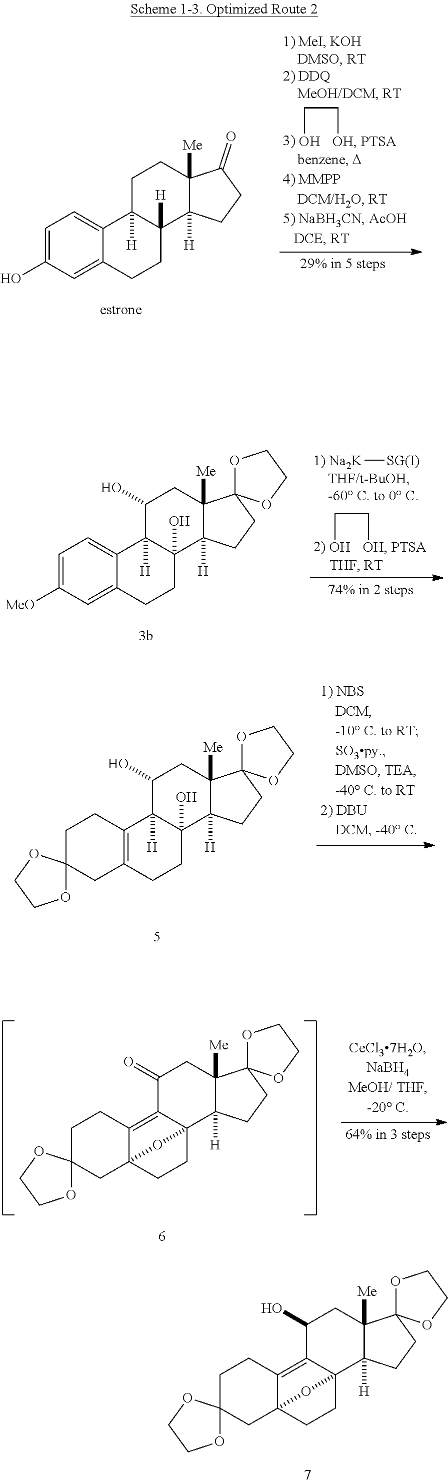

### Ketal Compound 3b

To a solution of estrone (195 g, 721 mmol, 1.00 equiv) in DMSO (2.8 L) was added KOH pellet (85% technical grade, 162 g, 2.45 mol, 3.40 equiv) and CH₃I (89.8 mL, 1.44 mol, 2.00 equiv). The reaction mixture was stirred for 3.5 hours at room temperature and distilled water (2 L) was slowly added at 0° C. The aqueous layer was extracted with dichloromethane (3×1.5 L) and the combined organic layer was washed with brine (1.5 L). The organic layer was concentrated under nitrogen flow to give white crystalline, which was washed with cold methanol. The 180 g of crude mixture was used in the next step without further purification.

To a solution of the crude mixture (100 g, 352 mmol, 1.00 equiv) in methanol (750 mL) and dichloromethane (750 mL) was added NaHCO₃ (93.8 g, 1.05 mmol, 3.00 equiv). DDQ (120 g, 527 mmol, 1.50 equiv) was added in four portions with 5 min interval and the reaction mixture was stirred for 2 hours and then quenched with the 10% aqueous Na₂S₂O₃(500 mL). The reaction flask was stirred for additional 30 min and filtered through Celite, washed with chloroform. The 2 M NaOH solution (500 mL) was added and the organic and aqueous layers were separated and the aqueous phase was extracted with chloroform (3×700 mL). The combined organic phases were washed with brine (700 mL) and dried (Na₂SO₄). The solvent was evaporated under reduced pressure and the 89 g of crude mixture was used in the next step without further purification. The DDQ oxidation step was conducted in two batches.

To a solution of the crude mixture (151 g, 480 mmol, 1.00 equiv) in benzene (2 L) was added ethylene glycol (268 mL, 4.80 mol, 10 equiv) and PTSA (27.4 g, 144 mmol, 0.30 equiv). The reaction mixture was warmed to reflux and water was trapped by Dean-Stark apparatus. After 36 hours, the reaction was allowed to cool to room temperature and saturated NaHCO₃ solution (1 L) was applied. The aqueous phase was extracted with ethyl acetate (3×500 mL) and the combined organic phases were washed with brine (1 L). The organic phase was dried (Na₂SO₄) and the solvent was evaporated under reduced pressure. The 170 g of crude product was used in the next step without further purification.

To a solution of crude mixture (480 mmol, 1.00 equiv) in dichloromethane (2.5 L) was added magnesium monoperoxyphthalate hexahydrate (˜80% technical grade, 683 g, 1.10 mol, 2.30 equiv) and water (50 mL). The reaction mixture was stirred for 16 hours at room temperature and then filtered through Celite pad. To the filtrate was added saturated NaHCO₃ solution (1.5 L) and the organic and aqueous layers were separated and the aqueous phase was extracted with dichloromethane (3×1.4 L). The combined organic phases were washed with brine (1.4 L) and dried (Na₂SO₄). The solvent was evaporated under reduced pressure and the crude mixture was used in the next step without further purification.

To a solution of crude mixture (480 mmol, 1.00 equiv) in 1,2-dichloroethane (2 L) was added NaBH₃CN (60.3 g, 960 mmol, 2.00 equiv) and AcOH (55 mL, 960 mmol, 2.00 equiv) sequentially at room temperature. After 2.5 hours, saturated NaHCO₃ solution (1.4 L) was added and the organic and aqueous layers were separated. The aqueous phase was extracted with dichloromethane (3×1.4 L). The combined organic phases were washed with brine (1.5 L), dried over Na₂SO₄, and concentrated under reduced pressure. The residue was purified by flash chromatography (silica gel, eluent: 2:1 Hexanes:EtOAc -1:1→1:2→1:3-100% EtOAc) to provide compound 3b (75 g, 29% in 5 steps).

¹H NMR (500 MHz, CDCl₃) Shift=7.21 (d, J=8.8 Hz, 1H), 6.75 (dd, J=2.4, 8.3 Hz, 1H), 6.72 (d, J=2.4 Hz, 1H), 3.98-3.82 (m, 4H), 3.79 (s, 3H), 3.80-3.76 (m, 1H), 3.54 (dt, J=4.4, 10.5 Hz, 1H), 3.03-2.91 (m, 1H), 2.81 (td, J=4.4, 18.1 Hz, 1H), 2.33 (d, J=9.8 Hz, 1H), 2.23 (dd, J=6.8, 13.2 Hz, 1H), 2.09-1.98 (m, 1H), 1.90 (ddd, J=5.9, 9.8, 14.6 Hz, 1H), 1.85 (dd, J=4.6, 9.5 Hz, 2H), 1.82-1.77 (m, 1H), 1.77-1.70 (m, 1H), 1.65 (dq, J=6.3, 12.7 Hz, 1H), 1.02 (s, 3H). HRMS (ESI) (m/z) calculated for C₂₁H₂₈O₅ [M+H]⁺: 361.2010, found 361.2022.

### Diol Compound 5

To a slurry of Na₂K-SG(I) (200 g) in THF and t-BuOH (500 mL and 200 mL of each solvent, sequentially added at −60° C.) was cannulated compound 3b (40 g, 111 mmol, 1.00 equiv) in THF (500 mL) at -60° C. and allowed to warm to 0° C. The reaction was followed by MS. After stirring for 7 hours at 0° C. the reaction was quenched by slow addition of MeOH (150 mL) and H₂O (250 mL) and was allowed to warm to room temperature. After decanting the solution to separate out the silica gel, EtOAc (1 L) was added and the organic layer and the aqueous layers were separated. The aqueous phase was extracted with EtOAc (3×500 ml). The combined organic phases were washed with brine (2×1 L), dried over Na₂SO₄, and concentrated under reduced pressure. The product was used without further purification. Ketalization condition is the same as was described for compound 3b to give compound 5 (32 g, 74% in 2 steps).

### Allylic Alcohol 7

To a solution of diol 7 (7.1 g, 18.1 mmol, 1.00 equiv) in dichloromethane (230 mL) was added NBS (3.54 g, 19.9 mmol, 1.10 equiv) at one portion at −10° C. and the reaction mixture was warmed to room temperature. The reaction was monitored by TLC (about 2 h min for the completion). Once the reaction is done, the reaction mixture was cooled to −40° C. and triethylamine (30.3 mL, 217 mmol, 12.0 equiv) was added. Pre-stirred SO₃.Py (28.8 g, 181 mmol, 10.0 equiv) in DMSO (200 mL) for 20 min at room temperature was added to the reaction mixture at −40° C., which was subsequently allowed to warm slowly to room temperature. After 3 hours, saturated NH₄Cl solution (200 mL) was added and the reaction was allowed to warm to room temperature. The organic and aqueous layers were separated and the aqueous phase was extracted with dichloromethane (2×350 mL). The combined organic phases were washed with brine (350 mL), dried over Na₂SO₄, and concentrated under reduced pressure. The crude mixture was used without further purification.

The crude mixture was dissolved in dichloromethane (600 mL) and the reaction mixture was cooled to −40° C. followed by the slow addition of DBU (6.76 mL, 45.3 mmol, 2.50 equiv). After 15 min, saturated NH₄Cl solution (200 mL) was added and the reaction was allowed to warm to room temperature. The organic and aqueous layers were separated and the aqueous phase was extracted with dichloromethane (2×200 mL). The combined organic phases were washed with brine (150 mL), dried over Na₂SO₄, and concentrated under reduced pressure.

To a solution of crude mixture (6.50 g, 16.7 mmol, 1.00 equiv) in MeOH (250 mL) and THF (30 mL) was added CeCl₃.7H₂O (18.7 g, 50.2 mmol, 3.00 equiv) at room temperature. After stirring 5 min, the reaction was cooled to −20° C. followed by the addition of NaBH₄ (1.26 g, 33.4 mmol, 2.00 equiv). After 30 min, saturated NH₄Cl solution (100 mL) and water (100 mL) was added, which was allowed to warm to room temperature. The aqueous phase was extracted with ethyl acetate (3×250 mL) and the combined organic phases were washed with brine (200 mL), dried over Na₂SO₄, and concentrated under reduced pressure. The residue was purified by flash chromatography (silica gel, eluent: 20:1 DCM:MeOH) to afford allylic alcohol 7 (4.20 g, 60% in 3 steps).

¹H NMR (500 MHz, C₆D₆) Shift=4.39-4.30 (m, 1H), 3.58-3.36 (m, 8H), 3.22 (dd, J=3.7, 16.4 Hz, 1H), 2.94 (dd, J=7.1, 12.5 Hz, 1H), 2.66 (d, J=13.2 Hz, 1H), 2.49-2.41 (m, 1H), 2.39 (dd, J=2.2, 12.9 Hz, 1H), 2.07-1.99 (m, 1H), 1.96-1.79 (m, 6H), 1.73 (br. s, 3H), 1.66-1.57 (m, 1H), 1.15-1.07 (m, 1H), 0.86 (s, 3H); ¹³C NMR (500 MHz, C₆D₆) Shift=140.6, 139.1, 118.7, 109.5, 88.3, 86.2, 67.1, 65.4, 64.6, 64.2, 47.9, 46.5, 41.3, 40.9, 34.7, 34.2, 33.9, 30.0, 20.4, 19.8, 15.6; HRMS (ESI) (m/z) calculated for C₂₂H₃₀NaO₆ [M+Na]⁺: 413.1935, found 413.1942.

### Cyclopropane 8

To a solution of C1CH₂I (5.74 mL, 78.9 mmol, 4.00 equiv) in 1,2-dichloroethane (400 mL) was added a solution of Et2Zn in diethyl ether (1M, 39.4 mL, 39.4 mmol, 2.00 equiv) at −10° C. After stirring 5 min, allylic alcohol 7 (7.70 g, 19.7 mmol, 1.00 equiv) in 1,2-dichloroethane (200 mL) was added to the reaction flask at −10° C. After 30 min, the reaction was quenched by saturated NH₄Cl solution (300 mL) and allowed to warm to room temperature. The organic and aqueous layers were separated and the aqueous phase was extracted with dichloromethane (2×350 mL). The combined organic phases were washed with brine (300 mL), dried over Na₂SO₄, and concentrated under reduced pressure. The residue was purified by flash chromatography (silica gel, eluent: 2:1→1:1 Hexanes:EtOAc) to afford cyclopropane 8 (6.93 g, 87%).

¹H NMR (500 MHz, C₆D₆) Shift=3.92 (dd, J=3.7, 11.0 Hz, 1H), 3.51-3.40 (m, 8H), 2.72 (dd, J=7.1, 12.9 Hz, 1H), 2.39 (dd, J=5.4, 17.6 Hz, 1H), 2.38 (d, J=12.2 Hz, 1H), 2.15 (d, J=12.2 Hz, 1H), 2.12 (dt, J=4.9, 12.2 Hz, 1H), 2.02 (ddd, J=2.9, 11.2, 14.6 Hz, 1H), 1.92-1.82 (m, 3H), 1.82-1.73 (m, 2H), 1.69-1.54 (m, 5H), 1.52 (dd, J=6.1, 12.0 Hz, 1H), 1.49-1.44 (m, 1H), 0.98 (s, 3H), 0.86 (d, J=2.4 Hz, 1H), 0.15 (d, J=2.9 Hz, 1H); ¹³C NMR (500 MHz, C₆D₆) Shift=118.5, 110.4, 85.4, 84.0, 65.3, 64.9, 64.7, 64.6, 64.1, 48.1, 45.4, 41.5, 40.0, 39.9, 35.4, 34.8, 33.7, 32.7, 29.1, 22.1, 19.3, 16.5, 4.0; HRMS (ESI) (m/z) calculated for C23H₃₂NaO₆ [M+Na]⁺: 427.2091, found 427.2088.

Oxabicyclo[3.2.1]octene Skeleton 9

Cyclopropane 8 (6.90 g, 17.1 mmol, 1.00 equiv) and 2,6-di-tert-butyl-4-methylpyridine (12.3 g, 59.7 mmol, 3.50 equiv) were azeotropically dried with benzene and dissolved in dichloromethane (330 mL). 4A molecular sieves (8.6 g) were added and the reaction flask was cooled to 0° C. A solution of triflic anhydride in dichloromethane (1 M, 34.1 mL, 34.1 mmol, 2.00 equiv) was added dropwise and the ice bath was removed to warm the reaction flask to room temperature. After 2 hours, the reaction was quenched with triethylamine (55 mL) and the filtered through a pad of Celite. Saturated NaHCO₃ solution (300 mL) was added and the aqueous phase was extracted with dichloromethane (2×350 mL). The combined organic phases were washed with brine (300 mL), dried over Na₂SO₄, and concentrated under reduced pressure. The residue was purified by flash chromatography (silica gel, eluent: 9:1→4:1 Benzene:Diethyl ether) to afford oxabicyclo[3.2.1]octene core skeleton 9 (3.76 g, 57%).

¹H NMR (500 MHz, CDCl₃) Shift=5.73 (s, 1H), 5.29-5.26 (m, 1H), 4.04-3.76 (m, 8H), 2.58-2.50 (m, 1H), 2.46 (t, J=15.1 Hz, 1H), 2.31-2.24 (m, 2H), 2.19 (t, J=11.2 Hz, 1H), 2.09 (d, J=13.2 Hz, 1H), 1.99 (dt, J=4.4, 13.2 Hz, 1H), 1.94 (dd, J=2.4, 13.2 Hz, 1H), 1.91-1.84 (m, 1H), 1.83-1.71 (m, 3H), 1.71-1.53 (m, 5H), 0.88 (s, 3H); ³C NMR (500 MHz, CDCl₃) Shift=140.6, 139.9, 119.9, 119.8, 118.5, 108.9, 81.5, 80.0, 65.2, 64.6, 64.5, 64.2, 46.2, 45.9, 42.4, 39.8, 34.0, 33.2, 32.4, 31.1, 28.0, 18.5, 17.0; HRMS (ESI) (m/z) calculated for C₂₃H₃₁O₅ [M+H]⁺: 387.2166, found 387.2180.

### Monoketone 10

To a solution of oxabicyclo[3.2.1]octene core skeleton 9 (3.24 g, 8.38 mmol, 1.00 equiv) in acetone (400 mL) and water (100 mL) was added PTSA (797 mg, 4.19 mmol, 0.50 equiv) and the reaction mixture was stirred for 3 days. Saturated NaHCO₃ solution (210 mL) and ethyl acetate (300 mL) were sequentially added to the reaction. The layers were separated and the aqueous layer was extracted with ethyl acetate (2×200 mL). The organic layers were combined, washed with brine (150 mL), dried over Na₂SO₄ and concentrated under reduced pressure. The resulting residue was then purified by flash chromatography (silica gel, eluent: 4:1 Hexanes:EtOAc) to afford monoketone 10 (2.50 g, 87%).

¹H NMR (500 MHz, CDCl₃) Shift=5.73 (s, 1H), 5.29-5.25 (m, 1H), 3.98-3.90 (m, 4H), 2.48 (dd, J=8.8, 19.5 Hz, 1H), 2.46-2.40 (m, 1H), 2.36 (dd, J=5.9, 12.7 Hz, 1H), 2.34-2.25 (m, 2H), 2.24-2.08 (m, 5H), 2.09 (d, J=13.2 Hz, 1H), 1.95 (dd, J=2.4, 13.2 Hz, 1H), 1.90-1.81 (m, 1H), 1.79-1.70 (m, 2H), 1.70-1.61 (m, 2H), 0.89 (s, 3H); ³C NMR (500 MHz, CDCl₃) Shift=220.9, 141.5, 140.6, 119.7, 118.6, 108.8, 81.1, 80.5, 64.7, 64.3, 47.9, 47.3, 42.5, 39.9, 36.0, 34.0, 33.9, 31.7, 28.1, 18.9, 17.0; HRMS (ESI) (m/z) calculated for C₂₁H₂₇₀₄ [M+H]⁺: 343.1909, found 343.1919.

### 1-Chloroisoquinoline Adduct 11

CeCl₃ (565 mg, 2.30 mmol, 10.0 equiv) in reaction flask was heated at 140° C. under vacuum for 2 h. The flask was charged with Ar and cooled to 0° C. After 30 min, THF (2.8 mL) was added and stirred at 0° C. for 2 h. The flask was then allowed to warm to room temperature and stirred for additional 16 h.

To a solution of CeCl₃/THF complex was added 1-chloro-7-iodoisoquinoline (396 mg, 1.40 mmol, 6.00 equiv) in THF (1.4 mL) followed by stirring for 10 min at room temperature, which was then allowed to cool to −78° C. A solution of n-butyllithium in hexanes (1.6 M, 716 μL, 1.10 mmol, 5.00 equiv) was then added dropwise. The reaction mixture was stirred additional 30 min at the same temperature and monoketone 10 (78.5 mg, 229 μmol) was cannulated in THF (1.4 mL). After additional 30 min, saturated NH₄Cl solution (5 mL) was added to the stirred reaction mixture, which was then allowed to warm to room temperature. The mixture was diluted with EtOAc (5 mL) and the layers were separated. The aqueous layer was extracted with EtOAc (3×5 mL) and the organic layers were combined, washed with brine (5 mL), and dried over Na₂SO₄ and concentrated under reduced pressure. The resulting residue was then purified by flash chromatography (silica gel, eluent: 2:1 Hexanes:EtOAc) to provide 1-chloroisoquinoline adduct 11 (115 mg, 97%).

¹H NMR (500 MHz, CDCl₃) Shift=8.34 (br. s, 1H), 8.24 (d, J=5.9 Hz, 1H), 7.89-7.83 (m, 1H), 7.76 (d, J=8.3 Hz, 1H), 7.56 (d, J=5.9 Hz, 1H), 5.63 (s, 1H), 5.16-4.99 (m, 1H), 4.02-3.87 (m, 4H), 2.62 (ddd, J=4.4, 9.8, 14.2 Hz, 1H), 2.48-2.38 (m, 2H), 2.36-2.26 (m, 3H), 2.26-2.19 (m, 1H), 2.18-2.08 (m, 2H), 1.96 (dd, J=2.4, 13.7 Hz, 1H), 1.88 (dd, J=5.1, 17.8 Hz, 1H), 1.82-1.70 (m, 2H), 1.67-1.57 (m, 3H), 1.49 (d, J=17.6 Hz, 1H), 1.20-1.08 (m, 3H); HRMS (ESI) (m/z) calculated for C₂₂H₂₆NaO₅ [M+Na]⁺: 393.1673, found 393.1657.

### Isoquinoline 12

A solution of 1-chloroisowuinoline adduct 11 (115 mg, 227 μmol) in dichloromethane (20 mL) was cooled to 0° C. Pyridine (183 μL, 2.30 mmol, 10.0 equiv) and DMAP (13.9 mg, 114 μmol, 0.50 equiv) were then added sequentially to the solution. After 5 min, trifluoroacetic anhydride (158 μL, 1.14 mmol, 5.00 equiv) was added dropwise and stirred additional 30 min, at which point pH 7 phosphate buffer (15 mL) was added followed by warming the reaction flask to room temperature. The organic and aqueous layers were separated and the aqueous layer was extracted with dichloromethane (2×15 mL). The organic layers were combined, washed with brine (25 mL), dried over Na₂SO₄, and concentrated under reduced pressure. The resulting residue was then purified by short flash column chromatography (silica gel, eluent: 2:1 Hexanes:EtOAc) to afford trifluoroacetylated product which was quickly used for the next step.

Trifluoroacetylated product (130 mg, 216 mmol) was azeotropically dried with benzene and dissolved in benzene (4.3 mL). AIBN (106 mg, 647 μmol, 3.00 equiv) was added and the reaction flask was degassed by the freeze-pump thaw process (3 cycles). Bu₃SnH (1.16 mL, 4.31 mmol, 20.0 equiv) was added and the reaction mixture was allowed to warm to reflux. After 3 h, the reaction mixture was cooled to room temperature and concentrated under reduced pressure. The resulting residue was then purified by flash column chromatography (silica gel, eluent: 4:1 to 3:1 to 1:1 Hexanes:EtOAc) to provide isoquinoline 12 (67.0 mg, 65% in two steps).

¹H NMR (500 MHz, CDCl₃) Shift=9.21 (s, 1H), 8.46 (d, J=5.9 Hz, 1H), 7.77 (s, 1H), 7.73 (d, J=8.3 Hz, 1H), 7.61 (d, J=5.9 Hz, 1H), 7.57 (d, J=8.3 Hz, 1H), 5.74 (s, 1H), 5.29-5.23 (m, 1H), 4.00-3.90 (m, 4H), 3.11 (t, J=10.0 Hz, 1H), 2.49 (dd, J=8.3, 11.2 Hz, 1H), 2.47-2.41 (m, 1H), 2.38-2.24 (m, 4H), 2.24-2.14 (m, 2H), 2.12 (d, J=13.2 Hz, 1H), 2.06-1.95 (m, 2H), 1.91 (dd, J=5.4, 17.6 Hz, 1H), 1.83 (dq, J=4.9, 11.7 Hz, 1H), 1.77 (td, J=2.3, 12.9 Hz, 1H), 1.72-1.59 (m, 3H), 0.52 (s, 3H); ³C NMR (500 MHz, CDCl₃) Shift=152.4, 142.6, 141.2, 140.6, 140.2, 134.7, 132.1, 128.7, 126.4, 125.8, 120.2, 119.9, 119.3, 108.9, 81.4, 80.3, 64.7, 64.3, 57.1, 51.8, 44.9, 42.6, 40.1, 39.8, 34.2, 30.9, 28.2, 26.5, 20.7, 15.3; HRMS (ESI) (m/z) calculated for C₃₀H₃₃NaNO₃ [M+Na]⁺: 478.2353, found 478.2347.

### Ketone 13

To a solution of isoquinoline 12 (365 mg, 0.801 mmol, 1.00 equiv) in acetone and water (4:1, 0.025 M) was added PTSA (412 mg, 2.16 mmol, 2.70 equiv) and the reaction mixture was warmed to 55° C. After 14.5 hours, the reaction was cooled to room temperature and saturated NaHCO₃ solution and ethyl acetate were sequentially added to the reaction. The layers were separated and the aqueous layer was extracted with ethyl acetate. The organic layers were combined, washed with brine, dried over Na₂SO₄ and concentrated under reduced pressure. The resulting residue was then purified by flash chromatography (silica gel, eluent: 3:2→1:2 Hexanes:EtOAc) to afford ketone 13 (289 mg, 87%).

¹H NMR (500 MHz, CDCl₃) Shift=9.23 (s, 1H), 8.48 (d, J=5.9 Hz, 1H), 7.80 (s, 1H), 7.78 (d, J=8.3 Hz, 1H), 7.65 (d, J=5.9 Hz, 1H), 7.61 (d, J=8.3 Hz, 1H), 5.91 (s, 1H), 5.40-5.35 (m, 1H), 3.15 (t, J=10.0 Hz, 1H), 2.94 (d, J=15.1 Hz, 1H), 2.68 (d, J=15.1 Hz, 1H), 2.67-2.59 (m, 1H), 2.58-2.41 (m, 4H), 2.41-2.24 (m, 3H), 2.24-2.10 (m, 2H), 2.04 (tt, J=4.6, 13.2 Hz, 1H), 1.96 (dd, J=5.4, 17.6 Hz, 1H), 1.86 (dq, J=5.1, 12.1 Hz, 1H), 1.80-1.67 (m, 2H), 0.55 (s, 3H); ³C NMR (500 MHz, CDCl₃) Shift=208.9, 152.2, 142.2, 140.3, 140.2, 139.4, 134.9, 132.3, 128.7, 126.5, 126.0, 121.5, 120.9, 120.4, 82.8, 80.4, 57.1, 51.7, 49.2, 44.8, 40.1, 40.0, 39.8, 30.8, 29.8, 28.1, 26.5, 20.7, 15.4; HRMS (ESI) (m/z) calculated for C₂₈H₃₀NO₂ [M+H]⁺: 412.2271, found 412.2288.

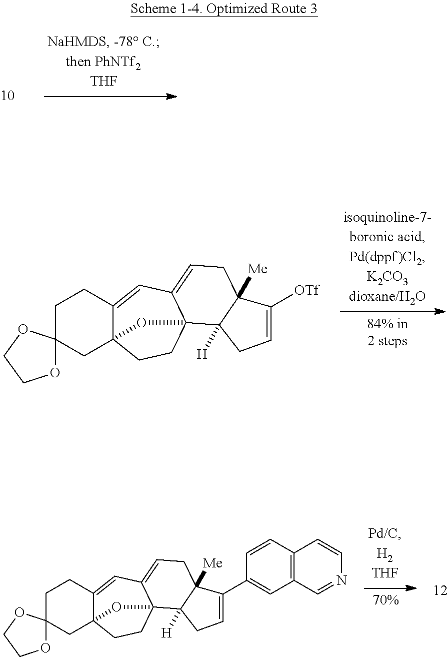

### Triflate

To a solution of monoketone 10 (2.50 g, 7.30 mmol, 1.00 equiv) in THF (45 mL) was added NaHMDS (1 M, 8.76 mL, 8.76 mmol, 1.20 equiv) at −78° C., dropwise. After stirring 1.5 hours, PhNTf₂ (3.91 g, 11.0 mmol, 1.50 equiv) in THF (20 mL) was cannulated and the reaction mixture was warmed up to 0° C. After additional 30 min, saturated NH₄Cl solution (50 mL) was added to the stirred reaction mixture and diluted with EtOAc (70 mL). The layers were separated and the aqueous layer was extracted with EtOAc (2×45 mL) and the organic layers were combined, washed with brine (80 mL), dried over Na₂SO₄, and concentrated under reduced pressure. The resulting residue was then purified by flash column chromatography (silica gel, eluent: 8:1→5:1 Hexanes:EtOAc) to provide triflate (3.33 g, yield was calculated after cross-coupling due to the inseparable minor impurity).

¹H NMR (500 MHz, CDCl₃) Shift=5.76 (s, 1H), 5.67 (br. s., 1H), 5.32 (dd, J=2.0, 4.9 Hz, 1H), 4.02-3.94 (m, 4H), 2.67 (dd, J=6.8, 10.7 Hz, 1H), 2.49 (t, J=14.6 Hz, 1H), 2.45 (ddd, J=3.7, 6.5, 15.2 Hz, 1H), 2.38-2.28 (m, 4H), 2.17 (ddd, J=1.5, 10.7, 12.7 Hz, 1H), 2.12 (d, J=13.2 Hz, 1H), 2.10 (dd, J=5.9, 17.6 Hz, 1H), 1.98 (dd, J=2.7, 13.4 Hz, 1H), 1.88 (ddd, J=7.6, 8.9, 12.8 Hz, 1H), 1.80 (tdd, J=2.4, 4.8, 12.7 Hz, 1H), 1.74-1.63 (m, 2H), 1.03 (s, 3H); HRMS (ESI) (m/z) calculated for C₂₂H₂₆O₆F₃S [M+H]⁺: 475.1397, found 475.1411.

### C16-C17 Unsaturated Isoquinoline

To a solution of triflate (3.33 mg, 7.02 mmol, 1.00 equiv) and isoquinoline-7-boronic acid (3.64 g, 21.1 mmol, 3.00 equiv) in 1,4-dioxane (300 mL) and H₂O (30 mL) was added K₂CO₃ (2.91 g, 21.1 mmol, 3.00 equiv) and the solution was bubbled through inert Ar for 5 min. Pd(dppf)Cl₂.CH₂C12 (286 mg, 350 μmol, 0.05 equiv) was added and the reaction mixture was stirred at 80° C. for 1 hour. The mixture was allowed to cool to room temperature and saturated NaHCO₃ solution (200 mL) was applied. The mixture was diluted with EtOAc (350 mL) and the layers were separated. The aqueous layer was extracted with EtOAc (2×300 mL) and the combined organic layers were washed with brine (500 mL), dried over Na₂SO₄, and concentrated under reduced pressure. The crude mixture was purified by flash column chromatography (silica gel, eluent: 2:1→1:1→1:2 Hexanes:EtOAc) to provide C16-C17 unsaturated isoquinoline (2.67 mg, 84% over 2 steps).

¹H NMR (500 MHz, CDCl₃) Shift=9.23 (s, 1H), 8.49 (d, J=5.4 Hz, 1H), 7.94 (s, 1H), 7.85-7.81 (m, 1H), 7.80-7.75 (m, 1H), 7.63 (d, J=5.4 Hz, 1H), 6.26 (br. s., 1H), 5.82 (s, 1H), 5.40 (d, J=3.4 Hz, 1H), 4.08-3.90 (m, 4H), 2.76 (dd, J=7.1, 11.0 Hz, 1H), 2.58 (dt, J=5.4, 17.6 Hz, 1H), 2.56-2.40 (m, 3H), 2.40-2.28 (m, 4H), 2.16 (d, J=13.2 Hz, 1H), 2.02 (dd, J=2.0, 13.2 Hz, 1H), 1.94 (td, J=8.8, 13.2 Hz, 1H), 1.81 (td, J=2.0, 12.7 Hz, 1H), 1.76-1.67 (m, 2H), 1.18 (s, 3H; HRMS (ESI) (m/z) calculated for C₃₀H₃₂NO₃ [M+H]⁺: 454.2377, found 454.2366.

### Isoquinoline 12

To a solution of 17,18-unsaturated isoquinoline (534 mg, 1.17 mmol, 1.00 equiv) in THF (48 mL) was added 10 wt % Pd/C (374 mg, 351 μmol, 0.30 equiv) and H₂ balloon was installed. After 3 h, the reaction mixture was filtered through a pad of Celite and washed with 0.2 M NH₃ solution in MeOH (50 mL), concentrated under reduced pressure. The residue was purified by flash column chromatography (silica gel, eluent: 40:1→30:1 DCM:MeOH) to provide isoquinoline 12 (452 mg, 84%).

### Example S2. Synthesis of Lactams of Formula (A-1), (A-1′), or (A-′″) and (A-2′) or (A-2″)

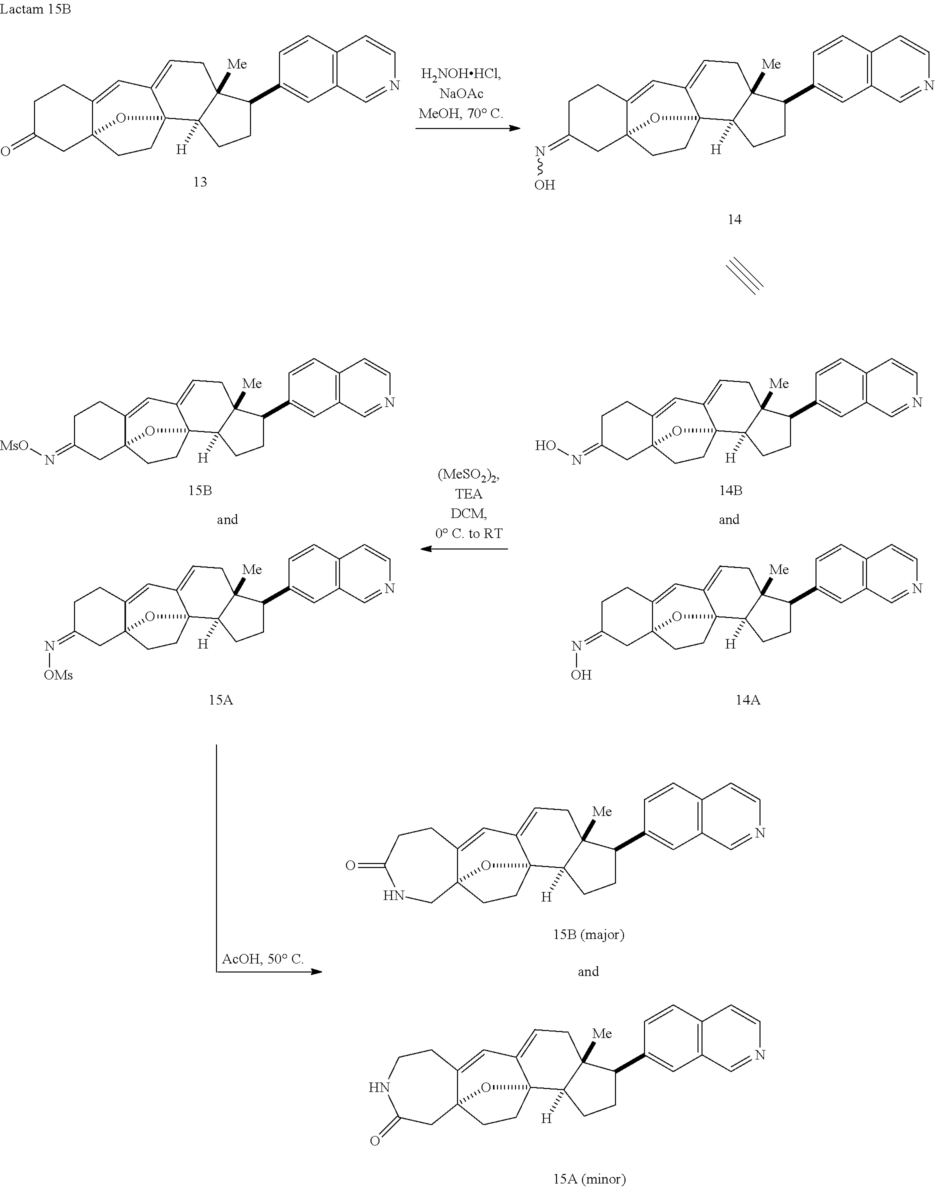

To a solution of ketone 13 (5.5 mg, 13.6 μmol, 1.00 equiv) in MeOH (350 μL) was added H₂NOH.HCl (2.5 mg, 27.2 μmol, 2.00 equiv) and NaOAc (4.9 mg, 27.2 μmol, 2.00 equiv). After stirring 1.5 h at 70° C., the reaction mixture was cooled to room temperature and roughly concentrated. H₂O (300 μL) was added and extracted with ethyl acetate (3×300 μL), and the combined organic phases were washed with brine (300 μL), dried over Na₂SO₄, and concentrated under reduced pressure. The crude mixture was used in the next step without further purification.

To a solution of crude mixture (13.6 μmol, 1.00 equiv) in DCM (350 μL) was added trimethylamine (11.4 μL, 81.6 μmol, 6.00 equiv). At 0° C., methanesulfonic anhydride (4.7 mg, 27.2 μmol, 2.00 equiv) was added. The reaction mixture was stirred 15 min at 0° C. and warmed up to room temperature for additional 15 min stirring. The reaction mixture was quenched with NaHCO₃ (300 μL) and extracted with DCM (3×300 μL), and the combined organic phases were washed with brine (300 μL), dried over Na₂SO₄, and concentrated under reduced pressure. The crude mixture was used in the next step without further purification.

The crude mixture was dissolved in AcOH (300 μL) and stirred at 50° C. for 16 h. The reaction mixture was roughly concentrated and NaHCO₃ (300 μL) was applied. It was extracted with ethyl acetate (3×300 tL), and the combined organic phases were washed with brine (300 L), dried over Na₂SO₄, and concentrated under reduced pressure. The crude mixture was purified by preparative TLC (silica gel, eluent: 5:5:1 EtOAc:DCM:TEA) to afford lactam 15B (1.5 mg, 26% in three steps).

¹H NMR (500 MHz, CDCl₃) Shift=9.22 (s, 1H), 8.49 (d, J=5.9 Hz, 1H), 7.79 (s, 1H), 7.76 (d, J=8.2 Hz, 1H), 7.63 (d, J=5.3 Hz, 1H), 7.58 (dd, J=1.5, 8.5 Hz, 1H), 5.87 (s, 1H), 5.78 (t, J=6.5 Hz, 1H), 5.34 (dd, J=2.6, 5.0 Hz, 1H), 3.57 (dd, J=5.6, 15.0 Hz, 1H), 3.33 (dd, J=7.6, 15.3 Hz, 1H), 3.15 (dd, J=9.1, 10.9 Hz, 1H), 2.66 (ddd, J=4.7, 10.0, 14.7 Hz, 1H), 2.62-2.53 (m, 2H), 2.52-2.46 (m, 2H), 2.35 (br. s., 1H), 2.38-2.30 (m, 1H), 2.28-2.22 (m, 1H), 2.22-2.12 (m, 2H), 2.01 (qt, J=4.1, 9.4 Hz, 1H), 1.96 (dd, J=5.3, 17.6 Hz, 1H), 1.90-1.79 (m, J=5.3, 12.3, 12.3 Hz, 1H), 1.75 (td, J=8.2, 12.3 Hz, 1H), 1.68 (dt, J=7.3, 10.7 Hz, 1H), 0.54 (s, 3H). HRMS (ESI) (m/z) calculated for C₂₈H₃₁N₂O₂ [M+H]⁺: 427.2380, found 427.2395.

### Example S3. Reductive Amination

### Method A

To a solution of ketone 13 (1.00 equiv) in 1,2-dichloroethane (0.02 M) was sequentially added amine (4.00 equiv), AcOH (1.50 equiv), and NaBH₃CN (3.50 equiv) at room temperature. Triethylamine (4 equiv) was added if the reacting amine is a form of HCl salt (Method AA). Once the reaction is done, saturated NaHCO₃ solution was added and the layers were separated. The aqueous layer was extracted with dichloromethane. The organic layers were combined, washed with brine, dried over Na₂SO₄ and concentrated under reduced pressure. (α-NR₂: β-NR₂=˜1:1.2 to ˜1:5).

### Method B

To a solution of ketone 13 (1.00 equiv) in 1,2-dichloroethane (0.02 M) was sequentially added amine (2.00 equiv), AcOH (2.00 equiv), and NaBH(OAc)₃ (2.00 equiv) at room temperature. Triethylamine (2.00 equiv) was added if the reacting amine is a form of HCl salt (Method BB). Once the reaction is done, saturated NaHCO₃ solution was added and the layers were separated. The aqueous layer was extracted with dichloromethane. The organic layers were combined, washed with brine, dried over Na₂SO₄ and concentrated under reduced pressure. (α-NR₂: β-NR₂=˜1:1.2 to ˜1:5).

### Method C

To a solution of secondary amine (1.00 equiv) in dichloromethane (0.02 M) was added formaldehyde or acetaldehyde (5.00 equiv) and stirred 1 h at room temperature before the addition of NaBH(OAc)₃ (2.00 equiv). Once the reaction is done, saturated NaHCO₃ solution was added and the layers were separated. The aqueous layer was extracted with dichloromethane. The organic layers were combined, washed with brine, dried over Na₂SO₄ and concentrated under reduced pressure.

### Method D: General Method for Favoring α-Amine

To a solution of ketone 13 (1.00 equiv) in THF and t-BuOH (4:1, 0.02 M) was added amine (5.00 equiv) and Ti(Oi-Pr)₄ (3.00 equiv) sequentially, and stirred at room temperature for 15 hours (4 hours for Me₂NH, MeNH₂, and NH₃). The reaction mixture was cooled to −20° C. and NaBH₄ (1.50 equiv) was added. Once the reaction is done, saturated NaHCO₃ solution was added and the layers were separated. The aqueous layer was extracted with EtOAc. The organic layers were combined, washed with brine, dried over Na₂SO₄ and concentrated under reduced pressure. (α-NR₂: β-NR₂=−1.1:1 to ˜3.7:1).

### Method E: General Method for Methanesulfonamide Formation

To a solution of amine (1.00 equiv) in dichloromethane (0.013 M) was added trimethylamine (4.00 equiv) and the reaction mixture was cooled to ˜20° C. Methanesulfonic anhydride (2.50 equiv) was added as a solution in dichloromethane and stirred 30 min at the same temperature. 2 N NaOH solution was added and the layers were separated. The aqueous layer was extracted with dichloromethane. The organic layers were combined, washed with brine, dried over Na₂SO₄ and concentrated under reduced pressure.

### β-Dimethylamine 14B and α-Dimethylamine 14A

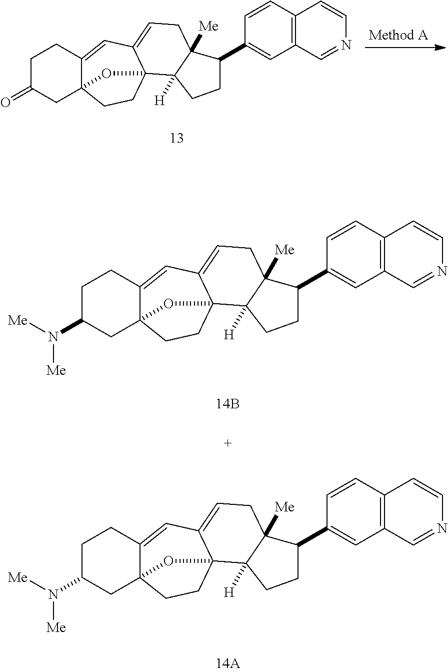

The crude mixture was purified sequentially by flash chromatography (silica gel, eluent: 20:1 EtOAc:2M NH₃ solution in MeOH) to afford β-dimethylamine 14B (21.5 mg, 65%). ca. 0.6 mg of α-dimethylamine 14A was prepared from 3 mg of 13 by HPLC (Eclipse XDB-C8 column, 9.4 mm×25 cm; gradient=0%→35% MeCN (0.1% formic acid):H₂O (0.1% formic acid) over 30 min)

β-dimethylamine 14B: ¹H NMR (500 MHz, C₆D₆) Shift=9.31 (s, 1H), 8.61 (d, J=5.4 Hz, 1H), 7.43 (s, 1H), 7.39 (d, J=8.8 Hz, 1H), 7.25 (d, J=5.4 Hz, 1H), 7.23 (d, J=8.8 Hz, 1H), 5.73 (br. s, 1H), 5.18 (s, 1H), 2.74 (t, J=10.0 Hz, 1H), 2.63 (dd, J=8.8, 11.2 Hz, 1H), 2.48-2.28 (m, 2H), 2.27-2.20 (m, 1H), 2.19-2.03 (m, 6H), 2.00 (br. s, 6H), 1.95-1.84 (m, 2H), 1.83-1.66 (m, 5H), 1.41 (tt, J=5.4, 13.2 Hz, 1H), 0.45 (s, 3H). HRMS (ESI) (m/z) calculated for C₃₀H₃₇N₂O [M+H]⁺: 441.2900, found 441.2910.

α-dimethylamine 14A: ¹H NMR (600 MHz, C₆D₆) Shift=9.26 (s, 1H), 8.56 (d, J=5.9 Hz, 1H), 7.44-7.39 (m, 1H), 7.36 (d, J=8.2 Hz, 1H), 7.21-7.20 (m, 1H), 7.20 (d, J=5.9 Hz, 1H), 5.68-5.65 (m, 1H), 5.15-5.11 (m, 1H), 2.72-2.66 (m, J=10.0 Hz, 1H), 2.59 (dd, J=8.8, 11.2 Hz, 1H), 2.34 (tt, J=2.9, 12.1 Hz, 1H), 2.16 (td, J=3.2, 16.0 Hz, 1H), 2.09 (s, 6H), 2.13-1.92 (m, 8H), 1.85 (ddd, J=5.0, 9.0, 13.6 Hz, 1H), 1.73 (dt, J=5.3, 12.3 Hz, 1H), 1.72-1.66 (m, 2H), 1.60-1.57 (m, 1H), 1.57-1.49 (m, 1H), 1.20 (dq, J=4.1, 12.3 Hz, 1H), 0.40 (s, 3H). HRMS (ESI) (m/z) calculated for C₃₀H₃₇N₂O [M+H]⁺: 441.2900, found 441.2909.

### ββ-Morpholine 15B and α-Morpholine 15A

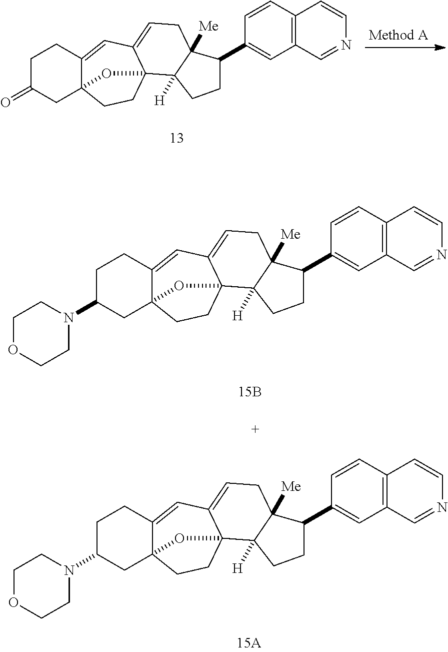

β-Morpholine 15B: The crude mixture was purified by flash chromatography (silica gel, eluent: 100% EtOAc→35:1→20:1→10:1 EtOAc:MeOH) to afford β-morpholine 15B (21 mg, 66%). ¹H NMR (500 MHz, CDCl₃) Shift=9.22 (s, 1H), 8.48 (d, J=5.9 Hz, 1H), 7.79 (s, 1H), 7.75 (d, J=8.3 Hz, 1H), 7.62 (d, J=5.9 Hz, 1H), 7.59 (dd, J=1.0, 8.8 Hz, 1H), 5.71 (s, 1H), 5.24 (d, J=2.9 Hz, 1H), 3.73 (br. s, 4H), 3.13 (t, J=10.0 Hz, 1H), 2.65-2.28 (m, 11H), 2.23-2.11 (m, 3H), 2.06 (d, J=13.2 Hz, 1H), 2.01 (dt, J=4.4, 9.0 Hz, 1H), 1.93 (dd, J=4.9, 17.1 Hz, 1H), 1.89-1.79 (m, 1H), 1.75-1.53 (m, 4H), 0.54 (s, 3H). HRMS (ESI) (m/z) calculated for C₃₂H₃₉N₂O₂ [M+H]⁺: 483.3006, found 483.3012.

### β-N-Methylpiperazine 16B and α-N-Methylpiperazine 16A

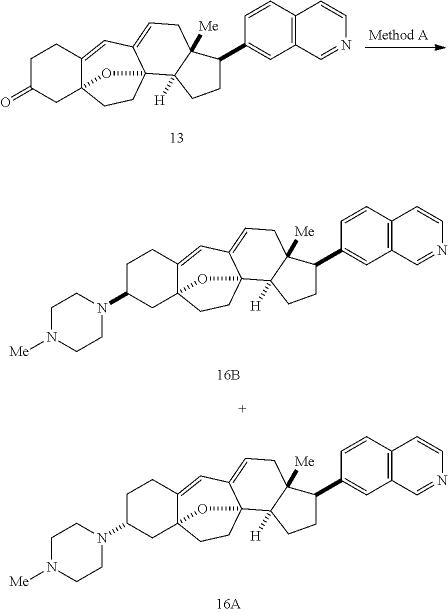

β-N-Methylpiperazine 16B: The crude mixture was purified sequentially by flash chromatography (silica gel, 1^{st} column: eluent: 100% MeOH→10:1 EtOAc:2M NH₃ solution in MeOH/2^{nd} column: eluent: 20:1 EtOAc:2M NH₃ solution in MeOH)) to afford β-N-methylpiperazine 16B (20 mg, 55%). ¹H NMR (600 MHz, CDCl₃) Shift=9.22 (s, 1H), 8.48 (d, J=5.9 Hz, 1H), 7.79 (s, 1H), 7.75 (d, J=8.8 Hz, 1H), 7.62 (d, J=5.9 Hz, 1H), 7.59 (d, J=8.2 Hz, 1H), 5.70 (s, 1H), 5.25-5.22 (m, 1H), 3.13 (t, J=9.7 Hz, 1H), 2.53 (br. s., 1H), 2.50 (dd, J=8.8, 11.7 Hz, 1H), 2.41 (t, J=12.9 Hz, 1H), 2.38-2.33 (m, 3H), 2.32 (br. s, 3H), 2.22-2.11 (m, 3H), 2.10-1.95 (m, 3H), 1.95-1.89 (m, 2H), 1.84 (dq, J=5.3, 11.7 Hz, 1H), 1.79-1.50 (m, 11H), 0.62-0.43 (m, 3H). HRMS (ESI) (m/z) calculated for C₃₃H₄₂N₃O [M+H]⁺: 496.3322, found 496.3337.

### β-Azetidine 18B and α-Azetidine 18A

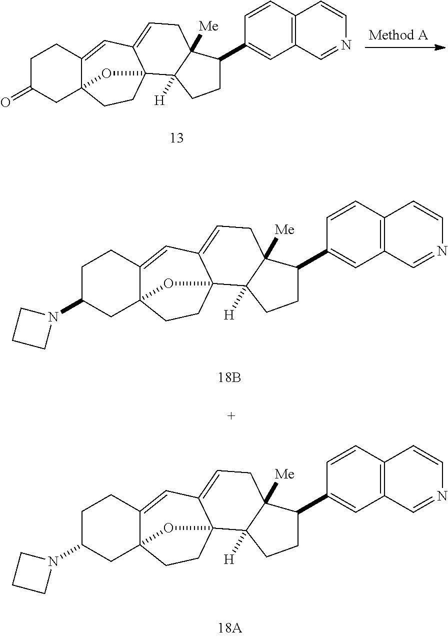

β-Azetidine 18B: The crude mixture was purified by preparative TLC (eluent: 1:1 EtOAc:MeOH) to afford β-azetidine 18B (2.7 mg, 50%). ¹H NMR (500 MHz, CDCl₃) Shift=9.22 (s, 1H), 8.48 (d, J=5.9 Hz, 1H), 7.79 (s, 1H), 7.75 (d, J=8.8 Hz, 1H), 7.62 (d, J=5.4 Hz, 1H), 7.59 (d, J=8.3 Hz, 1H), 5.69 (s, 1H), 5.22 (d, J=2.4 Hz, 1H), 3.20-3.05 (m, 5H), 2.59-2.43 (m, 4H), 2.39-2.28 (m, 2H), 2.23-2.12 (m, 2H), 2.07-1.96 (m, 4H), 1.92 (dd, J=5.1, 17.3 Hz, 1H), 1.89-1.79 (m, 3H), 1.75-1.55 (m, 3H), 1.40 (t, J=13.2 Hz, 1H), 0.54 (s, 3H). HRMS (ESI) (m/z) calculated for C₃₁H₃₇N₂O [M+H]⁺: 453.2906, found 453.2916.

### β-Pyrrolidine 19B and α-Pyrrolidine 19A

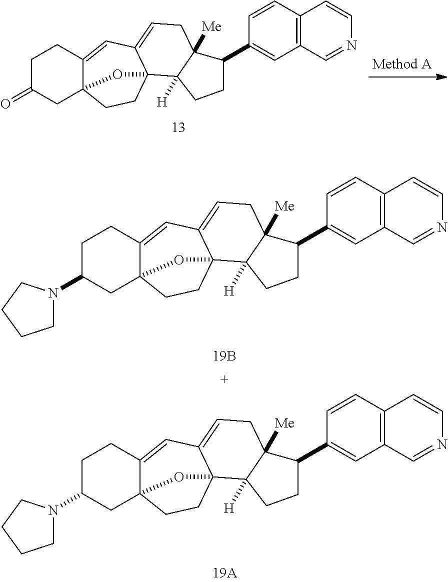 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CC[C@H](N5CCCC5)C[C@]45CC[C@]3(O5)[C@@H]1CC[C@@H]2c1ccc2ccncc2c1 ; C[C@]12CC=C3C=C4CC[C@@H](N5CCCC5)C[C@]45CC[C@]3(O5)[C@@H]1CC[C@@H]2c1ccc2ccncc2c1]

β-Pyrrolidine 19B: The crude mixture was purified by preparative TLC (eluent: 20:10:3 EtOAc:Hexane: 2M NH₃ solution in MeOH) to afford β-pyrrolidine 19B (2.0 mg, 40%). ¹H NMR (500 MHz, CDCl₃) Shift=9.22 (s, 1H), 8.48 (d, J=5.4 Hz, 1H), 7.79 (s, 1H), 7.75 (d, J=8.3 Hz, 1H), 7.62 (d, J=5.9 Hz, 1H), 7.59 (d, J=8.3 Hz, 1H), 5.70 (br. s., 1H), 5.22 (br. s., 1H), 3.13 (t, J=9.8 Hz, 1H), 2.59-2.46 (m, 6H), 2.44 (br. s., 1H), 2.41-2.28 (m, 3H), 2.23-2.12 (m, 2H), 2.11-2.00 (m, 2H), 2.00-1.82 (m, 4H), 1.79-1.65 (m, 6H), 1.64-1.51 (m, 2H), 0.54 (s, 3H). HRMS (ESI) (m/z) calculated for C₃₂H₃₉N₂O [M+H]⁺: 467.3057, found 467.3053.

### β-Dimethylamine 17,18-Unsaturated Isoquinoline 23B and α-Dimethylamine 17,18-Unsaturated Isoquinoline 23A

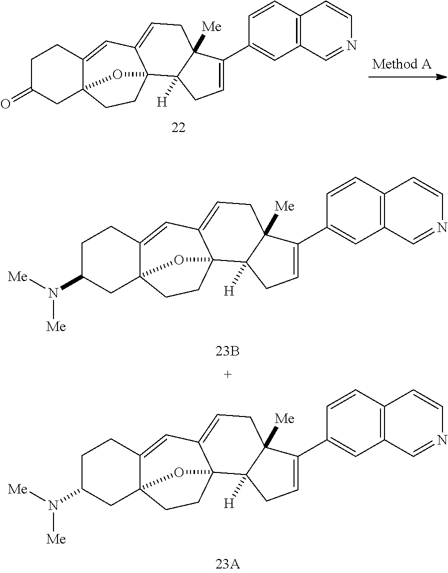 [SMILES: C[C@]12CC=C3C=C4CCC(=O)C[C@]45CC[C@]3(O5)[C@@H]1CC=C2c1ccc2ccncc2c1 ; CN(C)[C@H]1CCC2=CC3=CC[C@]4(C)C(c5ccc6ccncc6c5)=CC[C@H]4[C@@]34CC[C@]2(C1)O4 ; CN(C)[C@@H]1CCC2=CC3=CC[C@]4(C)C(c5ccc6ccncc6c5)=CC[C@H]4[C@@]34CC[C@]2(C1)O4]

β-Dimethylamine 17,18-unsaturated isoquinoline 23B: The crude mixture was purified sequentially by flash chromatography (silica gel, eluent: 20:1 EtOAc:2M NH₃ solution in MeOH) to afford β-dimethylamine 17,18-unsaturated isoquinoline 23B (6.5 mg, 74%). ¹H NMR (500 MHz, CDCl₃) Shift=9.24 (br. s., 1H), 8.51 (d, J=5.4 Hz, 1H), 7.94 (s, 1H), 7.84-7.76 (m, 2H), 7.63 (d, J=5.4 Hz, 1H), 6.27 (br. s., 1H), 5.97 (s, 1H), 5.50 (dd, J=2.4, 4.9 Hz, 1H), 2.98 (d, J=14.6 Hz, 1H), 2.78 (dd, J=6.8, 11.2 Hz, 1H), 2.71 (d, J=14.6 Hz, 1H), 2.72-2.63 (m, 1H), 2.61 (d, J=5.4 Hz, 1H), 2.59-2.54 (m, 2H), 2.54-2.50 (m, 2H), 2.50-2.42 (m, 2H), 2.39 (ddd, J=1.5, 11.0, 12.9 Hz, 1H), 2.20 (ddd, J=1.5, 9.5, 11.5 Hz, 1H), 2.01 (ddd, J=7.3, 8.8, 12.7 Hz, 1H), 1.79 (dt, J=7.3, 11.2 Hz, 1H), 1.18 (s, 3H). HRMS (ESI) (m/z) calculated for C₃₀H₃₅N₂O [M+H]⁺: 439.2744, found 439.2753.

### β-Monomethylamine 24B and α-Monomethylamine 24A

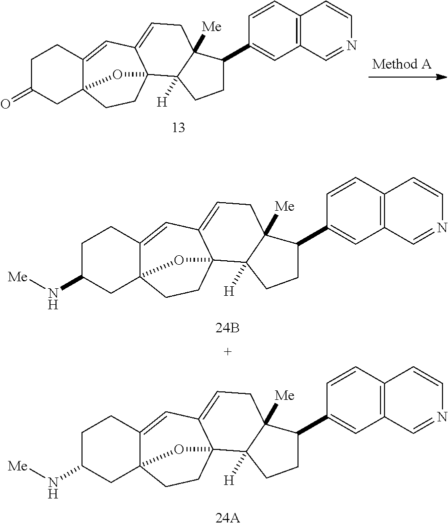 [SMILES: C[C@]12CC=C3C=C4CCC(=O)C[C@]45CC[C@]3(O5)[C@@H]1CC[C@@H]2c1ccc2ccncc2c1 ; CN[C@H]1CCC2=CC3=CC[C@]4(C)[C@@H](c5ccc6ccncc6c5)CC[C@H]4[C@@]34CC[C@]2(C1)O4 ; CN[C@@H]1CCC2=CC3=CC[C@]4(C)[C@@H](c5ccc6ccncc6c5)CC[C@H]4[C@@]34CC[C@]2(C1)O4]

β-Monomethylamine 24B:

The crude mixture was purified by preparative TLC (eluent: 10:1 EtOAc:2M NH₃ solution in MeOH) to afford β-monomethylamine 24B (ca. 1.5 mg, 58%). ¹H NMR (500 MHz, CDCl₃) Shift=9.22 (s, 1H), 8.48 (d, J=5.4 Hz, 1H), 7.79 (s, 1H), 7.75 (d, J=8.8 Hz, 1H), 7.62 (d, J=5.9 Hz, 1H), 7.59 (dd, J=1.0, 8.3 Hz, 1H), 5.72 (d, J=1.0 Hz, 1H), 5.24 (dd, J=2.2, 5.1 Hz, 1H), 3.13 (t, J=10.0 Hz, 1H), 3.03-2.98 (m, 1H), 2.57-2.50 (m, 1H), 2.51 (dd, J=8.3, 11.7 Hz, 1H), 2.44 (s, 3H), 2.36 (d, J=15.2 Hz, 1H), 2.36-2.28 (m, 2H), 2.26-2.13 (m, 2H), 2.09 (dd, J=3.7, 16.4 Hz, 1H), 2.07-1.99 (m, 2H), 1.98-1.92 (m, 1H), 1.93 (dd, J=5.9, 17.6 Hz, 1H), 1.85 (dq, J=4.9, 11.7 Hz, 1H), 1.82-1.76 (m, 1H), 1.76-1.58 (m, 3H), 0.54 (s, 3H). HRMS (ESI) (m/z) calculated for C₂₉H₃₅N₂O [M+H]⁺: 427.2744, found 427.2740.

### β-Deuterodimethylamine 26B and α-Deuterodimethylamine 26A

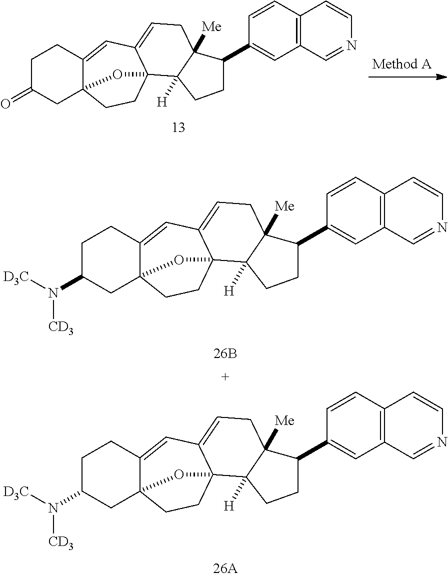 [SMILES: C[C@]12CC=C3C=C4CCC(=O)C[C@]45CC[C@]3(O5)[C@@H]1CC[C@@H]2c1ccc2ccncc2c1 ; [2H]C([2H])([2H])N([C@H]1CCC2=CC3=CC[C@]4(C)[C@@H](c5ccc6ccncc6c5)CC[C@H]4[C@@]34CC[C@]2(C1)O4)C([2H])([2H])[2H] ; [2H]C([2H])([2H])N([C@@H]1CCC2=CC3=CC[C@]4(C)[C@@H](c5ccc6ccncc6c5)CC[C@H]4[C@@]34CC[C@]2(C1)O4)C([2H])([2H])[2H]]

β-Deuterodimethylamine 26B:

Triethylamine was added The crude mixture was purified sequentially by flash chromatography (silica gel, eluent: 20:1 EtOAc:2M NH₃ solution in MeOH) to afford β-deuterodimethylamine 26B (4 mg, 62%). ¹H NMR (500 MHz, CDCl₃) Shift=9.24 (s, 1H), 8.50 (d, J=5.9 Hz, 1H), 7.80 (s, 1H), 7.77 (d, J=8.3 Hz, 1H), 7.64 (d, J=5.9 Hz, 1H), 7.60 (d, J=8.3 Hz, 1H), 5.74 (br. s., 1H), 5.26 (br. s., 1H), 3.15 (t, J=10.0 Hz, 1H), 2.51 (dd, J=8.8, 11.2 Hz, 1H), 2.50-2.42 (m, 1H), 2.37 (d, J=17.1 Hz, 1H), 2.38-2.26 (m, 2H), 2.26-2.09 (m, 4H), 2.08-1.98 (m, 2H), 1.95 (dd, J=5.1, 17.3 Hz, 1H), 1.87 (dq, J=5.4, 12.2 Hz, 1H), 1.80-1.68 (m, 3H), 1.62 (br. s., 2H), 0.63-0.50 (s, 3H). HRMS (ESI) (m/z) calculated for C₃₀H₃₁D₆N₂O [M+H]⁺: 447.3277, found 447.3281.

### β-2-Methoxyethylmethylamine 27B and α-2-Methoxyethylmethylamine 27A

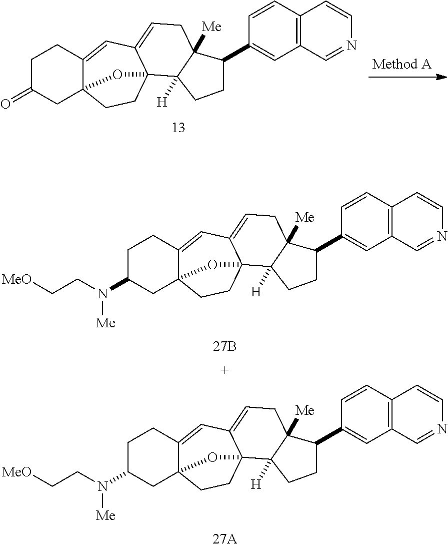 [SMILES: C[C@]12CC=C3C=C4CCC(=O)C[C@]45CC[C@]3(O5)[C@@H]1CC[C@@H]2c1ccc2ccncc2c1 ; COCCN(C)[C@H]1CCC2=CC3=CC[C@]4(C)[C@@H](c5ccc6ccncc6c5)CC[C@H]4[C@@]34CC[C@]2(C1)O4 ; COCCN(C)[C@@H]1CCC2=CC3=CC[C@]4(C)[C@@H](c5ccc6ccncc6c5)CC[C@H]4[C@@]34CC[C@]2(C1)O4]

β-2-Methoxyethylmethylamine 27B:

The crude mixture was purified by preparative TLC (eluent: 10:10:1 Hexanes:EtOAc:2M NH₃ solution in MeOH) to afford β-2-methoxyethylmethylamine 27B (ca. 1.2 mg, 20%). ¹H NMR (500 MHz, CD₃OD) Shift=9.21 (s, 1H), 8.40 (d, J=5.4 Hz, 1H), 7.99 (s, 1H), 7.90 (d, J=8.8 Hz, 1H), 7.81 (d, J=5.9 Hz, 1H), 7.77 (s, 1H), 5.80 (s, 1H), 5.35-5.27 (m, 1H), 3.60 (t, J=5.4 Hz, 2H), 3.39 (s, 2H), 3.24 (t, J=10.0 Hz, 1H), 3.15-2.88 (m, 2H), 2.56 (br. s., 3H), 2.51 (dd, J=9.3, 10.7 Hz, 1H), 2.48-2.39 (m, 3H), 2.35-2.28 (m, 1H), 2.25-2.09 (m, 4H), 2.02-1.84 (m, 7H), 1.76 (s, 2H), 0.59 (s, 3H). HRMS (ESI) (m/z) calculated for C₃₂H₄₁N₂O₂ [M+H]⁺: 485.3163, found 485.3170.

β-Bis-2-methoxyethylamine 28B and α-Bis-2-methoxyethylamine 28A

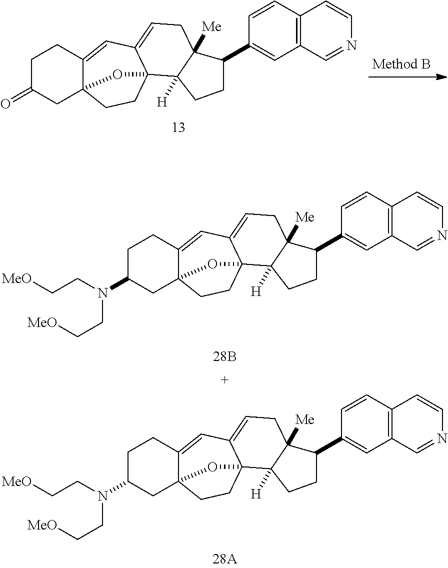 [SMILES: C[C@]12CC=C3C=C4CCC(=O)C[C@]45CC[C@]3(O5)[C@@H]1CC[C@@H]2c1ccc2ccncc2c1 ; COCCN(CCOC)[C@H]1CCC2=CC3=CC[C@]4(C)[C@@H](c5ccc6ccncc6c5)CC[C@H]4[C@@]34CC[C@]2(C1)O4 ; COCCN(CCOC)[C@@H]1CCC2=CC3=CC[C@]4(C)[C@@H](c5ccc6ccncc6c5)CC[C@H]4[C@@]34CC[C@]2(C1)O4]

p-Bis-2-methoxyethylamine 28B:

The crude mixture was purified by preparative TLC (eluent: 10:1 Dichloromethane:MeOH) to afford β-bis-2-methoxyethylamine 28B (ca. 1.1 mg, 19%). ¹H NMR (500 MHz, CD₃OD) Shift=9.21 (s, 1H), 8.39 (d, J=5.9 Hz, 1H), 7.99 (s, 1H), 7.90 (d, J=8.8 Hz, 1H), 7.81 (d, J=5.9 Hz, 1H), 7.77 (s, 1H), 5.74 (s, 1H), 5.29-5.24 (m, 1H), 3.47 (t, J=6.1 Hz, 4H), 3.36 (s, 6H), 3.24 (t, J=10.5 Hz, 1H), 3.06-2.93 (m, 1H), 2.78 (d, J=5.9 Hz, 4H), 2.52 (dd, J=9.0, 11.5 Hz, 1H), 2.49-2.43 (m, 1H), 2.44 (d, J=17.6 Hz, 1H), 2.40-2.35 (m, 1H), 2.35-2.26 (m, 1H), 2.24-2.10 (m, 3H), 2.07-1.94 (m, 3H), 1.91 (dd, J=5.4, 17.6 Hz, 1H), 1.85 (d, J=14.6 Hz, 1H), 1.82-1.67 (m, 3H), 0.59 (s, 3H). HRMS (ESI) (m/z) calculated for C₃₄H₄₅N₂O₃ [M+H]⁺: 529.3425, found 529.3434.

### β-2-Fluoroethylmethylamine 29B and α-2-Fluoroethylmethylamine 29A

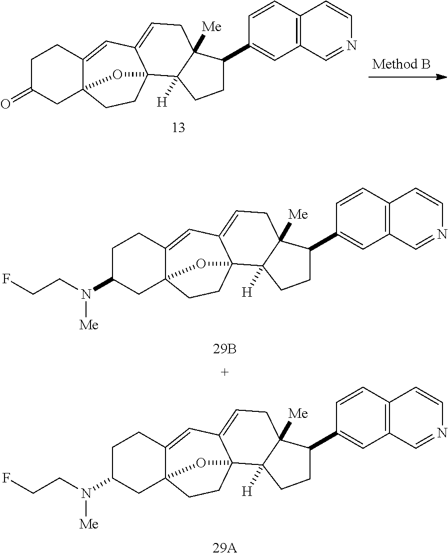 [SMILES: C[C@]12CC=C3C=C4CCC(=O)C[C@]45CC[C@]3(O5)[C@@H]1CC[C@@H]2c1ccc2ccncc2c1 ; CN(CCF)[C@H]1CCC2=CC3=CC[C@]4(C)[C@@H](c5ccc6ccncc6c5)CC[C@H]4[C@@]34CC[C@]2(C1)O4 ; CN(CCF)[C@@H]1CCC2=CC3=CC[C@]4(C)[C@@H](c5ccc6ccncc6c5)CC[C@H]4[C@@]34CC[C@]2(C1)O4]

β-2-Fluoroethylmethylamine 29B:

The crude mixture was purified by preparative TLC (eluent: 20:1 Dichloromethane:MeOH) to afford β-2-fluoroethylmethylamine 29B (2.7 mg, 51%). ¹H NMR (500 MHz, CDCl₃) Shift=9.24 (s, 1H), 8.50 (d, J=5.9 Hz, 1H), 7.80 (s, 1H), 7.77 (d, J=8.8 Hz, 1H), 7.64 (d, J=5.9 Hz, 1H), 7.60 (dd, J=1.0, 8.3 Hz, 1H), 5.74 (br. s., 1H), 5.26 (br. s., 1H), 4.68-4.46 (m, 2H), 3.15 (t, J=9.8 Hz, 1H), 2.99-2.69 (m, 3H), 2.52 (dd, J=8.8, 11.2 Hz, 1H), 2.47-2.30 (m, 6H), 2.29-2.16 (m, 4H), 2.16-2.00 (m, 3H), 2.01-1.92 (m, 1H), 1.94 (dd, J=5.1, 17.3 Hz, 1H), 1.86 (dq, J=5.4, 12.2 Hz, 1H), 1.79-1.64 (m, 3H), 0.56 (s, 3H). HRMS (ESI) (m/z) calculated for C₃₁H38N20F [M+H]: 473.2963, found 473.2971.

### β-2,2-Difluoroethylmethylamine 30B and α-2,2-Difluoroethylmethylamine 30A

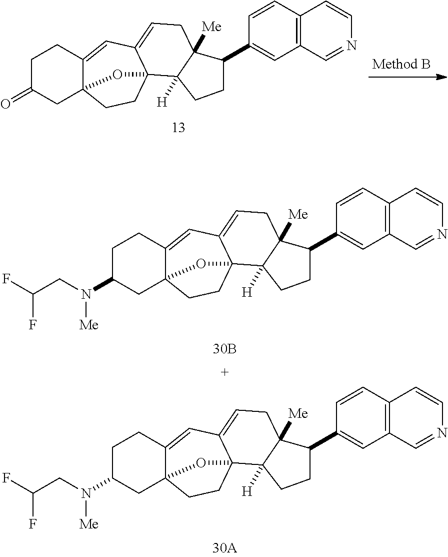 [SMILES: C[C@]12CC=C3C=C4CCC(=O)C[C@]45CC[C@]3(O5)[C@@H]1CC[C@@H]2c1ccc2ccncc2c1 ; CN(CC(F)F)[C@H]1CCC2=CC3=CC[C@]4(C)[C@@H](c5ccc6ccncc6c5)CC[C@H]4[C@@]34CC[C@]2(C1)O4 ; CN(CC(F)F)[C@@H]1CCC2=CC3=CC[C@]4(C)[C@@H](c5ccc6ccncc6c5)CC[C@H]4[C@@]34CC[C@]2(C1)O4]

β-2,2-Difluoroethylmethylamine 30B:

The crude mixture was purified by preparative TLC (eluent: 1:1 Hexanes:EtOAc) to afford β-2,2-difluoroethylmethylamine 30B (ca. 1.1 mg, 19%). ¹H NMR (500 MHz, CDCl₃) Shift=9.28 (s, 1H), 8.50 (d, J=5.9 Hz, 1H), 7.86 (s, 1H), 7.83 (d, J=8.3 Hz, 1H), 7.75 (d, J=5.4 Hz, 1H), 7.69 (d, J=8.3 Hz, 1H), 6.15-5.83 (m, 1H), 5.75 (s, 1H), 5.27 (dd, J=2.4, 5.4 Hz, 1H), 3.17 (t, J=10.0 Hz, 1H), 2.91 (br. s., 2H), 2.52 (dd, J=8.8, 11.7 Hz, 1H), 2.47 (br. s, 3H), 2.45-2.30 (m, 4H), 2.27-2.11 (m, 6H), 2.11-1.99 (m, 2H), 1.95 (dd, J=5.1, 17.3 Hz, 1H), 1.88 (dq, J=6.3, 12.7 Hz, 1H), 1.77-1.68 (m, 3H), 0.56 (s, 3H). HRMS (ESI) (m/z) calculated for C₃₁H₃₇N₂OF2 [M+H]⁺: 491.2868, found 491.2879.

β-7-Azabicyclo[2.2.1]heptane 31B and α-7-Azabicyclo[2.2.1]heptane 31A

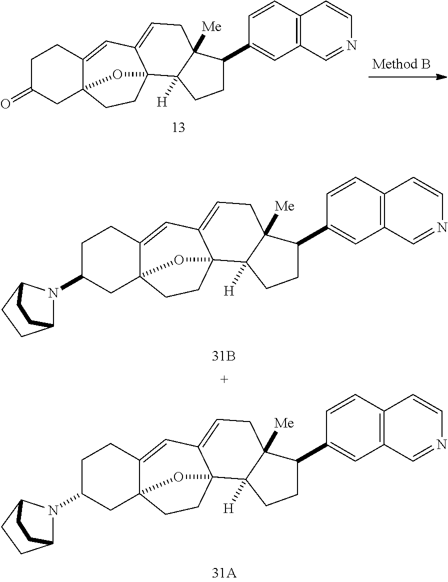

β-7-Azabicyclo[2.2.1]heptane 31B:

The crude mixture was purified by flash chromatography (silica gel, eluent: 10:10:1→10:10:2 Hexanes:EtOAc:2M NH₃ solution in MeOH) to afford β-7-azabicyclo[2.2.1]heptane 31B (3 mg, 50%). ¹H NMR (500 MHz, CDCl₃) Shift=9.24 (s, 1H), 8.50 (d, J=5.9 Hz, 1H), 7.81 (s, 1H), 7.77 (d, J=8.3 Hz, 1H), 7.64 (d, J=5.9 Hz, 1H), 7.61 (d, J=8.3 Hz, 1H), 5.71 (br. s., 1H), 5.24 (br. s., 1H), 3.43 (br. s., 2H), 3.15 (t, J=9.8 Hz, 1H), 2.69 (br. s., 2H), 2.52 (t, J=9.8 Hz, 1H), 2.46 (br. s., 1H), 2.37 (d, J=17.6 Hz, 1H), 2.38-2.29 (m, 1H), 2.27-2.13 (m, 3H), 2.11-1.92 (m, 6H), 1.87 (dq, J=5.9, 12.7 Hz, 1H), 1.86-1.79 (m, 1H), 1.78-1.60 (m, 8H), 1.55 (t, J=13.2 Hz, 1H), 0.56 (s, 3H). HRMS (ESI) (m/z) calculated for C₃₄H₄₁N₂O [M+H]⁺: 493.3213, found 493.3224.

### β-Isopropylamine 32B and α-Isopropylamine 32A

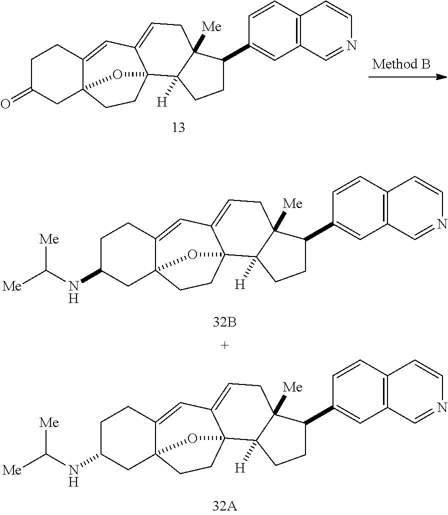 [SMILES: C[C@]12CC=C3C=C4CCC(=O)C[C@]45CC[C@]3(O5)[C@@H]1CC[C@@H]2c1ccc2ccncc2c1 ; CC(C)N[C@H]1CCC2=CC3=CC[C@]4(C)[C@@H](c5ccc6ccncc6c5)CC[C@H]4[C@@]34CC[C@]2(C1)O4 ; CC(C)N[C@@H]1CCC2=CC3=CC[C@]4(C)[C@@H](c5ccc6ccncc6c5)CC[C@H]4[C@@]34CC[C@]2(C1)O4]

p-Isopropylamine 32B:

The crude mixture was purified by flash chromatography (silica gel, eluent: 10:1 EtOAc:2M NH₃ solution in MeOH) to afford β-isopropylamine 32B (5 mg, 70%). ¹H NMR (600 MHz, CDCl₃) Shift=9.22 (s, 1H), 8.48 (d, J=5.9 Hz, 1H), 7.79 (s, 1H), 7.75 (d, J=8.8 Hz, 1H), 7.62 (d, J=5.9 Hz, 1H), 7.59 (d, J=8.2 Hz, 1H), 5.71 (s, 1H), 5.24 (d, J=2.9 Hz, 1H), 3.24 (br. s., 1H), 3.13 (t, J=9.7 Hz, 1H), 2.90 (br. s., 1H), 2.50 (dd, J=8.5, 11.4 Hz, 2H), 2.41-2.26 (m, 3H), 2.24-2.13 (m, 3H), 2.09 (dd, J=2.9, 15.3 Hz, 1H), 2.06-1.98 (m, 2H), 1.93 (dd, J=5.3, 17.6 Hz, 1H), 1.85 (dq, J=5.3, 12.3 Hz, 1H), 1.75-1.62 (m, 4H), 1.07 (br. s., 6H), 0.54 (s, 3H). HRMS (ESI) (m/z) calculated for C₃₁H₃₉N₂O [M+H]⁺: 455.3057, found 493.3049.

### β-Isopropylmethylamine 33B

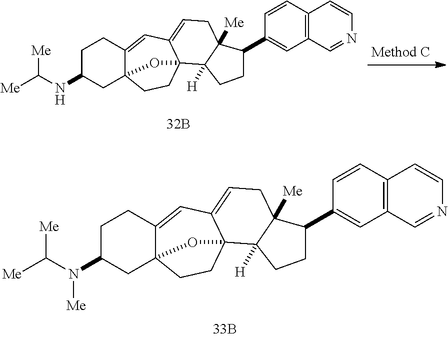 [SMILES: CC(C)N[C@H]1CCC2=CC3=CC[C@]4(C)[C@@H](c5ccc6ccncc6c5)CC[C@H]4[C@@]34CC[C@]2(C1)O4 ; CC(C)N(C)[C@H]1CCC2=CC3=CC[C@]4(C)[C@@H](c5ccc6ccncc6c5)CC[C@H]4[C@@]34CC[C@]2(C1)O4]

The crude mixture was purified by preparative TLC (eluent: 20:10:3 Hexanes:EtOAc:2M NH₃ solution in MeOH) to afford β-isopropylmethylamine 33B (4 mg, 78%). ¹H NMR (600 MHz, CDCl₃) Shift=9.22 (br. s., 1H), 8.49 (d, J=4.7 Hz, 1H), 7.79 (s, 1H), 7.75 (d, J=8.8 Hz, 1H), 7.62 (d, J=5.3 Hz, 1H), 7.59 (d, J=8.2 Hz, 1H), 5.70 (br. s., 1H), 5.22 (br. s., 1H), 3.22 (br. s., 1H), 3.13 (t, J=10.0 Hz, 1H), 2.77 (br. s., 1H), 2.50 (t, J=8.2 Hz, 1H), 2.43 (t, J=12.9 Hz, 1H), 2.36 (d, J=17.6 Hz, 1H), 2.35-2.28 (m, 2H), 2.22-2.14 (m, 2H), 2.17 (dt, J=4.4, 9.0 Hz, 1H), 2.11 (br. s., 3H), 2.08-1.99 (m, 2H), 1.98-1.91 (m, 1H), 1.93 (dd, J=4.1, 17.0 Hz, 1H), 1.85 (dq, J=5.3, 12.3 Hz, 1H), 1.69 (br. s., 2H), 1.59 (br. s., 1H), 1.56 (br. s., 1H), 0.96 (br. s., 6H), 0.54 (s, 3H). HRMS (ESI) (m/z) calculated for C₃₂H₄₁N₂O [M+H]⁺: 455.3057, found 493.3049.

### β-Isopropylethylamine 34B

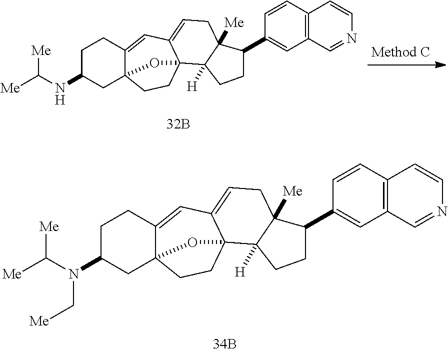 [SMILES: CC(C)N[C@H]1CCC2=CC3=CC[C@]4(C)[C@@H](c5ccc6ccncc6c5)CC[C@H]4[C@@]34CC[C@]2(C1)O4 ; CCN(C(C)C)[C@H]1CCC2=CC3=CC[C@]4(C)[C@@H](c5ccc6ccncc6c5)CC[C@H]4[C@@]34CC[C@]2(C1)O4]

The crude mixture was purified by preparative TLC (eluent: 100% MeOH) to afford β-isopropylethylamine 34B (ca. 1.5 mg, 20%). ¹H NMR (600 MHz, CD₃OD) Shift=9.19 (s, 1H), 8.38 (d, J=5.9 Hz, 1H), 7.97 (s, 1H), 7.88 (d, J=8.8 Hz, 1H), 7.79 (d, J=5.9 Hz, 1H), 7.74 (d, J=8.2 Hz, 1H), 5.76 (br. s., 1H), 5.27 (br. s., 1H), 3.26-3.19 (m, 1H), 2.91-2.66 (m, 3H), 2.50 (dd, J=9.4, 11.2 Hz, 1H), 2.48-2.36 (m, 3H), 2.29 (t, J=10.6 Hz, 1H), 2.23-2.06 (m, 4H), 2.00-1.86 (m, 5H), 1.86-1.79 (m, 1H), 1.79-1.66 (m, 2H), 1.21-1.08 (m, 9H), 0.57 (s, 3H). HRMS (ESI) (m/z) calculated for C₃₃H₄₃N₂O [M+H]⁺: 483.3370, found 483.3382.

### β-(R)-3-Fluoropyrrolidine 35B and α-(R)-3-Fluoropyrrolidine 35A

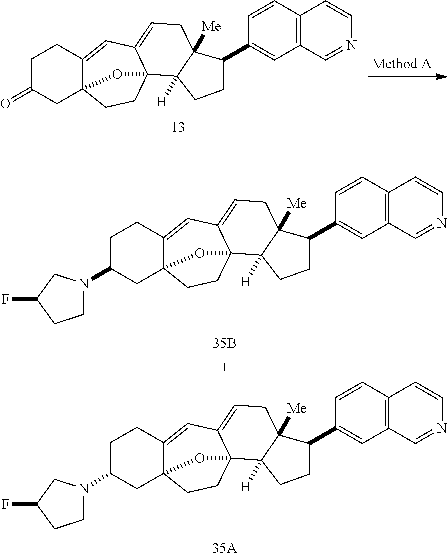 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CC[C@H](N5CC[C@@H](F)C5)C[C@]45CC[C@]3(O5)[C@@H]1CC[C@@H]2c1ccc2ccncc2c1 ; C[C@]12CC=C3C=C4CC[C@@H](N5CC[C@@H](F)C5)C[C@]45CC[C@]3(O5)[C@@H]1CC[C@@H]2c1ccc2ccncc2c1]

β-(R)-3-Fluoropyrrolidine 35B:

The crude mixture was purified by preparative TLC (eluent: 47.5:47.5:5 EtOAc:Hexanes:2M NH₃ solution in MeOH) to afford β-(R)-3-fluoropyrrolidine 35B (2.2 mg, 38%). ¹H NMR (500 MHz, CDCl₃) Shift=δ 9.22 (s, 1H), 8.49 (d, J=5.4 Hz, 1H), 7.79 (s, 1H), 7.75 (d, J=8.8 Hz, 1H), 7.62 (d, J=5.4 Hz, 1H), 7.59 (d, J=8.8 Hz, 1H), 5.71 (d, J=2.0 Hz, 1H), 5.23 (m, 1H), 5.11 (m, 1H), 3.40 (m, 1H), 3.13 (dd, J=9.3, 9.3 Hz, 1H), 2.88-2.96 (m, 2H), 2.66-2.77 (m, 1H), 2.48-2.58 (m, 3H), 2.29-2.45 (m, 3H), 2.12-2.23 (m, 3H), 1.99-2.09 (m, 5H), 1.83-1.97 (m, 2H), 1.67-1.74 (m, 2H), 1.59 (m, 1H), 0.55 (s, 3H). HRMS (ESI) (m/z) calculated for C₃₂H38FN₂O [M+H]⁺: 485.2968, found 485.2915.

### β-(S)-3-Fluoropyrrolidine 36B and α-(S)-3-Fluoropyrrolidine 36A

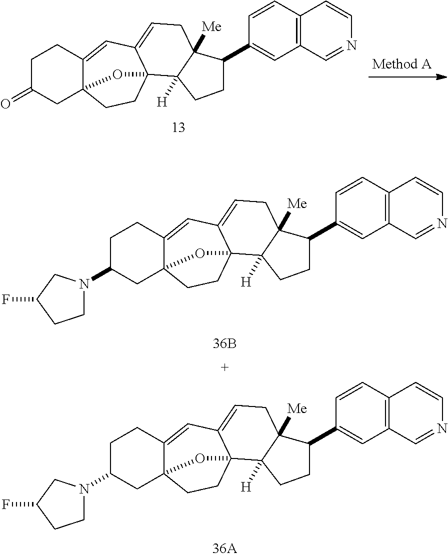 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CC[C@H](N5CC[C@H](F)C5)C[C@]45CC[C@]3(O5)[C@@H]1CC[C@@H]2c1ccc2ccncc2c1 ; C[C@]12CC=C3C=C4CC[C@@H](N5CC[C@H](F)C5)C[C@]45CC[C@]3(O5)[C@@H]1CC[C@@H]2c1ccc2ccncc2c1]

β-(S)-3-Fluoropyrrolidine 36B:

The crude mixture was purified by preparative TLC (eluent: 47.5:47.5:5 EtOAc:Hexanes:2M NH₃ solution in MeOH) to afford β-(S)-3-fluoropyrrolidine 36B (2.7 mg, 46%). ¹H NMR (500 MHz, CD₃OD) Shift=9.21-9.18 (m, 1H), 8.37 (d, J=5.87 Hz, 1H), 7.98-7.96 (m, 1H), 7.88 (d, J=8.80 Hz, 1H), 7.80-7.77 (m, 1H), 7.74 (dd, J=8.56, 1.71 Hz, 1H), 5.71 (d, J=1.47 Hz, 1H), 5.23 (m, 1H), 5.22-5.07 (m, 2H), 3.22 (t, J=9.8 Hz, 1H), 3.08-2.97 (m, 1H), 2.90 (td, J=8.19, 5.62 Hz, 1H), 2.70-2.64 (m, 1H), 2.62-2.58 (m, 1H), 2.52-2.34 (m, 6H), 2.33-2.24 (m, 1H), 2.23-2.03 (m, 3H), 2.03-1.85 (m, 6H), 1.77-1.67 (m, 2H), 1.67-1.56 (m, 1H), 0.57 (s, 3H). HRMS (ESI) (m/z) calculated for C₃₂H₃₈FN₂O [M+H]⁺: 485.6553, found 485.6551.

### β-3,3-Difluoropyrrolidine 37B and α-3,3-Difluoropyrrolidine 37A

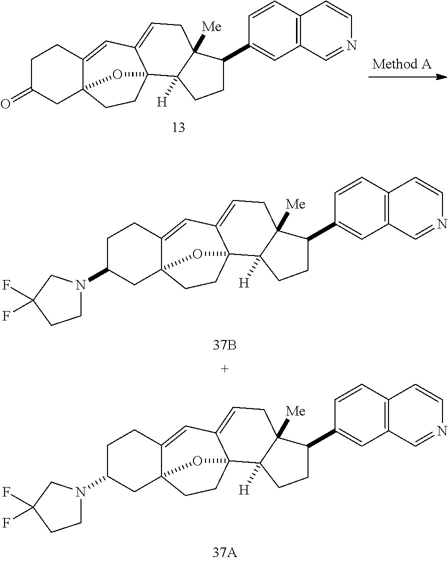 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CC[C@H](N5CCC(F)(F)C5)C[C@]45CC[C@]3(O5)[C@@H]1CC[C@@H]2c1ccc2ccncc2c1 ; C[C@]12CC=C3C=C4CC[C@@H](N5CCC(F)(F)C5)C[C@]45CC[C@]3(O5)[C@@H]1CC[C@@H]2c1ccc2ccncc2c1]

β-3,3-Difluoropyrrolidine 37B: The crude mixture was purified by preparative TLC (eluent: 80:15:5 EtOAc:Hexanes:2M NH₃ solution in MeOH) to afford β-3,3-difluoropyrrolidine 37B (2.9 mg, 40%). ¹H NMR (500 MHz, CDCl₃) Shift=δ 9.22 (s, 1H), 8.49 (d, J=5.4 Hz, 1H), 7.79 (s, 1H), 7.75 (d, J=8.3 Hz, 1H), 7.62 (d, J=5.4 Hz, 1H), 7.59 (d, J=8.8 Hz, 1H), 5.72 (d, J=2.0 Hz, 1H), 5.25 (dd, J=5.4 Hz, 2.0 Hz, 1H), 3.13 (dd, J=9.3 Hz, 9.3 Hz, 1H), 2.86-3.03 (m, 2H), 2.14 (dd, J=6.9, 6.9 Hz, 2H), 2.58 (m, 1H), 2.50 (dd, J=11.7, 8.3 Hz, 2H), 2.13-2.44 (m, 6H), 1.98-2.10 (m, 2H, 1.90-1.96 (m, 1H), 1.83-1.89 (m, 2H), 1.66-1.74 (m, 2H), 1.53-1.61 (m, 1H), 0.55 (s, 3H). HRMS (ESI) (m/z) calculated for C₃₂H37F2N20 [M+H]⁺: 503.2874 found 503.2814.

α-3,3-Difluoropyrrolidine 37A:

The crude mixture was purified by preparative TLC (eluent: 80:15:5 EtOAc:Hexanes:2M NH₃ solution in MeOH) to afford α-3,3-difluoropyrrolidine 37A (2.9 mg from 6.0 mg, 40%). ¹H NMR (500 MHz, CDCl₃) Shift=δ 9.22 (s, 1H), 8.49 (d, J=5.4 Hz, 1H), 7.79 (s, 1H), 7.75 (d, J=8.8 Hz, 1H), 7.62 (d, J=5.4 Hz, 1H), 7.59 (d, J=8.8 Hz, 1H), 5.74 (s, 1H), 5.28 (d, J=2.4 Hz, 1H), 3.15 (dd, J=9.3 Hz, 9.3 Hz, 1H), 3.01 (dt, J=13.7, 2.4 Hz, 2H), 2.83 (dd, J=6.8, 6.8 Hz, 2H), 2.52 (dd, J=11.2, 8.3 Hz, 2H), 2.15-2.40 (m, 6H), 2.02-2.07 (m, 3H), 1.79-1.97 (m, 3H), 1.72 (m, 3H), 1.61 (m, 3H), 0.54 (s, 3H). HRMS (ESI) (m/z) calculated for C₃₂H37F2N20 [M+H]⁺: 503.2874 found 503.2807.

β-2-oxa-6-azaspiro[3.4]octane 38B and α-2-oxa-6-azaspiro[3.4]octane 38A

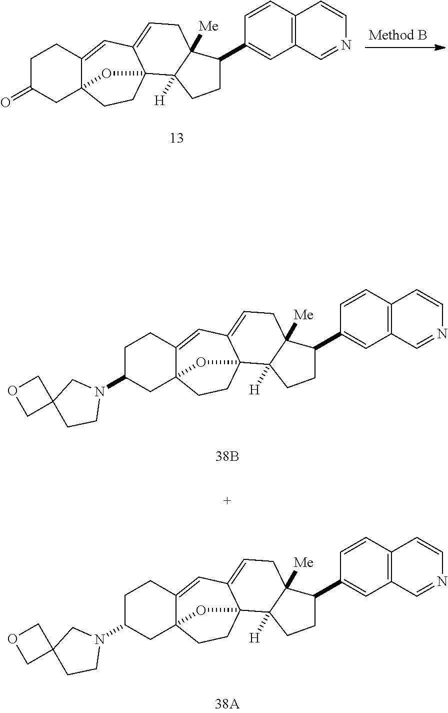

β-2-oxa-6-azaspiro[3.4]octane 38B: The crude mixture was purified by preparative TLC (eluent: 47.5:47.5:5 EtOAc:Hexanes:2M NH₃ solution in MeOH) to afford β-2-oxa-6-azaspiro[3.4]octane 38B (3.4 mg, 55%). ¹H NMR (500 MHz, CD₃OD) Shift=9.21 (s, 1H), 8.39 (d, J=5.4 Hz, 1H), 7.99 (s, 1H), 7.90 (d, J=8.8 Hz, 1H), 7.81 (d, J=5.9 Hz, 1H), 7.76 (dd, J=1.7, 8.5 Hz, 1H), 5.73-5.70 (m, 1H), 5.28-5.23 (m, 1H), 4.63 (d, J=2.4 Hz, 4H), 3.28-3.21 (m, 1H), 2.90 (dd, J=9.3, 49.8 Hz, 2H), 2.62 (t, J=7.3 Hz, 2H), 2.54-2.40 (m, 5H), 2.36-2.27 (m, 2H), 2.23-2.16 (m, 1H), 2.13 (s, 2H), 2.10-2.05 (m, 1H), 2.02-1.88 (m, 6H), 1.81-1.66 (m, 2H), 1.66-1.57 (m, 1H), 0.58 (s, 3H). HRMS (ESI) (m/z) calculated for C₃₄H₄₁N₂O₂ [M+H]⁺: 509.7015, found 509.7013.

### β-Cyclopropylamine 39B and α-Cyclopropylamine 39A

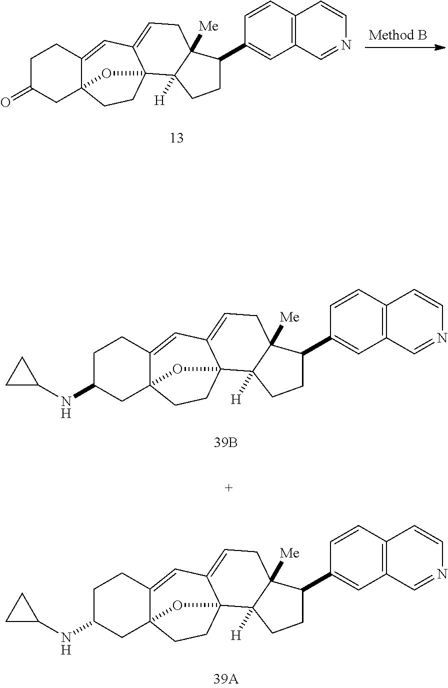

β-cyclopropylamine 39B: The crude mixture was purified by preparative TLC (eluent: 47.5:47.5:5 EtOAc:Hexanes:2M NH₃ solution in MeOH) to afford β-cyclopropylamine 39B (3.7 mg, 55%). ¹H NMR (500 MHz, CD₃OD) Shift=9.21 (s, 1H), 8.39 (d, J=5.9 Hz, 1H), 7.99 (s, 1H), 7.90 (d, J=8.8 Hz, 1H), 7.81 (d, J=5.4 Hz, 1H), 7.76 (dd, J=1.7, 8.5 Hz, 1H), 5.73 (d, J=2.0 Hz, 1H), 5.28-5.24 (m, 1H), 3.24 (t, J=20.0 Hz, 1H), 3.18-3.12 (m, 1H), 2.54-2.40 (m, 4H), 2.36-2.27 (m, 2H), 2.18 (s, 3H), 2.05-2.00 (m, 2H), 2.00-1.95 (m, 2H), 1.94-1.91 (m, 1H), 1.91-1.84 (m, 2H), 1.77-1.66 (m, 3H), 0.58 (s, 3H), 0.51 (d, J=4.4 Hz, 2H), 0.39 (dd, J=2.2, 3.7 Hz, 2H). HRMS (ESI) (m/z) calculated for C₃₁H₃₇N₂O [M+H]⁺: 453.6383, found 453.6381.

### β-Cyclopropylmethylamine 40B

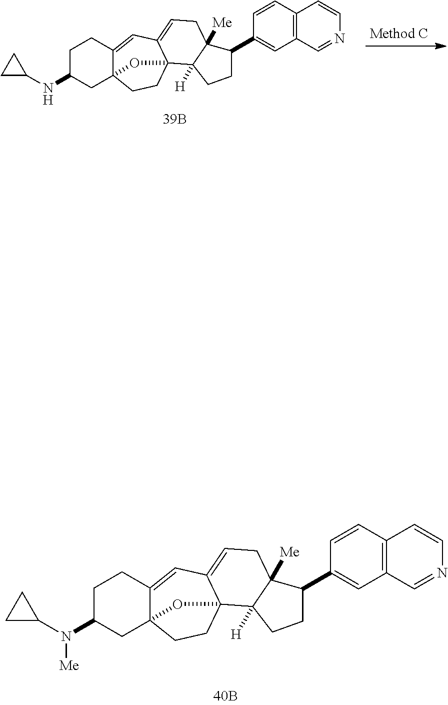

The crude mixture was purified by preparative TLC (eluent: 47.5:47.5:5 EtOAc:Hexanes:2M NH₃ solution in MeOH) to afford β-methylcyclopropylamine 40B (1.1 mg, 85%). ¹H NMR (500 MHz, CD₃OD) Shift=9.21 (s, 1H), 8.39 (d, J=5.4 Hz, 1H), 8.00-7.98 (m, 1H), 7.90 (d, J=7.8 Hz, 1H), 7.81 (d, J=5.9 Hz, 1H), 7.76 (dd, J=1.5, 9.3 Hz, 1H), 5.78-5.74 (m, 1H), 5.29-5.25 (m, 1H), 3.26-3.24 (m, 1H), 3.23 (t, J=9.8 Hz, 1H), 3.27-3.21 (m, 1H), 2.88 (t, J=1.0 Hz, 1H), 2.56-2.49 (m, 1H), 2.48-2.41 (m, 2H), 2.37 (s, 3H), 2.33-2.26 (m, 1H), 2.23-2.10 (m, 3H), 2.03 (d, J=7.3 Hz, 2H), 2.01-1.96 (m, 1H), 1.96-1.88 (m, 2H), 1.88-1.82 (m, 1H), 1.79-1.68 (m, 2H), 0.59 (m, 5H), 0.50-0.46 (m, 2H). HRMS (ESI) (m/z) calculated for C₃₂H₃₉N₂O [M+H]⁺: 467.6649, found 467.6645.

β-3-Methyl-3-oxetanamine 41B and α-3-Methyl-3-oxetanamine 41A

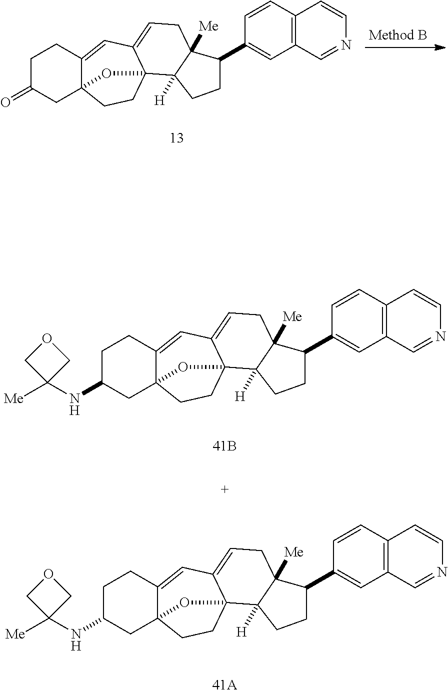

β-3-Methyl-3-oxetanamine 41B:

The crude mixture was purified by preparative TLC (eluent: 47.5:47.5:5 EtOAc:Hexanes:2M NH₃ solution in MeOH) to afford β-3-methyl-3-oxetanamine 41B (4.7 mg, 81%). ¹H NMR (500 MHz, CD₃OD) Shift=9.21 (s, 1H), 8.39 (d, J=5.4 Hz, 1H), 7.99 (s, 1H), 7.90 (d, J=8.8 Hz, 1H), 7.81 (d, J=5.9 Hz, 1H), 7.78-7.74 (m, 1H), 5.75 (d, J=1.5 Hz, 1H), 5.29-5.26 (m, 1H), 4.61 (dd, J=3.4, 5.9 Hz, 2H), 4.36 (dd, J=5.9, 8.3 Hz, 2H), 3.27-3.21 (m, 1H), 3.19-3.11 (m, 1H), 2.50 (d, J=8.3 Hz, 4H), 2.29 (s, 2H), 2.23-2.13 (m, 2H), 2.02-1.95 (m, 2H), 1.94-1.87 (m, 3H), 1.80-1.67 (m, 4H), 1.55 (br. s., 4H), 0.59 (s, 3H). HRMS (ESI) (m/z) calculated for C₃₂H₃₉N₂O₂ [M+H]⁺: 483.6643, found 483.6640.

β-N-Methyl-1-(3-methyl-3-oxetanyl)methanamine 42B and α-N-Methyl-l-(3-methyl-3-oxetanyl)methanamine 42A

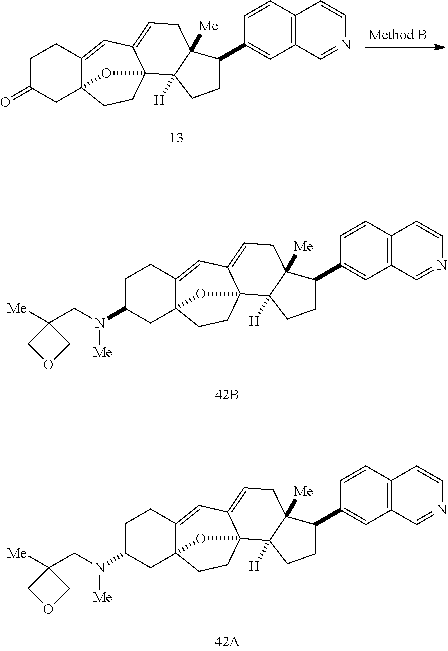

β-N-Methyl-l-(3-methyl-3-oxetanyl)methanamine 42B:

The crude mixture was purified by preparative TLC (eluent: 47.5:47.5:5 EtOAc:Hexanes:2M NH₃ solution in MeOH) to afford β-N-methyl-1-(3-methyl-3-oxetanyl)methanamine 42B (1.5 mg, 24%). ¹H NMR (500 MHz, CD₃OD) Shift=9.22 (s, 1H), 8.39 (d, J=5.9 Hz, 1H), 7.99 (s, 1H), 7.90 (d, J=8.3 Hz, 1H), 7.81 (d, J=5.9 Hz, 1H), 7.76 (dd, J=1.7, 8.5 Hz, 1H), 5.75-5.72 (m, 1H), 5.31-5.27 (m, 1H), 4.57-4.51 (m, 2H), 4.32 (dd, J=1.5, 5.9 Hz, 2H), 3.28-3.22 (m, 1H), 2.69 (s, 2H), 2.57-2.31 (m, 5H), 2.30-2.15 (m, 4H), 2.04-1.86 (m, 5H), 1.82 (t, J=24.9 Hz, 1H), 1.77-1.69 (m, 1H), 1.68-1.57 (m, 2H), 1.39 (d, J=2.9 Hz, 6H), 0.58 (s, 3H). HRMS (ESI) (m/z) calculated for C₃₄H₄₃N₂O₂ [M+H]⁺: 511.7174, found 511.7173.

β-t-Butylamine 43B and α-t-Butylamine 43A

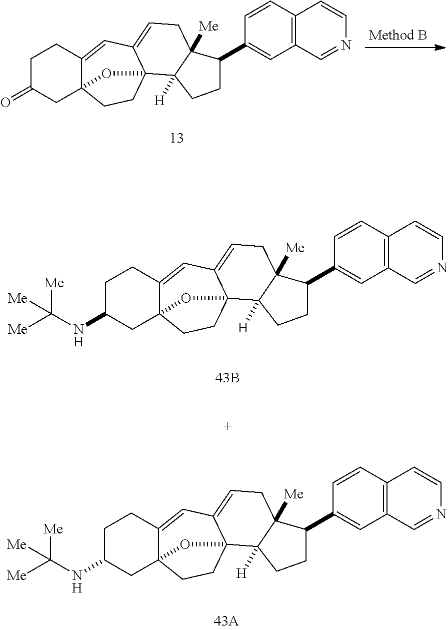

β-t-Butylamine 43B:

The crude mixture was purified by flash chromatography (silica gel, eluent: 50:1 EtOAc:triethylamine) to afford β-t-butylamine 43B (3.4 mg, 60%). ¹H NMR (500 MHz, CDCl₃) Shift=9.22 (s, 1H), 8.49 (d, J=5.9 Hz, 1H), 7.79 (s, 1H), 7.75 (d, J=8.3 Hz, 1H), 7.62 (d, J=5.9 Hz, 1H), 7.59 (d, J=8.3 Hz, 1H), 5.75 (d, J=2.0 Hz, 1H), 5.29 (m, 1H), 3.14 (dd, J=10.8, 10.8 Hz, 1H), 2.52 (dd, J=11.7, 8.8 Hz, 1H), 2.32-2.38 (m, 2H), 2.13-2.26 (m, 5H), 2.01-2.08 (m, 2H), 1.94 (dd, J=17.6, 5.4 Hz, 1H), 1.84-1.87 (m, 3H), 1.62-1.74 (m, 3H), 1.25 (br s, 9H). HRMS (ESI) (m/z) calculated for C₃₂H₄₁N₂O [M+H]⁺: 469.3219, found 469.3265.

### β-Aziridine 78B and α-Aziridine 78A

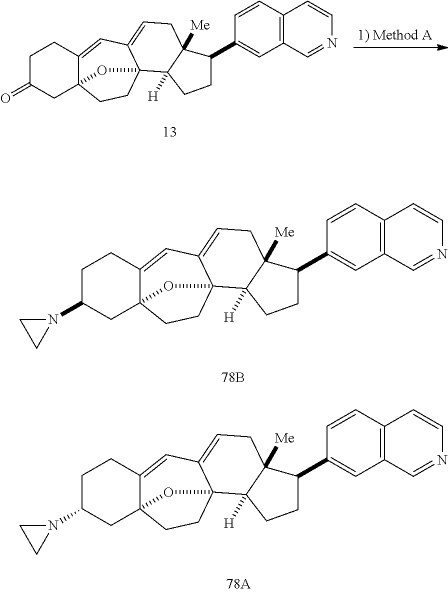

To a solution of ketone 13 (6 mg, 0.0146 mmol) in methanol (0.5 mL) was added 2-chloroethylamine hydrochloride (5.1 mg, 0.0437 mmol), followed by triethylamine (0.006 mL, 0.0437 mmol). This mixture was stirred at room temperature for 15 minutes. Glacial acetic acid (0.0025 mL, 0.0437 mmol) was added and this mixture was stirred at room temperature for 20 minutes. This mixture was cooled to 0° C. and sodium cyanoborohydride (3.2 mg, 0.0510 mml) was added. The reaction was allowed to warm to room temperature over 16 hours and then quenched with saturated solution of ammonium chloride (5 mL). This mixture was extracted with ethyl acetate (3×8 mL). The combined organic fractions were dried over anhydrous magnesium sulfate, filtered and concentrated. The crude was purified by silica gel chromatography (ethylacetate, 2% triethylamine as eluent) to afford the desired β-aziridine 78B (4.5 mg, 72% yield).

¹H NMR (500 MHz, CDCl₃) δ=9.22 (s, 1H), 8.48 (d, J=5.87 Hz, 1H), 7.79 (s, 1H), 7.75 (d, J=8.80 Hz, 1H), 7.63 (d, J=5.38 Hz, 1H), 7.59 (d, J=8.80 Hz, 1H), 5.74 (d, J=1.96 Hz, 1H), 5.25 (m, 1H), 3.34 (m, 2H), 3.14 (dd, J=10.27, 10.27 Hz, 1H), 2.75 (m, 3H), 2.51 (dd, J=11.25, 8.31, 1H), 2.44 (m, 1H), 2.29-2.37 (m, 3H), 2.12-2.27 (m, 3H), 1.81-2.08 (m, 3H), 1.54-1.74 (m, 4H), 1.15 (m, 1H), 1.05 (m, 1H), 0.55 (s, 3H). HRMS (ESI) (m/z) calculated for C₃₀H₃₅N₂O [M+H]⁺: 439.2749 found 439.2721.

### β-Hydroxyproline 65B and α-Hydroxyproline 65A

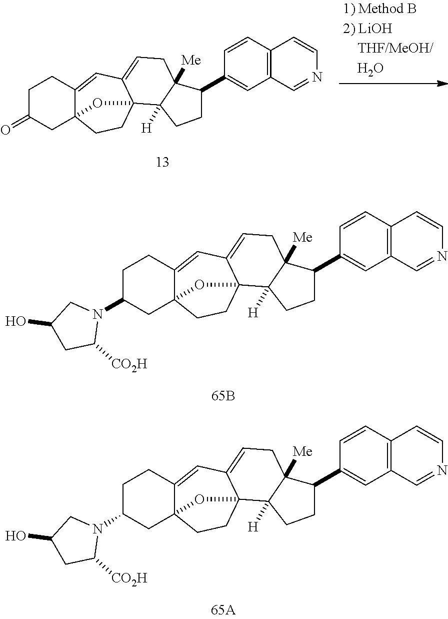

Ketone 13 was reacted with hydroxyproline methyl ester under condition ‘Method B’. The crude mixture was dissolved in THF:MeOH:1 M LiOH in H₂O=3:3:1 and stirred at 55° C. for 1.5 hours. The crude mixture was roughly concentrated and pH 3.7 sodium acetate buffer was applied, followed by the extraction with chloroform three times. The crude mixture was purified by proparative TLC (eluent: 5:1 CHCl₃:MeOH) to afford β-hydroxyproline 65B (3.7 mg, 58% in 2 steps).

¹H NMR (500 MHz, CDCl₃) Shift=9.22 (br. s., 1H), 8.47 (br. s., 1H), 7.77 (br. s., 1H), 7.75 (d, J=8.2 Hz, 1H), 7.63 (br. s., 1H), 7.57 (d, J=8.2 Hz, 1H), 5.77 (s, 1H), 5.30 (br. s., 1H), 4.47 (br. s., 1H), 4.21-4.08 (m, 1H), 4.01-3.90 (m, 0H), 3.55 (br. s., 1H), 3.19-3.11 (m, 1H), 3.12 (t, J=8.8 Hz, 1H), 2.51-2.44 (m, J=10.6, 10.6 Hz, 1H), 2.44-2.37 (m, 2H), 2.35 (d, J=17.6 Hz, 2H), 2.31-2.14 (m, 6H), 2.11 (dd, J=6.2, 13.2 Hz, 1H), 2.06-1.96 (m, 2H), 1.92 (dd, J=4.7, 17.6 Hz, 1H), 1.87-1.68 (m, 3H), 0.53 (s, 3H). HRMS (ESI) (m/z) calculated for C₃₃H₃₉N₂O₄ [M+H]⁺: 527.2904, found 527.2921.

### α-Dimethylamine 14A and β-Dimethylamine 14B

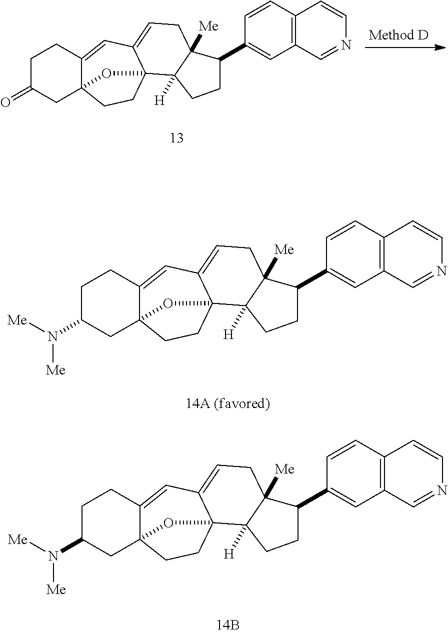

The crude mixture was purified sequentially by flash chromatography (silica gel, eluent: 20:1 EtOAc:2M NH₃ solution in MeOH) to afford α-dimethylamine 14A (2.2 mg, 68%). ¹H NMR (600 MHz, C₆D₆) Shift=9.26 (s, 1H), 8.56 (d, J=5.9 Hz, 1H), 7.44-7.39 (m, 1H), 7.36 (d, J=8.2 Hz, 1H), 7.21-7.20 (m, 1H), 7.20 (d, J=5.9 Hz, 1H), 5.68-5.65 (m, 1H), 5.15-5.11 (m, 1H), 2.72-2.66 (m, J=10.0 Hz, 1H), 2.59 (dd, J=8.8, 11.2 Hz, 1H), 2.34 (tt, J=2.9, 12.1 Hz, 1H), 2.16 (td, J=3.2, 16.0 Hz, 1H), 2.09 (s, 6H), 2.13-1.92 (m, 8H), 1.85 (ddd, J=5.0, 9.0, 13.6 Hz, 1H), 1.73 (dt, J=5.3, 12.3 Hz, 1H), 1.72-1.66 (m, 2H), 1.60-1.57 (m, 1H), 1.57-1.49 (m, 1H), 1.20 (dq, J=4.1, 12.3 Hz, 1H), 0.40 (s, 3H). HRMS (ESI) (m/z) calculated for C₃₀H₃₇N₂O [M+H]⁺: 441.2900, found 441.2909.

### α-Monomethylamine 24A and β-Monomethylamine 24B

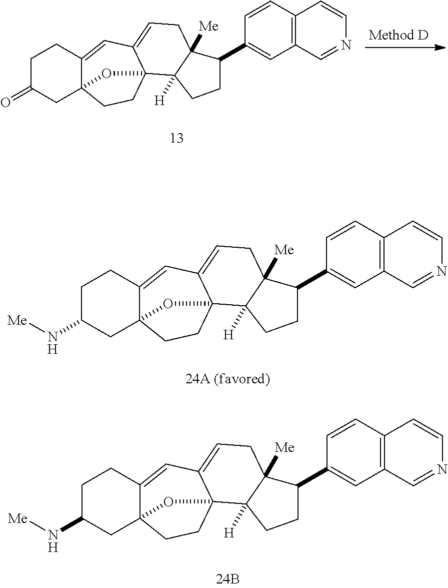

The crude mixture was purified by preparative TLC (silica gel, eluent: 10:1 EtOAc:2M NH₃ solution in MeOH) to afford α-monomethylamine 24A (ca. 1.2 mg, 37%). ¹H NMR (500 MHz, CDCl₃) Shift=9.24 (s, 1H), 8.50 (d, J=5.9 Hz, 1H), 7.81 (s, 1H), 7.77 (d, J=8.8 Hz, 1H), 7.64 (d, J=5.4 Hz, 1H), 7.61 (dd, J=1.0, 8.8 Hz, 1H), 5.76 (s, 1H), 5.29 (d, J=2.4 Hz, 1H), 3.16 (t, J=10.0 Hz, 1H), 2.61-2.50 (m, 3H), 2.49 (s, 3H), 2.43-2.30 (m, 3H), 2.30-2.15 (m, 4H), 2.13-1.99 (m, 2H), 1.95 (dd, J=5.4, 17.6 Hz, 1H), 1.88 (dq, J=4.9, 11.7 Hz, 1H), 1.79-1.60 (m, 3H), 1.19 (dq, J=4.4, 12.7 Hz, 1H), 0.56 (s, 3H). HRMS (ESI) (m/z) calculated for C₂₉H₃₅N₂O [M+H]⁺: 427.2744, found 427.2759.

α-Primary amine 62A and β-Primary amine 62B

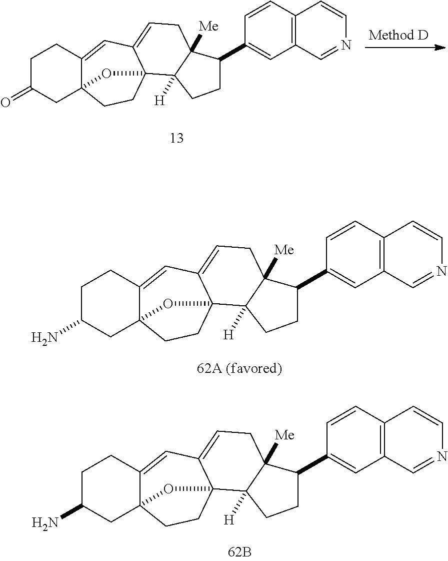

The crude mixture was purified by preparative TLC (silica gel, eluent: 25:1 EtOAc:2M NH₃ solution in MeOH) to afford α-primaryamine 62A (3.7 mg, 38%) and β-primaryamine 62B (2.5 mg, 26%).

α-primaryamine 62A:

¹H NMR (500 MHz, CDCl₃) Shift=9.22 (s, 1H), 8.48 (d, J=5.9 Hz, 1H), 7.79 (s, 1H), 7.75 (d, J=8.8 Hz, 1H), 7.62 (d, J=5.3 Hz, 1H), 7.59 (dd, J=1.2, 8.2 Hz, 1H), 5.74 (s, 1H), 5.27 (d, J=2.9 Hz, 1H), 3.14 (t, J=10.0 Hz, 1H), 2.85 (tt, J=3.2, 11.7 Hz, 1H), 2.51 (dd, J=8.5, 11.4 Hz, 1H), 2.40-2.28 (m, 3H), 2.27-2.12 (m, 4H), 2.10-1.96 (m, 3H), 1.93 (dd, J=5.3, 17.6 Hz, 1H), 1.92-1.81 (m, 2H), 1.76-1.62 (m, 2H), 1.22 (dtd, J=4.1, 11.7, 13.5 Hz, 1H), 0.53 (s, 3H). HRMS (ESI) (m/z) calculated for C₂₈H₃₃N₂O [M+H]⁺: 413.2587, found 413.2590.

β-primaryamine 62B: ¹H NMR (500 MHz, CDC3) Shift=9.22 (s, 1H), 8.48 (d, J=5.9 Hz, 1H), 7.79 (s, 1H), 7.75 (d, J=8.2 Hz, 1H), 7.62 (d, J=5.3 Hz, 1H), 7.59 (dd, J=1.5, 8.5 Hz, 1H), 5.73 (d, J=1.2 Hz, 1H), 5.25 (d, J=2.9 Hz, 1H), 3.47 (td, J=3.7, 7.9 Hz, 1H), 3.13 (t, J=10.0 Hz, 1H), 2.58 (dt, J=5.3, 14.1 Hz, 1H), 2.51 (dd, J=8.5, 11.4 Hz, 1H), 2.39-2.21 (m, 5H), 2.17 (dq, J=5.3, 9.4 Hz, 1H), 2.13 (ddd, J=2.3, 4.7, 15.8 Hz, 1H), 2.10-1.99 (m, 2H), 1.93 (dd, J=5.3, 17.0 Hz, 1H), 1.86 (dq, J=5.3, 12.3 Hz, 1H), 1.82 (ddd, J=1.8, 4.1, 13.5 Hz, 1H), 1.78-1.66 (m, 3H), 0.54 (s, 3H). HRMS (ESI) (m/z) calculated for C₂₈H₃₃N₂O [M+H]⁺: 413.2587, found 413.2599.

### Morpholine 15A and Morpholine 15B

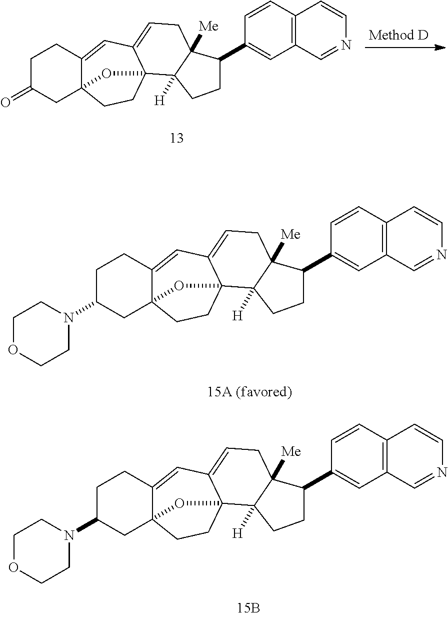

The crude mixture was purified by preparative TLC (silica gel, eluent: 40:1 EtOAc:MeOH) to afford α-morpholine 15A (ca. 1.5 mg, 38%). ¹H NMR (600 MHz, CDCl₃) Shift=9.23 (s, 1H), 8.49 (d, J=5.4 Hz, 1H), 7.80 (s, 1H), 7.76 (d, J=8.3 Hz, 1H), 7.63 (d, J=5.4 Hz, 1H), 7.60 (d, J=8.3 Hz, 1H), 5.74 (s, 1H), 5.29 (d, J=2.9 Hz, 1H), 3.73 (br. s., 4H), 3.15 (t, J=10.0 Hz, 1H), 2.60 (br. s., 4H), 2.52 (dd, J=8.5, 11.5 Hz, 2H), 2.46-2.29 (m, 3H), 2.29-2.13 (m, 4H), 2.12-1.99 (m, 2H), 1.94 (dd, J=5.1, 17.3 Hz, 1H), 1.94-1.81 (m, 3H), 1.73 (td, J=8.2, 12.3 Hz, 1H), 1.70-1.63 (m, 1H), 1.38 (dq, J=4.4, 12.2 Hz, 1H), 0.54 (s, 3H). HRMS (ESI) (m/z) calculated for C₃₂H₃₉N₂O₂ [M+H]⁺: 483.3006, found 483.3000.

### α-Pyrrolidine 19A and β-Pyrrolidine 19B

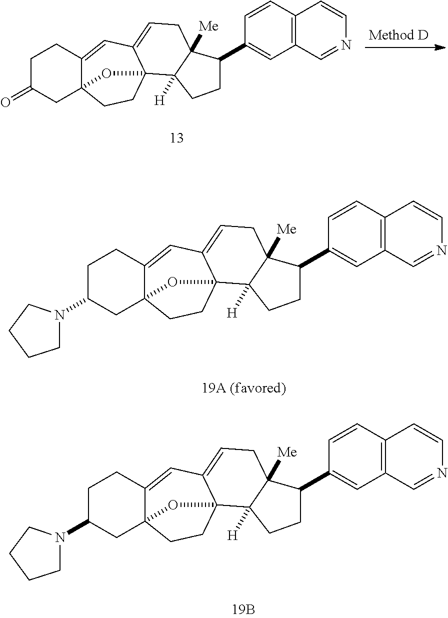

The crude mixture was purified by preparative TLC (silica gel, eluent: 20:10:3 EtOAc:Hexanes:2M NH₃ solution in MeOH) to afford α-pyrrolidine 19A (2.5 mg, 55%). ¹H NMR (500 MHz, CDCl₃) Shift=9.22 (s, 1H), 8.48 (d, J=5.9 Hz, 1H), 7.79 (s, 1H), 7.75 (d, J=8.2 Hz, 1H), 7.62 (d, J=5.3 Hz, 1H), 7.59 (d, J=8.8 Hz, 1H), 5.72 (s, 1H), 5.27 (d, J=2.9 Hz, 1H), 3.14 (t, J=10.0 Hz, 1H), 2.63 (br. s., 4H), 2.52 (dd, J=8.8, 11.2 Hz, 1H), 2.42-2.29 (m, 3H), 2.28-2.15 (m, 5H), 2.12 (d, J=12.3 Hz, 1H), 2.10-2.00 (m, 2H), 1.93 (dd, J=5.3, 17.0 Hz, 1H), 1.90-1.83 (m, 2H), 1.80 (br. s., 4H), 1.72 (td, J=8.8, 12.9 Hz, 1H), 1.63 (br. s., 1H), 1.37 (dq, J=3.5, 11.7 Hz, 1H), 0.53 (s, 3H). HRMS (ESI) (m/z) calculated for C₃₂H₃₉N₂O [M+H]⁺: 467.3057, found 467.3064.

### α-Azetidine 18A and β-Azetidine 18B

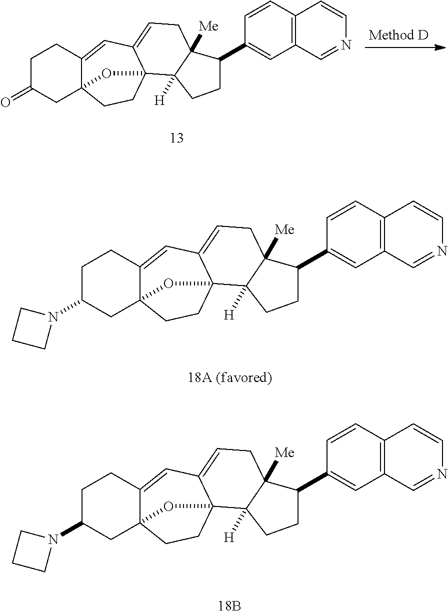

The crude mixture was purified by preparative TLC (silica gel, eluent: 1:1 EtOAc:MeOH) to afford α-azetidine 18A (ca. 1.5 mg, 38%). ¹H NMR (500 MHz, CDCl₃) Shift=9.24 (s, 1H), 8.50 (d, J=5.4 Hz, 1H), 7.80 (s, 1H), 7.77 (d, J=8.8 Hz, 1H), 7.64 (d, J=5.9 Hz, 1H), 7.60 (d, J=8.8 Hz, 1H), 5.74 (s, 1H), 5.28 (br. s., 1H), 3.24 (br. s., 4H), 3.16 (t, J=9.8 Hz, 1H), 2.54 (dd, J=8.8, 11.2 Hz, 1H), 2.42-2.30 (m, 3H), 2.30-2.13 (m, 5H), 2.12-2.00 (m, 2H), 1.95 (dd, J=5.4, 18.1 Hz, 1H), 1.93-1.78 (m, 3H), 1.74 (td, J=8.3, 12.2 Hz, 1H), 1.67-1.54 (m, 3H), 1.11 (q, J=12.2 Hz, 1H), 0.55 (s, 3H). HRMS (ESI) (m/z) calculated for C₃₁H₃₇N₂O [M+H]⁺: 453.2906, found 453.2900.

α-t-Butylamine 43A and β-t-Butylamine 43B

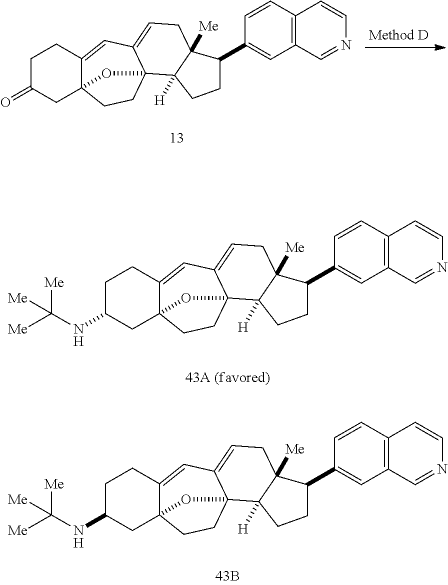

The crude mixture was purified by flash chromatography (silica gel, eluent: 10:1 CHCl3:i-PrOH) to afford α-t-butylamine 43A (2.4 mg, 42%) and β-t-butylamine 43B (1.6 mg, 28%).

α-t-Butylamine 43A:

¹H NMR (500 MHz, CDCl₃) Shift=9.22 (br. s., 1H), 8.48 (d, J=5.9 Hz, 1H), 7.78 (s, 1H), 7.75 (d, J=8.8 Hz, 1H), 7.62 (d, J=5.3 Hz, 1H), 7.58 (dd, J=1.2, 8.2 Hz, 1H), 5.73 (s, 1H), 5.28 (d, J=2.3 Hz, 1H), 3.14 (t, J=9.7 Hz, 1H), 2.49 (dd, J=8.8, 11.2 Hz, 1H), 2.46-2.40 (m, 1H), 2.39-2.29 (m, 3H), 2.28-2.12 (m, 5H), 2.08-1.99 (m, 1H), 1.93 (dd, J=5.3, 17.0 Hz, 1H), 1.83 (dq, J=5.0, 12.2 Hz, 2H), 1.76-1.60 (m, 3H), 1.55 (br. s., 9H), 1.26-1.18 (m, 1H), 0.54 (s, 3H). HRMS (ESI) (m/z) calculated for C₃₂H₄₁N₂O [M+H]⁺: 469.3213, found 469.3223.

β-t-Butylamine 43B:

¹H NMR (500 MHz, CDCl₃) Shift=9.22 (s, 1H), 8.48 (d, J=5.3 Hz, 1H), 7.78 (s, 1H), 7.75 (d, J=8.8 Hz, 1H), 7.62 (d, J=5.9 Hz, 1H), 7.59 (d, J=9.4 Hz, 1H), 5.73 (br. s., 1H), 5.25 (br. s., 1H), 3.38-3.23 (m, 1H), 3.13 (t, J=10.0 Hz, 1H), 2.57-2.48 (m, 1H), 2.49 (dd, J=8.5, 10.9 Hz, 1H), 2.40-2.27 (m, 2H), 2.25-2.08 (m, 4H), 2.07-1.98 (m, 2H), 1.93 (dd, J=5.3, 17.6 Hz, 2H), 1.84 (dq, J=5.3, 12.3 Hz, 1H), 1.76-1.66 (m, 2H), 1.65-1.47 (m, 3H), 1.42-0.94 (br. s., 9H), 0.54 (s, 3H). HRMS (ESI) (m/z) calculated for C₃₂H₄₁N₂O [M+H]⁺: 469.3213, found 469.3225.

### α-Hydroxyazetidine 70A and β-Hydroxyazetidine 70B

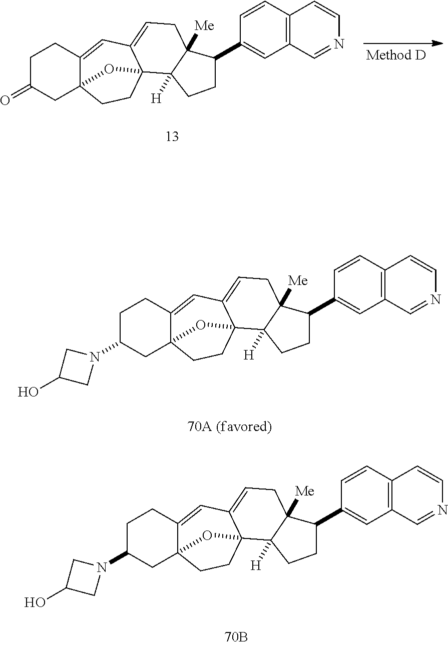 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CC[C@@H](N5CC(O)C5)C[C@]45CC[C@]3(O5)[C@@H]1CC[C@@H]2c1ccc2ccncc2c1 ; C[C@]12CC=C3C=C4CC[C@H](N5CC(O)C5)C[C@]45CC[C@]3(O5)[C@@H]1CC[C@@H]2c1ccc2ccncc2c1]

The crude mixture was purified by preparative TLC (silica gel, eluent: 2:3 EtOAc:MeOH) to afford α-hydroxyazetidine 70A (1.2 mg, 30%). ¹H NMR (500 MHz, CDCl₃) Shift=9.24 (s, 1H), 8.50 (d, J=5.4 Hz, 1H), 7.80 (s, 1H), 7.77 (d, J=8.3 Hz, 1H), 7.64 (d, J=5.9 Hz, 1H), 7.60 (d, J=8.3 Hz, 1H), 5.77 (s, 1H), 5.32 (d, J=2.4 Hz, 1H), 4.66-4.55 (m, 1H), 3.94 (br. s., 2H), 3.34 (br. s., 2H), 3.16 (t, J=9.8 Hz, 1H), 2.53 (dd, J=8.5, 11.5 Hz, 1H), 2.41 (dd, J=15.6, 28.3 Hz, 2H), 2.34 (dt, J=4.9, 11.2 Hz, 1H), 2.28 (t, J=11.2 Hz, 1H), 2.25-2.14 (m, 3H), 2.10-1.98 (m, 2H), 1.95 (dd, J=5.4, 17.6 Hz, 1H), 1.96-1.89 (m, 1H), 1.86 (dd, J=5.4, 12.2 Hz, 1H), 1.84-1.76 (m, 2H), 1.74 (td, J=8.4, 12.4 Hz, 1H), 1.65 (dt, J=7.8, 10.5 Hz, 1H), 1.40-1.27 (m, 1H), 0.55 (s, 3H). HRMS (ESI) (m/z) calculated for C₃₁H₃₇N₂O₂ [M+H]⁺: 469.2850, found 469.2872.

### α-Hydroxymethylazetidine 69A and β-Hydroxymethylazetidine 69B

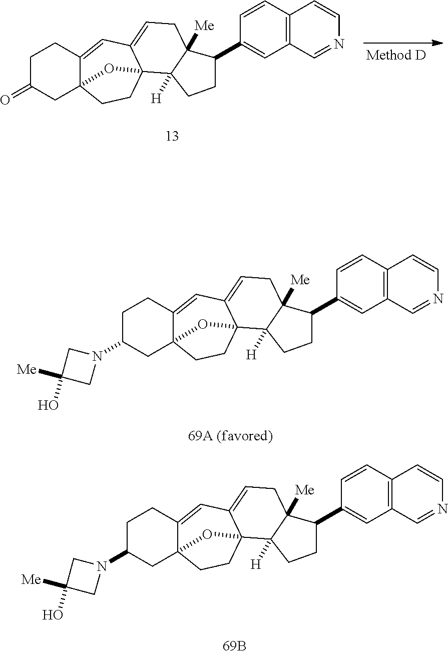

The crude mixture was purified by preparative TLC (silica gel, eluent: 1:1 EtOAc:MeOH) to afford α-hydroxymethylazetidine 69A (2.3 mg, 53%). ¹H NMR (600 MHz, CDCl₃) Shift=9.23 (s, 1H), 8.49 (d, J=5.9 Hz, 1H), 7.80 (s, 1H), 7.76 (d, J=8.2 Hz, 1H), 7.63 (d, J=5.9 Hz, 1H), 7.59 (d, J=8.8 Hz, 1H), 5.76 (s, 1H), 5.30 (d, J=2.9 Hz, 1H), 3.65-3.35 (m, 4H), 3.15 (t, J=10.0 Hz, 1H), 2.52 (dd, J=8.5, 11.4 Hz, 1H), 2.40 (dd, J=16.4, 25.8 Hz, 2H), 2.33 (dt, J=4.1, 11.7 Hz, 1H), 2.26 (t, J=11.4 Hz, 1H), 2.24 (m, 3H), 2.09-2.00 (m, 1H), 1.98 (br. s., 1H), 1.94 (dd, J=5.0, 17.3 Hz, 1H), 1.92-1.77 (m, 4H), 1.73 (td, J=8.2, 12.9 Hz, 1H), 1.67-1.59 (m, 1H), 1.58-1.50 (br. s., 3H), 1.39-1.28 (m, 1H), 0.58-0.51 (s, 3H). HRMS (ESI) (m/z) calculated for C₃₂H₃₉N₂O₂ [M+H]⁺: 483.3006, found 483.3000.

### α-Aminoethylsulfonamide 71A and β-Aminoethylsulfonamide 71B

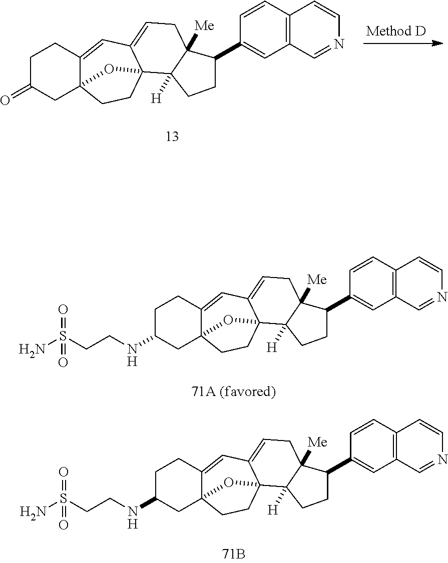 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CC[C@@H](NCCS(N)(=O)=O)C[C@]45CC[C@]3(O5)[C@@H]1CC[C@@H]2c1ccc2ccncc2c1 ; C[C@]12CC=C3C=C4CC[C@H](NCCS(N)(=O)=O)C[C@]45CC[C@]3(O5)[C@@H]1CC[C@@H]2c1ccc2ccncc2c1]

The crude mixture was purified by preparative TLC (silica gel, eluent: 100% EtOAc) to afford β-aminoethylsulfonamide 71B (0.7 mg, 15%) and α-aminoethylsulfonamide 71A (1.1 mg, 24%).

β-Aminoethylsulfonamide 71B:

¹H NMR (500 MHz, CD₃OD) Shift=9.21-9.17 (m, 1H), 8.37 (d, J=5.9 Hz, 1H), 7.97 (s, 1H), 7.88 (d, J=8.3 Hz, 1H), 7.79 (d, J=5.4 Hz, 1H), 7.74 (dd, J=1.5, 8.8 Hz, 1H), 5.74-5.69 (m, 1H), 5.28-5.22 (m, 1H), 3.26-3.18 (m, J=9.8 Hz, 1H), 3.14-3.00 (m, 4H), 2.59-2.38 (m, 4H), 2.37-2.26 (m, 2H), 2.23-2.06 (m, 3H), 2.03-1.86 (m, 5H), 1.85-1.76 (m, 1H), 1.76-1.59 (m, 3H), 0.57 (s, 3H). HRMS (ESI) (m/z) calculated for C₃₀H₃₈N₃O₃S [M+H]⁺: 520.2628, found 520.2640.

α-Aminoethylsulfonamide 71A:

¹H NMR (500 MHz, CD₃OD) Shift=9.19 (s, 1H), 8.37 (d, J=5.9 Hz, 1H), 7.97 (s, 1H), 7.88 (d, J=8.8 Hz, 1H), 7.79 (d, J=5.9 Hz, 1H), 7.74 (dd, J=1.7, 8.6 Hz, 1H), 5.77-5.71 (m, 1H), 5.30-5.24 (m, 1H), 3.29-3.24 (m, 2H), 3.23 (dd, J=9.0, 11.0 Hz, 1H), 3.13 (dt, J=2.7, 6.7 Hz, 2H), 2.73 (tt, J=3.1, 12.0 Hz, 1H), 2.50 (dd, J=8.6, 11.5 Hz, 1H), 2.47-2.38 (m, 2H), 2.39-2.34 (m, 1H), 2.32 (d, J=11.2 Hz, 1H), 2.30-2.22 (m, 2H), 2.17 (dtd, J=5.9, 9.3, 14.7 Hz, 2H), 2.10-2.03 (m, 1H), 2.01 (s, 1H), 2.00-1.92 (m, 1H), 1.90 (dd, J=6.4, 17.1 Hz, 1H), 1.72 (td, J=8.3, 12.3 Hz, 1H), 1.68-1.60 (m, 2H), 1.18 (dq, J=4.4, 12.2 Hz, 1H), 0.56 (s, 3H). HRMS (ESI) (m/z) calculated for C₃₀H₃₈N₃O₃S [M+H]⁺: 520.2628, found 520.2643.

α-Hydroxyaminomethyloxetane 72A and β-Hydroxyaminomethyloxetane 72B

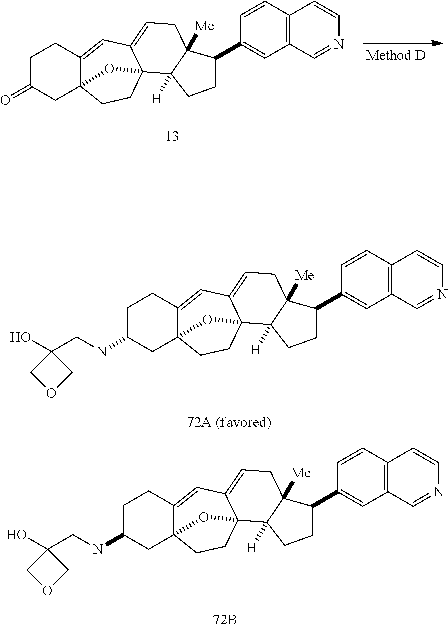 [SMILES: C[C@]12CC=C3C=C4CCC(=O)C[C@]45CC[C@]3(O5)[C@@H]1CC[C@@H]2c1ccc2ccncc2c1 ; C[C@]12CC=C3C=C4CC[C@@H](NCC5(O)COC5)C[C@]45CC[C@]3(O5)[C@@H]1CC[C@@H]2c1ccc2ccncc2c1 ; C[C@]12CC=C3C=C4CC[C@H](NCC5(O)COC5)C[C@]45CC[C@]3(O5)[C@@H]1CC[C@@H]2c1ccc2ccncc2c1]

The crude mixture was purified by preparative TLC (silica gel, eluent: 100% EtOAc) to afford α-hydroxyaminomethyloxetane 72A (1.5 mg, 34%). ¹H NMR (500 MHz, CD₃OD) Shift=9.19 (s, 1H), 8.38 (d, J=5.4 Hz, 1H), 7.97 (s, 1H), 7.88 (d, J=8.3 Hz, 1H), 7.79 (d, J=5.9 Hz, 1H), 7.74 (dd, J=1.5, 8.8 Hz, 1H), 5.84-5.78 (m, 1H), 5.35-5.30 (m, 1H), 4.64 (d, J=7.3 Hz, 2H), 4.57 (dd, J=3.7, 7.1 Hz, 2H), 3.48-3.40 (m, 2H), 3.24 (dd, J=9.0, 11.0 Hz, 2H), 2.54-2.48 (m, J=8.8 Hz, 1H), 2.48-2.40 (m, 3H), 2.40-2.24 (m, 4H), 2.24-2.13 (m, 3H), 2.01-1.85 (m, 4H), 1.80-1.64 (m, 2H), 1.46 (dq, J=4.9, 12.2 Hz, 1H), 0.57 (s, 3H). HRMS (ESI) (m/z) calculated for C₃₂H₃₉N₂O₃ [M+H]⁺: 499.2955, found 499.2933.

### β-PEGamine 75B and α-PEGamine 75A

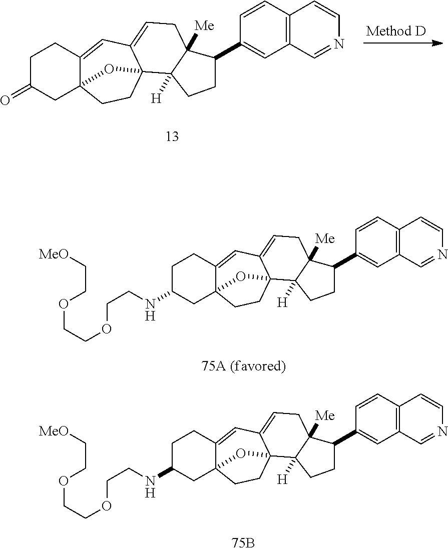 [SMILES: C[C@]12CC=C3C=C4CCC(=O)C[C@]45CC[C@]3(O5)[C@@H]1CC[C@@H]2c1ccc2ccncc2c1 ; COCCOCCOCCN[C@@H]1CCC2=CC3=CC[C@]4(C)[C@@H](c5ccc6ccncc6c5)CC[C@H]4[C@@]34CC[C@]2(C1)O4 ; COCCOCCOCCN[C@H]1CCC2=CC3=CC[C@]4(C)[C@@H](c5ccc6ccncc6c5)CC[C@H]4[C@@]34CC[C@]2(C1)O4]

β-PEGamine 75B: The crude mixture was purified by preparative TLC (silica gel, eluent: 5:5:1 EtOAc:Dichloromethane:2M NH₃ solution in MeOH) to afford □-PEGamine 75B (1.0 mg, 18%). ¹H NMR (500 MHz, CDCl₃) Shift=9.24 (s, 1H), 8.50 (d, J=5.9 Hz, 1H), 7.81 (s, 1H), 7.77 (d, J=8.3 Hz, 1H), 7.64 (d, J=5.9 Hz, 1H), 7.61 (dd, J=1.7, 8.5 Hz, 1H), 5.72 (d, J=1.5 Hz, 2H), 5.25 (dd, J=2.2, 5.1 Hz, 1H), 3.71-3.64 (m, 6H), 3.62 (t, J=5.4 Hz, 2H), 3.58 (dd, J=3.7, 5.6 Hz, 2H), 3.41 (s, 3H), 3.19-3.12 (m, J=10.7 Hz, 1H), 3.11 (t, J=3.9 Hz, 1H), 2.79 (t, J=5.4 Hz, 2H), 2.56 (t, J=16.1 Hz, 1H), 2.52 (dd, J=8.3, 11.2 Hz, 1H), 2.42-2.29 (m, 3H), 2.27-2.13 (m, 2H), 2.12-2.01 (m, 2H), 2.02 (dd, J=3.7, 13.9 Hz, 1H), 1.95 (br. s., 2H), 1.86 (dq, J=5.4, 12.2 Hz, 1H), 1.80-1.73 (m, 1H), 1.71 (dd, J=3.2, 11.5 Hz, 1H), 1.68-1.58 (m, 2H), 0.56 (s, 3H). HRMS (ESI) (m/z) calculated for C₃₅H₄₇N₂O₄ [M+H]⁺: 559.3530, found 559.3545.

α-PEGamine 75A: The crude mixture was purified by preparative TLC (silica gel, eluent: 100:5:1 EtOAc:MeOH:Triethylamine) to afford α-PEGamine 75A (1.1 mg, 20%). ¹H NMR (500 MHz, CDCl₃) Shift=9.24 (s, 1H), 8.50 (d, J=5.9 Hz, 1H), 7.81 (s, 1H), 7.77 (d, J=8.8 Hz, 1H), 7.64 (d, J=5.9 Hz, 1H), 7.61 (dd, J=1.5, 8.8 Hz, 1H), 5.75 (d, J=2.0 Hz, 1H), 5.28 (dd, J=2.2, 5.1 Hz, 1H), 3.70-3.65 (m, 7H), 3.64 (t, J=5.4 Hz, 2H), 3.61-3.55 (m, 2H), 3.41 (s, 3H), 3.16 (dd, J=9.0, 10.5 Hz, 1H), 2.87 (t, J=5.1 Hz, 2H), 2.67 (t, J=11.5 Hz, 1H), 2.54 (dd, J=8.3, 11.7 Hz, 1H), 2.38 (d, J=16.1 Hz, 3H), 2.24 (d, J=12.2 Hz, 2H), 2.22-2.14 (m, 2H), 2.11-2.02 (m, 2H), 1.95 (dd, J=5.4, 16.6 Hz, 1H), 1.88 (dq, J=5.4, 12.2 Hz, 1H), 1.78-1.69 (m, 2H), 1.68-1.62 (m, 1H), 1.27-1.19 (m, 1H), 0.55 (s, 3H). HRMS (ESI) (m/z) calculated for C₃₅H₄₇N₂O₄ [M+H]⁺: 559.3530, found 559.3542.

α-methylsulfonamide 73A

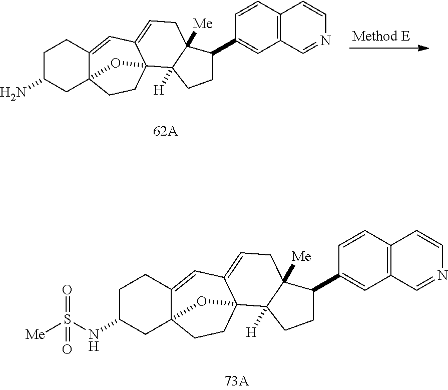 [SMILES: C[C@]12CC=C3C=C4CC[C@@H](N)C[C@]45CC[C@]3(O5)[C@@H]1CC[C@@H]2c1ccc2ccncc2c1 ; C[C@]12CC=C3C=C4CC[C@@H](NS(C)(=O)=O)C[C@]45CC[C@]3(O5)[C@@H]1CC[C@@H]2c1ccc2ccncc2c1]

The crude mixture was purified by preparative TLC (silica gel, eluent: 40:1 MeOH:Dichloromethane) to afford α-methylsulfonamide 73A (2.0 mg, 82%). ¹H NMR (500 MHz, CDCl₃) Shift=9.24 (br. s., 1H), 8.51 (d, J=4.9 Hz, 1H), 7.80 (s, 1H), 7.77 (d, J=8.3 Hz, 1H), 7.64 (d, J=5.9 Hz, 1H), 7.60 (dd, J=1.5, 8.3 Hz, 1H), 5.78 (d, J=1.5 Hz, 1H), 5.36-5.29 (m, 1H), 4.24 (d, J=7.8 Hz, 1H), 3.53 (tdt, J=3.9, 7.7, 11.7 Hz, 1H), 3.20-3.11 (m, J=10.7 Hz, 1H), 3.04 (s, 3H), 2.51 (dd, J=8.3, 11.7 Hz, 1H), 2.37 (d, J=16.6 Hz, 3H), 2.28 (br. s., 2H), 2.25-2.10 (m, 4H), 2.10-2.00 (m, 1H), 1.96 (dd, J=5.4, 17.6 Hz, 1H), 1.88 (dt, J=5.4, 11.7 Hz, 1H), 1.85 (t, J=12.2 Hz, 1H), 1.80-1.66 (m, 2H), 1.47-1.35 (m, J=4.4 Hz, 1H), 0.55 (s, 3H); HRMS (ESI) (m/z) calculated for C₂₉H₃₅N₂O₃S [M+H]⁺: 491.2363, found 491.2387.

β-methylsulfonamide 73B

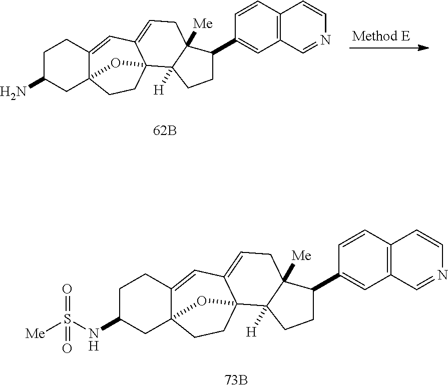 [SMILES: C[C@]12CC=C3C=C4CC[C@H](N)C[C@]45CC[C@]3(O5)[C@@H]1CC[C@@H]2c1ccc2ccncc2c1 ; C[C@]12CC=C3C=C4CC[C@H](NS(C)(=O)=O)C[C@]45CC[C@]3(O5)[C@@H]1CC[C@@H]2c1ccc2ccncc2c1]

The crude mixture was purified by preparative TLC (silica gel, eluent: 40:1 MeOH:Dichloromethane) to afford β-methylsulfonamide 73B (1.7 mg, 90%). ¹H NMR (500 MHz, CDCl₃) Shift=9.24 (s, 1H), 8.50 (d, J=5.9 Hz, 1H), 7.80 (s, 1H), 7.78 (d, J=8.8 Hz, 1H), 7.64 (d, J=5.9 Hz, 1H), 7.60 (dd, J=1.5, 8.3 Hz, 1H), 5.79 (d, J=1.5 Hz, 1H), 5.35-5.30 (m, 1H), 4.31 (d, J=6.3 Hz, 1H), 4.00 (quind, J=3.6, 6.7 Hz, 1H), 3.16 (dd, J=9.0, 10.5 Hz, 1H), 3.03 (s, 3H), 2.52 (dd, J=8.5, 11.5 Hz, 1H), 2.41 (t, J=16.1 Hz, 1H), 2.39 (br. s., 2H), 2.32-2.26 (m, 2H), 2.26-2.14 (m, 3H), 2.12-1.99 (m, 2H), 1.96 (dd, J=5.1, 17.3 Hz, 2H), 1.86 (dq, J=5.4, 12.2 Hz, 1H), 1.83-1.72 (m, 3H), 0.56 (s, 3H); HRMS (ESI) (m/z) calculated for C29H₃₅N₂O₃S [M+H]⁺: 491.2363, found 491.2376.

α-methyl-methylsulfonamide 76A

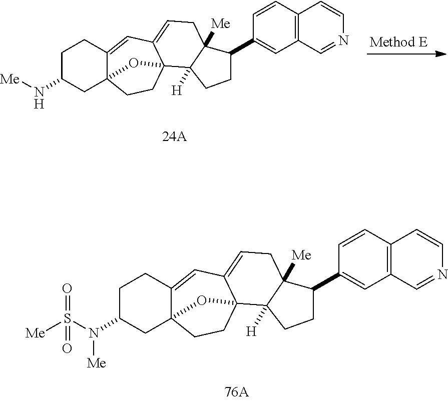 [SMILES: CN[C@@H]1CCC2=CC3=CC[C@]4(C)[C@@H](c5ccc6ccncc6c5)CC[C@H]4[C@@]34CC[C@]2(C1)O4 ; CN([C@@H]1CCC2=CC3=CC[C@]4(C)[C@@H](c5ccc6ccncc6c5)CC[C@H]4[C@@]34CC[C@]2(C1)O4)S(C)(=O)=O]

The crude mixture was purified by preparative TLC (silica gel, eluent: 40:1 MeOH:Dichloromethane) to afford α-methyl-methylsulfonamide 76A (0.7 mg, 57%). ¹H NMR (500 MHz, CDCl₃) Shift=9.24 (s, 1H), 8.51 (d, J=5.9 Hz, 1H), 7.81 (s, 1H), 7.78 (d, J=8.3 Hz, 1H), 7.64 (d, J=5.9 Hz, 1H), 7.60 (dd, J=1.5, 8.3 Hz, 1H), 5.79 (s, 1H), 5.33 (dd, J=2.2, 5.6 Hz, 1H), 3.98 (tt, J=3.4, 12.4 Hz, 1H), 3.16 (dd, J=9.0, 10.5 Hz, 1H), 2.89 (s, 3H), 2.80 (s, 3H), 2.52 (dd, J=8.5, 11.5 Hz, 1H), 2.44 (ddd, J=2.7, 4.1, 16.6 Hz, 1H), 2.36 (br. s., 3H), 2.28 (d, J=10.7 Hz, 2H), 2.21 (dq, J=4.9, 9.1 Hz, 1H), 2.14 (t, J=12.7 Hz, 1H), 2.10-2.00 (m, 0H), 1.97 (dd, J=5.4, 17.6 Hz, 1H), 1.93 (dd, J=2.9, 12.2 Hz, 1H), 1.88 (dd, J=5.4, 12.2 Hz, 1H), 1.87-1.81 (m, 1H), 1.80-1.60 (m, 3H), 0.56 (s, 3H). HRMS (ESI) (m/z) calculated for C30H₃₇N₂O₃S [M+H]⁺: 505.2519, found 505.2537.

### Example S4. Another Possible Route from Isoquinoline Compound 10 to 12

A new route to isoquinoline 12 was designed. See Scheme 2-1 below. Triflation/Suzuki cross-coupling reaction was achieved on a similar substrate with the designated reagents shown in the figure. See, e.g., Nicolaou et al., J. Am. Chem. Soc. 2009, 131, 10587-10597.

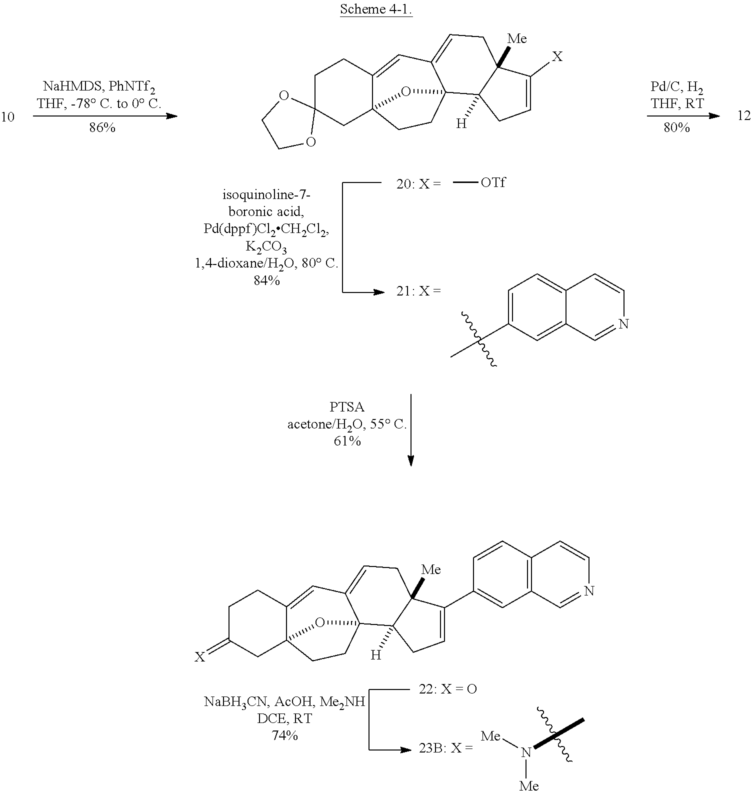

### Synthesis of Triflate 20

To a solution of monoketone 10 (200 mg, 584 μmol, 1.00 equiv) in THF (4 mL) was added NaHMDS (1 M, 701 μL, 701 μmol, 1.20 equiv) at −78° C. dropwise. After stirring 1.5h, PhNTf₂ (313 mg, 876 μmol, 1.50 equiv) in THF (2.5 mL) was cannulated and the reaction mixture was warmed up to 0° C. After additional 30 min, saturated NH₄Cl solution (8 mL) was added to the stirred reaction mixture and diluted with EtOAc (10 mL). The layers were separated and the aqueous layer was extracted with EtOAc (2×6 mL) and the organic layers were combined, washed with brine (15 mL), dried over Na₂SO₄, and concentrated under reduced pressure. The resulting residue was then purified by flash column chromatography (silica gel, eluent: 8:1→5:1 Hexanes:EtOAc) to provide triflate 20 (237 mg, 86%). ¹H NMR (500 MHz, CDCl₃) Shift=5.76 (s, 1H), 5.67 (br. s., 1H), 5.32 (dd, J=2.0, 4.9 Hz, 1H), 4.02-3.94 (m, 4H), 2.67 (dd, J=6.8, 10.7 Hz, 1H), 2.49 (t, J=14.6 Hz, 1H), 2.45 (ddd, J=3.7, 6.5, 15.2 Hz, 1H), 2.38-2.28 (m, 4H), 2.17 (ddd, J=1.5, 10.7, 12.7 Hz, 1H), 2.12 (d, J=13.2 Hz, 1H), 2.10 (dd, J=5.9, 17.6 Hz, 1H), 1.98 (dd, J=2.7, 13.4 Hz, 1H), 1.88 (ddd, J=7.6, 8.9, 12.8 Hz, 1H), 1.80 (tdd, J=2.4, 4.8, 12.7 Hz, 1H), 1.74-1.63 (m, 2H), 1.03 (s, 3H). HRMS (ESI) (m/z) calculated for C₂₂H₂₆O₆F₃S [M+H]⁺: 475.1397, found 475.1411.

Synthesis of Suzuki Cross-Coupling for 17,18-Unsaturated Isoquinoline 21 from Triflate 20

To a solution of triflate 20 (1.00 equiv) and isoquinoline-7-boronic acid (3.00 equiv) in 1,4-dioxane and H₂O (10:1, 0.02M) was added K₂CO₃ (3.00 equiv) and the solution was bubbled through inert Ar for 5 min. Pd(dppf)Cl₂.CH₂C12 (0.05 equiv) was added and the reaction mixture was stirred at 80° C. for 1 h. The mixture was allowed to cool to room temperature and saturated NaHCO₃ solution was applied. The mixture was diluted with EtOAc and the layers were separated. The aqueous layer was extracted with EtOAc and the combined organic layers were washed with brine dried over Na₂SO₄, and concentrated under reduced pressure.

The crude mixture was purified by flash column chromatography (silica gel, eluent: 2:1-1:1→1:2 Hexanes:EtOAc) to provide 17,18-unsaturated isoquinoline 21 (490 mg, 84%). ¹H NMR (500 MHz, CDCl₃) Shift=9.23 (s, 1H), 8.49 (d, J=5.4 Hz, 1H), 7.94 (s, 1H), 7.85-7.81 (m, 1H), 7.80-7.75 (m, 1H), 7.63 (d, J=5.4 Hz, 1H), 6.26 (br. s., 1H), 5.82 (s, 1H), 5.40 (d, J=3.4 Hz, 1H), 4.08-3.90 (m, 4H), 2.76 (dd, J=7.1, 11.0 Hz, 1H), 2.58 (dt, J=5.4, 17.6 Hz, 1H), 2.56-2.40 (m, 3H), 2.40-2.28 (m, 4H), 2.16 (d, J=13.2 Hz, 1H), 2.02 (dd, J=2.0, 13.2 Hz, 1H), 1.94 (td, J=8.8, 13.2 Hz, 1H), 1.81 (td, J=2.0, 12.7 Hz, 1H), 1.76-1.67 (m, 2H), 1.18 (s, 3H). HRMS (ESI) (m/z) calculated for C₃₀H₃₂NO₃ [M+H]⁺: 454.2377, found 454.2366.

Synthesis of Isoquinoline 12 from 17,18-Unsaturated Isoquinoline 21

To a solution of 17,18-unsaturated isoquinoline 21 (400 mg, 877 μmol, 1.0 equiv) in THF (36 mL) was added 10 wt % Pd/C (280 mg, 263 μmol, 0.30 equiv) and H₂ balloon was installed. After 3 h, the reaction mixture was filtered through a pad of Celite and washed with 0.2 M NH₃ solution in MeOH (40 mL), concentrated under reduced pressure. The residue was purified by flash column chromatography (silica gel, eluent: 1:1→1:2 Hexanes:EtOAc) to provide isoquinoline 12 (325 mg, 80%). Spectral data was consistent with isoquinoline 12 constructed from 1-chloroisoquinoline adduct 11.

### Example S5. Synthesis of Isoquinoline Analogs

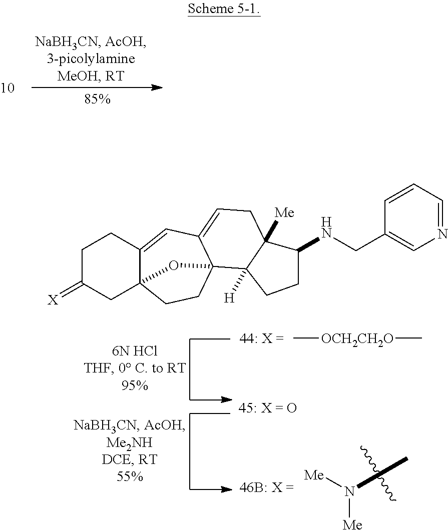

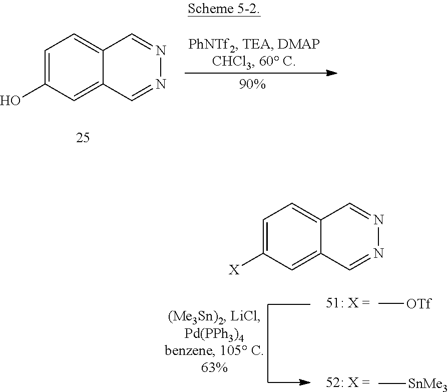

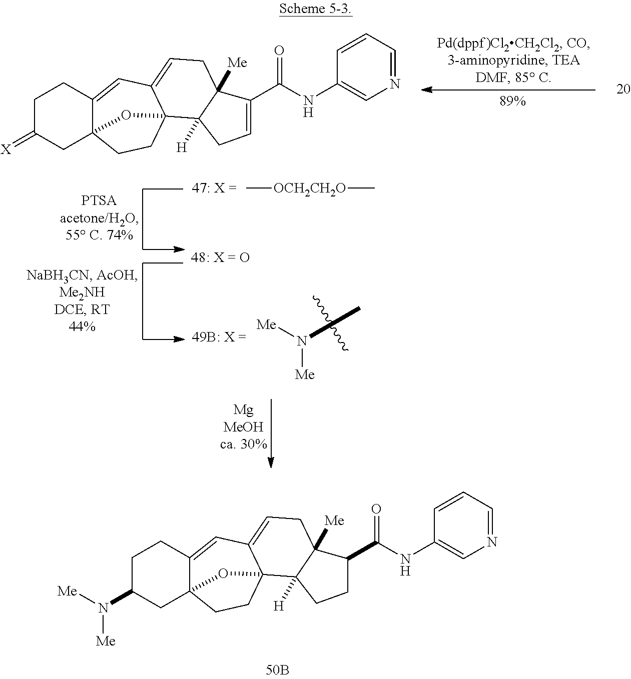

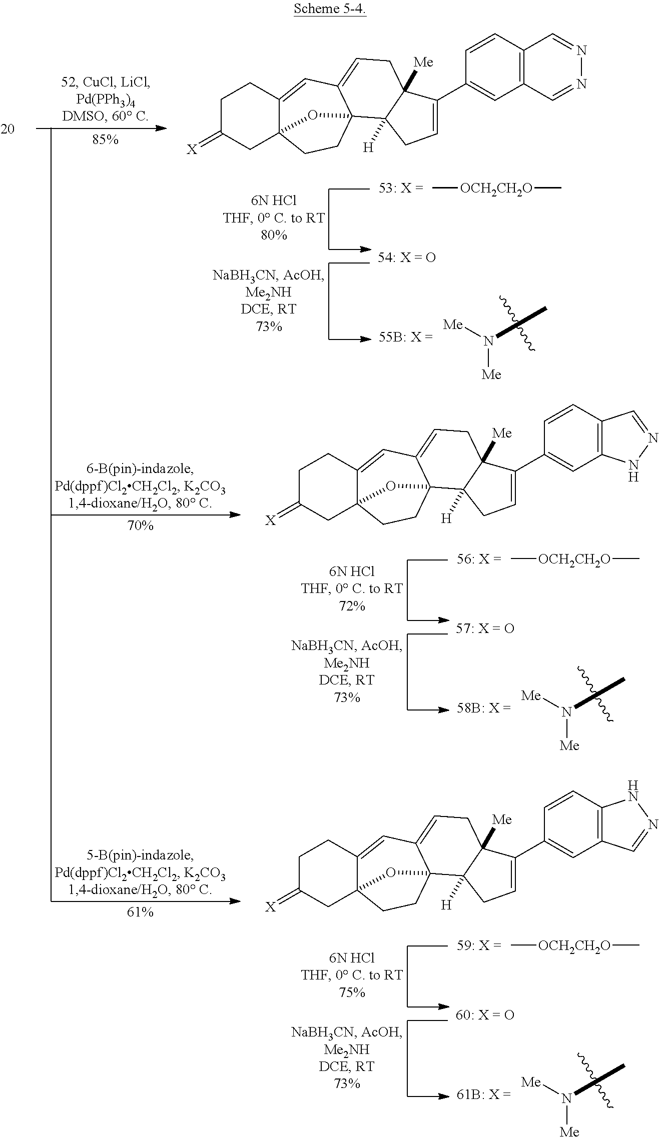

Suzuki Cross-Coupling for 17,18-Unsaturated 5-Indazole 56 from Triflate 20

To a solution of triflate 20 (1.00 equiv) and indazole-5-boronic ester (3.00 equiv) in 1,4-dioxane and H₂O (10:1, 0.02M) was added K₂CO₃ (3.00 equiv) and the solution was bubbled through inert Ar for 5 min. Pd(dppf)Cl₂.CH₂C12 (0.05 equiv) was added and the reaction mixture was stirred at 80° C. for 1 h. The mixture was allowed to cool to room temperature and saturated NaHCO₃ solution was applied. The mixture was diluted with EtOAc and the layers were separated. The aqueous layer was extracted with EtOAc and the combined organic layers were washed with brine dried over Na₂SO₄, and concentrated under reduced pressure.

The crude mixture was purified by flash column chromatography (silica gel, eluent: 1:3→1:1 EtOAc:Hexanes) to afford 17,18-unsaturated 6-indazole 56 (13 mg, 70%). ¹H NMR (500 MHz, CDCl₃) Shift=8.04 (s, 1H), 7.67 (d, J=10.0 Hz, 1H), 7.49 (s, 1H), 7.27 (d, J=10.0 Hz, 1H), 6.12 (br. s., 1H), 5.78 (br. s., 1H), 5.36 (t, J=5.0 Hz, 1H), 3.99 (m, 4H), 2.72 (m, 1H), 2.53-2.44 (m, 3H), 2.40 (br. d., J=10.0 Hz, 1H), 2.36-2.25 (m, 4H), 2.14 (d, J=10.0 Hz, 1H), 2.00 (dd, J=10.0, 5.0 Hz, 1H), 1.93-1.87 (m, 1H), 1.79 (m, 1H), 1.72-1.66 (m, 2H), 1.10 (s, 3H). HRMS (ESI) (m/z) calculated for C₂₈H₃₁N₂O₃ [M+H]⁺: 443.5573, found 443.5571.

Suzuki Cross-Coupling for 17,18-Unsaturated 6-Indazole 59 from Triflate 20

To a solution of triflate 20 (1.00 equiv) and indazole-6-boronic ester (3.00 equiv) in 1,4-dioxane and H₂O (10:1, 0.02M) was added K₂CO₃ (3.00 equiv) and the solution was bubbled through inert Ar for 5 min. Pd(dppf)Cl₂.CH₂C12 (0.05 equiv) was added and the reaction mixture was stirred at 80° C. for 1 h. The mixture was allowed to cool to room temperature and saturated NaHCO₃ solution was applied. The mixture was diluted with EtOAc and the layers were separated. The aqueous layer was extracted with EtOAc and the combined organic layers were washed with brine dried over Na₂SO₄, and concentrated under reduced pressure.

The crude mixture was purified by flash column chromatography (silica gel, eluent: 1:4→3:1 EtOAc:Hexanes) to afford 17,18-unsaturated 5-indazole 59 (5.9 mg, 65%). ¹H NMR (500 MHz, CDCl₃) Shift=10.04 (br s, 1H), 8.05 (s, 1H), 7.75 (s, 1H), 7.46 (ABq, J_{AB}=8.8 Hz, Δv=33.5 Hz, 2H), 6.02 (s, 1H), 5.79 (s, 1H), 5.38 (dd, J=3.4, 3.4 Hz, 1H), 3.94-4.01 (m, 4H), 2.73 (dd, J=10.7, 6.8 Hz, 1H), 2.44-2.53 (m, 3H), 2.40 (d, 12.2 Hz, 1H), 2.28-2.36 (m, 3H), 2.15 (d, J=13.2 Hz, 1H), 2.03 (par obs d, J=11.2 Hz, 1H), 2.01 (par obs dd, J=13.2, 2.4 Hz, 1H), 1.91 (dt, J=12.21, 9.3 Hz, 1H), 1.77-1.82 (m, 1H), 1.66-1.73 (m, 2H), 1.10 (s, 3H). HRMS (ESI) (m/z) calculated for C₂₈H₃₁N₂O₃ [M+H]⁺: 443.2335, found 443.4956.

### β-Dimethylamine Aminomethylpyridine 46B and α-Dimethylamine Aminomethylpyridine 46A

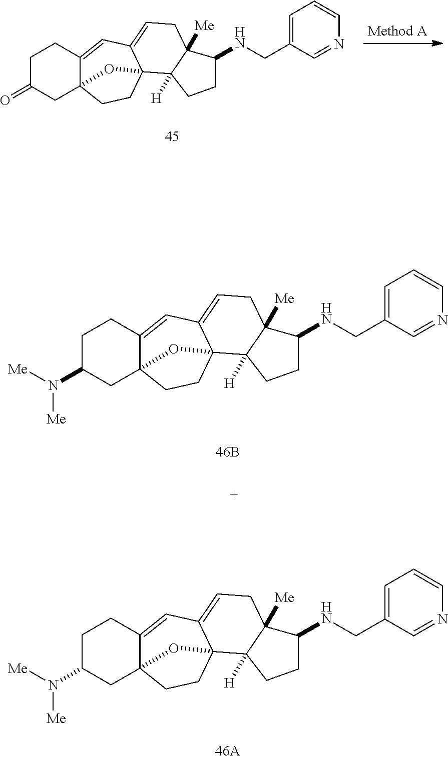 [SMILES: C[C@]12CC=C3C=C4CCC(=O)C[C@]45CC[C@]3(O5)[C@@H]1CC[C@@H]2NCc1cccnc1 ; CN(C)[C@H]1CCC2=CC3=CC[C@]4(C)[C@@H](NCc5cccnc5)CC[C@H]4[C@@]34CC[C@]2(C1)O4 ; CN(C)[C@@H]1CCC2=CC3=CC[C@]4(C)[C@@H](NCc5cccnc5)CC[C@H]4[C@@]34CC[C@]2(C1)O4]

β-Dimethylamine aminomethylpyridine 46B:

The crude mixture was purified by flash chromatography (silica gel, eluent: 10:1 EtOAc:2M NH₃ solution in MeOH) to afford β-dimethylamine aminomethylpyridine 46B (5 mg, 55%). ¹H NMR (500 MHz, CDCl₃) Shift=8.58 (s, 1H), 8.51 (dd, J=1.5, 4.9 Hz, 1H), 7.71 (td, J=2.0, 7.8 Hz, 1H), 7.26 (dd, J=4.9, 7.8 Hz, 1H), 5.71 (s, 1H), 5.24 (dd, J=2.4, 4.4 Hz, 1H), 3.88-3.80 (m, 2H), 2.81 (t, J=9.0 Hz, 1H), 2.45 (dt, J=6.3, 13.7 Hz, 1H), 2.46-2.37 (m, 1H), 2.30 (br. s., 6H), 2.26-2.19 (m, 3H), 2.17-2.07 (m, 5H), 1.92 (dd, J=2.9, 13.7 Hz, 2H), 1.79 (dddd, J=4.9, 8.3, 10.3, 13.2 Hz, 1H), 1.74-1.59 (m, 4H), 1.39 (ddt, J=4.4, 9.8, 12.7 Hz, 1H), 0.80 (s, 3H). HRMS (ESI) (m/z) calculated for C₂₇H₃₈N₃O [M+H]⁺: 420.3009, found 420.2999.

### β-Dimethylamine 17,18-Unsaturated Amidepyridine 49B and α-Dimethylamine 17,18-Unsaturated Amidepyridine 49A

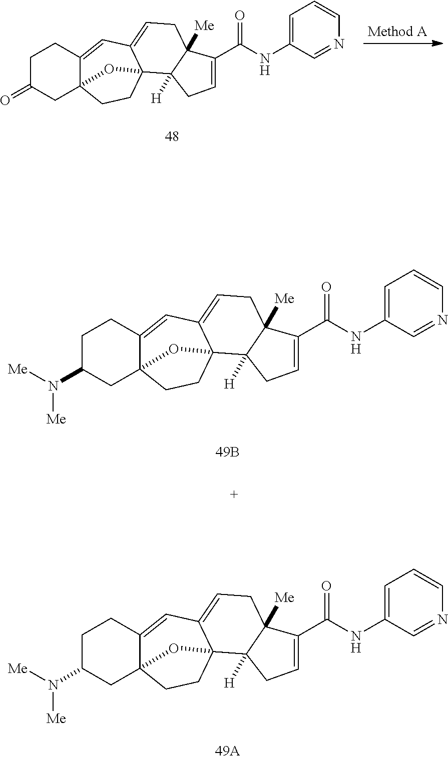 [SMILES: C[C@]12CC=C3C=C4CCC(=O)C[C@]45CC[C@]3(O5)[C@@H]1CC=C2C(=O)Nc1cccnc1 ; CN(C)[C@H]1CCC2=CC3=CC[C@]4(C)C(C(=O)Nc5cccnc5)=CC[C@H]4[C@@]34CC[C@]2(C1)O4 ; CN(C)[C@@H]1CCC2=CC3=CC[C@]4(C)C(C(=O)Nc5cccnc5)=CC[C@H]4[C@@]34CC[C@]2(C1)O4]

### β-Dimethylamine 17,18-Unsaturated Amidepyridine 49B:

The crude mixture was purified by flash chromatography (silica gel, eluent: 8:1 EtOAc:2M NH₃ solution in MeOH) to afford β-dimethylamine 17,18-unsaturated amidepyridine 49B (2.1 mg, 44%). ¹H NMR (500 MHz, CDCl₃) Shift=8.58 (d, J=2.4 Hz, 1H), 8.36 (dd, J=1.0, 4.9 Hz, 1H), 8.23 (td, J=2.0, 8.3 Hz, 1H), 7.49 (s, 1H), 7.29 (dd, J=4.9, 8.8 Hz, 1H), 6.57 (br. s., 1H), 5.73 (s, 1H), 5.33 (dd, J=2.0, 5.4 Hz, 1H), 2.66-2.56 (m, 2H), 2.54 (dd, J=3.2, 6.6 Hz, 1H), 2.51-2.32 (m, 5H), 2.28 (br. s., 6H), 2.20 (ddd, J=1.2, 11.0, 12.7 Hz, 1H), 2.10 (td, J=6.4, 13.1 Hz, 2H), 1.97-1.89 (m, 3H), 1.76 (dt, J=7.8, 11.2 Hz, 1H), 1.66-1.55 (m, 1H), 1.14 (s, 3H). HRMS (ESI) (m/z) calculated for C₂₇H₃₄N₃O₂ [M+H]⁺: 432.2646, found 432.2649.

β-Dimethylamine 17,18-unsaturated phthalazine 55B and α-Dimethylamine 17,18-unsaturated phthalazine 55A

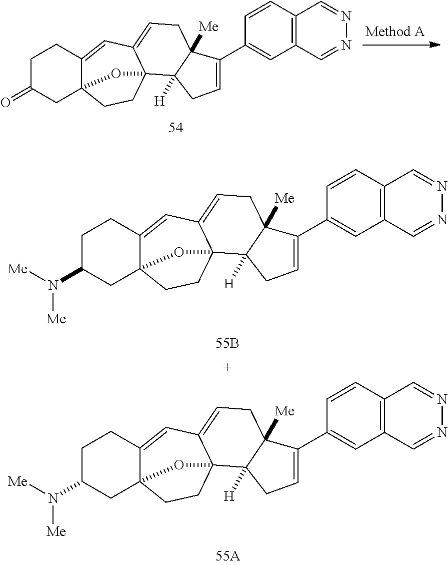

β-dimethylamine 17,18-unsaturated phthalazine 55B:

The crude mixture was purified by flash chromatography (silica gel, eluent: 9:1 EtOAc:2M NH₃ solution in MeOH) to afford β-dimethylamine 17,18-unsaturated phthalazine 55B (5.5 mg, 73%). ¹H NMR (500 MHz, CDCl₃) Shift=9.50 (s, 2H), 7.97-8.03 (i, 1H), 7.89 (d, J=4.39 Hz, 2H), 6.34-6.39 (i, 1H), 5.77-5.83 (m, 1H), 5.32-5.43 (m, 1H), 2.72 (dd, J=11.23, 6.84 Hz, 1H), 2.38-2.50 (m, 4H), 2.47 (br. m., 6H), 2.24-2.30 (m, 3H), 2.09-2.20 (m, 2H), 2.03 (d, J=10.25 Hz, 2H), 1.88-1.99 (m, 2H), 1.75-1.86 (m, 2H), 1.17 (s, 3H). HRMS (ESI) (m/z) calculated for C₂₈H₃₃N₃O [M+H]⁺: 440.5998, found 440.5995.

β-Dimethylamine 17,18-unsaturated 6-indazole 58B and α-Dimethylamine 17,18-unsaturated 6-indazole 58A

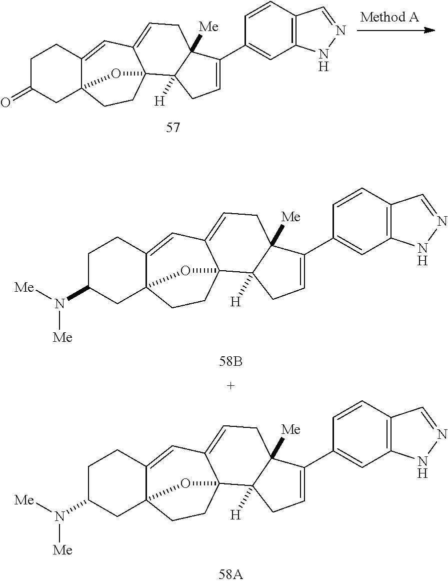

β-Dimethylamine 17,18-unsaturated 6-indazole 58B:

The crude mixture was purified by flash chromatography (silica gel, eluent: 9:1 EtOAc: 2M NH₃ solution in MeOH) to afford β-dimethylamine 17,18-unsaturated 6-indazole 58B (3.9 mg, 73%). ¹H NMR (500 MHz, CDCl₃) Shift=8.03 (s, 1H), 7.67 (d, J=8.30 Hz, 1H), 7.49 (s, 1H), 7.25-7.28 (m, 1H), 6.12 (t, J=2.44 Hz, 1H), 5.75 (s, 1H), 5.33 (t, J=3.42 Hz, 1H), 3.22-3.38 (m, 1H), 2.71 (dd, J=11.23, 6.84 Hz, 1H), 2.44-2.50 (m, 4H), 2.41 (br. m., 6H), 2.22-2.31 (m, 3H), 2.09-2.20 (m, 2H), 2.04 (d, J=2.93 Hz, 2H), 1.78 (td, J=11.35, 7.08 Hz, 2H), 1.52-1.64 (m, 1H), 1.07-1.15 (s, 3H). HRMS (ESI) (m/z) calculated for C₂₈H₃₃N₃O [M+H]⁺: 428.5891, found 428.5889.

### β-Dimethylamine 17,18-Unsaturated 5-Indazole 61B and α-Dimethylamine 17,18-Unsaturated 5-Indazole 61A

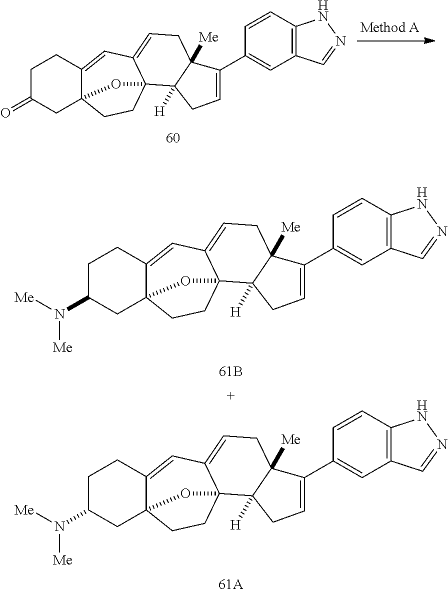

β-Dimethylamine 17,18-unsaturated 5-indazole 61B:

The crude mixture was purified by flash column chromatography (silica gel, eluent: 9:1 EtOAc:2M NH₃ solution in MeOH) to afford β-dimethylamine 17,18-unsaturated 5-indazole 61B (2.5 mg, 70%). ¹H NMR (500 MHz, CDCl₃) Shift=8.05 (s, 1H), 7.75 (s, 1H), 7.47 (ABq, J_{AB}=8.8 Hz, Δv=33.5 Hz, 2H), 6.02 (dd, J=2.0, 2.0 Hz, 1H), 5.74 (s, 1H), 5.33 (dd, J=3.4, 3.4 Hz, 1H), 2.72 (dd, J=11.2, 6.8 Hz, 1H), 2.43-2.48 (m, 5H), 2.37-2.41 (m, 2H), 2.26-2.35 (m, 8H), 2.11 (m, 2H), 2.04 (d, J=6.8 Hz, 1H), 1.88-1.98 (m, 3H), 1.77 (m, 1H), 1.10 (s, 3H). HRMS (ESI) (m/z) calculated for C₂₈H₃₄N₃O [M+H]⁺: 428.2702, found 428.2653.

### Synthesis of β-Dimethylamine Amidepyridine 50B

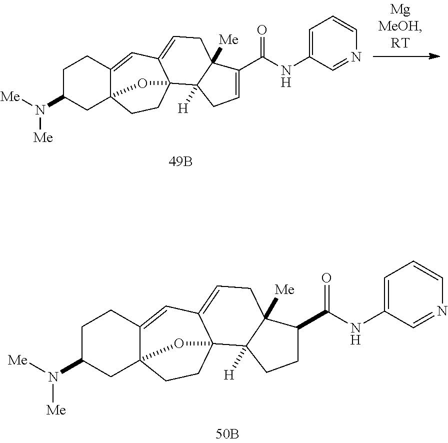

To a solution of β-dimethylamine 17,18-unsaturated amidepyridine 49B (ca. 1.2 mg) in MeOH (300 μL) was added Mg (ca. 1 mg) and stirred at room temperature for 48 h. The reaction mixture was added H₂O (700 μL) and diluted with EtOAc (700 μL). The aqueous phase was extracted with EtOAc (2×0.5 mL) and the combined organic phases were washed with brine (0.5 mL), dried over Na₂SO₄, and concentrated under reduced pressure. The residue was purified by HPLC (Eclipse XDB-C8 column, 9.4 mm×25 cm; gradient=0%→35% MeCN (0.1% formic acid):H₂O (0.1% formic acid) over 30 min) to provide β-dimethylamine amidepyridine 50B (ca. 0.3 mg, 25%). Due to the small quantity, only diagnostic peaks were assigned. ¹H NMR (500 MHz, CDCl₃) Shift=8.56 (br. s, 1H), 8.37 (d, J=3.4 Hz, 1H), 8.21 (d, J=8.3 Hz, 1H), 7.09 (br. s., 1H), 5.81 (s, 1H), 5.39-5.33 (m, 1H), 2.78 (br. s., 6H), 0.83 (s, 3H). HRMS (ESI) (m/z) calculated for C₂₇H₃₅N₃O₂ [M+H]⁺: 434.2802, found 434.2815.

### Synthesis of Phthalazine 6-Triflate 51

To a solution of 6-phthalazinol (588.4 mg, 4.26 mmol, 1.0 equiv) in CHC13 was added N-Phenyl-bis(trifluoromethanesulfonimide) (1.73 g, 4.83 mmol, 1.2 equiv), Et3N (0.9 mL, 6.04 mmol, 1.5 equiv) and DMAP (cat.). The mixture was warmed to 60° C. and stirred for 3 h. The reaction was cooled to room temperature and quenched with sat. NaHCO₃ and CH₂C12 and the layers were separated. The aqueous layer was extracted with CH₂C12. The organic layers were combined dried over Na₂SO₄ and concentrated under reduced pressure. The crude mixture was purified by flash column chromatography (silica gel, eluent: 9:1 Dichloromethane:MeOH) to afford phthalazine 6-triflate 51 (995 mg, 90%). ¹H NMR (500 MHz, CDCl₃) Shift=9.68 (d, J=3.91 Hz, 2H) 8.19 (d, J=8.79 Hz, 1H) 7.96 (br. s., 1H) 7.84-7.89 (m, 1H). HRMS (ESI) (m/z) calculated for C₉H₆F₃N₂O₃S [M+H]⁺: 279.2157, found 279.2152.

### Synthesis of Phthalazine 6-Trimethyltin 52

To a solution of phthalazine 6-triflate 52 (992 mg, 3.57 mmol, 1.0 equiv) in C₆H₆ was added LiCi (907 mg, 21.59 mmol, 6.0 equiv), Pd(PPh₃)₄ (412 mg, 0.3565 mmol, 0.1 equiv) and (Me₃Sn)₂ (0.78 mL, 3.743 mmol, 1.05 equiv). The solution was bubbled with argon in a sonicator for 10 mins and the mixture was warmed to 105° C. and stirred for 1 h. The reaction was cooled to rt, diluted with ethyl acetate and filtered over Celite. The organic portion was washed with sat NaHCO₃ and dried over Na₂SO₄ and then concentrated under reduced pressure. The crude mixture was purified by flash cloumn chromatography (silica gel, eluent: 1:1 EtOAc:Hexanes) to afford phthalazine 6-trimethyltin 52 (656 mg, 63%). ¹H NMR (500 MHz, CDCl₃) Shift=9.54 (d, J=4.39 Hz, 2H) 8.12 (br. s., 1H) 8.08 (d, J=7.81 Hz, 1H) 7.92 (d, J=7.81 Hz, 1H) 0.43 (s, 9H). HRMS (ESI) (m/z) calculated for C₁₁H15N2Sn [M+H]⁺: 293.9602, found 293.9601.

### Synthesis of 17,18-Unsaturated Phthalazine 53

To a solution of triflate 20 (20 mg, 42.15 μmol, 1.0 equiv) in DMSO was added (trimethylstannyl)phthalazine 52 (31 mg, 105.40 μmol, 2.0 equiv), CuCl (42 mg, 421.50 μmol, 10.0 equiv) and LiCl (18 mg, 421.50 μmol, 10.0 equiv). The mixture was deoxygenated by freeze-thaw method four times and Pd(PPh₃) (5 mg, 4.22 μmol, 0.1 equiv) was added. The mixture was heated to 60° C. and stir 1 h. The reaction was quenched with 5% NH₄OH and ethyl acetate and the layers were separated. The aqueous layer was extracted with ethyl acetate. The organic layers were combined, washed with brine, dried over Na₂SO₄ and concentrated under reduced pressure. The crude mixture was purified by flash chromatography (silica gel, eluent: 1:4→1:1→3:1 EtOAc:Hexanes) to afford 17,18-unsaturated phthalazine 53 (16.3 mg, 85%). ¹H NMR (500 MHz, CDCl₃) Shift=9.51 (d, J=5.0 Hz, 2H) 8.02 (d, J=5.0 Hz, 1H), 7.91 (s, 1H), 7.90 (d, J=5.0 Hz, 1H), 6.37 (br. s., 1H), 5.80 (br. s., 1H), 5.37 (br. m., 1H), 3.98 (m, 4H), 2.75 (m, 1H), 2.59-2.52 (m, 2H), 2.50-2.42 (m, 3H), 2.37-2.26 (m, 3H), 2.14 (d, J=15.0 Hz, 1H), 2.00 (dd, J=15.0, 2.5 Hz, 1H), 1.93 (m, 1H), 1.79 (m, 1H), 1.72-1.66 (m, 2H), 1.16 (s, 3H). HRMS (ESI) (m/z) calculated for C₂₉H₃₁N₂O₃ [M+H]⁺: 455.5680, found 455.5679.

### Synthesis of 17,18-Unsaturated Amidepyridine 47

To a solution of triflate 20 (20 mg, 42.1 μmol, 1.0 equiv) and 3-aminopyridine (19.8 mg, 210 μmol, 5.0 equiv) in DMF (1 mL) was added triethylamine (12 μL, 84.3 μmol, 2.0 equiv) and Pd(dppf)Cl₂.CH₂C12 (1.72 mg, 2.10 μmol, 0.05 equiv). Reaction flask was installed with CO balloon and the solution was purged for 5 min at room temperature. Then, the reaction mixture was heated up to 85° C. and stirred for 4 h. The mixture was allowed to cool to room temperature and EtOAc (3 mL) and H₂O was added. The layers were separated and the aqueous layer was extracted with EtOAc (3×2 mL), and the combined organic layers were washed with brine (3 mL), dried over Na₂SO₄, and concentrated under reduced pressure. The crude mixture was purified by flash column chromatography (silica gel, eluent: 10:10:1→10:10:2 Hexanes:EtOAc:MeOH) to provide 17,18-unsaturated amidepyridine 47 (17 mg, 89%). ¹H NMR (500 MHz, CDCl₃) Shift=8.57 (d, J=2.4 Hz, 1H), 8.33 (dd, J=1.2, 4.6 Hz, 1H), 8.20 (d, J=8.3 Hz, 1H), 7.70 (s, 1H), 7.26 (dd, J=4.9, 7.8 Hz, 1H), 6.55 (br. s., 1H), 5.77 (s, 1H), 5.36 (d, J=2.9 Hz, 1H), 4.03-3.89 (m, 4H), 2.63-2.56 (m, 2H), 2.52 (ddd, J=2.9, 7.3, 17.1 Hz, 1H), 2.52-2.37 (m, 3H), 2.36-2.27 (m, 2H), 2.20 (t, J=12.2 Hz, 1H), 2.12 (d, J=13.2 Hz, 1H), 1.97 (dd, J=2.2, 12.9 Hz, 1H), 1.89 (td, J=8.8, 13.2 Hz, 1H), 1.78 (tdd, J=2.4, 4.8, 12.8 Hz, 1H), 1.71-1.62 (m, 2H), 1.11 (s, 3H). HRMS (ESI) (m/z) calculated for C₂₇H₃₁N₂O₄ [M+H]⁺: 447.2278, found 447.2289.

### Example S6. General Method for Synthesis of Ketones

To a solution of ketal in THF at 0° C. was added 6N HCl (THF:6N HCl=1:1, 0.05M). The mixture was warmed to room temperature and stirred for 1 h. The reaction was quenched with 6 N NaOH and ethyl acetate and the layers were separated. The aqueous layer was extracted with ethyl acetate. The organic layers were combined dried over Na₂SO₄ and concentrated under reduced pressure.

### Synthesis of Ketone 45

The crude mixture was purified by flash column chromatography (silica gel, eluent: 15:1 Dichloromethane:MeOH) to afford ketone 45 (8.5 mg, 95%). ¹H NMR (500 MHz, CDCl₃) Shift=8.60 (s, 1H), 8.52 (d, J=3.9 Hz, 1H), 7.74 (br. s., 1H), 7.28 (dd, J=4.4, 8.3 Hz, 1H), 5.91 (s, 1H), 5.37 (dd, J=2.7, 4.6 Hz, 1H), 3.88 (d, J=13.7 Hz, 1H), 3.84 (d, J=13.7 Hz, 1H), 2.91 (d, J=14.6 Hz, 1H), 2.82 (t, J=9.0 Hz, 1H), 2.65 (d, J=15.1 Hz, 1H), 2.64 (dd, J=10.3, 14.6 Hz, 1H), 2.59-2.41 (m, 3H), 2.32-2.20 (m, 3H), 2.19-2.07 (m, 3H), 1.85-1.72 (m, 2H), 1.72-1.61 (m, 2H), 1.44 (br. s., 1H), 0.82 (s, 3H). HRMS (ESI) (m/z) calculated for C₂₅H₃₁N₂O₂ [M+H]⁺: 391.2380, found 391.2366.

### Synthesis of Ketone 54

The crude mixture was purified by flash column chromatography (silica gel, eluent: 1:1 EtOAc:Hexanes) to afford ketone 54 (8.7 mg, 80%). ¹H NMR (500 MHz, CDCl₃) Shift=9.54 (d, J=10.0 Hz, 2H) 8.04 (d, J=10.0 Hz, 1H), 7.93 (br. s., 2H), 6.40 (br. s., 1H), 5.95 (br. s., 1H), 5.47 (br. m., 1H), 2.95 (d, J=10.0 Hz, 1H), 2.77 (m, 1H), 2.71-2.62 (m, 2H), 2.59-2.44 (m, 7H), 2.34 (t, J=10.0 Hz, 1H), 2.19 (t, J=10.0 Hz, 1H), 2.00 (m, 1H), 1.78 (m, 1H), 1.18 (s, 3H). HRMS (ESI) (m/z) calculated for C₂₇H₂₇N₂O₂ [M+H]⁺: 411.5155, found 411.5152.

### Synthesis of Ketone 57

The crude mixture was purified by flash column chromatography (silica gel, eluent: 1:1 EtOAc:Hexanes) to afford ketone 57 (13.7 mg, 72%). ¹H NMR (500 MHz, CDCl₃) Shift=8.04 (br. s., 1H), 7.68 (d, J=10.0 Hz, 1H), 7.48 (br. s., 1H), 7.27 (d, J=10.0 Hz, 1H), 6.13 (br. s., 1H), 5.93 (br. s., 1H), 5.45 (br. t., J=5 Hz, 1H), 2.97 (d, J=15.0 Hz, 1H), 2.76-2.64 (m, 3H), 2.53-2.43 (m, 6H), 2.40-2.34 (m, 2H), 2.17 (t, J=10.0 Hz, 1H), 1.98 (m, 1H), 1.77 (m, 1H), 1.11 (s, 3H). HRMS (ESI) (m/z) calculated for C₂₆H₂₇N₂O₂ [M+H]⁺: 399.5048, found 399.5047.

### Synthesis of Ketone 60

The crude mixture was purified by flash column chromatography (silica gel, eluent: 1:1→3:1 EtOAc:Hexanes, buffered with 2% triethylamine) to afford ketone 60 (3.3 mg, 68%). ¹H NMR (500 MHz, CDCl₃) Shift=9.93-10.27 (br s, 1H), 8.06 (s, 1H), 7.75 (s, 1H), 7.47 (ABq, J_{AB}=8.8 Hz, Δv=29.5 Hz, 2H), 6.03 (dd, J=2.0, 2.0 Hz, 1H), 5.94 (s, 1H), 5.47 (dd, J=3.9, 3.9 Hz, 1H), 2.97 (d, J=15.1 Hz, 1H), 2.63-2.76 (m, 3H), 2.53-2.59 (m, 1H), 2.44-2.50 (m, 5H), 2.35-2.42 (m, 2H), 2.17 (dd, J=9.3, 9.3 Hz, 1H), 1.95-2.01 (m, 1H), 1.74-1.80 (m, 1H), 1.11 (s, 3H). HRMS (ESI) (m/z) calculated for C₂₆H₂₇N₂O₂ [M+H]⁺: 399.2073, found 399.2043.

### Synthesis of Ketone 22

For the method, see ‘Synthesis of Ketone 13’. The resulting residue was then purified by flash chromatography (silica gel, eluent: 3:2→1:2 Hexanes:EtOAc) to afford ketone 22 (8.2 mg, 61%). ¹H NMR (500 MHz, CDCl₃) Shift=9.24 (br. s., 1H), 8.51 (d, J=5.4 Hz, 1H), 7.94 (s, 1H), 7.84-7.76 (m, 2H), 7.63 (d, J=5.4 Hz, 1H), 6.27 (br. s., 1H), 5.97 (s, 1H), 5.50 (dd, J=2.4, 4.9 Hz, 1H), 2.98 (d, J=14.6 Hz, 1H), 2.78 (dd, J=6.8, 11.2 Hz, 1H), 2.71 (d, J=14.6 Hz, 1H), 2.72-2.63 (m, 1H), 2.61 (d, J=5.4 Hz, 1H), 2.59-2.54 (m, 2H), 2.54-2.50 (m, 2H), 2.50-2.42 (m, 2H), 2.39 (ddd, J=1.5, 11.0, 12.9 Hz, 1H), 2.20 (ddd, J=1.5, 9.5, 11.5 Hz, 1H), 2.01 (ddd, J=7.3, 8.8, 12.7 Hz, 1H), 1.79 (dt, J=7.3, 11.2 Hz, 1H), 1.18 (s, 3H). HRMS (ESI) (m/z) calculated for C₂₈H₂₈NO₂ [M+H]⁺: 410.2115, found 410.2111.

### Synthesis of Ketone 48

For the method, see ‘Synthesis of Ketone 13’. The resulting residue was then purified by flash chromatography (silica gel, eluent: 20:10:3 Hexanes:EtOAc:2M NH₃ solution in MeOH) to afford ketone 48 (5.0 mg, 74%). ¹H NMR (500 MHz, CDCl₃) Shift=8.60 (d, J=2.0 Hz, 1H), 8.37 (dd, J=1.0, 4.9 Hz, 1H), 8.24-8.20 (m, 1H), 7.55 (s, 1H), 7.30 (dd, J=4.9, 8.3 Hz, 1H), 6.57 (br. s., 1H), 5.94 (s, 1H), 5.48 (dd, J=2.2, 5.1 Hz, 1H), 2.95 (d, J=15.1 Hz, 1H), 2.69 (d, J=14.6 Hz, 1H), 2.68 (d, J=12.7 Hz, 1H), 2.66-2.61 (m, 2H), 2.61-2.53 (m, 2H), 2.52-2.44 (m, 3H), 2.41 (d, J=18.1 Hz, 1H), 2.29 (ddd, J=1.5, 11.1, 12.8 Hz, 1H), 2.22-2.15 (m, J=1.5, 9.4, 11.1 Hz, 1H), 1.98 (ddd, J=7.6, 9.0, 12.7 Hz, 1H), 1.76 (dt, J=7.3, 11.2 Hz, 1H), 1.14 (s, 3H). HRMS (ESI) (m/z) calculated for C₂₅H₂₇N₂O₃ [M+H]⁺:403.2016, found 403.2023.

### Example S7. General Method for Synthesis of N-Oxides

To a solution of amine (1.00 equiv) in methanol (0.028 M) was added H₂O₂ (32.0 equiv) at room temperature. After 25 h, saturated NaHCO₃ solution was added, diluted with dichloromethane, and the layers were separated. The aqueous layer was extracted with dichloromethane. The organic layers were combined, washed with brine, dried over Na₂SO₄ and concentrated under reduced pressure.

### Synthesis of 14B—N-Oxide (14BNO)

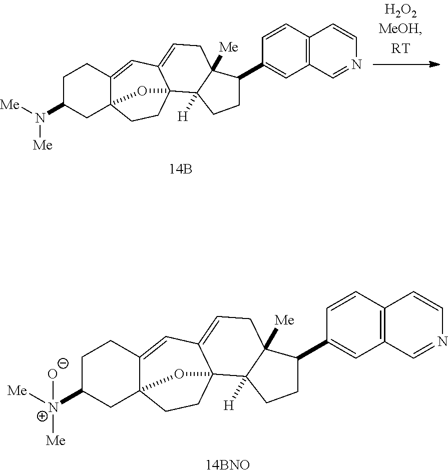

The crude mixture was purified by flash column chromatography (silica gel, eluent: 90:9:1→80:18:2 Chloroform:Methanol:5N NH₄OH solution in H₂O) to provide N-oxide 14BNO (23.5 mg, 95%). ¹H NMR (500 MHz, CDCl₃) Shift=9.21 (s, 1H), 8.48 (d, J=5.9 Hz, 1H), 7.77 (s, 1H), 7.75 (d, J=8.3 Hz, 1H), 7.61 (d, J=5.9 Hz, 1H), 7.57 (dd, J=1.0, 8.8 Hz, 1H), 5.76 (s, 1H), 5.28 (d, J=2.9 Hz, 1H), 3.44-3.36 (m, 1H), 3.22 (s, 3H), 3.12 (t, J=10.8 Hz, 1H), 3.10 (d, J=1.0 Hz, 3H), 2.47 (dd, J=8.8, 11.2 Hz, 1H), 2.44-2.29 (m, 5H), 2.28-2.13 (m, 4H), 2.09 (ddd, J=1.5, 9.3, 11.2 Hz, 1H), 2.06-1.97 (m, 2H), 1.94 (dd, J=5.1, 17.4 Hz, 1H), 1.85 (dq, J=5.4, 12.2 Hz, 1H), 1.83-1.76 (m, 1H), 1.72 (td, J=9.3, 12.2 Hz, 1H), 0.54 (s, 3H). HRMS (ESI) (m/z) calculated for C₃₀H₃₇N₂O₂ [M+H]⁺:457.2850, found 457.2842.

### Synthesis of 14A-N-Oxide (14ANO)

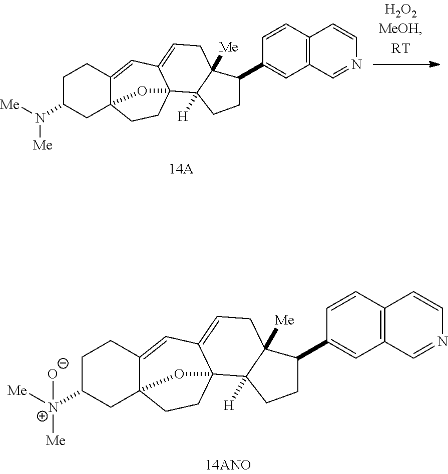

The crude mixture was purified by flash column chromatography (silica gel, eluent: 90:9:1→80:18:2 Chloroform:Methanol:5N NH₄OH solution in H₂O) to provide N-oxide 14ANO (3.6 mg, 77%). ¹H NMR (500 MHz, CDCl₃) Shift=9.24 (s, 1H), 8.50 (d, J=5.9 Hz, 1H), 7.80 (s, 1H), 7.77 (d, J=8.8 Hz, 1H), 7.64 (d, J=5.9 Hz, 1H), 7.60 (d, J=8.8 Hz, 1H), 5.81 (s, 1H), 5.36 (d, J=2.9 Hz, 1H), 3.46 (t, J=12.4 Hz, 1H), 3.24 (s, 3H), 3.18 (s, 3H), 3.16 (t, J=9.8 Hz, 1H), 2.64 (dd, J=3.2, 7.1 Hz, 1H), 2.59-2.47 (m, 3H), 2.43-2.28 (m, 4H), 2.28-2.15 (m, 2H), 2.09-1.99 (m, 2H), 1.97 (dd, J=5.1, 12.4 Hz, 1H), 1.88 (dq, J=5.4, 12.2 Hz, 1H), 1.81-1.71 (m, 2H), 1.51 (dq, J=4.1, 12.3 Hz, 1H), 0.56 (s, 3H). HRMS (ESI) (m/z) calculated for C₃₀H₃₇N₂O₂ [M+H]⁺:457.2850, found 457.2846.

### Cortistatin A N-Oxide Formation

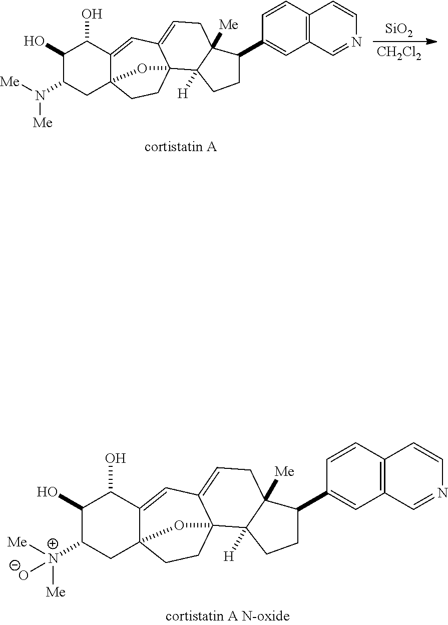

Cortistatin A N-Oxide:

To a solution of cortistatin A (2 mg) in ethyl acetate (1 mL) was added Aldrich silica gel Davisil™ (200 mesh) (200 mg) and this solution was stirred exposed to air for 1 hour. Silica gel was filtered and the filtrate was concentrated to give crude cortistatin A N-oxide that was further purified by SiO₂ chromatography (eluent: 50% methanol/ethyl acetate) to afford cortistatin A N-oxide (1.8 mg, 90% yield). ¹H NMR (500 MHz, CDCl₃) δ 9.22 (s, 1H), 8.50 (d, J=5.8 Hz, 1H), 7.78 (s, 1H), 7.76 (d, J=8.8 Hz, 1H), 7.63 (d, J=5.9 Hz, 1H), 7.58 (d, J=8.8 Hz, 1H), 6.28 (s, 1H), 5.49 (m, 1H), 4.15 (d, J=9.3 Hz, 1H), 3.83 (t, J=9.8, 9.8 Hz, 1H), 3.31-3.36 (m, 1H), 3.26 (s, 3H), 3.19 (s, 3H), 3.16 (dd, J=9.3, 9.3 Hz, 1H), 2.50 (dd, J=11.7, 8.8 Hz, 1H), 2.14-2.40 (m, 5H), 1.97-2.07 (m, 3H), 1.81-1.90 (m, 2H), 1.68-1.75 (m, 1H), 1.49-1.65 (m, 1H), 0.55 (s, 3H). HRMS (ESI) (m/z) calculated for C₃₀H₃₇N₂O₄ [M+H]⁺: 489.2753, found 489.5928.

### Example S8. Synthesis of C3-Alcohols and Substituted Analogs

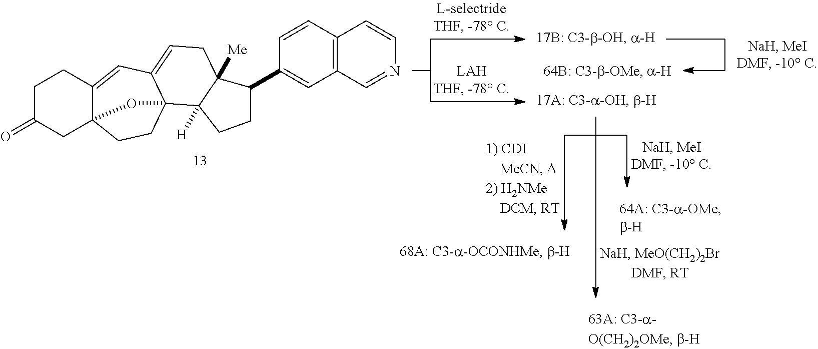

### Synthesis of β-Alcohol 17B

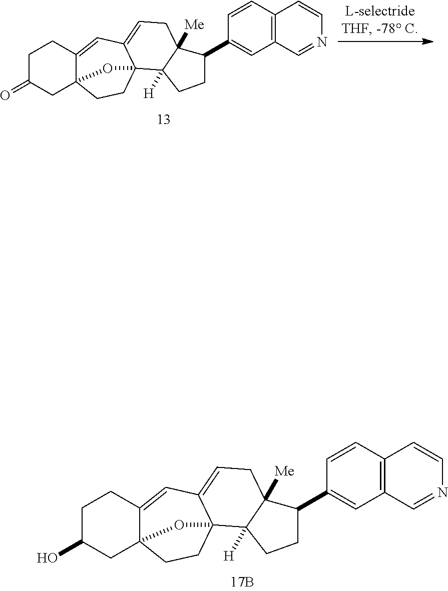

β-Alcohol 17B:

A solution of ketone 13 (2.00 mg, 4.85 μmol, 1.0 equiv) in THF (350 μL) was cooled to −78° C. and a solution of L-selectride in THF (1 M, 9.71 μL, 9.71 μmol, 2.0 equiv) was added. After 1 h, saturated NH₄Cl solution (400 μL) and ethyl acetate (300 μL) was added, which was allowed to warm to room temperature. The aqueous phase was extracted with ethyl acetate (3×1 mL) and the combined organic phases were washed with brine (1 mL), dried over Na₂SO₄, and concentrated under reduced pressure. The residue was purified by preparative TLC (eluent: 1:1 Hexanes:EtOAc) to afford β-alcohol 17B (ca. 1.2 mg, 60%).

¹H NMR (600 MHz, CDCl₃) Shift=9.26 (br. s, 1H), 8.49 (br. s, 1H), 7.82 (s, 1H), 7.78 (d, J=8.2 Hz, 1H), 7.69 (br. s, 1H), 7.63 (d, J=7.6 Hz, 1H), 5.75 (s, 1H), 5.26 (br. s, 1H), 4.36 (br. s, 1H), 3.15 (t, J=9.7 Hz, 1H), 2.63 (t, J=13.5 Hz, 1H), 2.51 (dd, J=9.1, 10.9 Hz, 1H), 2.42-2.28 (m, 2H), 2.24 (t, J=10.6 Hz, 1H), 2.21-2.12 (m, 2H), 2.12-1.97 (m, 3H), 1.93 (dd, J=5.0, 17.3 Hz, 2H), 1.90-1.80 (m, 2H), 1.79-1.58 (m, 3H), 0.54 (s, 2H). HRMS (ESI) (m/z) calculated for C₂₈H₃₂NO₂ [M+H]⁺: 414.2428, found 414.2436.

### α-Alcohol 17A

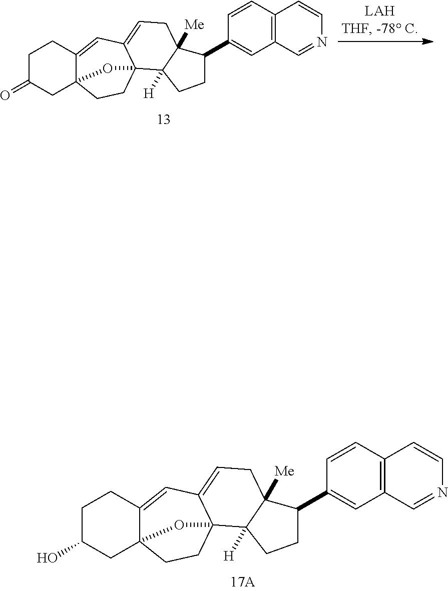

A solution of ketone 13 (9.6 mg, 23.3 μmol, 1.00 equiv) in THE (750 μL) was cooled to -78° C. and a solution of LAH in diethyl ether (1.0 M, 35.0 μL, 35.0 μmol, 1.50 equiv) was added. After 10 min, saturated NH₄Cl solution (500 μL) and ethyl acetate (500 μL) was added, which was allowed to warm to room temperature. The aqueous phase was extracted with ethyl acetate (3×1 mL) and the combined organic phases were washed with brine (1 mL), dried over Na₂SO₄, and concentrated under reduced pressure. The residue was purified by flash column chromatography (silica gel, eluent: 1:1→1:5 Hexanes:EtOAc -100% EtOAc) to provide α-alcohol 17A (8.5 mg, 88%). ¹H NMR (500 MHz, CDCl₃) Shift=9.22 (s, 1H), 8.47 (d, J=5.3 Hz, 1H), 7.79 (s, 1H), 7.75 (d, J=8.2 Hz, 1H), 7.63 (d, J=5.9 Hz, 1H), 7.59 (dd, J=1.2, 8.8 Hz, 1H), 5.75 (s, 1H), 5.28 (d, J=2.3 Hz, 1H), 3.78 (tt, J=4.0, 11.3 Hz, 1H), 3.14 (t, J=10.0 Hz, 1H), 2.51 (dd, J=8.5, 11.4 Hz, 1H), 2.40-2.33 (m, 2H), 2.32 (dt, J=4.7, 12.3 Hz, 1H), 2.28-1.98 (m, 7H), 1.93 (dd, J=5.0, 17.3 Hz, 1H), 1.90-1.81 (m, 2H), 1.74-1.62 (m, 2H), 1.40 (dtd, J=5.9, 11.6, 13.8 Hz, 1H), 0.53 (s, 3H). HRMS (ESI) (m/z) calculated for C₂₈H₃₂NO₂ [M+H]⁺: 414.2428, found 414.2437.

### α-Methylether 64A

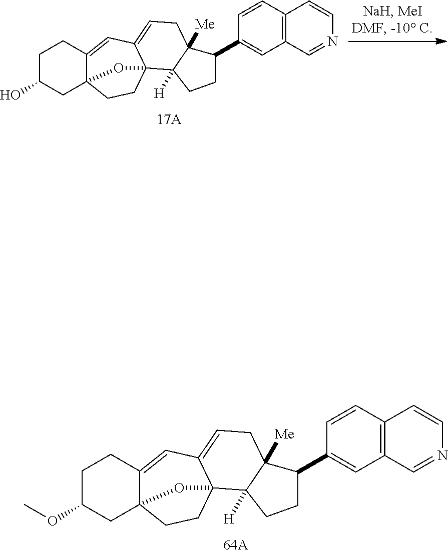

To a solution of α-alcohol 17A (2.0 mg, 4.83 μmol, 1.00 equiv) in DMF (300 μL) was added 60 wt % NaH (1.0 mg, 24.1 μmol, 5.00 equiv) at room temperature and pre-stirred 30 min. Temperature was lowered to −10° C. and MeI (2.0 μL, 29.0 μmol, 6.00 equiv) was added. After 2.5 hours, 2 M NaOH solution (200 μL) and ethyl acetate (500 μL) was added, which was allowed to warm to room temperature. The aqueous phase was extracted with ethyl acetate (3×1 mL) and the combined organic phases were washed with brine (1 mL), dried over Na₂SO₄, and concentrated under reduced pressure. The residue was purified by preparative TLC (eluent: 1:2 Hexanes:EtOAc) to afford α-methylether 64A (ca. 1.2 mg, 58%). ¹H NMR (600 MHz, CDCl₃) Shift=9.22 (s, 1H), 8.48 (d, J=5.9 Hz, 1H), 7.79 (s, 1H), 7.75 (d, J=8.8 Hz, 1H), 7.63 (d, J=5.9 Hz, 1H), 7.59 (dd, J=1.5, 8.5 Hz, 1H), 5.74 (s, 1H), 5.28 (d, J=2.9 Hz, 1H), 3.39 (s, 3H), 3.29 (tt, J=3.5, 11.4 Hz, 1H), 3.14 (t, J=10.0 Hz, 1H), 2.52 (dd, J=8.5, 11.4 Hz, 1H), 2.42-2.29 (m, 3H), 2.25 (t, J=11.7 Hz, 1H), 2.24-2.08 (m, 5H), 2.06 (td, J=4.1, 12.9 Hz, 1H), 1.93 (dd, J=5.3, 17.0 Hz, 1H), 1.86 (dq, J=5.3, 12.3 Hz, 1H), 1.79 (t, J=12.0 Hz, 1H), 1.75-1.61 (m, 2H), 1.32 (dtd, J=4.7, 11.5, 14.0 Hz, 1H), 0.53 (s, 3H). HRMS (ESI) (m/z) calculated for C₂₈H₃₂NO₂ [M+H]⁺: 428.2584, found 428.2573.

### β-Methylether 64B

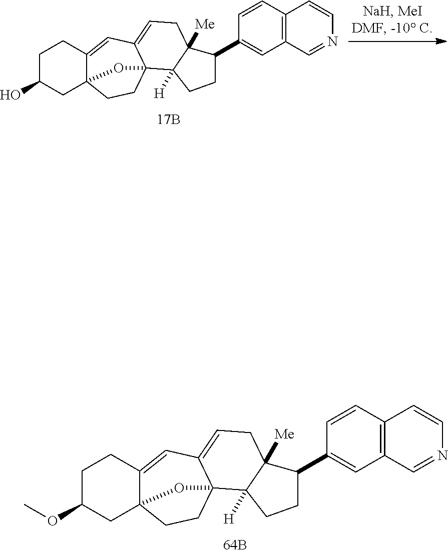

The reaction condition is same as the synthesis of α-methylether 64A. The residue was purified by preparative TLC (eluent: 1:2 Hexanes:EtOAc) to afford C3 β-methylether 64B (1.2 mg, 58%). ¹H NMR (500 MHz, CDCl₃) Shift=9.22 (s, 1H), 8.48 (d, J=5.9 Hz, 1H), 7.79 (s, 1H), 7.76 (d, J=8.8 Hz, 1H), 7.63 (d, J=5.9 Hz, 1H), 7.60 (dd, J=1.5, 8.3 Hz, 1H), 5.74-5.70 (m, 1H), 5.25 (dd, J=2.2, 5.1 Hz, 1H), 3.75-3.69 (m, 1H), 3.35 (s, 3H), 3.18-3.10 (m, J=9.3 Hz, 1H), 2.51 (dd, J=8.5, 11.5 Hz, 1H), 2.52-2.44 (m, 1H), 2.40-2.26 (m, 4H), 2.17 (s, 3H), 2.14-2.08 (m, 1H), 2.07-1.96 (m, 2H), 1.96-1.90 (m, 2H), 1.86 (dq, J=4.9, 12.0 Hz, 1H), 1.76-1.61 (m, 1H), 1.55-1.45 (m, 1H), 0.54 (s, 3H). HRMS (ESI) (m/z) calculated for C28H₃₂NO₂ [M+H]⁺: 428.2584, found 428.2599. α-Monomethylcarbamate 68A

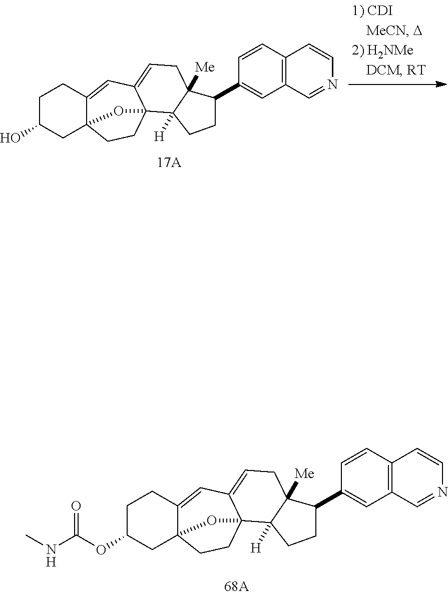

To a solution of α-alcohol 17A (3.5 mg, 8.5 μmol, 1.00 equiv) in CH₃CN (350 μL) was added CDI (2.1 mg, 12.7 μmol, 1.50 equiv) and the reaction mixture was refluxed for 4 hours. The crude mixture was concentrated under reduced pressure and used for the next reaction without further purification.

To a solution of the crude mixture in DCM (300 μL) was added MeNH₂ in THF (2 M, 50 pL) at room temperature and stirred 14 hours. The crude mixture was concentrated under reduced pressure and purified by preparative TLC (eluent: 1:1 Hexanes:EtOAc) to afford C3 α-monomethylcarbamate 68A (2.4 mg, 60% in 2 steps). ¹H NMR (500 MHz, CDCl₃) Shift=9.28 (s, 1H), 8.50 (d, J=5.9 Hz, 1H), 7.87 (s, 1H), 7.83 (d, J=8.8 Hz, 1H), 7.75 (d, J=5.9 Hz, 1H), 7.69 (d, J=8.8 Hz, 1H), 5.77 (s, 1H), 5.30 (dd, J=2.2, 4.6 Hz, 1H), 4.76 (t, J=11.7 Hz, 1H), 4.61 (br. s., 1H), 3.17 (t, J=10.0 Hz, 1H), 2.82 (d, J=4.4 Hz, 3H), 2.53 (dd, J=8.5, 11.5 Hz, 1H), 2.38 (d, J=17.1 Hz, 2H), 2.32 (dd, J=3.7, 11.5 Hz, 1H), 2.30-2.14 (m, 5H), 2.13-2.01 (m, 2H), 1.95 (dd, J=5.1, 17.3 Hz, 1H), 1.94-1.82 (m, 2H), 1.78-1.67 (m, 2H), 1.43 (dq, J=4.9, 12.7 Hz, 1H), 0.55 (s, 3H). HRMS (ESI) (m/z) calculated for C₃₀H₃₅N₂O₃ [M+H]⁺: 471.2642, found 471.2631.

### α-Methoxyethylether 63A

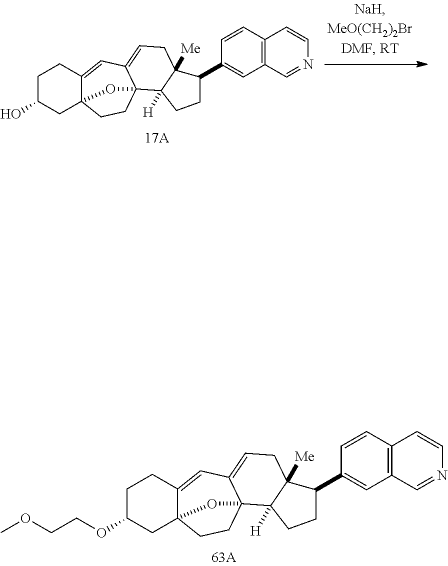

To a solution of α-alcohol 17A (2.0 mg, 4.83 μmol, 1.00 equiv) in DMF (300 μL) was added 60 wt % NaH (1.0 mg, 24.1 μmol, 5.00 equiv) at room temperature and pre-stirred 30 min before the addition of MeO(CH₂)₂Br (1.6 μL, 16.6 μmol, 3.00 equiv). After 36 hours, 2 M NaOH solution (200 μL) and ethyl acetate (500 μL) was added. The aqueous phase was extracted with ethyl acetate (3×1 mL) and the combined organic phases were washed with brine (1 mL), dried over Na₂SO₄, and concentrated under reduced pressure. The residue was purified by preparative TLC (eluent: 2:5 Hexanes:EtOAc) to afford C3 α-methoxyethylether 63A (ca. 0.7 mg, 27%). ¹H NMR (600 MHz, CDCl₃) Shift=9.24 (s, 1H), 8.50 (d, J=5.9 Hz, 1H), 7.81 (s, 1H), 7.77 (d, J=8.3 Hz, 1H), 7.65 (d, J=5.9 Hz, 1H), 7.61 (d, J=8.8 Hz, 1H), 5.75 (s, 1H), 5.29 (d, J=2.4 Hz, 1H), 3.74-3.63 (m, 2H), 3.61-3.51 (m, 2H), 3.44 (tt, J=3.9, 11.7 Hz, 1H), 3.44-3.39 (s, 3H), 3.16 (t, J=9.8 Hz, 1H), 2.54 (dd, J=8.5, 11.5 Hz, 1H), 2.43-2.30 (m, 3H), 2.30-2.17 (m, 4H), 2.17-2.02 (m, 3H), 1.95 (dd, J=5.1, 17.3 Hz, 1H), 1.92-1.82 (m, 2H), 1.78-1.62 (m, J=8.3 Hz, 2H), 1.41 (dtd, J=4.1, 11.9, 13.5 Hz, 1H), 0.55 (s, 3H). HRMS (ESI) (m/z) calculated for C28H₃₂NO₂ [M+H]⁺: 472.2846, found 472.2850.

### Example S9. Synthesis of Amines from Alcohols

### α-Dimethylhydantoin 74A

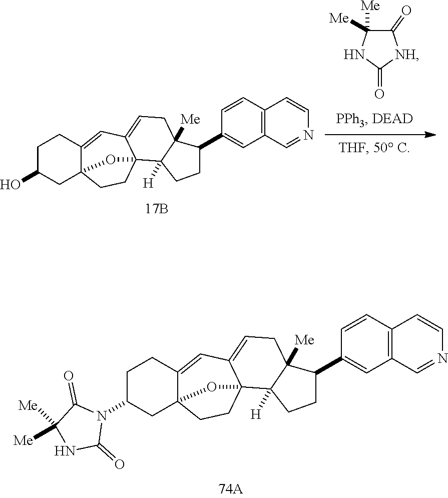

To a solution of β-alcohol 17B (5.0 mg, 12.3 μmol, 1.0 equiv) in THE (400 μL) was add dimethylhydantoin (7.8 mg, 61.6 μmol, 5.0 equiv) and PPh₃ (9.7 mg, 36.9 μmol, 3.0 equiv).

Reaction mixture was cooled to 0° C. and DEAD (16.1 μL of 40 wt % solution in toluene, 36.9 μmol, 3.0 equiv). Reaction was warmed up to 50° C. and stirred 17h. After cooling the reaction mixture to room temperature, IN NaOH solution (300 μL) and was added and the aqueous phase was extracted with ethyl acetate (3×0.5 mL) and the combined organic phases were washed with brine (1 mL), dried over Na₂SO₄, and concentrated under reduced pressure. The crude mixture was purified by preparative TLC (silica gel, eluent: 40:1 MeOH:Dichloromethane) to afford α-dimethylhydantoin 74A (0.9 mg, 14%). ¹H NMR (500 MHz, CDCl₃) Shift=9.24 (s, 1H), 8.50 (d, J=5.4 Hz, 1H), 7.81 (s, 1H), 7.77 (d, J=8.8 Hz, 1H), 7.64 (d, J=5.4 Hz, 1H), 7.61 (dd, J=1.5, 8.8 Hz, 1H), 5.79 (d, J=1.5 Hz, 1H), 5.32 (dd, J=2.7, 5.1 Hz, 1H), 5.18 (s, 1H), 4.10 (tdd, J=3.2, 11.3, 13.1 Hz, 1H), 3.16 (dd, J=9.3, 10.7 Hz, 1H), 2.86 (t, J=12.7 Hz, 1H), 2.54 (dd, J=8.3, 11.7 Hz, 1H), 2.45 (dd, J=2.9, 14.6 Hz, 1H), 2.30 (br. s., 6H), 2.19 (tq, J=4.4, 9.0 Hz, 1H), 2.09-2.00 (m, 1H), 1.96 (dd, J=5.4, 17.6 Hz, 1H), 1.92-1.80 (m, 2H), 1.80-1.64 (m, 3H), 1.42 (d, J=4.9 Hz, 6H), 0.56 (s, 3H). HRMS (ESI) (m/z) calculated for C₃₃H₃₈N₃O₃ [M+H]⁺: 524.2908, found 524.2892.

### II. CDK8/19 INHIBITORS

In any of the embodiments described herein, a CDK8/19 inhibitor other than cortistatin can be used in combination with the method of targeted selection of patients for therapy using the biomarkers identified herein. A range of CDK8/19 inhibitors are known in the art, including but not limited to those described in the following publications: Schiemann, K. et al. Discovery of potent and selective CDK8 inhibitors from an HSP90 pharmacophore. Bioorg. Med. Chem. Lett. 26, 1443-1451 (2016); Mallinger, A. et al. Discovery of Potent, Selective, and Orally Bioavailable Small-Molecule Modulators of the Mediator Complex-Associated Kinases CDK8 and CDK19. J. Med. Chem. 59, 1078-1101 (2016); Koehler, M., Bergeron, P. & Blackwood, E. M. Potent, Specific CDK8 Kinase Inhibitors Lack CDK8-dependent Antiproliferative Activity in HCT-116 Colon Cancer Cell Line. ACS Med. Chem. Lett. (2016). doi:10.1021/acsmedchemlett.5b00278; Dale, T. et al. A selective chemical probe for exploring the role of CDK8 and CDK19 in human disease. Nat. Chem. Biol. 11, 973-980 (2015).

Additional non-limiting examples of CDK8/19 inhibitors known in the art are provided in the following U.S. Patent Applications: US2013/0217014; US2015/027953; US2004/0180848; US2004/018844; US2014/0038958; US2012/0071477; US2011/0229484; US2005/0009846; US2008/0287439; US2010/0093769; US2005/0256142; US2003/0018058; US2001/0047019; US2002/002178; US2009/0318441; US2005/0192300; US2009/0325983; US2006/0235034; US2010/0215644; US2010/00120781; US2006/0183760; US2009/0270427; US20020165259; US2006/0241297; US2004/0186288; US2006/0241112; US2006/0270687; US2006/0270687; US2004/0254094; US2003/0176699; US2006/0148824; US2003/0114672; US2009/0215805; US2009; 0137572; and US2007/0161635.

In one embodiment the CDK8/19 inhibitor is an analog of Senexin. In another embodiment the CDK8/19 inhibitor is an analog of Selvita.

### III. TARGETED SELECTION OF PATIENTS FOR CDK8/19 INHIBITOR THERAPY

It has been discovered that certain patients with a tumor or cancer are more likely to respond to cortistatin therapy than other patients, and that these patients can be identified by the analysis of specific biomarkers in the patient's tumor or cancer, which are described in detail below. Using methods known in the art in combination with the disclosure herein, a healthcare provider can determine whether the patient will respond successfully to cortistatin treatment. This invention allows patients who will benefit from cortistatin therapy to be identified as candidates for treatment, and is a basis for excluding patients who are less likely to respond.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene or combination of genes. In some embodiments, a biomarker is a protein or combination of proteins. In other embodiments, a biomarker is a combination of genes and proteins. In some embodiments, the biomarker is the protein expressed by the gene. Biomarkers include, but are not limited to, polynucleotides (e.g., DNA, and/or RNA), polypeptides, polypeptide and polynucleotide modifications (e.g. posttranslational modifications), carbohydrates, and/or glycolipid-based molecular markers. In yet another embodiment, a biomarker is protein localization, for example an abundance of RUNX1 at certain loci to ascertain likelihood of patient response.

A. Selection of Patients Based on Impaired Runx1 Pathway

(RUNX1) is a master hematopoietic transcription factor (TF) which regulates the differentiation of hematopoietic stems cells into mature blood cells. It is sometimes alternatively referred to as acute myeloid leukemia 1 protein (AML1) or core-binding factor subunit alpha-2 (CBFA2). RUNX1 has been reported to regulate the differentiation of hematopoietic stem cells into mature blood cells, and over 35 mutations leading to RUNX1 inactivation have been identified to be implicated in various malignancies. Such inactivating mutations include, without limitation, RUNX1 point mutations, chromosomal translocations involving the RUNX1 gene, and mutations resulting in destabilization or increased degradation of the RUNX1 protein. For an overview of exemplary RUNX1 inactivating mutations known to be associated with cancer, see, e.g., Ito et al., The RUNX1 family: developmental regulators in cancer, Nature Reviews Cancer 15, 81-95 (2015), e.g., page 83, last paragraph to page 84, last paragraph, and Tables 1 and 2; and Ley et al., Genomic and Epigenomic Landscapes of Adult De Novo Acute Myeloid Leukemia, NEJM 368:22, 2059-74 (2013); the entire contents of which are incorporated herein by reference. While knowledge regarding RUNX1 mutations in cancer has yielded insights into the molecular pathology of various malignancies, the inactivation of a transcription factor, such as RUNX1, has been difficult to treat or correct by clinical intervention.

In non-hematopoetic malignancies such as breast cancer, inactivating RUNX1 mutations have also been observed and can contribute to solid tumor formation (Ito et al., The RUNX1 family: developmental regulators in cancer, Nature Reviews Cancer 15, 81-95 (2015); Ellis, M. J. et al. Whole-genome analysis informs breast cancer response to aromatase inhibition. Nature 486, 353-360 (2012); Banerji, S. et al. Sequence analysis of mutations and translocations across breast cancer subtypes. Nature 486, 405-409 (2012)). Furthermore, RUNX1 downregulation is evident in solid tumor metastasis compared to the primary tumors and its reduced expression is part of a 17-gene signature associated with metastasis (Ramaswamy, S., Ross, K. N., Lander, E. S. & Golub, T. R. A molecular signature of metastasis in primary solid tumors. Nature Genet. 33, 49-54 (2003)).

Confirmation that inhibition of CDK8/19 activates a RUNX1 program includes: 1) cortistatin A significantly increases expression of many RUNX1-target genes including CEBPA, IRF8, and NFE2 in AML cell lines including MOLM-14 which was derived from a patient diagnosed with myelodysplasia syndrome that transitioned to AML and 2) cortistatin A induced recruitment of RUNX1 to loci upregulated by cortistatin, suggesting that CDK8/19 kinase activity blocks accumulation of RUNX1 at target loci, and 3) cortistatin's anti-proliferative activity positively correlated with cell lines with impaired RUNX1-target gene expression including those harboring RUNX1 mutations.

Typically, a mutation in the RUNX1 gene that results in impaired RUNX1 activity is associated with a change in the amino acid sequence of the RUNX1 protein as compared to a wild type (non-mutated) RUNX1 sequence. Such mutations resulting in an abnormal RUNX1 protein include, for example, a substitution, deletion, or duplication of an amino acid or an amino acid sequence, a frameshift, or a premature stop codon in a protein-encoding sequence of RUNX1, or a fusion of the RUNX1 protein sequence, or a fragment thereof, to a heterologous protein, or fragment thereof. Such fusions are typically the result of a chromosomal translocation, resulting in a fusion of the genomic sequence encoding the RUNX1 protein, or a fragment thereof, to a genomic sequence encoding a different protein, or a fragment thereof.

Gene, transcript, and protein sequences of these and other RUNX1-binding partners or RUNX1 target genes are well known to those of skill in the art. Representative human RUNX1 binding partners and RUNX1 target genes are identified in Table 1 below.

TABLE 1
Representative human RUNX1 binding partners and RUNX1 target genes

| Name | Gene ID | Description | Location | Aliases |
|---|---|---|---|---|
| ACSL1 | 2180 | acyl-CoA synthetase long-chain | Chromosome 4, NC_000004.12 | ACS1, FACL1, |
| | | family member 1 | (184755595 . . . 184826114, | FACL2, LACS, |
| | | [Homo sapiens (human)] | complement) | LACS1, |
| | | | | LACS2 |
| ADORA2B | 136 | adenosine A2b receptor | Chromosome 17, NC_000017.11 | ADORA2 |
| | | [Homo sapiens (human)] | (15927783 . . . 15975896) | |
| ADRB1 | 153 | adrenoceptor beta 1 | Chromosome 10, NC_000010.11 | ADRB1R, |
| | | [Homo sapiens(human)] | (114044047 . . . 114046908) | B1AR, |
| | | | | BETA1AR, |
| | | | | RHR |
| AMPD3 | 272 | adenosine monophosphate | Chromosome 11, NC_000011.10 | N/A |
| | | deaminase 3 | (10450321 . . . 10507579) | |
| | | [Homo sapiens (human)] | | |
| ARRDC4 | 91947 | arrestin domain containing 4 | Chromosome 15, NC_000015.10 | N/A |
| | | [Homo sapiens (human)] | (97960703 . . . 97973838) | |
| BCL2 | 596 | B-cell CLL/lymphoma 2 | Chromosome 18, NC_000018.10 | Bcl-2, |
| | | [Homo sapiens(human)] | (63123346 . . . 63319778, | PPP1R50 |
| | | | complement) | |
| BCL2A1 | 597 | BCL2-related protein A1 | Chromosome 15, NC_000015.10 | ACC-1, ACC- |
| | | [Homosapiens (human)] | (79960890 . . . 79971301, | 2, ACC1, |
| | | | complement) | ACC2, |
| | | | | BCL2L5, |
| | | | | BFL1, GRS, |
| | | | | HBPA1 |
| CBFB | 865 | core-binding factor, beta subunit | Chromosome 16, NC_000016.10 | PEBP2B |
| | | [Homosapiens (human)] | (67029147 . . . 67101058) | |
| CCNA1 | 8900 | cyclin A1 [Homo sapiens (human)] | Chromosome 13, NC_000013.11 | CT146 |
| | | | (36431030 . . . 36442882) | |
| CD244 | 51744 | CD244 molecule, natural killer cell | Chromosome 1, NC_000001.11 | 2B4, NAIL, |
| | | receptor 2B4 | (160830158 . . . 160862902, | NKR2B4, Nmrk, |
| | | [Homosapiens(human)] | complement) | SLAMF4 |
| CD44 | 960 | CD44 molecule (Indian blood | Chromosome 11, NC_000011.10 | CDW44, |
| | | group) [Homo sapiens (human)] | (35138870 . . . 35232402) | CSPG8, |
| | | | | ECMR-III, |
| | | | | HCELL, |
| | | | | HUTCH-I, IN, |
| | | | | LHR, MC56, |
| | | | | MDU2, MDU3, |
| | | | | MIC4, Pgp1 |
| CDC42EP3 | 10602 | CDC42 effector protein (Rho | Chromosome 2, NC_000002.12 | BORG2, CEP3, |
| | | GTPase binding) 3 | (37641882 . . . 37672535, | UB1 |
| | | [Homo sapiens (human)] | complement) | |
| CEBPA | 1050 | CCAAT/enhancer binding protein | Chromosome 19, NC_000019.10 | C/EBP-alpha, |
| | | (C/EBP), alpha | (33299934 . . . 33302564, | CEBP |
| | | [Homo sapiens (human)] | complement) | |
| CECR6 | 27439 | cat eye syndrome chromosome | Chromosome 22, NC_000022.11 | N/A |
| | | region, candidate 6 | (17116299 . . . 17121367, | |
| | | [Homo sapiens (human)] | complement) | |
| CFLAR | 8837 | CASP8 and FADD-like apoptosis | Chromosome 2, NC_000002.12 | CASH, |
| | | regulator [Homo sapiens (human)] | (201116154 . . . 201172688) | CASP8AP1, |
| | | | | CLARP, |
| | | | | Casper, |
| | | | | FLAME1, |
| | | | | FLIP, I-FLICE, |
| | | | | MRIT, c-FLIP, |
| | | | | c-FLIPL, c- |
| | | | | FLIPR, c- |
| | | | | FLIPS |
| CISH | 1154 | cytokine inducible SH2-containing | Chromosome 3, NC_000003.12 | BACTS2, CIS, |
| | | protein [Homo sapiens (human)] | (50606454 . . . 50611831, | CIS-1, G18, |
| | | | complement) | SOCS |
| CSF1 | 1435 | colony stimulating factor 1 | Chromosome 1, NC_000001.11 | CSF-1, MCSF |
| | | (macrophage) | (109910611 . . . 109930994) | |
| | | [Homo sapiens (human)] | | |
| CXCL10 | 3627 | chemokine (C-X-C motif) ligand | Chromosome 4, NC_000004.12 | C7, IFI10, |
| | | 10 [Homo sapiens (human)] | (76021116 . . . 76023536, | INP10, IP-10, |
| | | | complement) | SCYB10, crg- |
| | | | | 2, gIP-10, mob- |
| | | | | 1 |
| CXCR4 | 7852 | chemokine (C-X-C motif) receptor | Chromosome 2, NC_000002.12 | CD184, |
| | | 4 [Homo sapiens (human)] | (136114349 . . . 136118155, | D2S201E, |
| | | | complement) | FB22, HM89, |
| | | | | HSY3RR, |
| | | | | LAP-3, LAP3, |
| | | | | LCR1, LESTR, |
| | | | | NPY3R, |
| | | | | NPYR, |
| | | | | NPYRL, |
| | | | | NPYY3R, |
| | | | | WHIM, |
| | | | | WHIMS |
| CYTIP | 9595 | cytohesin 1 interacting protein | Chromosome 2, NC_000002.12 | B3-1, CASP, |
| | | [Homo sapiens (human)] | (157414619 . . . 157444092, | CYBR, |
| | | | complement) | CYTHIP, HE, |
| | | | | PSCDBP |
| DUSP10 | 11221 | dual specificity phosphatase 10 | Chromosome 1, NC_000001.11 | MKP-5, MKP5 |
| | | [Homosapiens (human)] | (221701420 . . . 221742176, | |
| | | | complement) | |
| E2F8 | 79733 | E2F transcription factor 8 | Chromosome 11, NC_000011.10 | E2F-8 |
| | | [Homo sapiens (human)] | (19224063 . . . 19241655, | |
| | | | complement) | |
| EMB | 133418 | embigin [Homo sapiens (human)] | Chromosome 5, NC_000005.10 | GP70 |
| | | | (50396192 . . . 50443307, | |
| | | | complement) | |
| EMR2 | 30817 | adhesion G protein-coupled | chromosome 19, NC_000019.9 | ADGRE2, |
| | | receptor E2 | (14843509 . . . 14889353, | CD312 |
| | | | complement) | |
| ETS1 | 2113 | v-ets avian erythroblastosis virus | Chromosome 11, NC_000011.10 | ETS-1, |
| | | E26 oncogene homolog 1 | (128458761 . . . 128587584, | EWSR2, p54 |
| | | [Homo sapiens(human)] | complement) | |
| ETS2 | 2114 | v-ets avian erythroblastosis virus | Chromosome 21, NC_000021.9 | ETS2IT1 |
| | | E26 oncogene homolog 2 | (38805307 . . . 38824955) | |
| | | [Homo sapiens(human)] | | |
| FAM107B | 83641 | family with sequence similarity | Chromosome 10, NC_000010.11 | C10orf45 |
| | | 107, member B | (14518557 . . . 14774897, | |
| | | [Homo sapiens (human)] | complement) | |
| FAM46A | 55603 | family with sequence similarity 46, | Chromosome 6, NC_000006.12 | C6orf37, |
| | | member A | (81745730 . . . 81752711, | XTP11 |
| | | [Homo sapiens (human)] | complement) | |
| FCER1A | 2205 | Fc fragment of IgE, high affinity I, | Chromosome 1, NC_000001.11 | FCE1A, FcERI |
| | | receptor for; alpha polypeptide | (159283888 . . . 159308224) | |
| | | [Homo sapiens (human)] | | |
| FCGR1B | 2210 | Fc fragment of IgG, high affinity | Chromosome 1, NC_000001.11 | CD64b, IGFRB |
| | | Ib, receptor (CD64) | (121087345 . . . 121097161) | |
| | | [Homo sapiens (human)] | | |
| FLI1 | 2313 | Fli-1 proto-oncogene, ETS | Chromosome 11, NC_000011.10 | EWSR2, SIC-1 |
| | | transcription factor | (128686535 . . . 128813267) | |
| | | [Homo sapiens (human)] | | |
| FOG1 | 161882 | zinc finger protein, FOG family | Chromosome 16, NC_000016.10 | FOG, ZC2HC11A, |
| | | member 1 | (88451431 . . . 88535166) | ZNF408, |
| | | [Homo sapiens (human)] | | ZNF89A, |
| | | | | ZFPM1 |
| FOSL2 | 2355 | FOS-like antigen 2 | Chromosome 2, NC_000002.12 | FRA2 |
| | | [Homo sapiens(human)] | (28392759 . . . 28417312) | |
| GAB2 | 9846 | GRB2-associated binding protein 2 | Chromosome 11, NC_000011.10 | N/A |
| | | [Homo sapiens (human)] | (78215290 . . . 78417822, | |
| | | | complement) | |
| GAS7 | 8522 | growth arrest-specific 7 | Chromosome 17, NC_000017.11 | MLL/GAS7 |
| | | [Homo sapiens(human)] | (9910609 . . . 10198551, | |
| | | | complement) | |
| GATA1 | 2623 | GATA binding protein 1 (globin | Chromosome X, NC_000023.11 | ERYF1, |
| | | transcription factor 1) | (48786560 . . . 48794311) | GATA-1, GF- |
| | | [Homo sapiens(human)] | | 1, GF1, NF-E1, |
| | | | | NFE1, |
| | | | | XLANP, |
| | | | | XLTDA, |
| | | | | XLTT |
| GATA2 | 2624 | GATA binding protein 2 | Chromosome 3, NC_000003.12 | DCML, |
| | | [Homo sapiens(human)] | (128479422 . . . 128493187, | IMD21, |
| | | | complement) | MONOMAC, |
| | | | | NFE1B |
| GFI1B | 8328 | growth factor independent 1B | Chromosome 9, NC_000009.12 | BDPLT17 |
| | | transcription represser | (132945545 . . . 132993434) | |
| | | [Homo sapiens(human)] | | |
| GMPR | 2766 | guanosine monophosphate | Chromosome 6, NC_000006.12 | GMPR1 |
| | | reductase [Homo sapiens(human)] | (16238580 . . . 16295549) | |
| GPR18 | 2841 | G protein-coupled receptor 18 | Chromosome 13, NC_000013.11 | N/A |
| | | [Homo sapiens(human)] | (99254713 . . . 99262448, | |
| | | | complement) | |
| GPR183 | 1880 | G protein-coupled receptor 183 | Chromosome 13, NC_000013.11 | EBI2 |
| | | [Homo sapiens(human)] | (99294535 . . . 99307495, | |
| | | | complement) | |
| HBBP1 | 3044 | hemoglobin, beta pseudogene 1 | Chromosome 11, NC_000011.10 | HBH1, HBHP |
| | | [Homo sapiens (human)] | (5241954 . . . 5243592, | |
| | | | complement) | |
| HLX | 3142 | H2.0-like homeobox | Chromosome 1, NC_000001.11 | HB241, HLX |
| | | [Homo sapiens (human)] | (220879401 . . . 220885059) | |
| HMGCS1 | 3157 | 3-hydroxy-3-methylglutaryl-CoA | Chromosome 5, NC_000005.10 | HMGCS |
| | | synthase 1 (soluble) | (43287470 . . . 43313512, | |
| | | [Homo sapiens (human)] | complement) | |
| IGFBP4 | 3487 | insulin-like growth factor binding | Chromosome 17, NC_000017.11 | BP-4, HT29- |
| | | protein 4 [Homo sapiens (human)] | (40443424 . . . 40457730) | IGFBP, IBP4, |
| | | | | IGFBP-4 |
| IGFBP5 | 3488 | insulin-like growth factor binding | Chromosome 2, NC_000002.12 | IBP5 |
| | | protein 5 [Homosapiens (human)] | (216672105 . . . 216695549, | |
| | | | complement) | |
| IL17RA | 23765 | interleukin 17 receptor A | Chromosome 22, NC_000022.11 | CANDF5, |
| | | [Homo sapiens(human)] | (17084959 . . . 17115694) | CD217, |
| | | | | CDw217, IL- |
| | | | | 17RA, IL17R, |
| | | | | hIL-17R |
| IL1RAP | 3556 | interleukin 1 receptor accessory | Chromosome 3, NC_000003.12 | C3orf13, IL- |
| | | protein [Homosapiens (human)] | (190514051 . . . 190657197) | 1RAcP, IL1R3 |
| IPCEF1 | 26034 | interaction protein for cytohesin | Chromosome 6, NC_000006.12 | PIP3-E |
| | | exchange factors 1 | (154154483 . . . 154356802, | |
| | | [Homo sapiens(human)] | complement) | |
| IRF1 | 3659 | interferon regulatory factor 1 | Chromosome 5, NC_000005.10 | IRF-1, MAR |
| | | [Homosapiens (human)] | (132481609 . . . 132490777, | |
| | | | complement) | |
| IRF8 | 3394 | interferon regulatory factor 8 | Chromosome 16, NC_000016.10 | H-ICSBP, |
| | | [Homosapiens (human)] | (85899168 . . . 85922606) | ICSBP, |
| | | | | ICSBP1, |
| | | | | IMD32A, |
| | | | | IMD32B, IRF- |
| | | | | 8 |
| ITGA6 | 3655 | integrin, alpha 6 | Chromosome 2, NC_000002.12 | CD49fB, VLA- |
| | | [Homo sapiens (human)] | (172427586 . . . 172506453) | 6, ITGA6 |
| JAG1 | 182 | jagged 1 [Homo sapiens (human)] | Chromosome 20, NC_000020.11 | AGS, AHD, |
| | | | (10637684 . . . 10674046, | AWS, CD339, |
| | | | complement) | HJ1, JAGL1 |
| LCP2 | 3937 | lymphocyte cytosolic protein 2 | Chromosome 5, NC_000005.10 | SLP-76, SLP76 |
| | | (SH2 domain containing leukocyte | (170247754 . . . 170297818, | |
| | | protein of 76 kDa) | complement) | |
| | | [Homosapiens (human)] | | |
| LDLR | 3949 | low density lipoprotein receptor | Chromosome 19, NC_000019.10 | FH, FHC, |
| | | [Homosapiens (human)] | (11089362 . . . 11133830) | LDLCQ2 |
| LIMA1 | 51474 | LIM domain and actin binding 1 | Chromosome 12, NC_000012.12 | EPLIN, |
| | | [Homo sapiens(human)] | (50175780 . . . 50283570, | SREBP3 |
| | | | complement) | |
| LMO2 | 4005 | LIM domain only 2 (rhombotin- | Chromosome 11, NC_000011.10 | RBTN2, |
| | | like 1) [Homosapiens (human)] | (33858576 . . . 338922S9, | RBTNL1, |
| | | | complement) | RHOM2, |
| | | | | TTG2 |
| LTB | 4050 | lymphotoxin beta (TNF | Chromosome 6, NC_000006.12 | TNFC, |
| | | superfamily, member 3) | (31580558 . . . 31582425, | TNFSF3, p33 |
| | | [Homo sapiens (human)] | complement) | |
| MBP | 4155 | myelin basic protein | Chromosome 18, NC_000018.10 | N/A |
| | | [Homo sapiens (human)] | (76978833 . . . 77133708, | |
| | | | complement) | |
| MICAL2 | 9645 | microtubule associated | Chromosome 11, NC_000011.10 | MICAL-2PV1, |
| | | monooxygenase, calponin and | (12110576 . . . 12281016) | MICAL2PV2, |
| | | LIM domain containing 2 | | MICAL2 |
| | | [Homo sapiens (human)] | | |
| MYCN | 4613 | v-myc avian myelocytomatosis | Chromosome 2, NC_000002.12 | MODED, N- |
| | | viral oncogene neuroblastoma | (15940438 . . . 15947007) | myc, NMYC, |
| | | derived homolog | | ODED, |
| | | [Homo sapiens (human)] | | bHLHe37 |
| MYO1G | 64005 | myosin IG | Chromosome 7, NC_000007.14 | HA2, HLA- |
| | | [Homo sapiens (human)] | (44962661 . . . 44979105, | HA2, MHAG |
| | | | complement) | |
| NFE2 | 4778 | nuclear factor, erythroid 2 | Chromosome 12, NC_000012.12 | NF-E2, p45 |
| | | [Homo sapiens (human)] | (54292107 . . . 54301037, | |
| | | | complement) | |
| NOTCH2 | 4853 | notch 2 [Homo sapiens (human)] | Chromosome 1, NC_000001.11 | AGS2, HJCYS, |
| | | | (119911553 . . . 120069703, | hN2 |
| | | | complement) | |
| NRP1 | 8829 | neuropilin 1 | Chromosome 10, NC_000010.11 | BDCA4, |
| | | [Homo sapiens(human)] | (33177491 . . . 33334905, | CD304, NP1, |
| | | | complement) | NRP, |
| | | | | VEGF165R |
| NRROS | 375387 | negative regulator of reactive | Chromosome 3, NC_000003.12 | ELLP3030, |
| | | oxygen species | (196639696 . . . 196662004) | GARPL1, LRRC33, |
| | | [Homo sapiens(human)] | | UNQ3030 |
| P2RY2 | 5029 | purinergic receptor P2Y, G-protein | Chromosome 11, NC_000011.10 | HP2U, P2RU1, |
| | | coupled, 2 | (73216448 . . . 73242427) | P2U, P2U1, |
| | | [Homo sapiens (human)] | | P2UR, P2Y2, |
| | | | | P2Y2R |
| PAG1 | 55824 | phosphoprotein membrane anchor | Chromosome 8, NC_000008.11 | CBP, PAG |
| | | with glycosphingolipid | (80967810 . . . 81112068, | |
| | | microdomains 1 | complement) | |
| | | [Homo sapiens(human)] | | |
| PLAC8 | 51316 | placenta-specific 8 | Chromosome 4, NC_000004.12 | C15, DGIC, |
| | | [Homosapiens (human)] | (83090048 . . . 83114758, | onzin |
| | | | complement) | |
| PLEK | 5341 | pleckstrin [Homo sapiens(human)] | Chromosome 2, NC_000002.12 | P47 |
| | | | (68365190 . . . 68397453) | |
| PLXNC1 | 10154 | plexin C1 [Homo sapiens (human)] | Chromosome 12, NC_000012.12 | CD232, PLXN- |
| | | | (94148723 . . . 94307675) | C1, VESPR |
| PMP22 | 5376 | peripheral myelin protein 22 | Chromosome 17, NC_000017.11 | CMT1A, |
| | | [Homosapiens (human)] | (15229777 . . . 15265357, | CMT1E, DSS, |
| | | | complement) | GAS-3, |
| | | | | HMSNIA, |
| | | | | HNPP, Sp110 |
| PTPRE | 5791 | protein tyrosine phosphatase, | Chromosome 10, NC_000010.11 | HPTPE, PTPE, |
| | | receptor type, E | (127907061 . . . 128085916) | R-PTP- |
| | | [Homo sapiens (human)] | | EPSILON |
| PU.1 | 6688 | Spi-1 proto-oncogene | Chromosome 11, NC_000011.10 | OF, SFPI1, |
| | | [Homo sapiens(human)] | (47354858 . . . 47395640, | SPI1, SPI-1, |
| | | | complement) | SPI-A |
| PXK | 54899 | PX domain containing | Chromosome 3, NC_000003.12 | MONaKA |
| | | serine/threonine kinase | (58332890 . . . 58426127) | |
| | | [Homo sapiens (human)] | | |
| RAB27A | 5873 | RAB27A, member RAS oncogene | Chromosome 15, NC_000015.10 | GS2, |
| | | family [Homosapiens (human)] | (55202966 . . . 55289828, | HsT18676, |
| | | | complement) | RAB27, RAM |
| RASA3 | 22821 | RAS p21 protein activator 3 | Chromosome 13, NC_000013.11 | GAP1IP4BP, |
| | | [Homo sapiens(human)] | (113977783 . . . 114132620, | GAPIII |
| | | | complement) | |
| RGS16 | 6004 | regulator of G-protein signaling 16 | Chromosome 1, NC_000001.11 | A28-RGS14, |
| | | [Homosapiens (human)] | (182598623 . . . 182604413, | A28-RGS14P, |
| | | | complement) | RGS-R |
| RHOH | 399 | ras homolog family member H | Chromosome 4, NC_000004.12 | ARHH, TTF |
| | | [Homo sapiens(human)] | (40191011 . . . 40244764) | |
| RNF24 | 11237 | ring finger protein 24 | Chromosome 20, NC_000020.11 | G1L |
| | | [Homo sapiens (human)] | (3929918 . . . 4015569, | |
| | | | complement) | |
| RXRA | 6256 | retinoid X receptor, alpha | Chromosome 9, NC_000009.12 | NR2B1 |
| | | [Homo sapiens (human)] | (134326463 . . . 134440586) | |
| SELPLG | 6404 | selectin P ligand | Chromosome 12, NC_000012.12 | CD162, CLA, |
| | | [Homosapiens (human)] | (108621895 . . . 108633954, | PSGL-1, |
| | | | complement) | PSGL1 |
| SLA | 6503 | Src-like-adaptor | Chromosome 8, NC_000008.11 | SLA1P, SLA |
| | | [Homo sapiens(human)] | (133036728 . . . 133103066, | |
| | | | complement) | |
| SLC7A11 | 23657 | solute carrier family 7 (anionic | Chromosome 4, NC_000004.12 | CCBR1, xCT |
| | | amino acid transporter light chain, | (138164094 . . . 138312660, | |
| | | xc- system), member 11 | complement) | |
| | | [Homo sapiens (human)] | | |
| SLC7A5 | 8140 | solute carrier family 7 (amino acid | Chromosome 16, NC_000016.10 | 4F2LC, CD98, |
| | | transporter light chain, L system), | (87830023 . . . 87869497, | D16S469E, |
| | | member 5 | complement) | E16, LAT1, |
| | | [Homo sapiens (human)] | | MPE16, |
| | | | | hLAT1 |
| SOCS1 | 8651 | suppressor of cytokine signaling 1 | Chromosome 16, NC_000016.10 | CIS1, CISH1, |
| | | [Homo sapiens (human)] | (11254417 . . . 11256182, | JAB, SOCS-1, |
| | | | complement) | SSI-1, SSI1, |
| | | | | TIP3 |
| ST3GAL4 | 6484 | ST3 beta-galactoside alpha-2,3- | Chromosome 11, NC_000011.10 | CGS23, |
| | | sialyltransferase 4 | (126355645 . . . 126652917) | NANTA3, |
| | | [Homo sapiens (human)] | | SAT3, SIAT4, |
| | | | | SIAT4C, |
| | | | | ST3GalIV, |
| | | | | STZ |
| STK17B | 9262 | serine/threonine kinase 17b | Chromosome 2, NC_000002.12 | DRAK2 |
| | | [Homosapiens (human)] | (196133583 . . . 196176503, | |
| | | | complement) | |
| TAL1 | 6886 | T-cell acute lymphocyte leukemia | Chromosome 1, NC_000001.11 | SCL, TCL5, |
| | | 1 [Homosapiens (human)] | (47216290 . . . 47232373, | bHLHa17, tal-1 |
| | | | complement) | |
| TCF12 | 6938 | transcription factor 12 | Chromosome 15, NC_000015.10 | CRS3, HEB, |
| | | [Homo sapiens(human)] | (56918289 . . . 57291129) | HTF4, |
| | | | | HsT17266, |
| | | | | bHLHb20 |
| TIMP3 | 7078 | TIMP metallopeptidase inhibitor 3 | Chromosome 22, NC_000022.11 | HSMRK222, |
| | | [Homo sapiens (human)] | (32800816 . . . 32863041) | K222, |
| | | | | K222TA2, SFD |
| TMEM104 | 54868 | transmembrane protein 104 | Chromosome 17, NC_000017.11 | N/A |
| | | [Homo sapiens(human)] | (74776483 . . . 74839783) | |
| TNF | 7124 | tumor necrosis factor | Chromosome 6, NC_000006.12 | DIF-alpha, |
| | | [Homosapiens (human)] | (31575567 . . . 31578336) | TNFA, |
| | | | | TNFSF2, TNF |
| TSC22D1 | 8848 | TSC22 domain family, member 1 | Chromosome 13, NC_000013.11 | Ptg-2, |
| | | [Homosapiens (human)] | (44432143 . . . 44576565, | TGFB1I4, |
| | | | complement) | TSC22 |
| TSC22D3 | 1831 | TSC22 domain family, member 3 | Chromosome X, NC_000023.11 | DIP, DSIPI, |
| | | [Homo sapiens(human)] | (107713221 . . . 107777329, | GILZ, TSC- |
| | | | complement) | 22R, hDIP |
| ZBTB16 | 7704 | zinc finger and BTB domain | Chromosome 11, NC_000011.10 | PLZF, ZNF145 |
| | | containing 16 | (114059579 . . . 114250676) | |
| | | [Homo sapiens (human)] | | |
| ZCCHC5 | 203430 | zinc finger, CCHC domain | Chromosome X, NC_000023.11 | Mar3, Mart3, |
| | | containing 5 | (78656069 . . . 78659502, | ZHC5 |
| | | [Homo sapiens (human)] | complement) | |

Those of ordinary skill in the art will be able to identify wild-type sequences of the RUNX1 binding partners and RUNX1 target genes based on the identifications provided above.

In some embodiments, the cancer comprises a RUNX1-RUNX1T1 translocation. RUNX1-RUNX1T1 translocations are well known in the art. See, e.g., Kim et al., Acute myeloid leukemia with a RUNX1-RUNX1T1 t(1; 21; 8)(q21; q22; q22) novel variant: a case report and review of the literature. Acta Haematol. 125(4):237-41 (2011), the entire contents of which are incorporated by reference. Additional RUNX1 translocations associated with cancer are also known to those of skill in the art, e.g., RUNX1-ETO ETV6-RUNX1 and RUNX1-EVI1 translocations.

In some embodiments, the cancer comprises an A142_A149dup, A142fsX170, A149fsX, A251fsX, A338fsX482, A63fsX, D160Y, D326fsX481, E223fsX, E422fsX, F411fsX482, G165R, G170fsX201, G394_L406dup, G394fsX482, G409fsX482, G439fsX482, H105_F116dup, H105fsX541, H427fsX, Il14fsX117, I342fsX, K215fsX269, L112fsX117, L144fsX170, L210fsX269, L313fsX323, L382fsX482, L98fsX, N448_V452dup, P113A, P345R, P464P, P95fsX117, Q335_L339dup, Q438fsX482, R107C, R107S, R166Q, R201G, R201Q, R201X, R232W, R320X, R346fsX, R346fsX482, S141fsX, S226fsX269, S256fsX269, S322fsX323, S331fsX, S388fsX481, T148fsX170, Y355fsX, or Y380fsX482 mutation, or any combination thereof, in a RUNX1 protein encoded by the genome of the cancer. Locations of the mutations listed above and elsewhere herein are defined for human RUNX1 according to the accession number NC_000021.8 (36160098 . . . 36421595, complement) of NCBI assembly GRCh37.p13 (GCF_000001405.25), annotation release 105 (accessible at the National Center for Biotechnology Information (NCBI) website at ncbi.org). Those of skill in the art will be able to identify homologous mutations in non-human subjects, e.g., by aligning the human and non-human RUNX1 sequences and identifying corresponding residues, as is routinely performed in the art. In addition, those of skill will be able to identify the locations of the mutations listed above in any new annotation releases of the NCBI database, e.g., in accession number NC_000021.9 (34787801 . . . 35049310, complement) of NCBI assembly GRCh38.p2 (GCF_000001405.28), of annotation release 107, based on sequence alignment and identification of homologous residues.

It has been discovered that a cortistatin, which is a CDK8/19 inhibitor, or a pharmaceutically acceptable salt, quaternary amine salt, or N-oxide thereof, can be used to counteract RUNX1 impairment and to treat RUNX1-mutated cancers and cancers in which a binding partner or RUNX1 target gene is mutated. CDK8 and CDK19 are sometimes referred to as “mediator kinases” since they assemble in multi-protein complexes that reversibly bind the Mediator complex. The Mediator complex links enhancer-bound transcription factors to promoter-bound RNA pol II holoenzyme and influences chromatin architecture to regulate transcription and gene expression through still poorly understood mechanisms. Recent comprehensive genome-wide sequencing of samples from 200 AML patients revealed that, remarkably, nearly all mutations in presumably cancer-driving proteins are associated with regulating gene expression. See, e.g., Aerts, et al., Nature (2013) 499:35-36; The Cancer Genome Atlas Research Network, 2013. Genomic and Epigenomic Landscapes of Adult De Novo Acute Myeloid Leukemia. N. Engl. J. Med. 368, 2059-2074. Some aspects of the present disclosure are thus based on the recognition that specific inhibition of Mediator kinases, and of inhibition of CDK8 and CDK19 in particular, constitutes a new means to disrupt the downstream effects of impairment of RUNX1 activity in various cancers, and in particular in hematologic cancers, such as, for example, AML.

In one example, in AML, RUNX1 regulates transcription together with other transcription factors and binding partners that may have a mutation, including CBFb, GATA1/2, PU.1, and ERG. RUNX1 impairment affects this pathway. Furthermore, other mutations in AML may repress the RUNX1 transcriptional program via RUNX1 protein degradation (MLL fusions) or gene repression through DNA methylation (IDH2 mutation). Thus the targeted selection of patients with RUNX1 impairment using cortistatin therapy represents a new, broadly useful mechanism of activating the RUNX1 transcriptional program in and consequently restoring more normal hematopoiesis, or rendering the cells more normal, less virulent or with induced maturation, with potential growth arrest and/or apoptosis.

In one aspect of the second embodiment, the biomarker is related directly or indirectly to the RUNX1 pathway. For example, a method is provided for determining whether a patient having a tumor or cancer can successfully be treated with a cortistatin by first assessing whether the patient carries an inactivating mutation of the RUNX1 gene, or of genes involved in RUNX1-mediated transcription (such as but not limited to GATA1, GATA2, C/EBPα, FLI1, FOG1, ETS1, PU.1, ERG, and CBFα). RUNX1 inhibition (partial or complete) can manifest itself through monoallelic inactivating mutations or translocation to RUNX1-RUNX1T1 (also called AML1-ETO), which blocks wild-type RUNX1 DNA association and transcription.

In some embodiments, the diagnostic or therapeutic methods provided herein includes detecting an expression level of RUNX1, of a RUNX1 binding partner, and/or of a RUNX1 target gene, and comparing it to a reference level, in order to determine whether a cancer exhibits impaired RUNX1 activity, wherein the RUNX1 target gene is one or a combination of: ACSL1, ADORA2B, ADRB1, AMPD3, ARRDC4, BCL2, BCL2A1, CBFβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5. In some embodiments, the RUNX1 target gene is one or a combination of BCL2, CCNA1, CD44, C/EBPα, CBFβ, CSF1, CXCL10, CXCR4, ETS1, ETS2, FLI1, FOG1, FCER1A, GATA1, GATA2, GFI1B, HEB, IRF1, IRF8, JAG1, LMO2, LTB, NFE2, NOTCH2, PU.1, SLA, SOCS1, TAL1, and TNF.

In certain embodiments, the RUNX1-impaired tumor or cancer is Acute lymphoblastic leukemia (ALL), Acute myeloid leukemia (AML), Chronic lymphoblastic leukemia (CLL), Chronic myeloid leukemia, B-cell acute lymphoblastic leukemia (B-ALL), childhood B-ALL, Acute monocytic leukemia, Acute megakaryoblastic leukemia, Hodgkin's lymphoma, Non-Hodgkin's lymphoma, Burkitt's lymphoma, AIDS-related lymphoma, Chronic myeloproliferative disorder, Primary central nervous system lymphoma, T-cell lymphoma, Hairy cell leukemia or Multiple myeloma (MM).

In another embodiment, a patient diagnosed with a myelodysplastic syndrome (MDS) can be treated using the present invention. Many recurrent somatic mutations that drive the MDS phenotype reside in transcription factors and epigenetic targets that regulate transcription. RUNX1 is a transcription factor and master regulator of hematopoiesis that is mutated in 10-20% of MDS patients, rendering it among the most frequently mutated genes in MDS. Mutations in RUNX1 attenuate expression of target genes that drive differentiation and this effect predicts higher risk and shorter time to secondary AML (sAML) transition. It has been found that inhibition of CDK8/19 increases expression of RUNX1-target genes and therefore the invention can be an effective therapeutic approach to treat MDS patients with RUNX1 mutations and other mutations that suppress this key differentiation program.

Impaired RUNX1 activity, resulting, for example, from loss-of-function mutations in the RUNX1 gene, are known to be associated with various forms of cancer, including, for example, various types of leukemia. Since RUNX1 is an activating transcription factor and no strategy for compensating the loss of transcriptional activation mediated by RUNX1 is available, no clinical intervention counteracting the impairment of RUNX1 activity exists. Some aspects of this disclosure are based on the identification of a group of RUNX1 binding partners and target genes. Some aspects of this disclosure are based on the recognition that modulating the expression or activity of RUNX1 binding partners and target genes via administration of certain compounds, such as, for example, a CDK8/19 inhibitor and/or a cortistatin or cortistatin analog thereof, either alone or in combination with addition compounds provided herein, constitutes an effective strategy to counteract impaired RUNX1 activity, for example, for treating subjects carrying a cancer exhibiting an impaired RUNX1 activity.

Accordingly, this disclosure provides methods, compositions, and kits for treating cancer exhibiting impaired RUNX1 activity. In addition, this disclosure also provides methods, for determining whether a cancer in a subject is sensitive to treatment with the compounds and compositions provided herein, and for selecting patients for treatment according to any of the therapeutic methods and strategies provided herein based on such determinations.

It will be appreciated by those of skill in the art that the present disclosure is not limited with respect to the RUNX1 mutations that result in impaired RUNX1 activity. For an overview of RUNX1 mutations that result in impaired RUNX1 activity, see, e.g., Ito et al., The RUNX family: developmental regulators in cancer, Nature Reviews Cancer 15, 81-95 (2015), e.g., page 83, last paragraph to page 84, last paragraph, and Tables 1 and 2; Ley et al., Genomic and Epigenomic Landscapes of Adult De Novo Acute Myeloid Leukemia, NEJM 368:22, 2059-74 (2013); Gelsi-Boyer et al., Genome profiling of chronic myelomonocytic leukemia: frequent alterations of RAS and RUNX1 genes. BMC Cancer 8, 299 (2008); and Kuo et al., RUNX1 mutations are frequent in chronic myelomonocytic leukemia and mutations at the C-terminal region can predict acute myeloid leukemia transformation. Leukemia 23, 1426-1431 (2009); the entire contents of each of which are incorporated herein by reference.

B. Selection of Patients Based on Biomarkers Other than Runx1

In another embodiment of the invention, a method for predicting the response of a patient with a tumor or cancer to treatment with cortistatin therapy, includes the steps of: obtaining a sample of the tumor or cancer from the patient; determining the expression level or amount of one or more biomarkers in the biological sample from a patient wherein the biomarker(s) is selected from the group consisting of ER-positive, loss of function of VHL mutation (VHL-negative), HER2 overexpression, EGFR mutation, MET mutation, a biomarker for neuroblastoma; EWS-FLI1, STAT1-pS727, STAT1 or an inactivating mutation in ETV1, FLI1, SMC3, SMC1A, RAD21, or STAG2; determining whether the expression level or amount is above or below that found in corresponding normal cells, for example, is above or below a certain quantity that is associated with an increased or decreased clinical benefit to a patient; and then optionally treating the patient with an effective amount of the CDK8/19 inhibitor, or its pharmaceutically acceptable salt, oxide or a pharmaceutically acceptable salt thereof. In an alternative embodiment, the observed gene expression is compared to the expression of the same genes in a control set of samples comprising a representative number of patients or a predictive animal model that exhibit response to a CDK8/19 inhibitor and a representative number of patients that exhibit no or a poor response to a CDK8/19 inhibitor to determine if the patient is likely to respond to cortistatin therapy. If the patient's biomarkers indicate, then the healthcare provider may assume that the patient is more likely to respond to therapy.

In one embodiment the neuroblastoma is further sensitive to a CDK8/19 inhibitor due to activation of the RUNX1 transcriptional program. See Inoue, K.-I. & Ito, Y. Neuroblastoma cell proliferation is sensitive to changes in levels of RUNX1 and RUNX3 protein. Gene 487, 151-155 (2011).

Examples of tumors and cancers with aberrant STAT1 or STAT1-pS727 levels include those described in: Timofeeva, O. A. et al. Serine-phosphorylated STAT1 is a prosurvival factor in Wilms' tumor pathogenesis. Oncogene 25, 7555-7564 (2006); Liu, W., Zhang, L. & Wu, R. Differential expression of STAT 1 and IFN-γ in primary and invasive or metastatic wilms tumors. J. Surg. Oncol. 108, 152-156 (2013); Arzt, L., Kothmaier, H., Halbwedl, I., Quehenberger, F. & Popper, H. H. Signal transducer and activator of transcription 1 (STAT1) acts like an oncogene in malignant pleural mesothelioma. Virchows Arch 465, 79-88 (2014). In one embodiment the tumor or cancer associated with the STAT1 or STAT1-pS727 biomarker is mesothelioma, and metastatic wilms tumor.

### IV. DIAGNOSTICS AND KITS

Methods for obtaining a cell or tissue sample from a subject comprising a cancer or tumor cell are well known to those of skill in the art. Such methods typically comprise obtaining a tumor biopsy from the subject, e.g., a tissue biopsy comprising cancer cells from a solid tumor, or a body fluid biopsy comprising tumor cells from a liquid tumor. Those of skill in the art will be aware of suitable sources of cancer cells in a subject, depending on the type of cancer the subject is carrying. For example, in some embodiments, the cancer is a leukemia and the cancer cell is a bone marrow cell, a peripheral blood cell, or a hematopoietic stem cell. In some such embodiments, the method comprises obtaining a blood or bone marrow sample from the subject comprising a leukemic cell.

In one embodiment, the diagnostic methods provided herein determine whether the cancer in the subject is associated with or exhibits impaired RUNX1 activity. Such impaired RUNX1 activity can be detected in different ways. For example, impaired RUNX1 activity is detected, in some embodiments, by detecting a mutation in the RUNX1 gene that has been reported to be associated with impaired RUNX1 activity. In some embodiment, impaired RUNX1 activity is detected by measuring the expression level of a RUNX1 gene product, for example, a RUNX1 transcript, mRNA, or protein level, in a cancer cell obtained from the subject, and comparing the measured level to a reference level measured or expected in a healthy cell of the same or similar cell type. In some embodiments, an impaired RUNX1 activity is detected if the RUNX1 expression level measured in the cancer cell is decreased as compared to the RUNX1 expression level in a healthy cell by more than 25%, more than 30%, more than 40%, more than 50%, more than 60%, more than 70%, more than 80%, more than 90%, more than 95%, or more than 98%. In the absence of a statement to the contrary, an impaired RUNX1 activity is detected if the RUNX1 expression level measured in the cancer cell is decreased as compared to the RUNX1 expression level in a healthy cell by more than 25%.

In some embodiments, the diagnostic methods provided herein comprise determining whether the cancer in the subject is associated with or exhibits impaired RUNX1 activity by detecting a mutation in the genome of the cancer which results in impaired RUNX1 activity. Numerous mutations resulting in impaired RUNX1 activity have been previously reported. Such mutations include, without limitation, those mutations disclosed in Appendix Table A2 of Gaidzik et al., RUNX1 mutations in acute myeloid leukemia: results from a comprehensive genetic and clinical analysis from the AML study group. J Clin Oncol. 29(10):1364-72 (2011); the entire contents of which are incorporated herein by reference. In some embodiments, the method comprises detecting a mutation in a gene encoding a RUNX1 protein, e.g., a mutation in the RUNX1 gene. In some embodiments, the method comprises detecting a mutation that results in a change in the amino acid sequence of the RUNX1 protein as compared to a wild type RUNX1 sequence. In some embodiments, the method comprises detecting a mutation that results in a substitution, deletion, or duplication of an amino acid or an amino acid sequence, a frameshift, or a premature stop codon in a protein-encoding sequence of RUNX1. In some embodiments, the mutation is a translocation that results in an abnormal RUNX1 protein. In some embodiments, the mutation results in a deletion of a fragment of the RUNX1 protein. In some embodiments, the mutation results in a fusion of the genomic sequence encoding the RUNX1 protein, or a fragment thereof, to a genomic sequence encoding a different protein, or a fragment thereof. In some embodiments, the mutation results in a fusion of the genomic sequence encoding a RUNX1 target protein, or a fragment thereof, to a genomic sequence encoding a different protein, or a fragment thereof. In some embodiments, the mutation is a RUNX1-RUNX1T1 translocation. In some embodiments, the mutation is an A142_A149dup, A142fsX170, A149fsX, A251fsX, A338fsX482, A63fsX, D160Y, D326fsX481, E223fsX, E422fsX, F411fsX482, G165R, G170fsX201, G394_L406dup, G394fsX482, G409fsX482, G439fsX482, H105_F116dup, H105fsX541, H427fsX, Il14fsX117, I342fsX, K215fsX269, L112fsX117, L144fsX170, L210fsX269, L313fsX323, L382fsX482, L98fsX, N448_V452dup, P113A, P345R, P464P, P95fsX117, Q335_L339dup, Q438fsX482, R107C, R107S, R166Q, R201G, R201Q, R201X, R232W, R320X, R346fsX, R346fsX482, S141fsX, S226fsX269, S256fsX269, S322fsX323, S331fsX, S388fsX481, T148fsX170, Y355fsX, or Y380fsX482 mutation, or any combination thereof. Locations of mutations are defined according to the accession number cDNA NC_000021.8 (accessible at the National Center for Biotechnology Information (NCBI) website at ncbi.org). Additional mutations that result in impaired RUNX1 activity will be apparent to those of skill in the art based on the present disclosure and the knowledge in the art. The disclosure is not limited in this respect.

In some embodiment, the diagnostic methods provided herein comprise determining whether the cancer in the subject is associated with or exhibits impaired RUNX1 activity by detecting a mutation in a gene encoding a RUNX1-binding partner or a RUNX1 target gene. For example, in some embodiments, the gene encoding a RUNX1 binding partner or a RUNX1 target gene is C/EBPα, CBFPβ, ETS1, FLI1, FOG1, GATA1, GATA2, PU.1, TAL1, LMO2 or HEB. In some embodiments, the method comprises determining whether the cancer in the subject is associated with or exhibits impaired RUNX1 activity by detecting an MLL-AF9 translocation, an MLL-AF4 translocation, a Bcr-Abl fusion, or a JAK2 V617F mutation in the cancer.

In some embodiments, the method comprises detecting an expression level of RUNX1, of a RUNX1 binding partner, and/or of a RUNX1 target gene, and comparing it to a reference level, in order to determine whether the cancer exhibits impaired RUNX1 activity. In some embodiments, the RUNX1 target gene is selected from the group consisting of ACSL1, ADORA2B, ADRB1, AMPD3, ARRDC4, BCL2, BCL2A1, CBFPβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5. In some embodiments, the RUNX1 target gene is selected from the group consisting of BCL2, CCNA1, CD44, C/EBPα, CBFβ, CSF1, CXCL10, CXCR4, ETS1, ETS2, FLI1, FOG1, FCER1A, GATA1, GATA2, GFI1B, HEB, IRF1, IRF8, JAG1, LMO2, LTB, NFE2, NOTCH2, PU.1, SLA, SOCS1, TAL1, and TNF.

In some embodiments, the cancer comprises an MLL-AF9 translocation, an MLL-AF4 translocation, a Bcr-Abl fusion, or a JAK2 V617F mutation. For an overview of these translocations, see, e.g., Horton et al., MLL-AF9-mediated immortalization of human hematopoietic cells along different lineages changes during ontogeny. Leukemia 27(5):1116-26 (2013); Bueno et al., Insights into the cellular origin and etiology of the infant pro-B acute lymphoblastic leukemia with MLL-AF4 rearrangement. Leukemia. 25(3):400-10 (2011); Press et al., BCR-ABL1 RT-qPCR for monitoring the molecular response to tyrosine kinase inhibitors in chronic myeloid leukemia. J Mol Diagn. 15(5):565-76 (2013); and Mata et al., JAK2 as a molecular marker in myeloproliferative diseases. Cardiovasc Hematol Agents Med Chem. 5(3): 198-203 (2007), the entire contents of each of which are incorporated herein by reference. In some embodiments, detecting impaired RUNX1 activity or impaired activity of a RUNX1 binding partner or a RUNX1 target gene, includes obtaining information about the presence or absence of one or more mutations in a RUNX1 gene, a gene encoding a RUNX1 binding partner or target gene, and/or an increase or decrease in expression levels of a gene product encoded by such a gene. Such methods may include, in some embodiments, obtaining a cancer cell from a subject in order to assess the genomic or expression status of RUNX1, a RUNX1 binding partner, or a RUNX1 target gene. In some embodiments, such methods include obtaining a biopsy of a tissue or a body fluid from the subject that comprises a cancer cell, e.g., a tumor biopsy, or a blood or bone marrow biopsy. In some embodiments, a cancer cell comprised in the biopsied sample is then subjected to an assay suitable for detecting a mutation or a gene expression level of RUNX1, a RUNX1 binding partner, or a RUNX1 target gene. In some embodiments, the biopsy may include normal cells or cells of an unwanted tissue type. For example, a peripheral blood or bone marrow sample may include cells that are typically not involved in the pathology of hematologic cancers, such as leukemias. In some embodiments, the biopsied sample is processed in order to enrich for cancer cells or for cells frequently associated with cancer pathology, such as hematopoietic stem cells, or to deplete cells that are not typically involved in carcinogenesis, such as differentiated cells. Methods for such enrichment and depletion of cells in samples obtained from a subject are well known to those of skill in the art.

In other embodiments, the biopsied samples are subjected to a detection assay without enrichment or depletion of specific cells or cell types.

Those of ordinary skill in the art will be able to detect mutations and/or expression levels of RUNX1 or of RUNX1 binding partners or RUNX1 target genes in samples biopsied from a subject by subjecting such samples to suitable assays well known in the art. For the detection of mutations in encoding genes, such assays typically include obtaining genomic sequence information from a cancer cell. In some embodiments, the detection method comprises an assay that includes amplification of the target sequence, e.g., a genomic sequence encoding RUNX1, or a RUNX1 binding partner or RUNX1 target gene, sequencing the amplified target sequences, and comparing the obtained sequence information to wild-type sequences in order to determine whether one or more mutations are present.

The term “expression level”, as used herein, refers to information about the level of one or more gene products (e.g., an mRNA, a protein, or a combination thereof) in a cell or tissue. In some embodiments, the detection of one or more gene mutations, and/or a decrease in expression levels as described herein may be based on one or more measurements or assays, for example, a quantitative or semi-quantitative value of expression of a single gene, for example, reflective of the signal obtained from a quantitative or semi-quantitative assay detecting the abundance of a gene product (e.g., a protein or a nucleic acid transcript encoded by a RUNX1 gene, a RUNX1 binding partner or RUNX1 target gene). Suitable assays for the detection of gene expression products are well known to those of skill in the art and include, for example, western blots, ELISA, RT-PCR (e.g., end-point RT-PCR, real-time PCR, or qPCR), protein or nucleic acid microarray, and massive parallel sequencing assays. However, any suitable assay may be used based on hybridization, specific binding (e.g., antibody binding), or any other technique, as aspects of the invention are not limited in this respect.

In some embodiments, the presence of one or more gene mutations, and/or a decrease in expression levels as described herein may involve a plurality of data points, for example, quantitative or semi-quantitative values of expression and/or one or sequence or mutation data points. In some embodiments, the presence of one or more gene mutations, and/or an increase or decrease in expression levels as described herein may be evaluated in a biopsy sample. Methods for the detection or for the generation of data for one or more gene mutations, and/or an increase or decrease in expression levels as described herein are well known to those in the art and include, for example, southern blot, western blot, ELISA, northern blot, reverse northern blot, RT-PCR (e.g. endpoint, real time, or qPCR), PCR, ddPCR (e.g. droplet digital PCR), microarray (for either protein or transcript detection), SNP analysis, PCR, hybridization assays, sequencing assays, etc., or any combination thereof (for exemplary detection methods, see, e.g., Sambrook et al., Molecular Cloning: A Laboratory Manual, Third Edition (3 Volume Set), Cold Spring Harbor Laboratory Press; 3rd edition (Jan. 15, 2001), ISBN-10: 0879695773; Robert Grützmann (Editor), Christian Pilarsky (Editor), Cancer Gene Profiling: Methods and Protocols (Methods in Molecular Biology), Humana Press; 1st edition (Nov. 6, 2009), ISBN-10: 1934115762, both incorporated herein by reference for disclosure of gene product detection and expression profiling methods).

In some embodiments, a quantitative expression value is a value reflecting the abundance of a gene transcript in the starting sample, for example, a tumor cell or tissue sample. In some embodiments, a semi-quantitative expression value is a value reflecting the abundance of a gene transcript in the starting sample in relation to a control or reference quantity, e.g., a quantity measured or expected in a healthy cell or in a cell of the same type obtained from a healthy individual. Methods of calculating semi-quantitative expression values are well known to those in the art. Appropriate control or reference quantities for the generation of semi-quantitative expression values are well known to those in the art and include, for example, expression values of housekeeping genes (e.g., beta-actin or GAPDH), external controls (e.g., spiked in RNA or DNA controls not usually expressed in the cell to be analyzed), overall expression values (e.g., all expression values obtained from a cell added together), or historic or empiric values.

In some embodiments, an expression level of RUNX1, a RUNX1 binding partner or a RUNX1 target gene (e.g., RNA and/or protein) that is determined for a sample is compared to a reference expression level. In some embodiments, the reference is a standard that is indicative of a normal expression level. In some embodiments, the reference is a standard that is indicative of a deficient expression level (and any test levels that are at or below the reference would be indicative of an impaired activity of RUNX1, the RUNX1 binding partner or the RUNX1 target gene, respectively). In some embodiments, a reference level is obtained by determining the expression level of RUNX1, a RUNX1 binding partner or a RUNX1 target gene in a sample of normal or healthy tissue. In some embodiments, the reference level is determined by assaying RUNX1, a RUNX1 binding partner or a RUNX1 target gene in a reference sample (e.g., a sample containing no malignant cells) obtained from the same subject from which a test sample was obtained. The reference sample may be obtained from a different region of the same tissue or from a different region of the subject's body as the test sample.

In some embodiments, a subject, or a biopsy or other biological sample obtained from a subject, is evaluated to determine whether an impairment in RUNX1 activity, or of the activity of a RUNX1 binding partner or a RUNX1 target gene, is present, for example, detected as a mutation in a gene encoding RUNX1, a RUNX1 binding partner or a RUNX1 target gene, (e.g., a deletion, loss of function, a frameshift, inversion, translocation, or other mutation) or as a decreased level of expression of RUNX1, a RUNX1 binding partner or a RUNX1 target gene. It should be appreciated that any of the genetic and/or expression information described herein may be used alone or in combination, with or without additional patient information to assist in a prognosis, therapeutic recommendation, or other diagnostic or predictive evaluation of the health, outcome, and/or treatment for the patient.

### V. METHODS AND PHARMACEUTICAL COMPOSITIONS

The invention includes first assessing a patient in need of tumor or cancer treatment by determining whether the patient has an abnormal level of biomarkers as specified herein, and then if the results warrant, then treating the patient with the cortistatin or CDK8/19 inhibitor therapy.

The cortistatin or other CDK8/19 inhibitor can be administered as the neat chemical, but are more typically administered as a pharmaceutical composition, that includes an effective amount for a host, typically a human, in need of such treatment of the selected cortistatin or the CDK8/19 inhibitor, as described herein. Accordingly, the disclosure provides pharmaceutical compositions comprising an effective amount of cortistatin or its pharmaceutically acceptable salt together with at least one pharmaceutically acceptable carrier for all of the uses described herein. The pharmaceutical composition may contain the cortistatin as the only active agent, or, in an alternative embodiment, the compound and at least one additional active agent. In certain embodiments the pharmaceutical composition is in a dosage form that contains from about 0.1 mg to about 2000 mg, from about 10 mg to about 1000 mg, from about 5 mg to about 200 mg, or from about 5 mg to about 100 mg of the active compound and optionally an appropriate dosage amount of an additional active agent, in a unit dosage form. Examples are dosage forms with at least 5, 10, 15, 25, 50, 75, 100, 200, 250, 300, 400, 500, 600, 700, or 750 mg of active compound. The cortistatin or CDK8/19 inhibitor may be administered orally, topically, parenterally, by inhalation or spray, sublingually, via implant, including ocular implant, transdermally, via buccal administration, rectally, as an ophthalmic solution, injection, intravenous, intra-aortal, intracranial, subdermal, intraperitoneal, subcutaneous, transnasal, sublingual, or rectal or by other means, in dosage unit formulations containing conventional pharmaceutically acceptable carriers.

In some embodiments, the present disclosure provides compositions comprising a cortistatin or CDK8/19 inhibitor, such as a compound of Formula (A-1) (A-1′), (A-1″), (A-2′), (A-2″), (A-3′), (A-3″), (D1′), (D1″), (D2′), (D2″), (E1′), (E1″), (E2′), (E2″), (G1′), or (G1″), or a pharmaceutically acceptable salt, quaternary amine salt, or N-oxide thereof, for administration to a subject having a cancer or tumor that exhibits impaired RUNX1 activity. In some embodiments, the composition comprises a CDK8/19 inhibitor. In some embodiments, the composition further comprises a Jak1/2 inhibitor.

Although the descriptions of pharmaceutical compositions provided herein are principally directed to pharmaceutical compositions which are suitable for administration to humans, it will be understood by the skilled artisan that such compositions are generally also suitable for administration to animals. If required, modification of pharmaceutical compositions suitable for administration to humans in order to render the compositions suitable for administration to various animals is well understood, and the ordinarily skilled person in the art will be able to design and perform such modification with merely ordinary, if any, experimentation. Subjects to which administration of the pharmaceutical compositions is contemplated include, but are not limited to, humans and/or other primates; mammals, e.g., cattle, pigs, horses, sheep, cats, dogs, rodents, mice, hamsters, and/or rats; birds, e.g., chickens, ducks, geese, and turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) of active ingredient, e.g. a CDK8/19 inhibitor, or a cortistatin or cortistatin analog thereof, such as a compound of Formula (A-1), (A-1′), (A-1″), (A-2′), (A-2″), (A-3′), (A-3″), (D1′), (D1″), (D2′), (D2″), (E1′), (E1″), (E2′), (E2″), (G1′), or (G1″), or a pharmaceutically acceptable salt, quaternary amine salt, or N-oxide thereof, and, optionally, any additional active ingredients, such as, for example, a JAK1/2 inhibitor. In some embodiments, the composition comprises between 0.1% and 1%, between 1% and 10%, between 10% and 20%, between 20% and 30%, between 30% and 40%, between 40% and 50%, between 50% and 60%, between 60% and 70%, between 70% and 80%, between 80% and 90%, or between 90% and 100% (w/w) of active ingredient, and more generally, between 0.1 and 100% (w/w) of active ingredient.

Pharmaceutical formulations as provided herein may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21 st Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the excipient is one already approved for use in humans and for veterinary use, for example, by United States Food and Drug Administration. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween®20], polyoxyethylene sorbitan [Tween®60], polyoxyethylene sorbitan monooleate [Tween®80], sorbitan monopalmitate [Span®40], sorbitan monostearate [Span®60], sorbitan tristearate [Span®65], glyceryl monooleate, sorbitan monooleate [Span®80]), polyoxyethylene esters (e.g. polyoxyethylene monostearate [Myrj®45], polyoxyethylene hydrogenated castor oil, polyethoxylated castor oil, polyoxymethylene stearate, and Solutol®), sucrose fatty acid esters, polyethylene glycol fatty acid esters (e.g. Cremophor®), polyoxyethylene ethers, (e.g. polyoxyethylene lauryl ether [Brij®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic®F 68, Poloxamer®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall®1l15, Germaben®II, Neolone™, Kathon™, and/or Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT Publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. In some embodiments, the formulation suitable for nasal administration comprises between 0.1% and 1%, between 1% and 10%, between 10% and 20%, between 20% and 30%, between 30% and 40%, between 40% and 50%, between 50% and 60%, between 60% and 70%, between 70% and 80%, between 80% and 90%, or between 90% and 100% (w/w) of active ingredient. In the absence of a statement to the contrary, the formulation suitable for nasal administration comprises between 0.1 and 100% (w/w) of active ingredient. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or atomized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Still further encompassed by the invention are pharmaceutical packs and/or kits. Pharmaceutical packs and/or kits provided may comprise a provided composition and a container (e.g., a vial, ampoule, bottle, syringe, and/or dispenser package, or other suitable container). In some embodiments, provided kits may optionally further include a second container comprising a suitable aqueous carrier for dilution or suspension of the provided composition for preparation of administration to a subject. In some embodiments, contents of provided formulation container and solvent container combine to form at least one unit dosage form.

Optionally, a single container may comprise one or more compartments for containing a provided composition, and/or appropriate aqueous carrier for suspension or dilution. In some embodiments, a single container can be appropriate for modification such that the container may receive a physical modification so as to allow combination of compartments and/or components of individual compartments. For example, a foil or plastic bag may comprise two or more compartments separated by a perforated seal which can be broken so as to allow combination of contents of two individual compartments once the signal to break the seal is generated. A pharmaceutical pack or kit may thus comprise such multi-compartment containers including a provided composition and appropriate solvent and/or appropriate aqueous carrier for suspension. Optionally, instructions for use are additionally provided in such kits of the invention. Such instructions may provide, generally, for example, instructions for dosage and administration. In other embodiments, instructions may further provide additional detail relating to specialized instructions for particular containers and/or systems for administration. Still further, instructions may provide specialized instructions for use in conjunction and/or in combination with additional therapy.

### VI. COMBINATIONS

In one aspect, if the biomarker diagnostic described herein predicts that a cortistatin or CDK8/19 inhibitor will successfully treat a patient, it may be desired to administer the active compound in combination with a second active agent.

In one embodiment, a sample taken from the patient is initially assessed for a predicted successful therapy using a cortistatin or a CDK8/19 inhibitor, and then the sample is assessed in a second assay to determine whether the patient will also benefit from administration of a second active agent. In another embodiment, the results from the first biomarker assay, as described in detail herein, also predicts that the patient may respond to combination therapy.

Therefore, using the selection method described herein, a treatment regimen is provided comprising the administration of a compound of the present invention or a pharmaceutically acceptable composition, salt, isotopic analog (such as a deuterated derivative), or prodrug thereof in combination or in alternation with at least one additional therapeutic agent. The combinations and/or alternations disclosed herein can be administered for beneficial, additive, or synergistic effect in the treatment of abnormal cellular proliferative disorders.

In specific embodiments, the treatment regimen includes the administration of a compound of the present invention or a pharmaceutically acceptable composition, salt, isotopic analog, or prodrug thereof in combination or alternation with at least one additional kinase inhibitor. In one embodiment, the at least one additional kinase inhibitor is selected from a phosphoinositide 3-kinase (PI3K) inhibitor, a Bruton's tyrosine kinase (BTK) inhibitor, another cyclin-dependent kinase inhibitor, or a spleen tyrosine kinase (Syk) inhibitor, or a combination thereof.

In one embodiment, a compound of the present invention or a pharmaceutically acceptable composition, salt, isotopic analog, or prodrug thereof is combined in a dosage form with the PIk3 inhibitor.

PI3k inhibitors that may be used in the present invention are well known. Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-l-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((+)-7-Methyl-2-(morpholin-4-yl)-9-(1-phenylaminoethyl)-pyrido[1,2-a]-pyrimidin-4-one), GSK2636771 (2-Methyl-i -(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF 1126 ((8 S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11 aS)-6-[[bis(prop-2-enyl)amino]methylidene]-5-hydroxy-9-(methoxymethyl)-9a, 11a-dimethyl-1,4,7-trioxo-2,3,3a,9,10,11-hexahydroindeno[4, 5h]isochromen-10-yl] acetate (also known as sonolisib)).

BTK inhibitors for use in the present invention are well known. Examples of BTK inhibitors include ibrutinib (also known as PCI-32765) (Imbmvica™) (1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4, 5-dihydropyrazin-2-yl)-2-methylphenyl)-4, 5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4, 5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference. In one embodiment, a compound of the present invention or a pharmaceutically acceptable composition, salt, isotopic analog, or prodrug thereof is combined in a dosage form with the BTK inhibitor.

In one embodiment the additional cyclin-dependent kinase inhibitor is a CDK7 inhibitor such as THZ1 (N-[3-[[5-chloro-4-(1H-indol-3-yl)pyrimidin-2-yl]amino]phenyl]-4-[[(E)-4-(dimethylamino)but-2-enoyl]amino]benzamide). In an alternative embodiment the additional cyclin-dependent kinase inhibitor is a CDK9 inhibitor such as flavopiridol (alvocidib).

Therefore in one embodiment, a method of treating a tumor or cancer is provided, comprising administration of an effective amount of Compound A or a pharmaceutically acceptable salt thereof in combination or alternation with an effective amount of a Syk inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a Syk inhibitor to a host in need thereof.

In one embodiment, a method of treating a tumor or cancer is provided, comprising administration of an effective amount of Compound A or a pharmaceutically acceptable salt thereof in combination or alternation with imatinib (Gleevec) to a host in need thereof. In another embodiment, a method of treating a tumor or cancer is provided, comprising administration of an effective amount of an analog of Compound A or a pharmaceutically acceptable salt thereof as provided herein in combination or alternation with imatinib (Gleevec) to a host in need thereof.

Syk inhibitors for use in the present invention are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevec; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306(see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein). In one embodiment a compound of the present invention or a pharmaceutically acceptable composition, salt, isotopic analog, or prodrug thereof is combined in a dosage form with the Syk inhibitor.

In specific embodiments, the method of treatment provided includes the administration of a compound of the present invention or a pharmaceutically acceptable composition, salt, isotopic analog, or prodrug thereof in combination or alternation with at least one additional chemotherapeutic agent.

In one embodiment, the at least one additional chemotherapeutic agent combined or alternated with a compound of the present invention is a protein cell death-1 (PD-1) inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech). In one embodiment, a compound of the present invention or a pharmaceutically acceptable composition, salt, isotopic analog, or prodrug thereof is combined in a dosage form with the PD-1 inhibitor. In one embodiment the PD-1 inhibitor is pembrolizumab.

In one embodiment, a method of treating a tumor or cancer is provided, comprising administration of an effective amount of Compound A or a pharmaceutically acceptable salt thereof in combination or alternation with an effective amount of a PD-1 inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a PD-1 inhibitor to a host in need thereof.

In one embodiment, a method of treating a tumor or cancer is provided, comprising administration of an effective amount of Compound A or a pharmaceutically acceptable salt thereof in combination or alternation with pembrolizumab (Keytruda). In another embodiment, a method of treating a tumor or cancer is provided, comprising administration of an effective amount of an analog of Compound A or a pharmaceutically acceptable salt thereof as provided herein in combination or alternation with pembrolizumab (Keytruda).

In one embodiment, the at least one additional chemotherapeutic agent combined or alternated with a compound of the present invention is a CTLA-4 inhibitor. CTLA-4 inhibitors are known in the art, and include, for example, ipilimumab (Yervoy) marketed by Bristol-Myers Squibb and tremelimumab marketed by Pfizer.

In one embodiment, the at least one additional chemotherapeutic agent combined or alternated with the compound of the present invention is a BET inhibitor. BET inhibitors are known in the art, and include, for example, JQ1, I-BET 151 (a.k.a. GSK1210151A), I-BET 762 (a.k.a. GSK525762), OTX-015 (a.k.a. MK-8268, IUPAC 6H-Thieno[3,2-f][1,2,4]triazolo[4,3-a][1,4]diazepine-6-acetamide, 4-(4-chlorophenyl)-N-(4-hydroxyphenyl)-2,3,9-trimethyl-), TEN-010, CPI-203, CPI-0610, RVX-208, and LY294002. In one embodiment the BET inhibitor used in combination or alternation with a compound of the present invention for treatment of a tumor or cancer is JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate). In an alternative embodiment the BET inhibitor used in combination or alternation with a compound of the present invention for treatment of a tumor or cancer is I-BET 151 (2H-Imidazo[4,5-c]quinolin-2-one, 7-(3,5-dimethyl-4-isoxazolyl)-1,3-dihydro-8-methoxy-1-[(1R)-1-(2-pyridinyl)ethyl]-).

In one embodiment, a method of treating a tumor or cancer is provided, comprising administration of an effective amount of Compound A or a pharmaceutically acceptable salt thereof in combination or alternation with an effective amount of a BET inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a BET inhibitor to a host in need thereof.

In one embodiment, a method of treating a tumor or cancer is provided, comprising administration of an effective amount of Compound A or a pharmaceutically acceptable salt thereof in combination or alternation with JQ1. In another embodiment, a method of treating a tumor or cancer is provided, comprising administration of an effective amount of an analog of Compound A or a pharmaceutically acceptable salt thereof as provided herein in combination or alternation with JQ1.

In one embodiment, a method of treating a tumor or cancer is provided, comprising administration of an effective amount of Compound A or a pharmaceutically acceptable salt thereof in combination or alternation with I-BET 151. In another embodiment, a method of treating a tumor or cancer is provided, comprising administration of an effective amount of an analog of Compound A or a pharmaceutically acceptable salt thereof as provided herein in combination or alternation with I-BET 151.

In one embodiment, the at least one additional chemotherapeutic agent combined or alternated with the compound of the present invention is a MEK inhibitor. MEK inhibitors for use in the present invention are well known, and include, for example, tametinib/GSK1 120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-l(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methyl sulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1, and 5-dimethyl-6-oxo-1,6-dihydropyridine-3-carboxamide). In one embodiment, a compound of the present invention or a pharmaceutically acceptable composition, salt, isotopic analog, or prodrug thereof is combined in a dosage form with the MEK inhibitor.

In one embodiment, the at least one additional chemotherapeutic agent combined or alternated with the compound of the present invention is a Raf inhibitor. Raf inhibitors for use in the present invention are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carb oxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-l H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3 (trifluoroMethyl)phenyl]aMino]carbonyl]aMino]phenoxy]-N-Methyl-2pyridinecarboxaMide 1-Oxide). In one embodiment, a compound of the present invention or a pharmaceutically acceptable composition, salt, isotopic analog, or prodrug thereof is combined in a dosage form with the Raf inhibitor.

In one embodiment, the at least one additional chemotherapeutic agent combined or alternated with the compound of the present invention is a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-i-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenyl sulfanylbutan-2-yl] amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-lH-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen). In one embodiment, a compound of the present invention or a pharmaceutically acceptable composition, salt, isotopic analog, or prodrug thereof is combined in a dosage form with the at least one BCL-2 inhibitor. In one embodiment the at least one BCL-2 inhibitor is ABT-199 (Venetoclax).

In one embodiment, a method of treating a tumor or cancer is provided, comprising administration of an effective amount of Compound A or a pharmaceutically acceptable salt thereof in combination or alternation with an effective amount of a BCL-2 inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a BCL-2 inhibitor to a host in need thereof.

In one embodiment, a method of treating a tumor or cancer is provided, comprising administration of an effective amount of Compound A or a pharmaceutically acceptable salt thereof in combination or alternation with ABT-199 to a host in need thereof. In another embodiment, a method of treating a tumor or cancer is provided, comprising administration of an effective amount of an analog of Compound A or a pharmaceutically acceptable salt thereof as provided herein in combination or alternation with ABT-199 to a host in need thereof.

In one embodiment, the treatment regimen includes the administration of a compound of the present invention or a pharmaceutically acceptable composition, salt, isotopic analog, or prodrug thereof in combination or alternation with at least one additional chemotherapeutic agent selected from, but are not limited to, Imatinib mesylate (Gleevac), Dasatinib (Sprycel), Nilotinib (Tasigna), Bosutinib (Bosulif), Trastuzumab (Herceptin), Pertuzumab (Perjeta™), Lapatinib (Tykerb), Gefitinib (Iressa), Erlotinib (Tarceva), Cetuximab (Erbitux), Panitumumab (Vectibix), Vandetanib (Caprelsa), Vemurafenib (Zelboraf), Vorinostat (Zolinza), Romidepsin (Istodax), Bexarotene (Tagretin), Alitretinoin (Panretin), Tretinoin (Vesanoid), Carfilizomib (Kyprolis™), Pralatrexate (Folotyn), Bevacizumab (Avastin), Ziv-aflibercept (Zaltrap), Sorafenib (Nexavar), Sunitinib (Sutent), Pazopanib (Votrient), Regorafenib (Stivarga), and Cabozantinib (Cometriq™).

In some embodiments, the pharmaceutical combination or composition described herein can be administered to the subject in combination or further combination with other chemotherapeutic agents for the treatment of a tumor or cancer. If convenient, the pharmaceutical combination or composition described herein can be administered at the same time as another chemotherapeutic agent, in order to simplify the treatment regimen. In some embodiments, the pharmaceutical combination or composition and the other chemotherapeutic can be provided in a single formulation. In one embodiment, the use of the pharmaceutical combination or composition described herein is combined in a therapeutic regime with other agents. Such agents may include, but are not limited to, tamoxifen, midazolam, letrozole, bortezomib, anastrozole, goserelin, an mTOR inhibitor, a PI3 kinase inhibitor as described above, a dual mTOR-PI3K inhibitor, a MEK inhibitor as described above, a RAS inhibitor, ALK inhibitor, an HSP inhibitor (for example, HSP70 and HSP 90 inhibitor, or a combination thereof), a BCL-2 inhibitor as described above, apopototic inducing compounds, an AKT inhibitor, including but not limited to, MK-2206 (1,2,4-Triazolo[3,4-f][1,6]naphthyridin-3 (2H)-one, 8-[4-(1-aminocyclobutyl)phenyl]-9-phenyl-), GSK690693, Perifosine, (KRX-0401), GDC-0068, Triciribine, AZD5363, Honokiol, PF-04691502, and Miltefosine, a PD-1 inhibitor as described above including but not limited to, Nivolumab, CT-011, MK-3475, BMS936558, and AMP-514 or a FLT-3 inhibitor, including but not limited to, P406, Dovitinib, Quizartinib (AC220), Amuvatinib (MP-470), Tandutinib (MLN518), ENMD-2076, and KW-2449, or a combination thereof. Examples of mTOR inhibitors include but are not limited to rapamycin and its analogs, everolimus (Afinitor), temsirolimus, ridaforolimus, sirolimus, and deforolimus. Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol. In a particular embodiment, a compound described herein is administered in combination with letrozole and/or tamoxifen. Other chemotherapeutic agents that can be used in combination with the compounds described herein include, but are not limited to, chemotherapeutic agents that do not require cell cycle activity for their anti-neoplastic effect.

In one embodiment, the treatment regimen includes the administration of a compound of the present invention or a pharmaceutically acceptable composition, salt, isotopic analog, or prodrug thereof in combination or alternation with at least one additional therapy, wherein the second therapy is an immunotherapy.

The combination agent can be conjugated to an antibody, radioactive agent, or other targeting agent that directs the active compound as described herein to the diseased or abnormally proliferating cell. In another embodiment, the pharmaceutical combination or composition is used in combination with another pharmaceutical or a biologic agent (for example an antibody) to increase the efficacy of treatment with a combined or a synergistic approach. In an embodiment, the pharmaceutical combination or composition can be used with T-cell vaccination, which typically involves immunization with inactivated autoreactive T cells to eliminate a cancer cell population as described herein. In another embodiment, the pharmaceutical combination or composition is used in combination with a bispecific T-cell Engager (BiTE), which is an antibody designed to simultaneously bind to specific antigens on endogenous T cells and cancer cells as described herein, linking the two types of cells.

In one embodiment, the additional therapy is a monoclonal antibody (MAb). Some MAbs stimulate an immune response that destroys cancer cells. Similar to the antibodies produced naturally by B cells, these MAbs “coat” the cancer cell surface, triggering its destruction by the immune system. For example, bevacizumab targets vascular endothelial growth factor(VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels.

Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

Another group of cancer therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a cancer cell, and the cell-killing substance is taken up by the cell. FDA-approved conjugated MAbs that work this way include ado-trastuzumab emtansine, which targets the HER-2 molecule to deliver the drug DM1, which inhibits cell proliferation, to HER-2 expressing metastatic breast cancer cells.

Immunotherapies with T cells engineered to recognize cancer cells via bispecific antibodies (bsAbs) or chimeric antigen receptors (CARs) are approaches with potential to ablate both dividing and non/slow-dividing subpopulations of cancer cells.

Bispecific antibodies, by simultaneously recognizing target antigen and an activating receptor on the surface of an immune effector cell, offer an opportunity to redirect immune effector cells to kill cancer cells. Another approach is the generation of chimeric antigen receptors by fusing extracellular antibodies to intracellular signaling domains. Chimeric antigen receptor-engineered T cells are able to specifically kill tumor cells in a MHC-independent way.

In certain aspects, the additional therapy is another therapeutic agent, for example, an anti-inflammatory agent, a chemotherapeutic agent, a radiotherapeutic agent, or an immunosuppressive agent.

Suitable chemotherapeutic agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds.

General anticancer pharmaceutical agents for administration as additional agents include: Vincristine (Oncovin) or liposomal vincristine (Marqibo), Daunorubicin (daunomycin or Cerubidine) or doxorubicin (Adriamycin), Cytarabine (cytosine arabinoside, ara-C, or Cytosar), L-asparaginase (E1spar) or PEG-L-asparaginase (pegaspargase or Oncaspar), Etoposide (VP-16), Teniposide (Vumon), 6-mercaptopurine (6-MP or Purinethol), Methotrexate, Cyclophosphamide (Cytoxan), Prednisone, Dexamethasone (Decadron), imatinib (Gleevec marketed by Novartis), dasatinib (Sprycel), nilotinib (Tasigna), bosutinib (Bosulif), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Suitable immunosuppressive agents include, but are not limited to: calcineurin inhibitors, e.g. a cyclosporin or an ascomycin, e.g. Cyclosporin A (NEORAL), FK506 (tacrolimus), pimecrolimus, a mTOR inhibitor, e.g. rapamycin or a derivative thereof, e.g. Sirolimus (RAPAMUNE), Everolimus (Certican), temsirolimus, zotarolimus, biolimus-7, biolimus-9, a rapalog, e.g.ridaforolimus, azathioprine, campath 1H, a S1P receptor modulator, e.g. fingolimod or an analog thereof, an anti IL-8 antibody, mycophenolic acid or a salt thereof, e.g. sodium salt, or a prodrug thereof, e.g. Mycophenolate Mofetil (CELLCEPT), OKT3 (ORTHOCLONE OKT3), Prednisone, ATGAM, THYMOGLOBULIN, Brequinar Sodium, OKT4, T10B9.A-3A, 33B3.1, 15-deoxyspergualin, tresperimus, Leflunomide ARAVA, CTLAI-Ig, anti-CD25, anti-IL2R, Basiliximab (SIMULECT), Daclizumab (ZENAPAX), mizorbine, methotrexate, dexamethasone, ISAtx-247, SDZ ASM 981 (pimecrolimus, Elidel), CTLA41g (Abatacept), belatacept, LFA31g, etanercept (sold as Enbrel by Immunex), adalimumab (Humira), infliximab (Remicade), an anti-LFA-1 antibody, natalizumab (Antegren), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In certain embodiments, a pharmaceutical combination or composition described herein is administered to the subject prior to treatment with another chemotherapeutic agent, during treatment with another chemotherapeutic agent, after administration of another chemotherapeutic agent, or a combination thereof.

In some embodiments, the selective pharmaceutical combination or composition can be administered to the subject such that the other chemotherapeutic agent can be administered either at higher doses (increased chemotherapeutic dose intensity) or more frequently (increased chemotherapeutic dose density). Dose-dense chemotherapy is a chemotherapy treatment plan in which drugs are given with less time between treatments than in a standard chemotherapy treatment plan. Chemotherapy dose intensity represents unit dose of chemotherapy administered per unit time. Dose intensity can be increased or decreased through altering dose administered, time interval of administration, or both.

In one embodiment of the invention, the pharmaceutical combination or composition described herein can be administered in a concerted regimen with another agent such as a non-DNA-damaging, targeted anti-neoplastic agent or a hematopoietic growth factor agent. It has recently been reported that the untimely administration of hematopoietic growth factors can have serious side effects. For example, the use of the EPO family of growth factors has been associated with arterial hypertension, cerebral convulsions, hypertensive encephalopathy, thromboembolism, iron deficiency, influenza like syndromes and venous thrombosis. The G-CSF family of growth factors has been associated with spleen enlargement and rupture, respiratory distress syndrome, allergic reactions and sickle cell complications. By combining the administration of the pharmaceutical combination or composition as described herein with the timely administration of hematopoietic growth factors, for example, at the time point wherein the affected cells are no longer under growth arrest, it is possible for the health care practitioner to decrease the amount of the growth factor to minimize the unwanted adverse effects while achieving the desired therapeutic benefit. As such, in one embodiment, the use of the pharmaceutical combination, composition, or methods described herein is combined with the use of hematopoietic growth factors including, but not limited to, granulocyte colony stimulating factor (G-CSF, for example, sold as Neupogen (filgrastin), Neulasta (peg-filgrastin), or lenograstin), granulocyte-macrophage colony stimulating factor (GM-CSF, for example sold as molgramostim and sargramostim (Leukine)), M-CSF (macrophage colony stimulating factor), thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-a as Darbopoetin, Epocept, Nanokine, Epofit, Epogin, Eprex and Procrit; epoetin-(3 sold as for example NeoRecormon, Recormon and Micera), epoetin-delta (sold as for example Dynepo), epoetin-omega (sold as for example Epomax), epoetin zeta (sold as for example Silapo and Reacrit) as well as for example Epocept, EPOTrust, Erypro Safe, Repoeitin, Vintor, Epofit, Erykine, Wepox, Espogen, Relipoeitin, Shanpoietin, Zyrop and EPIAO). In one embodiment, the pharmaceutical combination or composition is administered prior to administration of the hematopoietic growth factor. In one embodiment, the hematopoietic growth factor administration is timed so that the pharmaceutical combination or composition's effect on HSPCs has dissipated.

In one embodiment, the growth factor is administered at least 20 hours after the administration of a pharmaceutical combination or composition described herein.

If desired, multiple doses of a pharmaceutical combination or composition described herein can be administered to the subject. Alternatively, the subject can be given a single dose of a pharmaceutical combination or composition described herein.

In one embodiment, the activity of an active compound for a purpose described herein can be augmented through conjugation to an agent that targets the diseased or abnormally proliferating cell or otherwise enhances activity, delivery, pharmacokinetics or other beneficial property.

A selected compound described herein can be administered in conjugation or combination with a Fv fragment. Fv fragments are the smallest fragment made from enzymatic cleavage of IgG and IgM class antibodies. Fv fragments have the antigen-binding site made of the VH and VC regions, but they lack the CH₁ and CL regions. The VH and VL chains are held together in Fv fragments by non-covalent interactions.

In one embodiment, a selected compound as described herein can be administered in combination with an antibody fragment selected from the group consisting of an ScFv, domain antibody, diabody, triabody, tetrabody, Bis-scFv, minibody, Fab2, or Fab3 antibody fragment. In one embodiment, the antibody fragment is a ScFv. Genetic engineering methods allow the production of single chain variable fragments (ScFv), which are Fv type fragments that include the VH and VL domains linked with a flexible peptide. When the linker is at least 12 residues long, the ScFv fragments are primarily monomeric. Manipulation of the orientation of the V-domains and the linker length creates different forms of Fv molecules linkers that are 3-11 residues long yield scFv molecules that are unable to fold into a functional Fv domain. These molecules can associate with a second scFv molecule, to create a bivalent diabody. In one embodiment, the antibody fragment administered in combination with a selected compound described herein is a bivalent diabody. If the linker length is less than three residues, scFv molecules associate into triabodies or tetrabodies. In one embodiment, the antibody fragment is a triabody. In one embodiment, the antibody fragment is a tetrabody. Multivalent scFvs possess greater functional binding affinity to their target antigens than their monovalent counterparts by having binding to two more target antigens, which reduces the off-rate of the antibody fragment. In one embodiment, the antibody fragment is a minibody. Minibodies are scFv-CH₃ fusion proteins that assemble into bivalent dimers. In one embodiment, the antibody fragment is a Bis-scFv fragment. Bis-scFv fragments are bispecific. Miniaturized ScFv fragments can be generated that have two different variable domains, allowing these Bis-scFv molecules to concurrently bind to two different epitopes.

In one embodiment, a selected compound described herein is administered in conjugation or combination with a bispecific dimer (Fab2) or trispecific dimer (Fab3). Genetic methods are also used to create bispecific Fab dimers (Fab2) and trispecific Fab trimers (Fab3). These antibody fragments are able to bind 2 (Fab2) or 3 (Fab3) different antigens at once.

In one embodiment, a selected compound described herein is administered in conjugation or combination with an rIgG antibody fragment. rIgG antibody fragments refers to reduced IgG (75,000 daltons) or half-IgG. It is the product of selectively reducing just the hinge-region disulfide bonds. Although several disulfide bonds occur in IgG, those in the hinge-region are most accessible and easiest to reduce, especially with mild reducing agents like 2-mercaptoethylamine (2-MEA). Half-IgG are frequently prepared for the purpose of targeting the exposing hinge-region sulfhydryl groups that can be targeted for conjugation, either antibody immobilization or enzyme labeling.

In other embodiments, a selected active compound described herein can be linked to a radioisotope to increase efficacy, using methods well known in the art. Any radioisotope that is useful against cancer cells can be incorporated into the conjugate, for example, but not limited to, ¹³¹I, ¹²³I, ¹⁹²Ir, ³²P, ⁹⁰Sr, ¹⁹⁸Au, ²²⁶Ra, ⁹⁰Y, ²⁴¹Am, ²⁵²Cf, ⁶⁰Co, or ¹³⁷Cs.

Of note, the linker chemistry can be important to efficacy and tolerability of the drug conjugates. The thio-ether linked T-DM1 increases the serum stability relative to a disulfide linker version and appears to undergo endosomal degradation, resulting in intra-cellular release of the cytotoxic agent, thereby improving efficacy and tolerability, See, Barginear, M. F. and Budman, D. R., Trastuzumab-DM1: A review of the novel immune-conjugate for HER2-overexpressing breast cancer, The Open Breast Cancer Journal, 1: 25-30, (2009).

Examples of early and recent antibody-drug conjugates, discussing drugs, linker chemistries and classes of targets for product development that may be used in the present invention can be found in the reviews by Casi, G. and Neri, D., Antibody-drug conjugates: basic concepts, examples and future perspectives, J. Control Release 161(2):422-428, 2012, Chari, R. V., Targeted cancer therapy: conferring specificity to cytotoxic drugs, Acc. Chem. Rev., 41(1):98-107, 2008, Sapra, P. and Shor, B., Monoclonal antibody-based therapies in cancer: advances and challenges, Pharmacol. Ther., 138(3):452-69, 2013, Schliemann, C. and Neri, D., Antibody-based targeting of the tumor vasculature, Biochim. Biophys. Acta., 1776(2):175-92, 2007, Sun, Y., Yu, F., and Sun, B. W., Antibody-drug conjugates as targeted cancer therapeutics, Yao Xue Xue Bao, 44(9):943-52, 2009, Teicher, B. A., and Chari, R. V., Antibody conjugate therapeutics: challenges and potential, Clin. Cancer Res., 17(20):6389-97, 2011, Firer, M. A., and Gellerman, G. J., Targeted drug delivery for cancer therapy: the other side of antibodies, J. Hematol. Oncol., 5:70, 2012, Vlachakis, D. and Kossida, S., Antibody Drug Conjugate bioinformatics: drug delivery through the letterbox, Comput. Math. Methods Med., 2013; 2013:282398, Epub 2013 Jun. 19, Lambert, J. M., Drug-conjugated antibodies for the treatment of cancer, Br. J. Clin. Pharmacol., 76(2):248-62, 2013, Concalves, A., Tredan, O., Villanueva, C. and Dumontet, C., Antibody-drug conjugates in oncology: from the concept to trastuzumab emtansine (T-DM1), Bull. Cancer, 99(12):1183-1191, 2012, Newland, A. M., Brentuximab vedotin: a CD-30-directed antibody-cytotoxic drug conjugate, Pharmacotherapy, 33(1):93-104, 2013, Lopus, M., Antibody-DM1 conjugates as cancer therapeutics, Cancer Lett., 307(2):113-118, 2011, Chu, Y. W. and Poison, A., Antibody-drug conjugates for the treatment of B-cell non-Hodgkin's lymphoma and leukemia, Future Oncol., 9(3):355-368, 2013, Bertholjotti, I., Antibody-drug conjugate a new age for personalized cancer treatment, Chimia, 65(9): 746-748, 2011, Vincent, K. J., and Zurini, M., Current strategies in antibody engineering: Fc engineering and pH—dependent antigen binding, bispecific antibodies and antibody drug conjugates, Biotechnol. J., 7(12):1444-1450, 2012, Haeuw, J. F., Caussanel, V., and Beck, A., Immunoconjugates, drug-armed antibodies to fight against cancer, Med. Sci., 25(12):1046-1052, 2009 and Govindan, S. V., and Goldenberg, D. M., Designing immunoconjugates for cancer therapy, Expert Opin. Biol. Ther., 12(7):873-890, 2012.

In one embodiment the pharmaceutical composition or combination as described herein can be used to treat any disorder described herein.

In one aspect a compound of the present invention is dosed in a combination or composition with an effective amount of a nucleoside or nucleoside analog. Non-limiting examples of nucleosides include: azacitidine, decitabine, didanosine, vidarabine, BCX4430, cytarabine, emtricitabine, lamivudine, zalcitabine, abacavir, aciclovir, entecavir, stavudine, telbivudine, zidovudine, idoxuridine, trifluridine, apricitabine, elvucitabine, amdoxovir, and racivir. In one embodiment the compound of present invention is used in a combination or composition with an effective amount of a nucleoside or nucleoside analog to treat a viral infection. In an alternative embodiment the compound of present invention is used in a combination or composition with an effective amount of a nucleoside or nucleoside analog to treat a tumor or cancer. In one embodiment the nucleoside analog is azacitidine and the disorder is tumor or cancer.

In one embodiment, provided is a method of treating tumor or cancer in a subject comprising administration of Compound A or a pharmaceutically acceptable salt thereof in combination or alternation with an effective amount of a nucleoside analog to a host in need thereof. In another embodiment, provided is a method of treating tumor or cancer in a subject comprising administration of an analog of Compound A or a pharmaceutically acceptable salt thereof as provided herein in combination or alternation with an effective amount of a nucleoside analog to a host in need thereof.

In one embodiment, provided is a method of treating tumor or cancer in a subject comprising administration of Compound A or a pharmaceutically acceptable salt thereof in combination or alternation with azacitidine to a host in need thereof. In another embodiment, provided is a method of treating tumor or cancer in a subject comprising administration of an analog of Compound A or a pharmaceutically acceptable salt thereof as provided herein in combination or alternation with azacitidine to a host in need thereof.

### VII. EXAMPLES

### Example 1: Determination of Genes in Kasumi-1 Cells that are Related to RUNX1-RUNX1T1

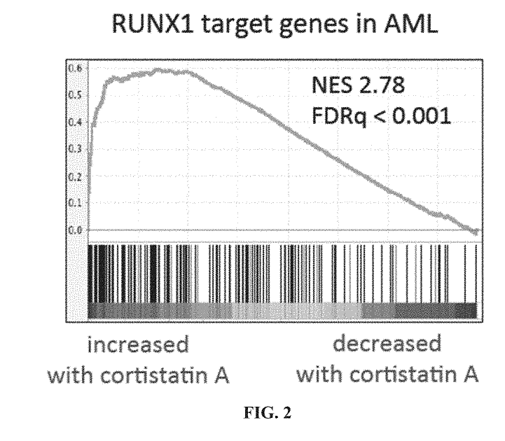

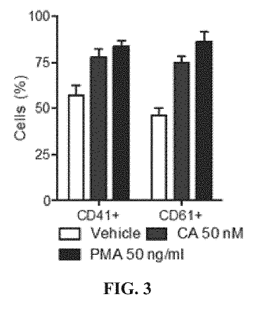

Kasumi-1 AML cells contain the RUNX1-RUNX1T1 fusion. A gene set was obtained that measured the genes in Kasumi-1 cells that increase in expression upon knockdown of RUNX1-RUNX1T1 (Ben-Ami, O. et al. Cell Reports 4, 1131-1143 (2013)). Using gene set enrichment analysis (Subramanian, A. et. al. Proc. Natl Acad. Sci. USA 102, 15545-15550 (2005)), this gene set, together with the Broad Molecular Signatures database (C2) was compared to genes differentially expressed in MOLM-14 cells upon treatment with 25 nM cortistatin A for 3 hrs (FIG. 2).

### Example 2: Determination of Gene Expression Levels

Leukemia cells were plated (12-well) in triplicate at 500,000-800,000 cells per ml and incubated in the presence of vehicle (0.1% DMSO) or CA (25 nM 3 h for K562, MOLM-14 and MV4;11; 10 nM 24 h for MOLM-14; 25 nM 4 h for SET-2, n=3 for each cell line). Cells were then washed twice with cold PBS, and snap frozen. RNA was isolated (RNeasy Plus Microkit, Qiagen or TRIzol, Life Technologies), processed, and, for K562, MOLM-14 and MV4;11, hybridized to the Human U133 Plus 2.0 microarray (Affymetrix). Microarrays were processed with Bioconductor packages affyQCReport for quality control and affy for background correction, summarization, and normalization using rma. Probe sets present in at least 1 sample (based on affy mas5call) and for which the interquartile range was >log₂(1.2) were retained for further analysis. The limma Bioconductor package was used for differential expression analysis of CA-treated versus DMSO control samples (Benjamini-Hochberg adjusted P<0.05). SET-2 and HCT 116 gene expression was measured by RNA-seq. SET-2 RNA-seq libraries were prepared and processed using the Ion Torrent workflow. Reads were aligned in two passes, first with rnaStar (v.2.3.0e) then with BWA (v.0.7.5a) for remaining unmapped reads, both using default parameters. Mapped reads were merged and counted using HTSeq (v.0.5.3p3) with -s yes -m intersection-strict. The Bioconductor package DESeq was used for DE analysis (FDR<0.05 and twofold change) and normalization. HCT116 cells were grown to approximately 80% confluence and were treated with either 100 nM CA or DMSO for 3 h (n=3). Cells were then washed twice with cold PBS and scraped into TRIzol reagent (Life Technologies). After collecting the RNA, it was further purified using an RNeasy mini kit (Qiagen) with an on-column DNase I digestion. Libraries for Illumina sequencing were generated via the Illumina TruSEQ stranded mRNA prep kit. Samples were run in a single lane on an Illumina HiSEQ 2000 sequencer with a single read flow cell using 1×50-bp reads and a 6-cycle index read. Reads were mapped to the hgl9 reference genome using Tophat2 v.2.0.6 with custom settings including the setting of—library-type fr—firstrand to appropriately account for the stranded nature of the protocol. HTSeq v.0.6.1 was used to obtain read counts over annotated genes and differentially expressed genes were called by DESeq v.1.10.1 with a padj value of less than 0.01. Counts were normalized for GSEA using the limma voom function. Expression data for the I-BET151 comparison were downloaded from ArrayExpress (https://www.ebi.ac.uk/arrayexpress, accession E-MTAB-774) and processed files used as is. Gene lists were submitted to the DAVID web server (http://david.abcc.ncifcrf.gov) for functional annotation. GSEA version 2.09 was carried out using signal-to-noise on natural values as the metric. Signatures included curated gene sets (C2, v.3) downloaded from the Broad's MSigDB as well as signatures curated from in-house and published data sets.

The results of the gene expression screening are summarized in Table 2 below.

TABLE 2
Cortistatin A Upregulates RUNX1 Target Genes. GSEA indicates
upregulation of RUNX1 target gene signatures with 3 h
25 nM cortistatin A treatment in MOLM-14 cells or 4 h
25 nM cortistatin A treatment in SET-2 cells.

| | | | Rank |
|---|---|---|---|
| Selected Gene Signatures | NES | FDRq | of 4,310 |
| Upregulated by RUNX1-RUNX1T1 | 2.78 | <0.001 | 1 |
| knockdown in Kasumi-1 cells* | | | |
| Upregulated by C/EBPα in | 2.56 | <0.001 | 5 |
| Burkitt lymphoma cells* | | | |
| Downregulated by RUNX1-RUNX1T1 | 2.50 | <0.001 | 9 |
| in CD34+ cord blood cells* | | | |
| Upregulated by C/EBPα in KCL22 cells* | 2.22 | <0.001 | 40 |
| Upregulated by C/EBPα in K562 cells* | 2.22 | <0.001 | 42 |
| Downregulated by RUNX1 | 2.21 | <0.001 | 43 |
| knockdown in Kasumi-1 cells* | | | |
| Downregulated by RUNX1 | 2.00 | 0.006 | 120 |
| knowndown in Jurkat cells* | | | |
| GATA1/SCL Binding Sites in Mks** | 2.27 | <0.001 | 12 |
| GATA1/GATA2/RunX1/FLI1/SCL | 2.03 | 0.006 | 100 |
| Binding Sites in Mks** | | | |

*MOLM-14 cells, 3 h CA treatment;
**SET-2 cells, 4 h CA treatment

Using gene set enrichment analysis, it is found that treatment of MOLM-14 cells or SET-2 cells increases expression of these RUNX1 target genes. The curated gene sets were analyzed together with over 4,000 signatures including the Broad Molecular Signatures database (C2).

### Example 3: Method to Determine the Differentiation of Studied Cells

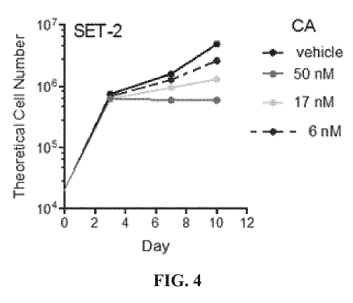

For the SET-2 differentiation assay, cells were plated (6-well) in triplicate at 150,000 cells per ml for 3-days with 50 nM CA, 50 ng ml⁻¹ PMA (positive control), or vehicle. Cell pellets were collected at 4° C., washed three times with cold PBS, and stained with anti-CD61-PE (ab91128) or anti-CD41-PerCP (ab134373) and analyzed by flow cytometry. For each experiment, n=3 biological replicates with two independent experiments and one shown (FIG. 3).

### Example 4: Cellular Proliferation Assay

All suspension cells were plated (96-well) in triplicate at 5,000-30,000 cells per well for testing (n=3). Viable cell number was estimated after 3, 7 and 10 days by counting viable cells from one vehicle well, generating a cell dilution series, transferring 20 ml per well in duplicate to a 384-well plate, and performing a linear regression to CellTiter-Glo (Promega) response (SPECTRAmax M3, Molecular Devices). Cells from all wells were also fourfold diluted in media and transferred in duplicate for CellTiter-Glo measurement. On days 3 and 7, an equal volume for all wells was split-back with fresh media and compound, such that the resulting cell density for the vehicle well matched the initial seeding density. For days 7 and 10, estimated cell number represents the split-adjusted theoretical cell number. HCT116 were plated (96-well) in triplicate at 250 cells per well. Cells were incubated in the presence of vehicle, 1 μM paclitaxel, or compound. On day 7, CellTiter-Blue (Promega) response was measured and values were normalized to vehicle (100% growth) and paclitaxel (0% growth). For growth assays with inhibitors, n=3 for each concentration with two independent experiments.

The results are summarized below in Table 3 with one graph of proliferation over time shown (FIG. 4).

TABLE 3
Selected Cortistatin A Cell Line Sensitivity.

| | | | GI50 |
|---|---|---|---|
| Cell Line | Malignancy | Selected Alteration | (nM) |
| MOLM-16 | AMkL | | <1 |
| JeKo-1 | Mantle Cell Lymphoma | | <1 |
| SKNO-1 | AML | RUNX1-RUNX1T1 | 1 |
| REH | pre B-ALL | ETV6-RUNX1 | 1 |
| ALL-SIL | T-ALL | TLX1 | 2 |
| | | overexpression | |
| RS4; 11 | B-ALL | MLL-AF4 | 3 |
| SET-2 | AMkL/MPN | JAK2V617F | 4 |
| MM1.S | MM | | 5 |
| MOLM-14 | AML | MLL-AF9 | 5 |
| MV4; 11 | AML | MLL-AF4 | 6 |
| UKE-1 | AML/MPN | JAK2V617F | 7 |
| HL-60 | AML/APL | | 7 |
| CMK-86 | AMkL | GATA1s | 8 |
| MEG-01 | CML/AMkL | BCR-ABL, | 9 |
| | | GATA1s | |
| ARH-77 | MM/PCL | | 16 |
| OCI-LY1 | DLBCL | | 37 |
| Kasumi-1 | AML | RUNX1-RUNX1T1 | 95 |
| Jurkat | T-ALL | | 155 |
| TF-1 | AML/Erythroleukemia | EpoR | 350 |
| GRANTA 519 | Matle Cell Lymphoma | | 400 |
| NB4 | AML/APL | PML-RARA | >1,000 |
| K562 | CML/Erythroleukemia | BCR-ABL | >1,000 |
| HEL | AML/Erythroleukemia | JAK2V617F | >1,000 |
| HCT116 | Colorectal | B-catenin | >1,000 |

Table 3 shows that many blood cancer cell lines are growth inhibited by CDK8/19 inhibitor cortistatin A. Among the highly sensitive cell lines are two that have mutations in RUNX1 itself (SKNO-1 and REH) as well as others that are likely to have reduced levels of RUNX1 (RS4;11, MV4;11 and MOLM-14; based on finding that MLL-fusions reduce protein levels of RUNX1(Zhao, X. et al. Downregulation of RUNX1/CBF(3 by MLL fusion proteins enhances hematopoietic stem cell self-renewal. Blood 123, 1729-1738 (2014))). Additional cell lines are predicted to be sensitive to cortistatin A because cortistatin A increases the RUNX1 transcriptional program. These include megakaryocytic cell lines MOLM-16, SET-2, MEG-01 and CMK-86 because RUNX1 is required for differentiation of megakaryoctyes (de Bruijn, M. F. & Speck, N. A. Core-binding factors in hematopoiesis and immune function. Oncogene 23, 423 8-4248 (2004)) and ALL cell lines ALL-SIL, which has overexpressed TLX1. TLX1 overexpression was shown to control the RUNX1 transcriptional program (Gatta, Della, G. et al. Reverse engineering of TLX oncogenic transcriptional networks identifies. Nat. Med. 18, 436-440 (2012)).

Cortistatins potently inhibit proliferation of a number of AML cell lines with 50% maximal growth inhibitory concentrations (G1sos) of less than 10 nM. Cell line sensitivity was consistent with RUNX1 transcriptional program dependence. Sensitive cell lines include those containing fusions that directly inhibit RUNX1 or transcription of its target genes (SKNO-1, ME-1, MOLM-14 as well as megakaryoblastic leukemia cell lines with truncated GATA-1 protein GATA-1s (CMK-86 and MEG-01). Unlike in megakaryopoieis, RUNX1 expression rapidly declines during terminal differentiation of erythrocytes, consistent with an insensitivity of erythroleukemia lines to CA. By means of example, it was determined that cortistatins increase a RUNX1 transcriptional program AML cell lines SET-2, MOLM-14 and MV4; 11. Cortistatins upregulated RUNX1 target genes including CEBPA, IRF8 and NFE2 and, by gene set enrichment analysis (GSEA), it was determined that (i) cortistatins upregulate genes in SET-2, MOLM-14 and MV4; 11 cell lines that are repressed by expression of RUNX1-RUNX1T1 in hematopoietic stem cells; (ii) cortistatins upregulate genes in MOLM-14 and MV4; 11 cells that are reduced in expression in the Kasumi-1 AML cell line upon siRNA-mediated knockdown of RUNX1; and (iii) cortistatins upregulate genes in MOLM-14 cells that increase in expression upon siRNA-mediated knockdown of RUNX1-RUNX1T1 in Kasumi-1 cells. RUNX1 was recruited to loci upregulated by cortistatin treatment.

### Example 5: SET-2/UKE-1 Synergy Assay

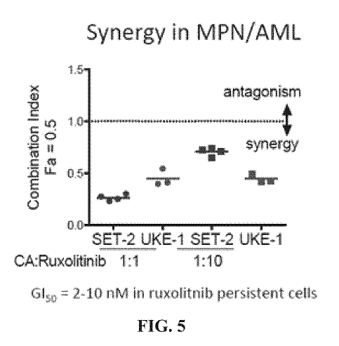

SET-2 and UKE-1 cells were co-treated with constant ratios of ruxolitinib to CA, 1 to 1 or 10 to 1, in a 96-well growth assay format with a range of 2-fold dose dilutions of compounds. SET-2 and UKE-1 cells were also treated with ruxolitinib alone or CA alone in a 2-fold dilution series. The Chou-Talalay combination index values at 50% growth inhibition (Fa=0.5) were determined using CalcuSyn software (see Chou, T. C. Cancer Res. 2010 Jan. 15; 70(2):440-6) (FIG. 5).

### Example 6: In Vivo Xenograft Study

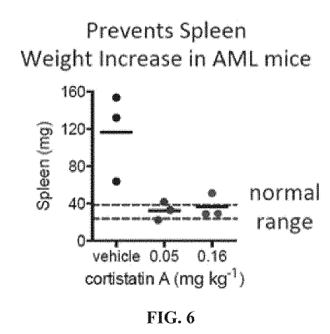

The MV4;11 xenograft model was performed as previously described (Etchin, J. et al. Antileukemic activity of nuclear export inhibitors that spare normal hematopoietic cells. Leukemia 27, 66-74 (2013)). Two-million MV4; 11-mCLP cells were injected into the tail vein of 7-week-old female non-obese diabetic-severe combined immuno-deficient (NOD-SCID)Il2rg^{-/-} (NSG) mice (The Jackson Laboratory) and tumour burden was assessed by bioluminescence imaging (BLI) using an IVIS Spectrum system (Caliper Life Sciences). Seven days after injection, leukaemia establishment was documented by BLI and mice were assigned to groups to achieve a similar mean BLI and treated intraperitoneally with vehicle (20% hydroxypropyl-P3-cyclodextrin) or CA once daily for 15 days. After 30 days, blood counts were obtained (Hemavet 950 F, Drew Scientific) and spleen, femur and peripheral blood cells were collected and analysed by flow cytometry (LSR Fortessa, BD Biosciences) from three mice per group, having the highest, lowest and median BLI value. The spleens were weighed (FIG. 6) and the mice and a portion of the spleen were preserved in bouins after body cavities were opened and visceral organs exposed. Samples from all organs were then dissected and placed in nine cassettes per mouse. Tissues were paraffin embedded, sectioned at 6 m and stained with haematoxylin and eosin. Survival was measured as the time from therapy initiation until moribund state. Statistical analyses were performed using GraphPad Prism 6.0. For P value determinations, two-way or one-way ANOVA was used with Dunnett's multiple comparison testing and P-value adjustment.

### Example 7: Native and Recombinate Kinase Profiling

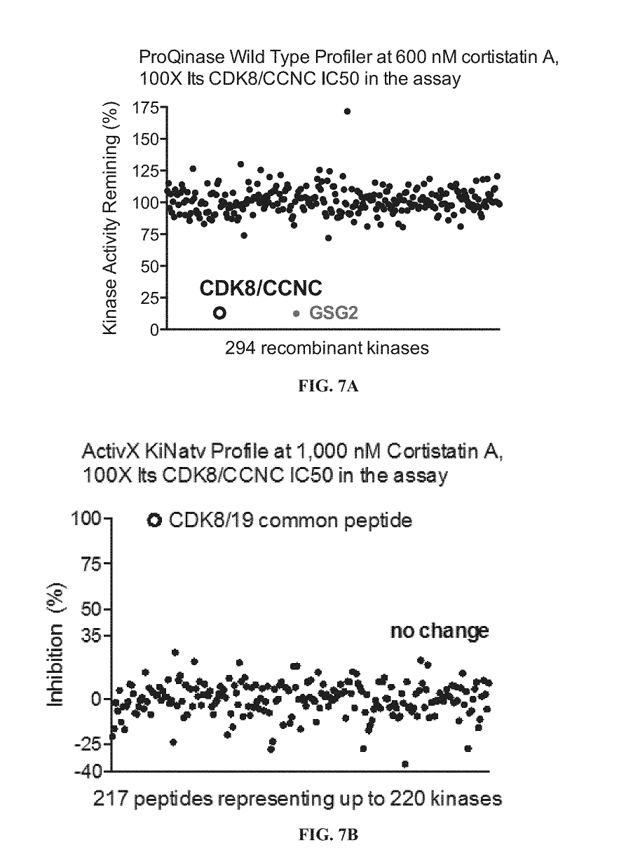

Native kinome profiling was performed with MOLM-14 cell lysate according to the KiNativ Method by ActivX Biosciences. For each peptide quantified, the change in mass spectrometry signal for the treated samples relative to the signal for the control samples was expressed as percentage inhibition. The results correspond to one experiment of duplicates for each cortistatin A (CA) concentration. The percentage changes in mass spectrometry signal reported are statistically significant (Student's t-test score <0.04) (FIG. 7A and FIG. 7B).

Recombinant kinome-wide selectivity profiling. A radiometric protein kinase assay was used (PanQinase activity assay; performed by ProQinase GmbH) as described (Hutterer, C. et al. Antimicrob. Agents Chemother. 59, 2062-2071 (2015)).

### Example 8. In Vitro Radiometric Protein Kinase Assay

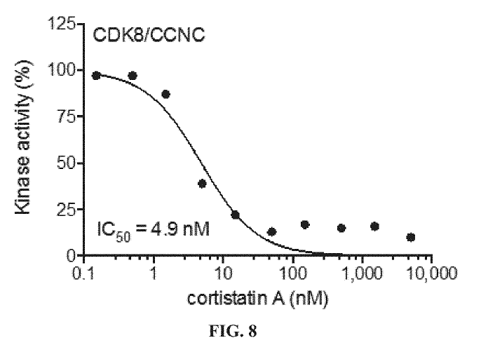

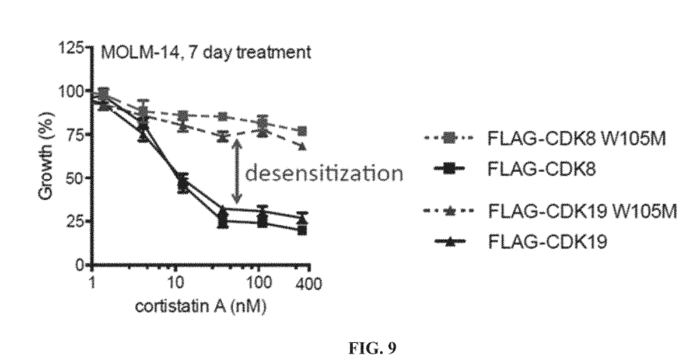

A radiometric protein kinase assay was used (PanQinase activity assay; performed by ProQinase GmbH) as described (Hutterer, C. et al. Antimicrob. Agents Chemother. 59, 2062-2071 (2015)). IC50 determination for CDK8-CCNC (8.3 nM with 1.0 M ATP and 1.0 μg/50 ml of substrate RBER-IRStide) was performed as duplicate measurements and IC50 was calculated using Prism 5.04 with sigmoidal response, top fixed at 100% and bottom at 0% with least-squares fitting (FIG. 8).

### Example 9: Screening Drug Resistant Alleles

5′-Flag-tagged CDK8 and CDK19 were cloned from pBabe.puro.CDK8.flag (Addgene 19758) and F-CDK8L (Addgene 24762) into pLVX-EF1alpha-IRES-mCherry and pLVX-EF1alpha-IRES-ZsGreen (Clontech) and transformed into E. coli (One Shot Stbl3, Invitrogen). Point mutations were introduced by whole-plasmid PCR (QuikChange II XL Site-Directed Mutagenesis Kit, Agilent). pLVX lentiviral vectors were co-transfected with psPASx and pMD2.G (Addgene) in 293T cells. After 48 h, viral supernatants were collected and passed through a 0.45 m filter (Millipore). For transductions, 24-well plates were coated with 500 μl of 20 μg ml RetroNectin (Clontech) at 4° C. overnight, blocked with 2% BSA for 30 min, washed with PBS, and 300-500 μl of viral supernatant was added. The plates were centrifuged (2,000 g, 1.5 h) and then set in an incubator. After 2 h, viral supernatant was removed and 500 μl per well of 200,000 cells per ml was added. After 1-3 days, the cells were expanded and isolated by FACS.

The drug resistant alleles confirm AML cell growth requires CDK8/19 kinase activity. This shows that CDK8/19 inhibitor cortistatin A inhibits the proliferation of MOLM-14 cells by inhibiting CDK8/19. Mutation of tryptophan 105 (W105) in CDK8 and CDK19 confers cortistatin A resistance to CDK8 and CDK19. Therefore, MOLM-14 cells are able to proliferate in the presence of cortistatin A upon expression of CDK8 W105M or CDK19 W105M.

### Example 10: CDK8/19 Inhibition Arrests Leukemia Cell Growth In Vivo

The MV4;11 xenograft model was performed as previously described (Etchin, J. et al. Antileukemic activity of nuclear export inhibitors that spare normal hematopoietic cells. Leukemia 27, 66-74 (2013)). Two-million MV4;11-mCLP cells were injected into the tail vein of 7-week-old female non-obese diabetic-severe combined immuno-deficient (NOD-SCID)Il2rg-^{-/-} (NSG) mice (The Jackson Laboratory) and tumour burden was assessed by bioluminescence imaging (BLI) using an IVIS Spectrum system (Caliper Life Sciences). Seven days after injection, leukaemia establishment was documented by BLI and mice were assigned to groups to achieve a similar mean BLI and treated intraperitoneally with vehicle (20% hydroxypropyl-β-cyclodextrin) or CA once daily for 15 days. After 30 days, blood counts were obtained (Hemavet 950 F, Drew Scientific) and spleen, femur and peripheral blood cells were collected and analysed by flow cytometry (LSR Fortessa, BD Biosciences) from three mice per group, having the highest, lowest and median BLI value. The spleens were weighed (FIG. 6) and the mice and a portion of the spleen were preserved in bouins after body cavities were opened and visceral organs exposed. Samples from all organs were then dissected and placed in nine cassettes per mouse. Tissues were paraffin embedded, sectioned at 6 μm and stained with haematoxylin and eosin. Survival was measured as the time from therapy initiation until moribund state. Statistical analyses were performed using GraphPad Prism 6.0. For P value determinations, two-way or one-way ANOVA was used with Dunnett's multiple comparison testing and P-value adjustment.

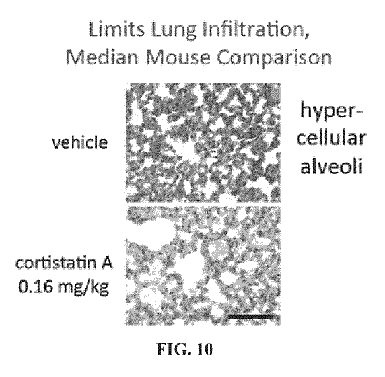

Analysis of MV4; 11 AML mice on Day 30 shows that treatment with CDK8/19 inhibitor cortistatin A has fewer leukemia cells in the lungs, as measured by haematoxylin and eosin staining (FIG. 10).

### Example 11: CDK8/19 Inhibition Increases Expression of RUNX1 Target Genes and Recruitment of RUNX1 to Specific Genomic Loci in AML/Megakaryocytic Cell Lines

It was determined that CA increased a RUNX1 transcriptional program in CA-sensitive cell lines SET-2, MOLM-14 and MV4;11. CA upregulated RUNX1 target genes including CEBPA, IRF8 and NFE2 and, by gene set enrichment analysis (GSEA), it was determined that:
1. CA upregulated genes in SET-2, MOLM-14 and MV4;11 cell lines that are repressed by expression of RUNX1-RUNX1T1 in hematopoietic stem cells (HSCs) (FIG. 2). The RUNX1-RUNX1T1 fusion acts in large part to repress transcription of RUNX1 target genes.
2. CA upregulated genes in MOLM-14 and MV4;11 cells that are reduced in expression in the Kasumi-1 AML cell line upon siRNA-mediated knockdown of RUNX1.
3. CA upregulated genes in MOLM-14 cells that increase in expression upon siRNA-mediated knockdown of RUNX1-RUNX1T1 in Kasumi-1 cells.

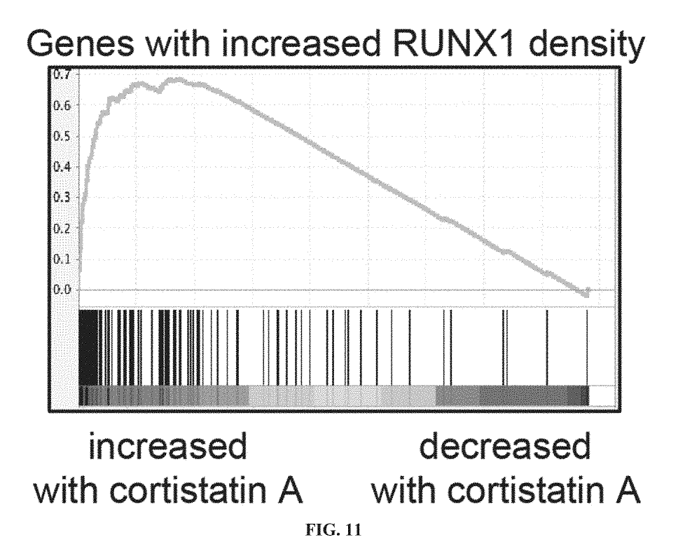

It was observed that mechanistically, RUNX1 was recruited to loci upregulated by CA treatment (FIG. 11), suggesting that CDK8/19 kinase activity restricts RUNX1 from target loci, preventing increased expression of RUNX1-target genes.

### Example 12: RUNX1 Alterations and Related Mutations as Predictive Biomarkers for CDK8/19 Inhibitor Therapy

The antiproliferative activity and effect on differentiation in primary patient samples of cortistatin A (CA) or analogs thereof can be measured. Patient samples (20-40) are obtained that have been characterized to contain mutations or translocations in RUNX1, including RUNX1-RUNX1T1 and monoallelic loss-of-function RUNX1 point mutants. 10-30 additional patient samples that have mutations in transcriptional regulators that modulate RUNX1-target genes together with RUNX1, including mutations in GATA1 exon 2, CBFb-MYH11 translocations, FUS-ERG translocations, and loss-of-function CEBPA indel mutants are also obtained. To assess sensitivity to CA or analogs thereof, both 3-day liquid culture and clonogenic assays on unfractionated and CD34+ leukemia cells from patients are ran. For clonogenic assays, 1,000-2,000 cells are plated per well in duplicate in Methocult (Stem Cell Tech, H4435) supplemented with human cytokines (rhSCF, rhG-CSF, rhGM-CSF, rhIL-3, rhIL-6, rhEpo), in the presence of vehicle, CA, or a CA analog (100 nM, 20 nM, and 4 nM). After 14 days of incubation at 37° C., the total colonies on each plate are counted. In parallel, CD34+ cells are treated for 5 days with vehicle, CA, or a CA analog in the presence of cytokines, then labeled with myeloid markers for differentiation analysis by flow cytometry. Given that each patient sample represents a finite and limited resource, during the initial testing cells that have undergone CDK8/19 inhibitor and vehicle treatment are collected for follow-up genomic analysis, such as gene expression studies. These samples will be useful in validating hits from the CRISPR-Cas9 screen.

Using the above method, a broad set of primary AML patient samples representing most of the common genetic alterations in AML7, including mutations in DNMT3A, NPM1, WT1, cohesion complex components, and FLT3 are evaluated.

### Example 13: CRISPR-Cas9. Cas9 Validation and Initial Screen in MOLM-14 Cells

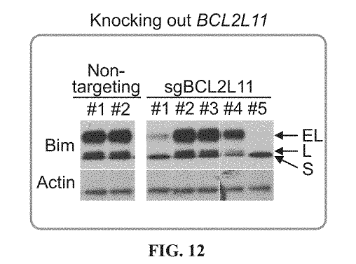

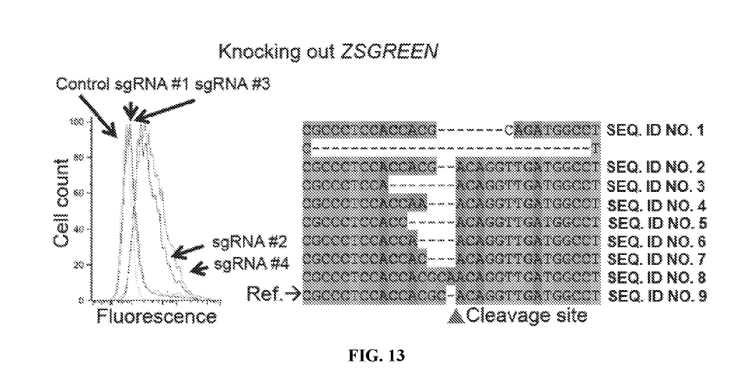

Humanized S. pyogenes Cas9 is expressed in the CA-sensitive AML cell line MOLM-14 and its ability to knock out two genes: ZsGREEN (a lentivirally integrated gene encoding a green fluorescent protein) and BCL2L11 (an endogenous gene encoding the pro-apoptotic protein Bim) is verified (FIG. 12 and FIG. 13)). Sequencing of the ZsGREEN locus (SEQ. ID NO. 1-9) in cells expressing sgRNA #1 revealed indels at the expected cleavage site.

(SEQ. ID NO. 1)
CGCCCTCCACCACGCAGATGGCCT
(SEQ. ID NO. 2)
CGCCCTCCACCACGACAGGTTGATGGCCT
(SEQ. ID NO. 3)
CGCCCTCCAACAGGTTGATGGCCT
(SEQ. ID NO. 4)
CGCCCTCCACCAAACAGGTTGATGGCCT
(SEQ. ID NO. 5)
CGCCCTCCACCACAGGTTGATGGCCT
(SEQ. ID NO. 6)
CGCCCTCCACCAACAGGTTGATGGCCT
(SEQ. ID NO. 7)
CGCCCTCCACCACACAGGTTGATGGCCT
(SEQ. ID NO. 8)
CGCCCTCCACCACGCAACAGGTTGATGGCCT
(SEQ. ID NO. 9)
CGCCCTCCACCACGCACAGGTTGATGGCCT

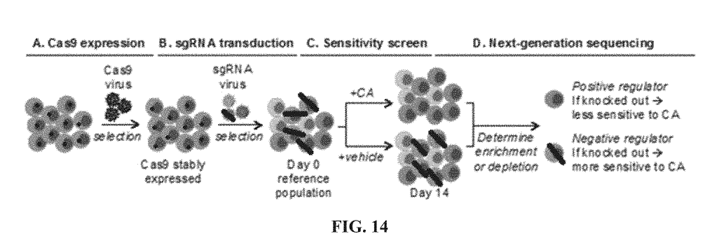

A CRISPR-Cas9 modifier screen in MOLM-14 cells to identify knocked out genes that confer resistance to CDK8/19 inhibition was conducted. The MOLM-14 cells were transduced in triplicate with the Broad lentiviral library encoding 80,000 sgRNAs against 18,000 genes in the human genome (4 sgRNAs/gene plus control sgRNAs), co-expressed with a puromycin resistance marker. Cells were selected expressing both Cas9 and sgRNA on blasticidin and puromycin for 7 days, and then began the screen. The workflow is described in FIG. 14. Changes in sgRNA distribution from day 0 to day 14 are compared between vehicle and CA treatment groups. Guides that are enriched in the CA group, but not the vehicle group, represent the positive regulators of CA sensitivity. Genes are ranked based on RIGER scores that take into account not only the fold-change difference between the two treatment groups, but also the reproducibility between the replicates and the similarity of effects among redundant sgRNAs. The top hits are validated by individually knocking out the gene using CRISPR-Cas9, verifying the knock-out using western blot or qPCR, and measuring resistance to CA.

### Example 14: Determining the Sensitivity of Primary Pediatric Patient AMKL Cells to CDK8/19 Inhibition In Vitro and in Mouse Xenograft Models

Cortistatin A's (CA's) or an analog thereof's antiproliferative activity and effect on differentiation in primary patient AMKL samples can be determined. First 10 to 30 patient samples are collected, including both pediatric DS-AMKL and pediatric non-DS-AMKL for which genetic lesions have been characterized such as GATA1 status and presence of common translocations in non-DS-AMKL, such as MLL-rearrangements, RBM15-MKL1, CBFA2T3-GLIS2 and NUP98-KDM5A. Samples are then prioritized that have been expanded first in sub-lethally irradiated NOD.Cg-Prkdc Il2rg¹/SzJ (NSG) mice, so that subsequent testing can be performed, if sensitive in vitro, in a mouse xenograft model. For patient samples that have not been expanded in vivo, first attempts to engraft and expand in NSG mice are undertaken.

For in vitro testing, AMKL cells are cultured with CA, an analog thereof, or vehicle for 3-5 days. The cell number is then measured over time and cellular effects are characterized using flow cytometry for (1) changes in megakaryocyte-specific markers CD41 and CD42 (2) changes in ploidy and (3) induction of apoptosis. These in vitro experiments were previously described for Aurora Kinase A inhibitor MLN8237. The in vivo xenograft models are subsequently tested for up to 5 patient samples that are sensitive to CDK8/19 inhibition in vitro and that have already engrafted and expanded in NSG mice, following the procedures described for testing of MLN8237. Specifically, the patient leukemic blasts from primary, secondary or tertiary recipient mice are injected intrafemoral into sub-lethally irradiated NSG mice (7 per treatment group). After a -10-day engraftment period, mice are treated with CA, an analog thereof, or vehicle over 15 days followed by monitoring with sampling of bone marrow (days 27 and 70) and peripheral blood (day 55) for disease burden and differentiation. Disease burden is measured by human CD45 expression by flow cytometry at all time points to assay for presence of human cells and differentiation is measured by human CD42 expression at 3 days-post treatment. In addition to sampling, mice are monitored for hind leg paralysis and survival.

### Example 15: Determine Whether CDK8/19 Inhibition Restores the RUNX1 Transcriptional Program in AMKL

For three to five CA-sensitive patient leukemic blasts described in Example 14 the gene expression and RUNX1 occupancy upon CA, an analog thereof, or vehicle treatment is measured. RUNX1 occupancy is measured by chromatin immunoprecipitation followed by sequencing (ChIP-seq). These experiments parallel those performed with the SET-2 megakaryocytic cell line and enable validation that (1) CA of an analog thereof stimulates the RUNX1 transcriptional program in these patient leukemia cells and (2) acts through relieving a block in RUNX1 recruitment to specific genomic loci that are also transcriptionally upregulated. In addition, the gene expression in up to three CA-resistant AMKL patient samples is measured to determine whether modulation of RUNX1 transcriptional program is selectively observed in sensitive cells. Comparing basal gene expression patterns in sensitive and insensitive AMKL patients allows the determination of whether certain gene expression programs correlate with sensitivity.

### Example 16: Genome-Wide CRISPR-Cas9 Modifier Screens to Identify Predictive Biomarkers for CDK8/19 Inhibitor Therapy

Genome-wide CRISPR-Cas9 modifier screens are performed to identify gene alterations in AML that may predict sensitivity. The screens occur in cortistatin A (CA)-sensitive AML cell lines (>50% growth inhibition at 100 nM CA) and CA-insensitive AML cell lines (<50% growth inhibition at 100 nM CA). With CA-sensitive cell lines, the genes are identified that when knocked out, confer CA-resistance and likewise, in CA-insensitive cell lines, the genes are identified that when knocked out, confer CA-sensitivity. By testing multiple cell lines, cell line-specific genetic alterations can be ruled-out, and by testing both CA-sensitive and CA-insensitive cell lines, patterns of genetic alterations that may predict sensitivity to CA or CA analogs are determined. The results of this screen can be used to evaluate expression levels of genes in AML patient samples that have been evaluated for CDK8/19 inhibitor sensitivity.

### Example 17: Screening of 62 Cells with Various Biomarkers for Sensitivity to Cortistatin A

For the 62 cell lines 96 well suspension cell culture plates were prepared. 100 μL of the soft agar bottom layer (0.6% final concentration in complete medium) was poured and left to solidify. 50 μL of the soft agar top layer (0.4% final concentration) containing the corresponding cells and cell number were then added on top, solidified and incubated at 37° C., 10% CO₂. After the soft agar had solidified, the test items were added at indicated final concentrations into the inner wells of the plate. Subsequently, the assay was incubated in cell culture incubators for 8 to 14 days. Finally, the assay was developed using Alamar Blue and upon 1-5 h of incubation at 37° C. fluorescence intensity was determined (excitation: 560 nm; emission: 590 nm). As low control, cells were treated with 1E-05M Staurosporine (6fold values). As high control, cells were treated with 0.1% DMSO (solvent control, 6 fold values).

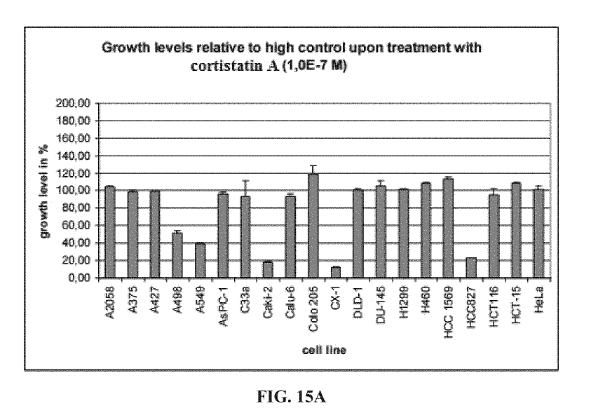

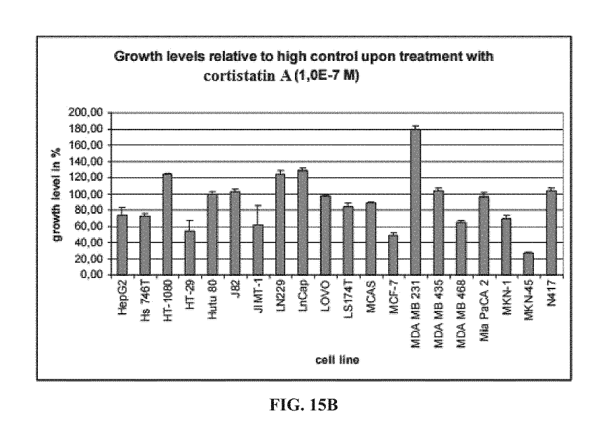

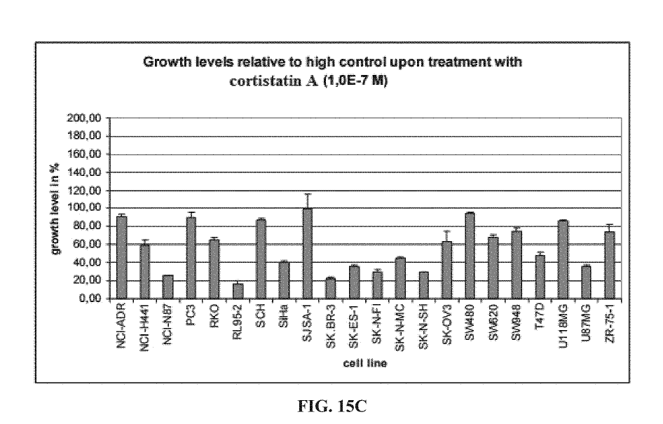

Raw data were converted into percent soft agar growth relative to high controls (solvent 0.1% DMSO) and low controls (1E-05M Staurosporine), which were set to 100% and 0%, respectively. The results are tabulated in FIGS. 15A, 15B, and 15C and Table 4 below.

TABLE 4
Growth % of 15 most inhibited cell lines tested,
and their corresponding biomarkers present.

| | | | % growth |
|---|---|---|---|
| | Cell line | | rel. to |
| Biomarker(s) | name | Origin | vehicle |
| ER-alpha positive | RL95-2 | Ovarian cancer | 16% |
| | T47D | Breast cancer | 48% |
| | MCF-7 | Breast cancer | 49% |
| Loss of function VHL | Caki-2 | clear cell renal | 18% |
| mutation (VHL negative) | A498 | cell carcinoma | 51% |
| HER2 overexpression | SK.BR-3 | Breast cancer | 22% |
| | NCI-N87 | Gastric cancer | 26% |
| EGFR mutation | HCC827 | Non small cell | 23% |
| | | lung carcinoma | |
| MET amplification | MKN-45 | Gastric cancer | 27% |
| 3/3 neuroblastoma lines reduce | SK-N-SH | Neuroblastoma | 29% |
| RUNX1 gene expression | SK-N-F1 | | 30% |
| | SK-N-MC | | 45% |
| EWS-FLI1 | SK-ES-1 | Osteosarcoma | 36% |
| | SK-N-MC | Neuroblastoma | 45% |
| Inactivating mutations in | U87MG | Glioma | 36% |
| ETV1, FLL1 and SMC3 | | | |

### Example 18: Cortistatins for the Treatment of Myelodysplastic Syndrome (MDS)

Cortistatin A significantly increases the expression of many RUNX1-target genes including CEBPA, IRF8, and NFE2 in AML cell lines including the MOLM-14 cell line which is derived from an MDS patient (FIG. 2, Example 1). Further RUNX1 is known to be mutated in 10-20% of MDS patients, and is the most frequently mutated gene in MDS. Therefore, cortistatins and analogs thereof can be effective treatments for MDS by increasing expression of RUNX1 genes. Further confimation is provided by the following experimental results: 1) CA induced recruitment of RUNX1 to loci upregulated by CA treatment, suggesting that CDK8/19 kinase activity blocks accumulation of RUNX1 at target loci (Example 11), and 2) CA's anti-proliferative activity positively correlated with cell lines with impaired RUNX1-target gene expression including those harboring RUNX1 mutations.

### Example 19: Megakaryocytic Cell Lines are Highly Sensitive to CDK8/19 Inhibitor Cortistatin a (CA) and CA Antiproliferative Activity is Consistent with Stimulation of the RUNX1 Transcriptional Program

It was found that cortistatin A (CA) potently inhibits proliferation of 5 out of 5 megakaryocytic cell lines tested with 50% maximal growth inhibitory concentrations (GI₅₀s) of less than 10 nM (Table 5). Among the cell lines tested was CMK-86, which contains the GATA1s truncated protein and was derived from a pediatric DS-AMKL patient

TABLE 5
Leukemia cell line sensitivity to CA is consistent with RUNX1
dependence, GI₅₀ shown after 10-day treatment. Note remarkable
sensitivity of many megakaryocytic cell lines.
Megakaryocy* c cell lines;
megakaryopoeisis is RUNX1-dependent

| | | |
|---|---|---|
| MKPL-1(RBM6-CSF1R), adult non- | 1 | nM |
| DS-AMKL, 66 year old male | | |
| MOLM-16, 77 year old female | 1 | nM |
| SET-2 (JAK2V617F), 71 year old | 4 | nM |
| female | | |
| CMK-86 (GATA1s), pediatric DS- | 8 | nM |
| AMKL, 10 month old male with | | |
| down syndrome | | |
| MEG-01 (BCR-Abl, GATA1s), adult | 9 | nM |
| non-DS-AMKL, 55 year old male | | |

Fusions that directly inhibit RUNX1 or
transcrip*on of its target genes

| | | |
|---|---|---|
| SKNO-1 (RUNX1-RUNX1T1) | 1 | nM |
| ME-1 (CBFB-MYH11) | 1 | nM |
| MOLM-14 (MLL-AF9) | 5 | nM |
| MV4; 11(MLL-AF4) | 6 | nM |

Erythroleukemia; terminal erythrocyte
differen*a*on notRUNX1-dependent

| | | |
|---|---|---|
| TF-1 (EpoR) | 350 | nM |
| K562 (BCR-Abl) | >1,000 | nM |
| HEL (JAK2V617F) | >1,000 | nM |

The antiproliferative activity of CA across leukemia cell lines matched their expected dependency on dysregulation of the RUNX1 transcriptional program. In addition to megakaryoblastic cell lines, CA potently inhibited the proliferation of cell lines containing chimeric proteins (fusions) that directly inhibit RUNX1 or transcription of its target genes (Table 5), including a cell line containing the RUNX1-RUNX1T1 fusion and cell lines containing MLL-fusions. Additionally, erythroleukemia cell lines are insensitive to CA, consistent with the decline of RUNX1 expression in erythrocyte differentiation (lack of RUNX1 dependence in terminal differentiation of erythrocytes). The strong lineage-dependent sensitivity of cell lines to CA is particularly evident in the result that megakaryocytic and erythroleukemia cell lines containing the same mutations (BCR-Abl or JAK2V617F) differ by more than 100-fold in sensitivity (compare K562 and HEL to MEG-01 and SET-2).

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

What is claimed is:

1. A method for the targeted selection and treatment of patients with a tumor or cancer mediated by CDK8 and/or CDK19, comprising (i) determining whether the patient has a RUNX1 pathway impairment; and if so (ii) administering an effective amount of a cortistatin or its pharmaceutically acceptable salt or oxide; optionally in a pharmaceutically acceptable composition, wherein the cortistatin is of Formula: 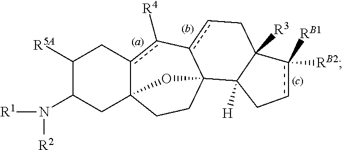
or a pharmaceutically acceptable salt or quaternary amine salt thereof;
wherein:
R¹ and R² are joined to form a heterocyclyl or heteroaryl each of which is optionally substituted with one or more R⁷ substituents;
R³ is hydrogen or alkyl;
R⁴ is hydrogen, halogen, alkyl, or —Si(R^{A})₃;
R^{5A} is hydrogen, halogen, alkyl, or —OR^{A};
each instance of [symbol] , designated as (a1), (b), and (c), represents a single or double bond, as valency permits, provided:
h. when [symbol] designated as (c) represents a double bond, then one of R^{B1} and R^{B2} is absent; and
i. when [symbol] designated as (c) represents a single bond, then both R^{B1} and R^{B2} are present,
each instance of R^{B1} and R^{B2} is, independently, hydrogen, -L₁-R^{B3}, or —X^{A}R^{A};
or R^{B1} and R^{B2} are joined to form an oxo group, provided that at least one of R^{B1} and R^{B2} is not hydrogen;
X^{A} is —O—, —S—, or —N(R^{A})—;
L₁ is a bond, —CH(CH₃)(CH₂)₂—, —CH(CH₃)—CH═CH—, —C(═O)—, —C(═O)O—, —C(═O)S—, —C(═O)N(R^{L})—, or —N(R^{L})—(R^{LL})₂)ₚ—;
R^{L} is hydrogen, optionally substituted alkyl, or a nitrogen protecting group;
each instance of R^{LL} is independently hydrogen, halogen, or alkyl;
p is 0, 1, or 2;
R^{B3} is hydrogen, alkyl, alkenyl, alkynyl, carbocyclyl, heterocyclyl, aryl, or optionally substituted heteroaryl, provided that when L₁ is a bond, then R^{B3} is not hydrogen;
each instance of R^{A} is independently hydrogen, alkyl, alkenyl, alkynyl, carbocyclyl, heterocyclyl, aryl, heteroaryl, carbonyl, silyl, an oxygen protecting group when attached to oxygen, a sulfur protecting group when attached to sulfur, or a nitrogen protecting group when attached to nitrogen; optionally when attached to N the two R^{A} groups may be joined to form a heterocyclyl or heteroaryl ring; and
each instance of R⁷ is independently halogen, alkyl, alkenyl, alkynyl, carbocyclyl, heterocyclyl, aryl, heteroaryl, amino, hydroxyl, thiol, or a nitrogen protecting group when attached to a nitrogen atom.

2. The method of claim 1, wherein R³ is —CH₃.

3. The method of claim 1, wherein R⁴ is hydrogen.

4. The method of claim 1, wherein R⁵ is hydrogen.

5. The method of claim 1, wherein each instance of [symbol] designated as (a) and (b) is a double bond.

6. The method of claim 1, wherein [symbol] designated as (c) represents a single bond.

7. The method of claim 1, wherein the cortistatin is of Formula: 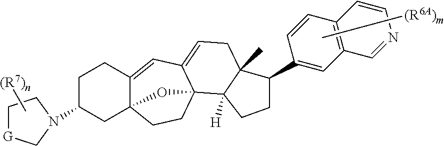
wherein:
each instance of R^{6A} is independently halogen, —NO₂, —CN, —OR^{6C}, SR^{6C}, N(R^{6C})₂, —C(═O)R^{6C}, —C(═O)OR^{6C}, —C(═O)N(R^{6C})₂, alkyl, alkenyl, alkynyl, carbocyclyl, heterocyclyl, aryl, or heteroaryl;
wherein each instance of R^{6C} is independently hydrogen, alkyl, alkenyl, alkynyl, carbocyclyl, heterocyclyl, aryl, heteroaryl, an oxygen protecting group when attached to oxygen, a sulfur protecting group when attached to sulfur, or a nitrogen protecting group when attached to nitrogen, optionally when attached to N the two R^{6C} groups may be joined to form an heterocyclyl or heteroaryl ring;
G is —O—, —S—, —NH—, —NR⁷—, —CH₂—, —CH(R⁷)—, or —C(R⁷)₂—;
m is 0, 1, 2, 3, or 4; and
n is 0, 1, 2, 3, or 4.

8. The method of claim 7, wherein m is 0.

9. The method of claim 1, wherein the cortistatin is of Formula: 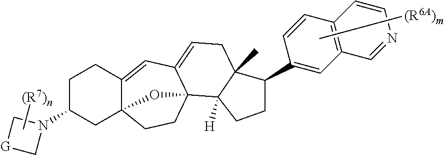
wherein:
each instance of R^{6A} is independently halogen, —NO₂, —CN, —OR^{6C}, SR^{6C}, N(R^{6C})₂, —C(═O)R^{6C}, —C(═)OR^{6C}, —C(═)N(R^{6C})₂, alkyl, alkenyl, alkynyl, carbocyclyl, heterocyclyl, aryl, or heteroaryl;
wherein each instance of R^{6C} is independently hydrogen, alkyl, alkenyl, alkynyl, carbocyclyl, heterocyclyl, aryl, heteroaryl, an oxygen protecting group when attached to oxygen, a sulfur protecting group when attached to sulfur, or a nitrogen protecting group when attached to nitrogen, optionally when attached to N the two R^{6C} groups may be joined to form an heterocyclyl or heteroaryl ring;
G is —O—, —S—, —NH—, —NR—, —CH₂—, —CH(R⁷)—, or —C(R⁷)₂—;
m is 0, 1, 2, 3, or 4; and
n is 0, 1, 2, 3, or 4.

10. The method of claim 9, wherein m is 0.

11. The method of claim 1, wherein the cortistatin is of Formula: 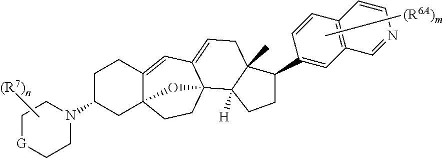
wherein:
each instance of R^{6A} is independently halogen, —NO₂, —CN, —OR^{6C}, SR^{6C}, N(R^{6C})₂, —C(═O)R^{6C}, —C(═O)OR^{6C}, —C(═O)N(R^{6C})₂, alkyl, alkenyl, alkynyl, carbocyclyl, heterocyclyl, aryl, or heteroaryl;
wherein each instance of R^{6C} is independently hydrogen, alkyl, alkenyl, alkynyl, carbocyclyl, heterocyclyl, aryl, heteroaryl, an oxygen protecting group when attached to oxygen, a sulfur protecting group when attached to sulfur, or a nitrogen protecting group when attached to nitrogen, optionally when attached to N the two R^{6C} groups may be joined to form an heterocyclyl or heteroaryl ring;
G is —O—, —S—, —NH—, —NR⁷—, —CH₂—, —CH(R⁷)—, or —C(R⁷)₂—;
m is 0, 1, 2, 3, or 4; and
n is 0, 1, 2, 3, or 4.

12. The method of claim 11, wherein m is 0.

13. A method for predicting the response of a patient with a tumor or cancer to treatment with a cortistatin, comprising:
i. Obtaining a sample of the tumor or cancer from the patient;
ii. Determining the expression level or amount of one or more RUNX1 target genes in the biological sample from a patient wherein the RUNX1 target gene(s) is selected from the group consisting of: ACSL1, ADORA2B, ADRB1, AMPD3, ARRDC4, BCL2, BCL2A1, CBFβ, CCNA1, CD244, CD44, CDC42EP3, CEBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR GPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5;
iii. Determining whether the expression level or amount assessed in step (ii) is outside of the range of corresponding normal cells, for example, above or below that found in corresponding normal cells or above or below a certain quantity that is associated with an increased or decreased clinical benefit to a patient; and
iv. Optionally treating the patient with an effective amount of the cortistatin, or its pharmaceutically acceptable salt, oxide or a pharmaceutically acceptable salt thereof;
wherein the cortistatin is of Formula: 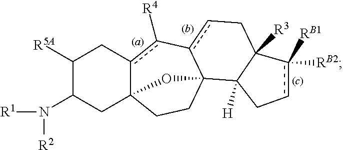
or a pharmaceutically acceptable salt or quaternary amine salt thereof;
wherein:
R¹ and R² are joined to form a heterocyclyl or heteroaryl each of which is optionally substituted with one or more R⁷ substituents;
R³ is hydrogen or alkyl;
R⁴ is hydrogen, halogen, alkyl, or —Si(R^{A})₃;
R^{5A} is hydrogen, halogen, alkyl, or —OR^{A};
each instance of [symbol] , designated as (a1), (b), and (c), represents a single or double bond, as valency permits, provided:
a. when [symbol] designated as (c) represents a double bond, then one of R^{B1} and R^{B2} is absent; and
b. when [symbol] designated as (c) represents a single bond, then both R^{B1} and R^{B2} are present,
each instance of R^{B1} and R^{B2} is, independently, hydrogen, -L₁-R^{B3}, or —X^{A}R^{A};
or R^{B1} and R^{B2} are joined to form an oxo group, provided that at least one of R^{B1} and R^{B2} is not hydrogen,
X^{A} is —O—, —S—, or —N(R^{A})—;
L₁ is a bond, —CH(CH₃)(CH₂)₂—, —CH(CH₃)—CH═CH—, —C(═)—, —C(═O)O—, —C(═O)S—, —C(═O)N(R^{L})—, or N(R^{L})—(C(R^{LL})₂)ₚ—;
R^{L} is hydrogen, optionally substituted alkyl, or a nitrogen protecting group;
each instance of R^{L} is independently hydrogen, halogen, or alkyl;
p is 0, 1, or 2;
R^{B3} is hydrogen, alkyl, alkenyl, alkynyl, carbocyclyl, heterocyclyl, aryl, or optionally substituted heteroaryl, provided that when L, is a bond, then R^{B3} is not hydrogen;
each instance of R^{A} is independently hydrogen, alkyl, alkenyl, alkynyl, carbocyclyl, heterocyclyl, aryl, heteroaryl, carbonyl, silyl, an oxygen protecting group when attached to oxygen, a sulfur protecting group when attached to sulfur, or a nitrogen protecting group when attached to nitrogen; optionally when attached to N the two R^{A} groups may be joined to form a heterocyclyl or heteroaryl ring; and
each instance of R is independently halogen, alkyl, alkenyl, alkynyl, carbocyclyl, heterocyclyl, aryl, heteroaryl, amino, hydroxyl, thiol, or a nitrogen protecting group when attached to a nitrogen atom.

14. The method of claim 13, wherein the RUNX1 target genes are selected from GATA1, GATA2, C/EBPα, FLI1, FOG1, ETS1, PU.1, RUNX1, and CBFα.

15. The method of claim 13, wherein the RUNX1 target genes are selected from BCL2, CCNA1, CD44, C/EBPα, CBFβ, CSF1, CXCL10, CXCR4, ETS1, ETS2, ETS2, FLI1, FOG1, FCER1A, GATA1, GATA2, GFI1B, HEB, IRF1, IRF8, JAG1, LMO2, LTB, NFE2, NOTCH2, PU.1, SLA, SOCS1, TAL1, and TNF.

16. The method of claim 13, wherein the expression level of at least two RUNX1 target genes is determined.

17. The method of claim 13, wherein the expression level of at least three RUNX1 target genes is determined.

18. A method for selecting a patient with a tumor or cancer for treatment with a cortistatin, comprising:
i. Obtaining a sample of the patient's tumor or cancer;
ii. Detecting the expression level or amount of one or more RUNX1 target genes in the biological sample from the patient wherein the RUNX1 target gene(s) is selected from the group consisting of: ACSL1, ADORA2B, ADRB1, AMPD3, ARRDC4, BCL2, BCL2A1, CBFPβ, CCNA1, CD244, CD44, CDC42EP3, C/EBPα, CECR6, CFLAR, CISH, CSF1, CXCL10, CXCR4, CYTIP, DUSP10, E2F8, EMB, EMR2, ETS1, ETS2, FAM107B, FAM46A, FCER1A, FCGR1B, FLI1, FOG1, FOSL2, GAB2, GAS7, GATA1, GATA2, GFI1B, GMPR, CGPR18, GPR183, HBBP1, HEB, HLX, HMGCS1, IGFBP4, IGFBP5, IL17RA, IL1RAP, IPCEF1, IRF1, IRF8, ITGA6, JAG1, LCP2, LDLR, LIMA1, LMO2, LRRC33, LTB, MBP, MICAL2, MYCN, MYO1G, NFE2, NOTCH2, NRP1, P2RY2, PAG1, PLAC8, PLEK, PLXNC1, PMP22, PTPRE, PU.1, PXK, RAB27A, RASA3, RGS16, RHOH, RNF24, RXRA, SELPLG, SLA, SLC7A11, SLC7A5, SOCS1, ST3GAL4, STK17B, TAL1, TIMP3, TMEM104, TNF, TSC22D1, TSC22D3, ZBTB16, and ZCCHC5;
iii. Comparing the expression determined in step (ii) to the expression of the same genes in a control set of samples comprising a representative number of patients or a predictive animal model that exhibit response to a cortistatin and a representative number of patients that exhibit no or a poor response to a cortistatin to determine if the patient responds to cortistatin therapy; and
iv. Administering an effective amount of the cortistatin, or its pharmaceutically acceptable salt, oxide or a pharmaceutically acceptable salt thereof if the patient is determined to respond to the therapy; 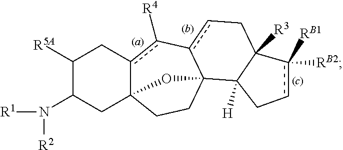
or a pharmaceutically acceptable salt or quaternary amine salt thereof;
wherein:
R¹ and R² are joined to form a heterocyclyl or heteroaryl each of which is optionally substituted with one or more R: substituents;
R³ is hydrogen or alkyl;
R⁴ is hydrogen, halogen, alkyl, or —Si(R^{A})₃;
R^{5A} is hydrogen, halogen, alkyl, or —OR^{A};
each instance of [symbol] , designated as (a1), (b), and (c), represents a single or double bond, as valency permits, provided:
a. when [symbol] designated as (c) represents a double bond, then one of R^{B1} and R^{B2} is absent; and
b. when [symbol] designated as (c) represents a single bond, then both R^{B1} and R^{B2} are present,
each instance of R^{B1} and R^{B2} is, independently, hydrogen, -L₁-R^{B3}, or —X^{A}R^{A};
or R^{B1} and R^{B2} are joined to form an oxo group, provided that at least one of R^{B1} and R^{B2} is not hydrogen;
X^{A} is —O—, —S—, or —N(R^{A})—;
L₁ is a bond, —CH(CH₃)(CH₂)₂—, —CH(CH₃)—CH═CH—, —C(═O)—, —C(═O)O—, —C(═O)S—, —C(═O)N(R^{L})—, or —N(R^{L})—(C(R^{LL})ₚ—;
R^{L} is hydrogen, optionally substituted alkyl, or a nitrogen protecting group;
each instance of R^{LL} is independently hydrogen, halogen, or alkyl;
p is 0, 1, or 2;
R^{B3} is hydrogen, alkyl, alkenyl, alkynyl, carbocyclyl, heterocyclyl, aryl, or optionally substituted heteroaryl, provided that when L, is a bond, then R^{B3} is not hydrogen;
each instance of R^{A} is independently hydrogen, alkyl, alkenyl, alkynyl, carbocyclyl, heterocyclyl, aryl, heteroaryl, carbonyl, silyl, an oxygen protecting group when attached to oxygen, a sulfur protecting group when attached to sulfur, or a nitrogen protecting group when attached to nitrogen; optionally when attached to N the two R^{A} groups may be joined to form a heterocyclyl or heteroaryl ring; and
each instance of R is independently halogen, alkyl, alkenyl, alkynyl, carbocyclyl, heterocyclyl, aryl, heteroaryl, amino, hydroxyl, thiol, or a nitrogen protecting group when attached to a nitrogen atom.

19. The method of claim 18, wherein the RUNX1 target genes are selected from GATA1, GATA2, C/EBPα, FLI1, FOG1, ETS1, PU.1, RUNX1, and CBFα.

20. The method of claim 18, wherein the RUNX1 target genes are selected from BCL2, CCNA1, CD44, C/EBPα, CBFβ, CSF1, CXCL10, CXCR4, ETS1, ETS2, FLI1, FOG1, FCER1A, GATA1, GATA2, GFI1B, HEB, IRF1, IRF8, JAG1, LMO2, LTB, NFE2, NOTCH2, PU.1, SLA, SOCS1, TAL1, and TNF.


SEQUENCE LISTING (9 sequences)

SEQ ID NO: 1 - DNA, 24 bp, Artificial Sequence
  ZsGREEN Cleaved Sequence 1
cgccctccaccacgcagatggcct

SEQ ID NO: 2 - DNA, 29 bp, Artificial Sequence
  ZsGREEN Cleaved Sequence 2
cgccctccaccacgacaggttgatggcct

SEQ ID NO: 3 - DNA, 24 bp, Artificial Sequence
  ZsGREEN Cleaved Sequence 3
cgccctccaacaggttgatggcct

SEQ ID NO: 4 - DNA, 28 bp, Artificial Sequence
  ZsGREEN Cleaved Sequence 4
cgccctccaccaaacaggttgatggcct

SEQ ID NO: 5 - DNA, 26 bp, Artificial Sequence
  ZsGREEN Cleaved Sequence 5
cgccctccaccacaggttgatggcct

SEQ ID NO: 6 - DNA, 27 bp, Artificial Sequence
  ZsGREEN Cleaved Sequence 6
cgccctccaccaacaggttgatggcct

SEQ ID NO: 7 - DNA, 28 bp, Artificial Sequence
  ZsGREEN Cleaved Sequence 7
cgccctccaccacacaggttgatggcct

SEQ ID NO: 8 - DNA, 31 bp, Artificial Sequence
  ZsGREEN Cleaved Sequence 8
cgccctccaccacgcaacaggttgatggcct

SEQ ID NO: 9 - DNA, 30 bp, Artificial Sequence
  ZsGREEN Uncleaved Sequence
cgccctccaccacgcacaggttgatggcct